(19) United States
(12) Patent Application Publication
(10) Pub. No.: US 2024/0191198 A1
(43) Pub. Date: Jun. 13, 2024

(54) CELL-CULTURED FOOD PRODUCTS AND RELATED CELLS, COMPOSITIONS, METHODS AND SYSTEMS

(71) Applicant: BlueNalu, Inc., San Diego, CA (US)

(72) Inventors: Courtney Anne BENSON, San Diego, CA (US); Lauran Rebecca MADDEN, San Diego, CA (US)

(21) Appl. No.: 18/555,123

(22) PCT Filed: Apr. 12, 2022
(86) PCT No.: PCT/US2022/024391

§ 371 Date: Oct. 12, 2023

Related U.S. Application Data
(60) Provisional application No. 63/174,444, filed on Apr. 13, 2021.

(51) Int. Cl. C12N 5/077; A23L 17/00

(52) U.S. Cl. CPC C12N 5/0653; A23L 17/00; C12N 5/0656; C12N 5/0658; C12N 2500/36

(57) ABSTRACT

Provided herein are culture medium, methods and systems to culture cells of an aquatic animal to increase lipid uptake, cell viability and/or cell differentiation in presence of one or more lipids at a concentration of at least 10 μg/ml, and cells obtainable and obtained thereby, related cell biomass and food products. Also provided herein are culture medium, methods and systems to culture cells of a terrestrial animal to increase lipid uptake, cell viability and/or cell differentiation in presence of one or more lipids at a concentration of at least 10 μg/ml, and cells obtainable and obtained thereby, related cell biomass and food products.

### RELATED APPLICATIONS

This application claims priority to U.S. Provisional Patent Application Ser. No. 63/174,444, filed Apr. 13, 2021, which is incorporated by reference in its entirety.

### BACKGROUND

Cell-cultured food products are part of food alternatives which have been the focus of development by numerous companies around the world as a means to address public health, environmental and animal welfare issues associated with animal farming and agriculture. Among the public health concerns are the relatively high saturated fatty acid content and essentially zero omega-3 polyunsaturated fatty acid content of meat from terrestrial animals, such as beef. Diets high in saturated fat and low in omega-3s have been implicated in elevated LDL cholesterol levels and increased risks of heart disease.

Despite the growing interest and efforts in producing animal-free food alternatives, challenges still remain for developing optimized formulations and methods to produce food from cell- and tissue-cultures, particularly cell-cultured food with enhanced fatty acids and nutrients.

### SUMMARY

This disclosure relates to aquatic animal cell-cultured food products and related cells, compositions, methods and systems for producing cells and cell-cultured food products, that can have a desired fatty acid profile. In several embodiments, the methods disclosed herein provide improved cell proliferation in culture and enhanced loading of desired fatty acids in the cellular biomass.

According to a first aspect, a culture medium, a method and a system are described to increase content of a monounsaturated fatty acid in a cell of an aquatic animal, and a cell of an aquatic animal obtainable and/or obtained thereby.

The culture medium comprises a cell basal medium for a cell of an aquatic animal supplemented with a monounsaturated fatty acid, typically at a concentration between about 0.1 μg/ml to about 1000 μg/ml, preferably about 0.1 μg/ml to about 500 μg/ml, about 1 μg/ml to about 100 μg/ml, or about 5 μg/ml to about 50 μg/ml.

The method comprises culturing the cell of the aquatic animal in the culture medium for a cell of an aquatic animal supplemented with a monounsaturated fatty acid, typically at a concentration between about 0.1 μg/ml to about 1000 μg/ml, for a time and under condition allowing uptake of the monounsaturated fatty acid by the cell of the aquatic animal, in accordance with the present disclosure.

The system comprises a culture medium for a cell of an aquatic animal in combination with a monounsaturated fatty acid and/or a cell of an aquatic animal, for simultaneous, combined or sequential use in the method to increase content of a monounsaturated fatty acid in a cell of an aquatic animal herein described.

According to a second aspect, a culture medium, a method and a system are described to increase content of a polyunsaturated fatty acid, a saturated fatty acid and/or a sterol in a cell of an aquatic animal, and a cell of an aquatic animal obtainable and/or obtained thereby.

The culture medium comprises a cell basal medium for a cell of an aquatic animal supplemented with a polyunsaturated fatty acid, a saturated fatty acid and/or the sterol, in combination with an effective amount of nervonic acid. In some embodiments, each supplemented lipid, e.g. polyunsaturated fatty acid, saturated fatty acid and/or the sterol, is present in a concentration of about 10 μg/ml or higher.

The method comprises culturing the cell of an aquatic animal in the culture medium for a cell of an aquatic animal herein described supplemented with at least 10 μg/ml of a polyunsaturated fatty acid, the saturated fatty acid and/or a sterol and culture medium further supplemented with an effective amount of nervonic acid, the culturing performed for a time and under condition allowing uptake of the polyunsaturated fatty acid, the saturated fatty acid and/or the sterol by the cell of the aquatic animal in accordance with the present disclosure.

The system comprises a basal cell culture medium for a cell of an aquatic animal in combination with a polyunsaturated fatty acid, a saturated fatty acid and/or a sterol, nervonic acid and/or cell of an aquatic animal for simultaneous, combined or sequential use in the method to increase content of a polyunsaturated fatty acid, a saturated fatty acid and/or a sterol in a cell of an aquatic animal herein described.

In preferred embodiments, the polyunsaturated fatty acid is or comprises an unsaturated fatty acid, such as an omega-3 poly-unsaturated fatty acid. According to a third aspect, a culture medium, a method and a system to increase content of a lipid in a cell of an aquatic animal are described, and a cell of an aquatic animal obtainable and/or obtained thereby.

The culture medium can comprise a cell basal medium for a cell of an aquatic animal supplemented with at least 10 μg/ml of the lipid, wherein when the lipid is or comprises a polyunsaturated fatty acid, a saturated fatty acid and/or a sterol, the culture medium further comprises a nervonic acid in an effective amount to allow upload of the polyunsaturated fatty acid and/or the sterol by the cell of the aquatic animal.

The method comprises culturing the cell of an aquatic animal in the culture medium for a cell of an aquatic animal herein described supplemented with at least 10 μg/ml of the lipid, the culturing performed for a time and under condition to allow uptake of the lipid by the cell of the aquatic animal. In the method, when the lipid is or comprises a polyunsaturated fatty acid and/or a sterol the culture medium further comprises an effective amount of nervonic acid.

The system comprises a culture medium for a cell of an aquatic animal in combination with one or more lipids and/or cell of an aquatic animal for simultaneous, combined or sequential use in the method to increase content of the lipid in a cell of an aquatic animal herein described. In embodiments, where the one or more lipids are or comprise one or more polyunsaturated fatty acid and/or a sterol, the system further comprises nervonic acid in an effective amount to upload the polyunsaturated fatty acid and/or the sterol in the cell of an aquatic animal.

In preferred embodiments, the lipid is or comprises an unsaturated fatty acid such as an omega-3 poly-unsaturated fatty acid.

According to a fourth aspect, a method and system are described and related culture medium, to increase lipid content in a myoblast cell and/or a fibroblast cell of an aquatic animal, as well as the myoblasts and/or fibroblasts of an aquatic animal obtainable and/or obtained thereby.

The method comprises culturing the myoblast cell and/or the fibroblast cell in a culture medium comprising at least 10 μg/ml of the lipid and when the lipid is or comprises polyunsaturated fatty acid and/or a sterol further comprising an effective amount of nervonic acid, the culturing performed for a time and under conditions allowing uptake of the lipid by the myoblast cell and/or fibroblast cell of the aquatic animal. In preferred embodiments, the lipid is or comprises an unsaturated fatty acid, such as an omega-3 poly-unsaturated fatty acid.

The system comprises a culture medium for cells of an aquatic animal, a lipid and/or cells selected from myoblast and/or fibroblast cells of an aquatic animal, for simultaneous, combined or sequential use in the method to increase lipid content in a myoblast and/or fibroblast cells of an aquatic animal herein described. When the lipid is or comprises a polyunsaturated fatty acid and/or a sterol, the system further comprises nervonic acid in an effective amount.

The culture medium comprises basal medium supplemented with a lipid and when the lipid is or comprises nervonic acid in an effective amount to increase lipid content of the myoblast cell and/or a fibroblast cell.

According to a fifth aspect, a method and system are described and related culture medium, to increase polyunsaturated fatty acid content in a cell of an aquatic animal, as well as cells of aquatic animals obtainable thereby.

The method comprises culturing the cell of the aquatic animal in a culture medium comprising the polyunsaturated fatty acids and an effective amount of nervonic acid.

The system comprises nervonic acid and polyunsaturated fatty acids, optionally in combination with culture medium, and/or a cell of the aquatic animal, for simultaneous, combined or sequential use in the method to increase polyunsaturated fatty acid content in a cell of an aquatic animal herein described.

The culture medium comprises basal medium, a polyunsaturated fatty acid and nervonic acid in an effective amount to increase uptake of the polyunsaturated fatty acid in a cell of an aquatic animal.

According to a sixth aspect, a method and system are described and related culture medium, to increase omega-3 content in a cell of an aquatic animal, as well as cells of aquatic animals obtainable thereby.

The method comprises culturing the cell of the aquatic animal in a culture medium comprising the omega-3 and an effective amount of nervonic acid.

The system comprises nervonic acid and omega-3 fatty acids, optionally in combination with culture medium, and/or a cell of the aquatic animal, for simultaneous, combined or sequential use in the method to increase omega-3 fatty acid content in a cell of an aquatic animal herein described.

The culture medium comprises basal medium, an omega-3 fatty acid and nervonic acid in an effective amount to increase intake of the omega-3 fatty acid in a cell of an aquatic animal.

According to a seventh aspect, a method and system are described and related culture medium, to increase viability of a cell of an aquatic animal in presence of polyunsaturated fatty acids, saturated fatty acids and/or sterols, as well as cells of aquatic animals obtainable thereby.

The method comprises culturing the cell of the aquatic animal in a culture medium comprising the polyunsaturated fatty acids, the saturated fatty acids and/or the sterols and an effective amount of nervonic acid.

The system comprises nervonic acid in combination with the polyunsaturated fatty acids, the saturated fatty acids and/or the sterols, optionally in combination with culture medium, and/or a cell of the aquatic animal, for simultaneous, combined or sequential use in the method to increase omega-3 fatty acid content in a cell of an aquatic animal herein described.

The culture medium comprises basal medium, the polyunsaturated fatty acids, the saturated fatty acids and/or the sterols and nervonic acid in an effective amount to increase cell viability in a cell of an aquatic animal herein described.

According to an eighth aspect, a preadipocyte cell of an aquatic animal is described, the preadipocyte cell comprising a desired lipid in an amount from 0.1% to 1% by weight of the cell. Typically, the total lipid content of the cell is greater than about 2.0% by weight. In preferred embodiments, the lipid content of the preadipocyte cell herein described can contain about 50% SFA, 25% PUFA, preferably including Omega 3, and 25% MUFA. Preferably, the SFA of the preadipocyte or adipocyte is low and the cell contains a higher percentage of unsaturated fatty acids as a percentage of total fat. For example, the SFA content can be about 30% or less, about 20% or less, about 10% or less, or about 5% or less.

According to a ninth aspect, a myoblast cell or myocyte of an aquatic animal is described and related biomass comprising said cell, the myoblast cell comprising a desired fatty acid in an amount of at least about 0.5% by weight. Typically, the total lipid content of the cell is greater than about 2.0% by weight. Preferably, the SFA of the myoblast or myocyte is low and the cell contains a higher percentage of unsaturated fatty acids as a percentage of total fat. For example, the SFA content can be about 30% or less, about 20% or less, about 10% or less, or about 5% or less.

According to a tenth aspect, a fibroblast cell of an aquatic animal is described and related biomass comprising said cell, the fibroblast cell comprising a desired fatty acid in an amount of at least about 0.5% by weight. Typically, the total lipid content of the cell is greater than about 2.0% by weight. Preferably, the SFA of the fibroblast is low and the cell contains a higher percentage of unsaturated fatty acids as a percentage of total fat. For example, the SFA content can be about 30% or less, about 20% or less, about 10% or less, or about 5% or less.

According to an eleventh aspect, an aquatic animal biomass is described, comprising any one of the aquatic animal cells herein described alone or in any possible combination of different cell types.

According to a twelfth aspect, an aquatic animal cell-cultured food product is described, comprising any one of the aquatic animal cells herein described alone or in any possible combination of different cell types and/or any one of the aquatic animal biomass herein described. The aquatic animal cell-cultured food product can contain one, two, three, four or more cell types, such as myocytes, fibroblasts, adipocytes, endothelial cells and any combination thereof. In other embodiments, the aquatic animal cell-cultured food product can contain one, two, three, four or more cell types, such as myocytes, myoblasts, fibroblasts, adipocytes, endothelial cells, epithelial cells, preadipocytes, keratinocytes, embryonic derived cells, induced pluripotent stem cells, mesenchymal stem cells, and any combination thereof. In more preferred embodiments, the aquatic animal cell-cultured food product is adipocyte-free. In most preferred embodiments, the food product is a cell-cultured fish product.

According to a thirteenth aspect, an aquatic animal cell-cultured food product is described comprising any one of the aquatic animal cells herein described, the aquatic animal cell having a lipid content of at least 0.2% by weight, preferably more than about 2.0% by weight, for example between about 2.0% and about 90% by weight. In some embodiments, the cell-cultured food product described herein comprises fish cells, particularly white fish cells, herein described having a lipid (e.g., fatty acid) content that is greater than about 2.0% by weight. In some embodiments, the lipid (e.g., fatty acids) content is at least about 10%, at least about 20%, at least about 30%, at least about 40%, at least about 50%, at least about 60%, at least about 70%, at least about 80%, between about 10% and about 90%, between about 20% and about 90%, between about 30% and about 90%, between about 40% and about 90%, between about 50% and about 90%, between about 60% and about 90%, between about 70% and about 90%. In preferred embodiments, the food product is adipocyte-free. In most preferred embodiments, the food product is a cell-cultured fish product.

According to a fourteenth aspect, an aquatic animal-based food product is described comprising any one of the cells herein described having an omega-3 fatty acid content of at least about 50%, at least about 60%, at least about 70%, at least about 80% or at least about 90% by weight. In preferred embodiments, the food product is adipocyte-free.

According to a fifteenth aspect, a composition for increasing lipid uptake and/or cell viability for an aquatic animal cell and/or cellular biomass is described. The composition comprises nervonic acid together with a suitable vehicle. In particular, the nervonic acid can be present in a concentration of about 1 μg/ml or higher, e.g., between about 1 μg/ml and about 10 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, and about 10 μg/ml.

The nervonic acid can be present in a concentration from about 10 μg/ml to about 50 μg/ml, such as about 10 μg/ml, about 11 μg/ml, about 12 μg/ml, about 13 μg/ml, about 14 μg/ml, about 15 μg/ml, about 16 μg/ml, about 17 μg/ml, about 18 μg/ml, about 19 μg/ml, about 20 μg/ml, about 21 μg/ml, about 22 μg/ml, about 23 μg/ml, about 24 μg/ml, about 25 μg/ml, about 26 μg/ml, about 27 μg/ml, about 28 μg/ml, about 29 μg/ml, about 30 μg/ml, about 31 μg/ml, about 32 μg/ml, about 33 μg/ml, about 34 μg/ml, about 35 μg/ml, about 36 μg/ml, about 37 μg/ml, about 38 μg/ml, about 39 μg/ml, about 40 μg/ml, about 41 μg/ml, about 42 μg/ml, about 43 μg/ml, about 44 μg/ml, about 45 μg/ml, about 46 μg/ml, about 47 μg/ml, about 48 μg/ml, about 49 μg/ml, or about 50 Ng/ml.

According to a sixteenth aspect, a method and system are described for uploading a lipid in an aquatic animal cell and related culture medium, cells obtainable thereby, cellular biomass and cell-cultured food.

The method comprises culturing the aquatic animal cell in presence of vaccenic acid for a time and under condition resulting in uptake of the vaccenic acid by the aquatic animal cell.

The systems comprises vaccenic acid in combination with an aquatic animal cell and/or culture medium for combined use in the method for uploading a lipid in an aquatic animal cell herein described.

The culture medium comprises basal medium and vaccenic acid in an effective amount to result in lipid upload by the aquatic animal cell.

The culture media, methods and systems herein described and related compositions, cells, cell biomass, and cell-cultured food products, achieve, in various embodiments, an increased lipid loading of cells of an aquatic animal such as fish myoblasts, fibroblasts and preadipocytes, which can have a controllable fat content from about 0.1% to about 90%, preferably from about 2.5% to about 90%, such as about 2.5% to about 20%.

The culture media, methods and systems herein described and related compositions, cells, cell biomass, and cell-cultured food products, allow in various embodiments control of lipid content in cells of an aquatic animal, and related food product with selection of one or more desired lipids, such as polyunsaturated fatty acids and more particular, omega-3 fatty acids, DHA and EPA, as well as additional fatty acids identifiable by a skilled person.

The culture media, methods and systems herein described and related compositions, cells, cell biomass, and cell-cultured food products, allow in various embodiments enhanced uptake of polyunsaturated fatty acids by cells of an aquatic animal, thus, resulting in generation of cell-cultured food product with high lipid content and increased levels of fatty acids, such as omega-3 fatty acids.

The compositions, methods and systems herein described and related cells, cell biomass, and cell-cultured food products, achieve in various embodiments an improved cell viability allowing, for example, faster generation of cell-cultured fish product with controlled composition and level of fatty acids.

The compositions, methods and systems herein described and related cells, cell biomass, and cell-cultured food products, achieve in various embodiments production of cell-cultured food product comprising cells of an aquatic animal and in particular, cell-cultured fish-products, such as containing adequate levels of lipids without using adipocytes (see e.g. a cell-cultured food product comprising white fish cell having about 2.0% to about 90% lipid content by weight, preferably greater than 2.0% to about 90% lipid content by weight).

The culture media, methods and systems herein described and related compositions, cells, cell biomass, and cell-cultured food products, allow in several embodiments, to increase viability, differentiation and/or lipid uptake of the cell of an aquatic animal.

The culture media, methods and systems herein described and related compositions, cells, cell biomass, and cell-cultured food products, can be performed with culture media which do not require presence (and therefore can be in absence) of dexamethasone, biotin, T3, pantothenate, IBMX, and/or insulin. Preferably, the culture media do not include at least one and preferably all of dexamethasone, biotin, T3, pantothenate, IBMX, and insulin.

The culture media, methods and systems herein described and related compositions, cells, cell biomass, and cell-cultured food products, herein described can be used in connection with various applications wherein cell viability, controlled proliferation and lipid content in cell and related cell-cultured food product is desired. For example, compositions, methods and systems herein described and related cells, cell biomass, and cell-cultured food products, herein described can be used to generate cell-cultured food products such as food products with a controlled lipid content. Accordingly, exemplary fields of applications comprise food manufacturing, food processing and commercialization. Additional exemplary applications include uses of the culture media, compositions, methods and systems and related cells and cell biomass cell-cultured food products herein described in several fields including basic biology research, applied biology, bioengineering, bioenergy, medical research, therapeutics, and in additional fields identifiable by a skilled person upon reading of the present disclosure.

For example, although the foregoing has referred to cells of aquatic animals, the media, methods, and systems are generally suitable for culturing cells of terrestrial animals and preparing cultured-cell food products therefrom. The fatty acid profile of the cultured terrestrial animal cells can exhibit an increase in omega-3 fatty acid content, a decrease in saturated fatty acid content, or both.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1 presents photomicrographs showing, in some embodiments, the loading of silver carp preadipocytes with fatty acid mixture. Carp preadipocytes were cultured in control media or media with reduced serum or no serum containing 1% fatty acid mixture. LM indicates Lipid Mixture 1000×from Millipore Sigma, FBS indicates Fetal Bovine Serum. Brightfield images show lipid accumulation at day two and day seven along with characteristic cell rounding.

FIG. 2A illustrates the results from immunofluorescent staining of silver carp preadipocytes loaded with fatty acid mixture. Silver carp preadipocytes were cultured in control media or lipid loading media without serum in the presence of lipid mixture for seven days. Cells were fixed and stained for nuclei (Hoechst), cytoskeleton protein F-actin (Phalloidin), and lipid droplets (BODIPY). Fluorescent images show lipid loading in treated conditions and not control conditions.

FIG. 2B quantifies and tabulates fluorescent staining data presented in FIG. 2A as the percent of lipid loading to total cell volume.

FIG. 3A shows, in some embodiments, the loading of Bluefin tuna preadipocytes with fatty acid mixture. Bluefin tuna preadipocytes were cultured in control media or media with reduced serum or no serum containing 1% fatty acid mixture. Brightfield images show lipid accumulation at day one and day six along with characteristic cell rounding. Presence of lipid droplets is confirmed by staining of BODIPY (green) within the cell that is significantly enhanced under lipid-treated conditions when compared to control.

FIG. 3B quantifies and tabulates fluorescent staining data presented in FIG. 3A as the percent of lipid loading to total cell volume.

FIG. 4 shows, in some embodiments, the loading of silver carp preadipocytes with single fatty acids. Silver carp preadipocytes were cultured in media with reduced serum in the presence of increasing concentrations of individual fatty acids—DHA, EPA, linoleic acid, or palmitoleic acid for six days. Brightfield images show concentration-dependent lipid accumulation along with characteristic cell rounding. Some concentration-related toxicity is observed by decreased cell number for Palmitoleic acid.

FIG. 5A shows the results from immunofluorescent staining of silver carp preadipocytes loaded with single fatty acids. Silver carp preadipocytes were cultured in media with reduced serum in the presence of increasing concentrations of individual fatty acids—DHA, EPA, linoleic acid, or palmitoleic acid for six days. BODIPY staining of lipid droplets confirms increased loading with concentration of DHA, EPA, and linoleic acid, while a peak for palmitoleic acid is reached at a lower concentration due to toxicity above 50 μg/ml.

FIG. 5B quantifies and tabulates fluorescent staining data presented in FIG. 5A as the percent of lipid loading to total cell volume.

FIG. 6 shows, in some embodiments, the loading of yellowtail fibroblasts with fatty acid mixture. Yellowtail fibroblasts were cultured in control media or reduced serum media with 1% fatty acid mixture for up to seven days. Brightfield microscopy images show accumulation of lipid droplets at two days with maintained loading out to day seven. Control cultures do not show any lipid accumulation.

FIG. 7A shows the results from immunofluorescent staining of yellowtail fibroblasts loaded with fatty acid mixture. Yellowtail fibroblasts were cultured in control media or reduced serum media with 1% fatty acid mixture for seven days. Staining with BODIPY for lipid droplets confirms the accumulation of lipids in cells treated with media containing lipid mixture but not control media.

FIG. 7B quantifies and tabulates fluorescent staining data presented in FIG. 7A as the fold increase in lipid loading to total cell volume compared to control.

FIG. 8A shows, in some embodiments, the loading of yellowtail myoblasts with fatty acid mixture. Yellowtail myoblasts were cultured in control media or reduced serum media containing 1% fatty acid mixture. Brightfield images show loading of lipids into cells at day two that maintains to day seven for cells cultured in the presence of lipid mixture but not control media. Immunofluorescent staining with BODIPY for lipid droplets confirms the loading and retention of lipids in treated cells at day seven.

FIG. 8B quantifies and tabulates fluorescent staining data presented in FIG. 8A as the fold increase in lipid loading to total cell volume compare d to control.

FIG. 9A shows, in some embodiments, the loading of Mahi Mahi myoblasts with fatty acid mixture. Mahi Mahi myoblasts were cultured in control media or reduced serum media containing 1% fatty acid mixture. Brightfield images show lipid loading of cells in the presence of fatty acid mixture by day four. Fluorescent staining of cells with BODIPY confirms the accumulation of lipids in cells treated with lipid mixture but not control cells.

FIG. 9B quantifies and tabulates fluorescent staining data presented in FIG. 9A as the fold increase in lipid loading to total cell volume compared to control.

FIG. 10A shows, in some embodiments, the loading of Bluefin tuna myoblasts with fatty acid mixture. Bluefin tuna myoblasts were cultured in control media or reduced serum media containing 1% fatty acid mixture. Brightfield images show loading of cells in the presence of fatty acid mixture by day three with maintained retention at day six. Fluorescent staining of cells with BODIPY confirms the accumulation of lipids in cells treated with lipid mixture but not control cells.

FIG. 10B quantifies and tabulates fluorescent staining data presented in FIG. 10A as the fold increase in lipid loading to total cell volume compared to control.

FIG. 11A shows, in some embodiments, the loading of yellowtail myoblasts with individual saturated fatty acids. Yellowtail myoblasts were cultured in media with reduced serum in the presence of increasing concentrations of individual saturated fatty acids—lauric acid, myristic acid, palmitic acid, or stearic acid for six days. Fluorescent images of cells stained with BODIPY confirm accumulation of fatty acids dependent on concentration. Highest lipid loading is observed for lauric acid, which shows accumulation at all concentrations tested with increased rounding and cell size at higher concentrations. Some lipid retention is observed for myristic, palmitic, and stearic acid but only above 50 μg/ml.

FIG. 11B quantifies and tabulates data presented in FIG. 11A as the percent of lipid loading to total cell volume.

FIG. 12A shows, in some embodiments, the loading of yellowtail myoblasts with individual monounsaturated fatty acids. Yellowtail myoblasts were cultured in media with reduced serum in the presence of increasing concentrations of individual saturated fatty acids—palmitoleic acid, oleic acid, vaccenic acid, or nervonic acid for six days. Fluorescent images of cells stained with BODIPY confirm accumulation of fatty acids dependent on concentration. Loading at all concentrations was observed for palmitoleic, oleic, and vaccenic acid with increased cell rounding and accumulation at increased lipid concentrations. No lipid loading was observed for nervonic acid.

FIG. 12B quantifies and tabulates data presented in FIG. 12A as the percent of lipid loading to total cell volume.

FIG. 13A shows, in some embodiments, the loading of yellowtail myoblasts with individual polyunsaturated fatty acids. Yellowtail myoblasts were cultured in media with reduced serum in the presence of increasing concentrations of individual saturated fatty acids—EPA, DHA, linolenic acid, or linoleic acid for six days. Fluorescent images of cells stained with BODIPY confirm accumulation of fatty acids dependent on concentration. Loading at all concentrations was observed for linolenic acid with increased cell rounding and accumulation at increased lipid concentrations. Lipid retention of EPA, DHA, and linoleic acid was only observed starting at 50 μg/ml and with limited total loading capacity. DHA showed some toxicity effects at highest concentrations tested as evidenced by lower cell number.

FIG. 13B quantifies and tabulates data presented in FIG. 13A as the percent of lipid loading to total cell volume.

FIG. 14A shows, in some embodiments, the dose-dependent loading of complex fatty acid mixtures into carp pre-adipocytes. Fluorescent images of cells stained with BODIPY confirm accumulation of fatty acids dependent on concentration.

FIG. 14B quantifies and tabulates data presented in FIG. 14A as the percent of lipid loading to total cell volume. The increase of lipid loading ranges up to 40 percentage points greater than control.

FIG. 15A shows, in some embodiments, the dose-dependent loading of complex fatty acid mixtures into bluefin tuna preadipocytes. Fluorescent images of cells stained with BODIPY confirm accumulation of fatty acids dependent on concentration.

FIG. 15B quantifies and tabulates data presented in FIG. 15A as the percent of lipid loading to total cell volume. The increase of lipid loading ranges up to 65 percentage points greater than control.

FIG. 16A shows, in some embodiments, the dose-dependent loading of complex fatty acid mixtures into bluefin tuna preadipocytes cultured and lipid loaded in serum free media. Fluorescent images of cells stained with BODIPY confirm accumulation of fatty acids dependent on concentration.

FIG. 16B quantifies and tabulates data presented in FIG. 16A as the percent of lipid loading to total cell volume. The increase of lipid loading ranges up to 60 percentage points greater than control.

FIG. 17A shows, in some embodiments, the dose-dependent loading of complex fatty acid mixtures into yellowfin tuna fibroblasts. Fluorescent images of cells stained with BODIPY confirm accumulation of fatty acids dependent on concentration.

FIG. 17B quantifies and tabulates data presented in FIG. 17A as the percent of lipid loading to total cell volume. The increase of lipid loading ranges up to 45 percentage points greater than control.

FIG. 18A shows, in some embodiments, the dose-dependent loading of complex fatty acid mixtures into bluefin tuna fibroblasts. Fluorescent images of cells stained with BODIPY confirm accumulation of fatty acids dependent on concentration.

FIG. 18B quantifies and tabulates data presented in FIG. 18A as the percent of lipid loading to total cell volume. The increase of lipid loading ranges up to 35 percentage points greater than control.

FIG. 19A shows, in some embodiments, the dose-dependent loading of complex fatty acid mixtures into bluegill fibroblasts. Fluorescent images of cells stained with BODIPY confirm accumulation of fatty acids dependent on concentration.

FIG. 19B quantifies and tabulates data presented in FIG. 19A as the percent of lipid loading to total cell volume. The increase of lipid loading ranges up to 35 percentage points greater than control.

FIG. 20A shows, in some embodiments, the dose-dependent loading of complex fatty acid mixtures into Mozambique tilapia brain derived cells. Fluorescent images of cells stained with BODIPY confirm accumulation of fatty acids dependent on concentration.

FIG. 20B quantifies and tabulates data presented in FIG. 20A as the percent of lipid loading to total cell volume. The increase of lipid loading ranges up to 25 percentage points greater than control.

FIG. 21A shows, in some embodiments, the dose-dependent loading of complex fatty acid mixtures into chicken embryonic fibroblasts. Fluorescent images of cells stained with BODIPY confirm accumulation of fatty acids dependent on concentration.

FIG. 21B quantifies and tabulates data presented in FIG. 21A as the percent of lipid loading to total cell volume. The increase of lipid loading ranges up to 35 percentage points greater than control.

FIG. 22A shows, in some embodiments, the dose-dependent loading of complex fatty acid mixtures into rabbit skin fibroblasts. Fluorescent images of cells stained with BODIPY confirm accumulation of fatty acids dependent on concentration.

FIG. 22B quantifies and tabulates data presented in FIG. 22A as the percent of lipid loading to total cell volume. The increase of lipid loading ranges up to 35 percentage points greater than control.

FIG. 23A shows, in some embodiments, the dose-dependent loading of complex fatty acid mixtures into red snapper myoblasts. Fluorescent images of cells stained with BODIPY confirm accumulation of fatty acids dependent on concentration.

FIG. 23B quantifies and tabulates data presented in FIG. 23A as the percent of lipid loading to total cell volume. The increase of lipid loading ranges up to 20 percentage points greater than control.

FIG. 24A shows, in some embodiments, the dose-dependent loading of complex fatty acid mixtures into mahi mahi myoblasts. Fluorescent images of cells stained with BODIPY confirm accumulation of fatty acids dependent on concentration.

FIG. 24B quantifies and tabulates data presented in FIG. 24A as the percent of lipid loading to total cell volume. The increase of lipid loading ranges up to 55 percentage points greater than control.

FIG. 25A shows, in some embodiments, the dose-dependent loading of complex fatty acid mixtures into bluefin tuna myoblasts. Fluorescent images of cells stained with BODIPY confirm accumulation of fatty acids dependent on concentration.

FIG. 25B quantifies and tabulates data presented in FIG. 25A as the percent of lipid loading to total cell volume. The increase of lipid loading ranges up to 40 percentage points greater than control.

FIG. 26A shows, in some embodiments, the dose-dependent loading of complex fatty acid mixtures into bluefin tuna myoblasts cultured and lipid loaded in serum free media. Fluorescent images of cells stained with BODIPY confirm accumulation of fatty acids dependent on concentration.

FIG. 26B quantifies and tabulates data presented in FIG. 26A as the percent of lipid loading to total cell volume. The increase of lipid loading ranges up to 30 percentage points greater than control.

FIG. 27A shows, in some embodiments, the dose-dependent loading of complex fatty acid mixtures into bovine muscle satellite cells. Fluorescent images of cells stained with BODIPY confirm accumulation of fatty acids dependent on concentration.

FIG. 27B quantifies and tabulates data presented in FIG. 27A as the percent of lipid loading to total cell volume. The increase of lipid loading ranges up to 15 percentage points greater than control.

FIG. 28A shows, in some embodiments, the dose-dependent loading of complex fatty acid mixtures into Atlantic salmon kidney epithelial cells. Fluorescent images of cells stained with BODIPY confirm accumulation of fatty acids dependent on concentration.

FIG. 28B quantifies and tabulates data presented in FIG. 28A as the percent of lipid loading to total cell volume. The increase of lipid loading ranges up to 15 percentage points greater than control.

FIG. 29A shows, in some embodiments, the dose-dependent loading of complex fatty acid mixtures into rainbow trout gill epithelial cells. Fluorescent images of cells stained with BODIPY confirm accumulation of fatty acids dependent on concentration.

FIG. 29B quantifies and tabulates data presented in FIG. 29A as the percent of lipid loading to total cell volume. The increase of lipid loading ranges up to 45 percentage points greater than control.

FIG. 30A shows, in some embodiments, the dose-dependent loading of complex fatty acid mixtures into dog kidney epithelial cells. Fluorescent images of cells stained with BODIPY confirm accumulation of fatty acids dependent on concentration.

FIG. 30B quantifies and tabulates data presented in FIG. 30A as the percent of lipid loading to total cell volume. The increase of lipid loading ranges up to 35 percentage points greater than control.

FIG. 31A shows, in some embodiments, the dose-dependent loading of complex fatty acid mixtures into pig kidney epithelial cells. Fluorescent images of cells stained with BODIPY confirm accumulation of fatty acids dependent on concentration.

FIG. 31B quantifies and tabulates data presented in FIG. 31A as the percent of lipid loading to total cell volume. The increase of lipid loading ranges up to 50 percentage points greater than control.

FIG. 32A shows, in some embodiments, the dose-dependent loading of complex fatty acid mixtures into fall army worm epithelial cells. Fluorescent images of cells stained with BODIPY confirm accumulation of fatty acids dependent on concentration.

FIG. 32B quantifies and tabulates data presented in FIG. 32A as the percent of lipid loading to total cell volume. The increase of lipid loading ranges up to 20 percentage points greater than control.

FIG. 33A shows, in some embodiments, the dose-dependent loading of complex fatty acid mixtures into quail neuroretina cells. Fluorescent images of cells stained with BODIPY confirm accumulation of fatty acids dependent on concentration.

FIG. 33B quantifies and tabulates data presented in FIG. 33A as the percent of lipid loading to total cell volume. The increase of lipid loading ranges up to 30 percentage points greater than control.

FIG. 34 shows yellowtail myoblasts in control and reduced serum media. Yellowtail myoblasts cultured in control or reduced serum media for six days show no lipid loading by brightfield or fluorescence imaging. Staining with BODIPY showed no lipid accumulation in either condition.

FIG. 35 shows yellowtail myoblasts in control media without added fatty acids. Yellowtail myoblasts cultured in control media for six days show no lipid loading by brightfield or fluorescence imaging. Cells stained with Hoechst for nuclei, Mitotracker for mitochondrial health, and Phalloidin for F-actin cytoskeletal proteins while staining with BODIPY showed no lipid accumulation.

FIG. 36 shows loading of yellowtail myoblasts with chemically defined fatty acid combinations. Highest lipid content was observed for media 16, 22, and 25 as shown by extensive BODIPY staining for fat droplets. Comparable cell number and viability were shown in comparison to control conditions that did not yield lipid loading.

FIG. 37A shows plots representing quantification of cell number after exposure to defined fatty acid combinations. Yellowtail myoblasts grown in control media (Media 28 and 29) or media with defined fatty acid combinations (Media 1-27) were quantified. Fluorescence intensity was quantified for each combination to evaluate effects on the cell phenotype. Media 16, 22, and 25 exhibited comparable cell number and health to control and significantly higher lipid content. Increased lipid content was observed in most fatty acid combinations tested with all three highest combinations containing nervonic acid at the highest level tested.

FIG. 37B shows plots representing quantification of lipid loading after exposure to defined fatty acid combinations. Yellowtail myoblasts grown in control media (Media 28 and 29) or media with defined fatty acid combinations (Media 1-27) were stained with BODIPY for lipid droplets. Fluorescence intensity was quantified for each combination to evaluate effects on the cell phenotype. Media 16, 22, and 25 exhibited comparable cell number and health to control and significantly higher lipid content. Increased lipid content was observed in most fatty acid combinations tested with all three highest combinations containing nervonic acid at the highest level tested.

FIG. 37C shows plots representing quantification of cell health after exposure to defined fatty acid combinations. Yellowtail myoblasts grown in control media (Media 28 and 29) or media with defined fatty acid combinations (Media 1-27) were stained with Mitotracker for mitochondrial health. Fluorescence intensity was quantified for each combination to evaluate effects on the cell phenotype. Media 16, 22, and 25 exhibited comparable cell number and health to control and significantly higher lipid content. Increased lipid content was observed in most fatty acid combinations tested with all three highest combinations containing nervonic acid at the highest level tested.

FIG. 38 shows the protective effect on bluefin tuna myoblasts of nervonic acid (NA) in the presence of toxic fatty acids (FA). A 70× increase in cell number is observed at toxic concentration of 50 μg/mL DHA when in the presence of nervonic acid.

FIG. 39 shows the protective effect on dog kidney cells of nervonic acid in the presence of toxic fatty acids. A 5.5× increase in cell number is observed at toxic concentration of 50 μg/mL DHA when in the presence of nervonic acid.

FIG. 40 shows the protective effect on rabbit skin fibroblasts of nervonic acid in the presence of toxic fatty acids. A 42× increase in cell number is observed at toxic concentration of 50 μg/mL DHA when in the presence of nervonic acid.

### DETAILED DESCRIPTION

Provided herein are compositions, methods and systems and related cells, cell biomass and cell-cultured food products, which in several embodiments allow improved proliferation and enhanced lipid loading in a cellular biomass.

The cells and cell biomass can comprise cells from any animal, defined herein as an organism of kingdom Animalia. The cell-cultured food products can comprise cells from any animal excluding any member of genus Homo. The term “aquatic animal” as used herein indicates an animal, either vertebrate or invertebrate, which lives in the water for most or all of its lifetime. Accordingly, aquatic animal indicates an animal that breathes air or extracts oxygen dissolved in water through specialized organs called gills, or directly through the skin. Aquatic animals can live in fresh water (freshwater animals) or salt water (marine animals) as will be understood by a skilled person.

Exemplary aquatic animals comprise fish (gill-bearing aquatic craniate animals that lack limbs with digits), and shellfish (aquatic invertebrate animal having a shell and belonging to the phylum Mollusca, the class Crustacea (phylum Arthropoda), or the phylum Echinodermata).

In particular, aquatic animals in the sense of the disclosure comprise fish such as cartilaginous fish, bony fish, ray-finned fish, lobe-finned fish and seafood such as various species of mollusks (e.g. bivalve mollusks such as clams, oysters, and mussels and cephalopods, such as octopus and squid), crustaceans (e.g. shrimp, crabs, and lobster), and echinoderms (e.g. sea cucumbers and sea urchins). Such animals are comprised of a variety of cells that have different morphology and functions, such as, myoblasts, myocytes, fibroblasts, adipocytes, preadipocytes, endothelial cells, stem cells, osteoblasts, osteocytes, keratinocytes, neurons and others identifiable to a person skilled in the art.

Exemplary aquatic animals comprise as basa, flounder, hake, scup, smelt, rainbow trout, hardshell clam, blue crab, peekytoe crab, spanner crab, cuttlefish, Eastern oyster, Pacific oyster, anchovy, herring, lingcod, moi, orange roughy, Atlantic Ocean perch, Lake Victoria perch, yellow perch, European oyster, Dover sole, sturgeon, tilefish, wahoo, yellowtail, sea urchin, Atlantic mackerel, sardines, black sea bass, European sea bass, hybrid striped bass, bream, cod, drum, haddock, hoki, Alaska pollock, rockfish, pink salmon, snapper, tilapia, turbot, walleye, lake whitefish, wolffish, hardshell clam, surf clam, cockle, Jonah crab, snow crab, crayfish, bay scallop, Chinese white shrimp, sablefish, Atlantic salmon, coho salmon, skate, dungeness crab, king crab, blue mussel, greenshell mussel, pink shrimp, Escolar, chinook salmon, chum salmon, American shad, Arctic char, carp, catfish, dory, grouper, halibut, monkfish, pompano, abalone, conch, stone crab, American lobster, spiny lobster, octopus, black tiger shrimp, freshwater shrimp, gulf shrimp, Pacific white shrimp, squid, barramundi, cusk, dogfish, kingklip, mahi-mahi, opah, mako shark, swordfish, albacore tuna, yellowfin tuna, geoduck clam, squat lobster, sea scallop, rock shrimp, barracuda, Chilean sea bass, cobia, croaker, eel, blue marlin, mullet, sockeye salmon, bluefin tuna, shrimp, crabs, lobster, and echinoderms (e.g. sea cucumbers and sea urchins). Some preferred aquatic animals include yellowtail (e.g., Seriola lalandi), mahi-mahi (Coryphaena hippurus), red snapper (Lutjanus campechanus), bluefin tuna (e.g., Thunnus orientalis and Thunnus thynnus), yellowfin tuna (Thunnus albacares), cod (e.g., Gadus morhua, Gadus Macrocephalus, Gadus ogac), flounder, halibut, herring, mackeral, pompano, salmon, sea bass, Patagonian toothfish (Dissostichus eleginoides), squid, clams, lobster, crabs, scallops, shrimp, eel, bass (e.g., Micropterus salmoides), bluegill (Lepomis macrochirus), and carp (e.g., Hypophthalmichthys molitrix).

For example, the finfish species can be a saltwater finish species or a freshwater finfish species. Exemplary saltwater finfish species include, but are not limited to, those selected from the group consisting of Mahi-mahi, bluefin tuna, Alaska pollock, albacore tuna, American shad, anchovy, Arctic char, Atlantic mackerel, Atlantic Ocean perch, Atlantic salmon, barracuda, barramundi, bass, black sea bass, blue marlin, bream, Chilean sea bass, chinook salmon, chum salmon, cobia, cod, coho salmon, croaker, drum, cusk, dogfish, dory, Dover sole, eel, Escolar, European sea bass, flounder, grouper, gulf shrimp, haddock, hake, halibut, herring, hoki, kingklip, lingcod, mackerel, mako shark, moi, monkfish, mullet, opah, orange roughy, Pacific white shrimp, Patagonian toothfish (Dissostichus eleginoides), pink salmon, pompano, rainbow trout, red snapper (Lutjanus campechanus), rockfish, sablefish, salmon, sardines, scup, sea bass, skate, smelt, snapper, sockeye salmon, sturgeon, swordfish, tilefish, turbot, wahoo, wolffish, yellowfin tuna, and yellowtail.

Exemplary freshwater finfish include, but are not limited to, those selected from the group consisting of Arctic char, barramundi, basa, bass, bluegill (Lepomis macrochirus), bream, carp, catfish, croaker, drum, dogfish, bowfin, eel, freshwater shrimp, hybrid striped bass, Lake Victoria perch, lake whitefish, mullet, rainbow trout, salmon, smelt, sturgeon, tilapia, walleye, and yellow perch.

Exemplary crustacean species include, but are not limited to, those selected from the group consisting of shrimp, crabs, crayfish, and lobsters, e.g., American lobster, black tiger shrimp, blue crab, Chinese white shrimp, crabs, crayfish, Dungeness crab, Jonah crab, king crab, lobster, peekytoe crab, pink shrimp, rock shrimp, shrimp, snow crab, spanner crab, spiny lobster, stone crab, and squat lobster.

Exemplary echinoderm species include, but are not limited to, those selected from the group consisting of sea cucumbers and sea urchins.

Exemplary cephalopod species include, but are not limited to, those selected from the group consisting of octopus and squid.

Exemplary mollusk species include, but are not limited to, those selected from the group consisting of clams, oysters, mussels, abalone, bay scallop, blue mussel, cockle, conch, cuttlefish, Eastern oyster, hardshell clam, Pacific oyster, European oyster, geoduck clam, greenshell mussel, scallops, and surf clam.

The term “terrestrial animal” as used herein indicates an animal, either vertebrate or invertebrate, which lives outside of water for most or all of its lifetime. Terrestrial animals consume oxygen from air, with the air acquired by breathing with lungs, entry of air into tracheae, or other mechanisms known to a person of ordinary skill in the art. Terrestrial animals include many mammals, birds, reptiles, amphibians, and insects, among others known to a person of ordinary skill in the art.

Exemplary terrestrial animals include, but are not limited to, army worm, cricket, grasshopper, frog, toad, newt, salamander, alligator, crocodile, snake, chicken, turkey, duck, goose, pheasant, game hen, quail, horse, rhinoceros, tapir, cattle, pig, giraffe, camel, sheep, deer, goat, rabbit, dog, and hippopotamus.

Exemplary terrestrial insect species include, but are not limited to, those selected from the group consisting of army worm, cricket, and grasshopper.

Exemplary terrestrial amphibian species include, but are not limited to, those selected from the group consisting of frog, toad, newt, and salamander.

Exemplary terrestrial reptile species include, but are not limited to, those selected from the group consisting of alligator, crocodile, and snake.

Exemplary terrestrial avian species include, but are not limited to, those selected from the group consisting of chicken, turkey, duck, goose, pheasant, game hen, and quail.

Exemplary terrestrial mammalian species include, but are not limited to, those selected from the group consisting of horse, rhinoceros, tapir, cattle, pig, giraffe, camel, sheep, deer, goat, rabbit, dog, and hippopotamus.

Whether aquatic or terrestrial, all animals are comprised of a variety of cells that have different morphology and functions, such as myoblasts, myocytes, fibroblasts, adipocytes, preadipocytes, endothelial cells, epithelial cells, embryonic stem cells, adult stem cells, induced pluripotent stem cells, osteoblasts, osteocytes, keratinocytes, neurons and others identifiable to a person skilled in the art.

The animal cells used herein may be from more than one animal species, such as two, three, four, or more of the aquatic and/or terrestrial animal species.

The term “myoblast” is a term of art that refers to precursors of myocytes, which are also called muscle cells. Myoblasts differentiate into muscle cells through myogenesis as will be understood by a person skilled in the art. Myoblasts can be classified as skeletal muscle myoblasts, smooth muscle myoblasts, and cardiac muscle myoblasts depending on the type of muscle cell that they will differentiate into. Exemplary myoblasts of aquatic and terrestrial animals comprise skeletal muscle myoblasts and smooth muscle myoblasts.

The term “fibroblast” is a term of art that refers to type of cell in the connective tissue of animals and that synthesize components of the extracellular matrix, such as collagen. Fibroblasts produce the structural framework for animal tissues and play a critical role in wound healing. Fibroblasts are the most common cells of connective tissue in animals. Fibroblasts have a branched cytoplasm surrounding an elliptical, speckled nucleus having two or more nucleoli. Active fibroblasts can be recognized by their abundant rough endoplasmic reticulum. Inactive fibroblasts, also called fibrocytes, are smaller, spindle-shaped, and have a reduced amount of rough endoplasmic reticulum. Although disjointed and scattered when they have to cover a large space, fibroblasts, when crowded, often locally align in parallel clusters. Exemplary fibroblasts include fibroblasts from muscle and other tissues such as brain, heart or skin.

The term “adipocyte” is a term of art that refers to fat cells, which are also known as lipocytes. Adipocytes are the cells that primarily compose adipose tissue, specialized in storing energy as fat. Adipocytes can be derived from mesenchymal stem cells which give rise to adipocytes through adipogenesis. In cell culture, adipocytes can also form osteoblasts, myocytes and other cell types. There are two types of adipose tissue, white adipose tissue (WAT) and brown adipose tissue (BAT), which are also known as white and brown fat, respectively, and comprise two types of fat cells. Adipocytes can arise either from preadipocytes resident in adipose tissue, or from bone-marrow derived progenitor cells that migrate to adipose tissue. Cells used herein typically comprise adipocytes from white adipose tissue.

The term “preadipocyte” is a term of art that indicates progenitors of mature differentiated adipocytes which can be stimulated to form adipocytes. Preadipocytes can be isolated from subcutaneous or visceral fatty tissue of an animal.

Preadipocytes can be grown in a preadipocyte growth medium which contains all the growth factors and supplements necessary for the optimal growth of undifferentiated preadipocytes. For example, the preadipocytes can be grown in a preadipocyte growth medium containing endothelial cell growth supplement, epidermal growth factor, hydrocortisone, and/or heparin.

The formation of adipocytes from preadipocytes involves a tightly regulated cell differentiation process, referred to as adipogenesis, in which mesenchymal stem cells commit to preadipocytes and preadipocytes differentiate into adipocytes. The terms “differentiate,” or “differentiation,” refer to a process of a change of expression patterns during which multipotent gene expression alters to cell type specific gene expression. Transcription factors, such as peroxisome proliferator-activated receptor y (PPARy) and CCAAT enhancer-binding proteins (C/EBPs) are main regulators of adipogenesis. Features of differentiated adipocytes are growth arrest, morphological change, high expression of lipogenic genes and production of adipokines such as adiponectin, leptin, resistin (in mouse, not in humans) and TNF-alpha, as will be understood by a person skilled in the art.

In embodiments of the instant disclosure, composition methods and systems are described and related cells, cells biomass and cell-cultured food products with a controllable cell lipid content and lipid uptake, and/or with an improved cell differentiation and/or cell viability in connection with set lipid content and lipid uptake.

The term “lipid” is a term of art that refers to any organic compound containing a linear or cyclic aliphatic chain of at least six carbon atoms and at least one oxygen or nitrogen atom bonded to one of the carbon atoms that at 20° C. has a solubility of at least 20% w/w in ether or ethanol and has a solubility of equal or less than 1% w/w in water. Lipids include fatty acids, glycerolipids, glycerophospholipids, sphingolipids, sterols, prenols, saccharolipids, and polyketides as will be understood by a skilled person.

“Fatty acids” are hydrophobic molecules consisting of a saturated or unsaturated aliphatic hydrocarbon chain terminating in a carboxylic acid moiety. “Saturated fatty acids” (SFA) contain no double bonds. SFA can be categorized according to length of their chain and typically contain 4-22 carbon atoms). For example, exemplary SFA can include lauric acid with 12 carbons, myristic acid with 14 carbons, palmitic acid with 16 carbons, stearic acid with 18 carbons and capric acid with 10 carbon atoms. Additionally, exemplary saturated fatty acids are well-known in the art. “Unsaturated fatty acids” (UFA) contain one or more double bonds in the fatty acid chain. Unsaturated fatty acids can be categorized based on the number of double bonds included in the chain. UF As include fatty acids with varying numbers of double bonds at various locations along the carbon chain. For example, monounsaturated (MUFA), if it contains one double bond, and polyunsaturated (PUFA) if it contains more than one double bond.

PUFA as a class include many nutritionally important compounds, such as essential fatty acids. Polyunsaturated fatty acids can be categorized based on, the length of their carbon backbone, in groups such as short chain polyunsaturated fatty acids (SC-PUFA), with 16 or 20 carbon atoms and long-chain polyunsaturated fatty acids (LC-PUFA) with more than 18 carbon atoms. Polyunsaturated fatty acids can also be categorized based on their chemical structure: in methylene-interrupted polyenes such as Omega 3, Omega-6 and Omega-9, conjugated fatty acids and other PUFA.

In particular, Omega-3 fatty acids are polyunsaturated fatty acids (PUFA) characterized by the presence of a double bond three atoms away from the terminal methyl group in their chemical structure. The three main omega-3 fatty acids are α-linolenic acid (ALA), eicosapentaenoic acid (EPA), and docosahexaenoic acid (DHA).

Unsaturated fatty acids can include fatty acids with varying carbon chain length from 12 to 22 carbons. Polyunsaturated fatty acids can include fatty acids with varying carbon chain length such as linoleic acid or α-linolenic acid with 18 carbons, EPA with 20 carbons or DHA with 22 carbons. Monounsaturated fatty acids (MUFA) can include fatty acids with varying carbon chain length such as palmitoleic acid with 16 carbons, vaccenic acid with 18 carbons, oleic acid with 18 carbons and nervonic acid with 24 carbons.

Exemplary unsaturated fatty acids include α-Linolenic acid, stearidonic acid, eicosapentaenoic acid, cervonic acid, linoleic acid, linolelaidic acid, palmitoleic acid, vaccenic acid, oleic acid, nervonic acid and others identifiable to a person skilled in the art. Additional exemplary SFA, MUFA, and PUFA are well-known in the art and are disclosed herein (see, e.g., Table 7 of Example 5).

Sterols are well-known in the art and are steroids with a hydroxyl group at the 3-position of the A-ring. Accordingly, sterols comprise the fused four-ring core structure of steroid substituted at the -position of the A-ring as will be understood by a person skilled in the art. Steroids can include the eighteen-carbon (C18) steroids such as estrogens, C19 steroids such as androgens (e.g. testosterone and androsterone), C21 such as progestogens, glucocorticoids and mineralocorticoids, as well as secosteroids, comprising various forms of vitamin D, characterized by cleavage of the B ring of the core structure. Exemplary sterols comprise cholesterol and its derivatives, which are important components of membrane lipids, along with the glycerophospholipids and sphingomyelins, phytosterols, such as ß-sitosterol, stigmasterol, and brassicasterol, ergosterol and additional sterols identifiable by a skilled person.

Phospholipids are well-known in the art and include, for example, phosphatidylserine, phosphatidylcholine, phosphatidylethanolamine, phosphatidylinositol, and the like. Phospholipids are present in and can be extracted from lecithins.

Glycerolipids are well-known in the art and are composed of mono-, di-, and tri-substituted glycerols. Accordingly, glycerolipids can comprise monoglycerides, diglycerides and triglycerides. In particular, triglycerides are fatty acid triesters of glycerol in which the three hydroxyl groups of glycerol are each esterified, typically by different fatty acids. Triglycerides are triesters consisting of a glycerol bound to three fatty acid molecules as will be understood by a person skilled in the art. Triglycerides can be classified into saturated and unsaturated types as will be understood by a skilled person.

In embodiments of the present disclosure, culture media, method and system to culture a cell of an aquatic animal are described, as well as cells of aquatic animals obtainable and/or obtained thereby. In embodiments of the present disclosure, culture media, method and system to culture a cell of a terrestrial animal are described, as well as cells of terrestrial animals obtainable and/or obtained thereby. In embodiments of the present disclosure, culture media, method and system to culture a cell of an aquatic or terrestrial animal are described, as well as cells of aquatic or terrestrial animals obtainable and/or obtained thereby.

As used herein, the term “media” refers to a composition in a liquid, solid or gel state comprising organic, inorganic and/or biogenic ingredients in which a cell is capable of surviving, maintaining vitality or proliferating. A medium typically comprises a basal medium.

The term “basal medium” as used herein indicates culture media comprising components essential for cell survival and growth such as amino acids, glucose, and ions such as calcium, magnesium, potassium, sodium, and phosphate, as will be understood by a person skilled in the art.

An example of basal medium is Basal Media Formulation (www.sigmaaldrich.com/life-science/cell-culture/learning-center/media-formulations/basal.html). Additional examples can be identifiable by a skilled person.

An exemplary biogenic ingredient includes serum. A media can be chemically defined. For example, Lipid Mixture 1, available from Sigma Aldrich (www.sigmaaldrich.com/catalog/product/sigma/10288?lang=en&region=US) contains non-animal derived fatty acids (2 μg/ml arachidonic and 10 μg/ml each linoleic, linolenic, myristic, oleic, palmitic and stearic), 0.22 mg/ml cholesterol from New Zealand sheep's wool, 2.2 mg/ml Tween-80, 70 μg/ml tocopherol acetate and 100 mg/ml Pluronic F-68 solubilized in cell culture water.

A media in the sense of the disclosure can have biogenic ingredients including Fetal Bovine Serum (FBS) or cod liver oil fatty acids. For example, Lipid Mixture (1000×), available from Sigma Aldrich (www.sigmaaldrich.com/catalog/product/sigma/15146?lang=en&region=US) contains cholesterol, 4.5 g/L, cod liver oil fatty acids (methyl esters), 10 g/L, polyoxyethylenesorbitan monooleate, 25 g/L, and D-α-tocopherol acetate, 2.0 g/L.

Embodiments of the present disclosure are based on the surprising finding that, lipids in culture media have different degrees of toxicity for cells of an aquatic animal, with MUFA being non-toxic while PUFA, SFA and/or sterols being toxic, as it reduced cell viability and/or proliferation relative to control media.

One way to detect toxicity in cell culture is to detect the percentage of area covered with cells under test condition (e.g., culture containing one or several lipids and or other components of interest) relative to control after a desired culture period. This can be accomplished using any suitable method, such as by obtaining an image of the culture vessel with cultured cells attached for example, a photomicrograph, and calculating the area covered by the cultured cells using a suitable image processing and analysis program, such as ImageJ. In one example, when lipids are tested for loading into cells, if the cell-covered surface in cultures that contain the test lipids is about 61-79% of the cell covered surface in control cultures, the lipids are considered slightly toxic, and if the cell-covered surface is 60% or less of the cell-covered surface in control cultures, the lipids are considered toxic.

Embodiments of the present disclosure are based on the surprising finding that addition of an effective amount of nervonic acid in culture medium for cells of an aquatic animal comprising desired lipids (e.g., PUFA, SFA and/or sterols), allows the uptake of the desired lipids by the cell while reducing and even minimizing the related levels of lipid toxicity on the cell.

“Nervonic acid” is known in the art and is the monounsaturated analog of lignoceric acid having formula C24H46O2 and IUPAC name (Z)-Tetracos-15-enoic acid. Nervonic acid is also known as selacholeate, nervonsaeure, cis-15-tetracosenoic acid, 24:1 cis, delta 15 or 24:1 omega 9 as will be understood by a skilled person. Nervonic acid is lipid of the glycosphingolipids, cerebrosides. Nervonic acid has one double bond in the fatty acid chain and all the remaining carbon atoms are single-bonded.

In embodiments, this disclosure relates to a culture medium for culturing cells of an aquatic animal, such as myoblasts, myocytes, preadipocytes, adipocytes, and/or fibroblasts for example. The media is a basal media, that is preferably serum free, and is supplemented with one or more lipids, such as a PUFA, SFA, sterol, or any combination of a PUFA, SFA and/or sterol. Such media can be used to expand cell populations in culture, e.g., through proliferation, and/or to induce cellular differentiation. Advantageously, the media that is supplemented with one or more lipids can be used to alter the lipid content of the cultured cells. For example, as described and exemplified herein, the relative amount of a desired fatty acid, such as palmitic acid, vaccenic acid, oleic acid, alpha-linolenic acid (ALA), eicosapentaenoic acid (EPA), docosahexaenoic acid (DHA), docosapentaenoic acid (DPA), and any combination of the foregoing can be increased in cells that are cultured in media supplemented with these fatty acids relative to the same cells as they occur in nature (i.e. as components of the aquatic animal from which they are isolated; as components of the terrestrial animal from which they are isolated) or as cultured in media that does not include the lipid supplements. Each desired lipid is typically present in the media at a concentration of at least about 0.01 μg/ml. In examples, each desired lipid is present in the media at a concentration of about 0.01 μg/ml to about 1000 μg/ml, about 0.01 μg/ml to about 100 μg/ml, about 0.1 μg/ml to about 100 μg/ml, about 1.0 μg/ml to about 100 μg/ml, about 10 μg/ml to about 200 μg/ml, about 10 μg/ml to about 150 μg/ml, and preferably about 10 μg/ml to about 100 μg/ml or about 10 μg/ml to about 50 μg/ml. As described herein, some lipids (e.g., fatty acids) are toxic to cultured cells from an aquatic animal at higher concentrations. A person of skill in the art will be able to discern toxicity levels for a lipid of interest and adjust the concentration of that lipid to achieve the desired degree of cellular lipid uptake, proliferation and/or survival. A person of skill in the art will be able to discern toxicity levels for a lipid of interest, cell type of interest, and/or a cell animal species of interest, and adjust the concentration of that lipid to achieve the desired degree of cellular lipid uptake, proliferation and/or survival.

The media, methods and systems disclosed herein can be used to create cells with desired lipid content, for example to enhance desired attributes, such as taste, aroma, shelf life and/or nutrient content. For example, the media, methods and systems disclosed herein can be used to make aquatic animal food products, such as cells and seafood products as disclosed herein, that contain a total fat content that is the same or similar to that of a wild caught animal of the same species (i.e., the same type of cell, fillet, etc. of the wild caught animal) but that has less saturated fat as a percentage of total fat and/or based on weight of the food product. In one aspect, the media, methods and systems disclosed herein can be used to make terrestrial animal food products, such as cells and terrestrial animal food as disclosed herein, that contain a total fat content that is the same or similar to that of a wild caught and/or farm raised animal of the same species (i.e., the same type of cell, fillet, etc. of the wild caught or farm raised animal) but that has less saturated fat as a percentage of total fat and/or based on weight of the food product. In examples, this disclosure relates to food products that can provide certain health benefits, including food products that have high unsaturated fatty acid content and low saturated fatty acid content, for example by having increased amounts of oleic acid and/or omega-3 fatty acids (by weight and/or as a percentage of total fat) in comparison to wild caught seafood of the same species. In examples, this disclosure relates to food products that can provide certain health benefits, including food products that have high unsaturated fatty acid content and low saturated fatty acid content, for example by having increased amounts of oleic acid and/or omega-3 fatty acids (by weight and/or as a percentage of total fat) in comparison to a wild caught and/or farm raised animal of the same species. Such food products are associated with certain health benefits, including reduced risk of cardiovascular disease. In another example, this disclosure relates to food products with phosphatidylserine content (by weight and/or as a percentage of total fat) in comparison to wild caught seafood of the same species. In another example, this disclosure relates to food products with phosphatidylserine content (by weight and/or as a percentage of total fat) in comparison to a wild caught and/or farm-raised animal of the same species. Such food products are associated with reduced risk of cognitive dysfunction or dementia.

In other examples, the media, methods and systems disclosed herein can be used to make aquatic animal food products, such as cells and seafood products as disclosed herein, that have desired sensory attributes. In other examples, the media, methods and systems disclosed herein can be used to make terrestrial animal food products, such as cells and terrestrial animal food products as disclosed herein, that have desired sensory attributes. For example, a pleasant aroma is associated with the content of fatty acids, in particular, with free fatty acid and PUFA content. Accordingly, the content of free fatty acids and PUFA can be adjusted using the media and methods disclosed herein to achieve a desired aroma profile. Similarly, lipid content can be varied to achieve a desired flavor profile, for example, increased or decreased fishiness of seafood products by altering the content of omega-3 fatty acids. Other sensory attributes, such as mouthfeel and texture, cookability/moistness and appearance can also be tuned by adjusting the lipid content of cells. See, e.g., Rosa et al., Nutrients 2020, 12, 3453; doi: 10.3390/nu12113454, regarding seafood sensory attributes and lipid composition. Similar considerations apply to terrestrial animal food products.

As described and exemplified herein, it was surprisingly discovered that including nervonic acid in the media supplemented with lipids (e.g., fatty acids) facilitated the uptake of the lipids by cultured aquatic animal cells, including at concentrations that were otherwise toxic to the cultured cells. This was a surprising discovery, in part, because aquatic animal cells did not substantially uptake nervonic acid when cultured in media that was supplemented with nervonic acid but not with other fatty acids. As described and exemplified herein, nervonic acid was the only MUFA tested that was not substantially taken up by aquatic animal cells that were cultured in media supplemented with a test MUFA. Without wishing to be bound by any particular theory, it is believed that nervonic acid facilitates the uptake of other fatty acids, and that nervonic acid reduces or blocks fatty acid toxicity in cultured aquatic animal cells, such as fish cells.

Accordingly, in preferred aspects, the media further includes nervonic acid in an amount effective to increase uptake of lipids (e.g., PUFA, MUFA, SFA and/or sterols) by cultured aquatic animal cells and/or reduce toxic effects of the PUFA, MUFA, SFA and/or sterol on the cultured cells of an aquatic animal, e.g., an amount sufficient to improve cell viability and/or proliferation in the culture. Typically, nervonic acid is included in the media at a concentration of at least about 1 μg/ml, and is preferably included from about 1 μg/ml to about 1000 μg/ml, about 1 μg/ml to about 200 μg/ml, about 1 μg/ml to about 150 μg/ml, and preferably about 1 μg/ml to about 100 μg/ml, about 1 μg/ml to about 50 μg/ml, about 10 μg/ml to about 50 μg/ml, about 50 μg/ml to about 100 μg/ml or about 50 μg/ml to about 75 μg/ml. In further aspects, the media described herein (e.g., but not necessarily limited to, media supplemented with an effective amount of nervonic acid and/or one or more other lipids (e.g., PUFA, MUFA, SFA, sterols and combinations thereof) is further supplemented with an antioxidant. When aquatic animal cells are cultured in such media, e.g., fish myoblasts, myocytes, preadipocytes, adipocytes, fibroblasts and the like, the cells can take up lipids and antioxidants. This can reduce lipid oxidation in the cultured cell, which can contribute to loss of food quality of the cells, particularly cells that are enriched in unsaturated fatty acids such as PUFA and MUFA produced using the methods disclosed herein. Oxidation of lipids in cells cultured according to the methods described herein can contribute to altered flavor profiles, fishy odor, a shorter shelf life and reduced nutritional value. Accordingly, in these aspects, the media contains an amount of antioxidants effective to reduce or prevent lipid oxidation in the cultured cells, and products that contain the cultured cells. An effective amount of antioxidant in culture is typically an amount between about 10 ng/ml and about 1000 mg/ml, depending on the particular antioxidant selected and can be determined for any desired lipid using suitable methods. For example, the lipid oxidation product malondialdehyde (MDA) can be assessed in cells using suitable methods and expressed as thiobarbituric acid reactive substances (TBARS; ug MDA/mg cells). See, e.g., Secci, G. and Parisi, G. Italian Journal of Animal Science 15(1): 124-136 (2016).

Suitable antioxidants for use in the media described herein include, for example, ascorbic acid, mitoquinol, creatine, pinostrobin, catalase, N-acetylcysteine, thiazolidine, lipoic acid, butylated hydroxyanisole, baicalein, epicatechin gallate, rutin, myricetin, apigenin, sauchinone, propionyl-L-carnitine, tocopherols, including alpha-tocopherol, beta-tocopherol, gamma-tocopherol and delta-tocopherol, butylated hydroxyanisole (BHA), butylated hydroxy toluene (BHT), tert-butylhydroquinone (TBHQ), phenolics, carotenoids, anoxomer, dilauryl thiodipropionate, resveratrol, ethyoxyquin, propyl gallate, 2,4,5-trihydroxybutyrophenone (THBP), quercetin, carnosol, thymol, catechin, morin, proanthocyanidin dimer B2, algal extracts, botanical extracts (e.g., rosemary extract, grape seed extract, green tea extract, ginsing extract, blueberry extract, goji berry extract) and the like, and combinations thereof. Tocopherols, such as alpha-tocopherol, are generally preferred antioxidants.

Accordingly, in embodiments herein described, culture media for a cell of an aquatic animal comprises a basal medium supplemented with one or more lipids each in an amount of at least about 0.01 μg/ml and when one or more lipids are or comprise polyunsaturated fatty acids, saturated fatty acids and/or sterols, the culture medium further comprises nervonic acid in a concentration of at least about 1 μg/ml. Preferably, each lipid supplement is present in the media a concentration between about 0.1 μg/ml to about 1000 μg/ml, preferably about 0.1 μg/ml to about 500 μg/ml, about 1 μg/ml to about 100 μg/ml, or about 5 μg/ml to about 50 μg/ml Preferably, the total concentration of the lipids in the culture media is about 1 mg/ml or less, for example, the total concentration of lipids in the culture media can be about 0.5 mg/ml or less or about 0.1 mg/ml or less.

In some embodiment, the culture media can contain individual fatty acids or a mixture of fatty acids at a total concentration of 1 mg/ml of the total fatty acids in the lipid loading culture. Table 1 below provides an exemplary list of types and ranges of fatty acids that can be included in lipid loading culture medium.

TABLE 1
an exemplary list of types and ranges of fatty acids
that can be included in lipid loading culture medium

| | | Min | | Max | | Preferred | |
|---|---|---|---|---|---|---|---|
| | | concentration | | concentration | | Concentration | |
| | | 100 ng/ml | | 1 mg/ml | | Range | |
| MUFA (non-toxic) | | | | | | | |
| Myristoleic acid | C14:1 ω-5 | 100 | ng/ml | 1 | mg/ml | | |
| Palmitoleic acid | C16:1 ω-7 | 100 | ng/ml | 1 | mg/ml | 50-100 | μg/ml |
| Sapienic acid | C16:1 ω-10 | 100 | ng/ml | 1 | mg/ml | | |
| Vaccenic acid | C18:1 ω-7 | 100 | ng/ml | 1 | mg/ml | 50-75 | μg/ml |
| (18:1 w-9 found in most | C18:1 ω-9c | 100 | ng/ml | 1 | mg/ml | | |
| Phospholipids) | C18:1 ω-9t | 100 | ng/ml | 1 | mg/ml | | |
| Oleic acid | C18:1 ω-9 | 100 | ng/ml | 1 | mg/ml | 75 μg/ml-100 μg/ml | |
| Petroselinic acid | C18:1 ω-12 | 100 | ng/ml | 1 | mg/ml | | |
| Paullinic acid | C20:1 ω-7 | 100 | ng/ml | 1 | mg/ml | | |
| Gondoic acid | C20:1 ω-9 | 100 | ng/ml | 1 | mg/ml | | |
| Gadoleic acid | C20:1 ω-11 | 100 | ng/ml | 1 | mg/ml | | |
| Erucic | C22:1 ω-9c | 100 | ng/ml | 1 | mg/ml | | |
| Brassidic acid | C22:1 ω-9t | 100 | ng/ml | 1 | mg/ml | | |
| Nervonic acid | C24:1 ω-9 | 100 | ng/ml | 1 | mg/ml | 50-75 | μg/ml |
| PUFA (toxic) | | | | | | | |
| Hexadecatrienoic acid (HTA) | C16:3 ω-3 | 100 | ng/ml | 1 | mg/ml | | |
| Linoleic acid | C18:2 ω-6 | 100 | ng/ml | 1 | mg/ml | 50-75 | μg/ml |
| Alpha linolenic acid | C18:3 ω-3 | 100 | ng/ml | 1 | mg/ml | 50-100 | μg/ml |
| Gamma linolenic acid | C18:3 ω-6 | 100 | ng/ml | 1 | mg/ml | | |
| Stearidonic acid | C18:4 ω-3 | 100 | ng/ml | 1 | mg/ml | | |
| Eicosadienoic acid | C20:2 ω-6 | 100 | ng/ml | 1 | mg/ml | | |
| Eicosatrienoic acid (ETE) | C20:3 ω-3 | 100 | ng/ml | 1 | mg/ml | | |
| Dihomo-gamma-linolenic acid | C20:3 ω-6 | 100 | ng/ml | 1 | mg/ml | | |
| Mead acid | C20:3 ω-9 | 100 | ng/ml | 1 | mg/ml | | |
| Arachidonic acid | C20:4 ω-6 | 100 | ng/ml | 1 | mg/ml | | |
| Eicosapentaenoic acid (EPA) | C20:5 ω-3 | 100 | ng/ml | 1 | mg/ml | 10-75 | μg/ml |
| | C20:5 ω-6 | 100 | ng/ml | 1 | mg/ml | | |
| Heneicosapentaenoic acid | C21:5 ω-3 | 100 | ng/ml | 1 | mg/ml | | |
| (HPA) | | | | | | | |
| Docosatetraenoic acid | C22:4 ω-6 | 100 | ng/ml | 1 | mg/ml | | |
| DPA | C22:5 ω-3 | 100 | ng/ml | 1 | mg/ml | | |
| Docasahexaenoic acid (DHA) | C22:6 ω-3 | 100 | ng/ml | 1 | mg/ml | 10-25 | μg/ml |
| Tetracosapentaenoic acid | C24:5 ω-3 | 100 | ng/ml | 1 | mg/ml | | |
| Tetracosahexaenoic acid | C24:6 ω-3 | 100 | ng/ml | 1 | mg/ml | | |
| (Nisinic acid) | | | | | | | |
| SFA (toxic) | | | | | | | |
| Capric acid | C10:0 | 100 | ng/ml | 1 | mg/ml | | |
| Undecylic acid | C11:0 | 100 | ng/ml | 1 | mg/ml | | |
| Lauric acid | C12:0 | 100 | ng/ml | 1 | mg/ml | 25-75 | μg/ml |
| Tridecylic acid | C13:0 | 100 | ng/ml | 1 | mg/ml | | |
| Myristic acid | C14:0 | 100 | ng/ml | 1 | mg/ml | 25-75 | μg/ml |
| Pentadecylic acid | C15:0 | 100 | ng/ml | 1 | mg/ml | | |
| Palmitic acid | C16:0 | 100 | ng/ml | 1 | mg/ml | 25-50 | μg/ml |
| Margaric acid | C17:0 | 100 | ng/ml | 1 | mg/ml | | |
| Stearic acid | C18:0 | 100 | ng/ml | 1 | mg/ml | 25-75 | μg/ml |
| Nonadecylic acid | C19:0 | 100 | ng/ml | 1 | mg/ml | | |
| Arachidic acid | C20:0 | 100 | ng/ml | 1 | mg/ml | | |
| Heneicosylic acid | C21:0 | 100 | ng/ml | 1 | mg/ml | | |
| Behenic acid | C22:0 | 100 | ng/ml | 1 | mg/ml | | |
| Tricosylic acid | C23:0 | 100 | ng/ml | 1 | mg/ml | | |
| Lignoceric acid | C24:0 | 100 | ng/ml | 1 | mg/ml | | |

In some embodiments, culture medium methods and systems are described that allow upload of MUFA at a concentration of at least 10 μg/ml or higher, and in particular, at a centration from 10 μg/ml to 1000 μg/ml.

In some embodiments, the lipids comprised in the lipid loading medium are monounsaturated fatty acids (MUFA) provided in a concentration non-toxic to naturally existing fish cells between 100 ng/ml to 1 mg/ml.

Exemplary MUFA that can be used in culture medium methods and systems herein described, comprise myristoleic acid (C14:1 ω-5), palmitoleic acid, C16:1 ω-7, sapienic acid, C16:1 ω-10, vaccenic acid (C18:1 ω-7), C18:1 ω9c found in most phospholipids, oleic acid (C18:1 ω-9) petroselinic acid (C18:1 ω-12), paullinic acid (C20:1 ω-7), gondoic acid (C20:1 ω-11) erucic (C22:1 ω-9c), brassidic acid (C22:1 ω-9t), nervonic acid (C24:1 ω-9) and any desired combinations thereof.

In some embodiments, MUFA that can be used in culture medium methods and systems herein described comprise MUFA such as palmitoleic acid in a concentration of 50-100 μg/ml, MUFA such as vaccenic acid are provided at a concentration of 50-75 μg/ml, MUFA such as oleic acid are provided at a concentration of 75-100 μg/ml, and MUFA such as nervonic acid are provided at a concentration of 50-75 μg/ml.

In some embodiments, the lipids comprised in the lipid loading medium are polyunsaturated fatty acids (PUFA) provided in a concentration at which the PUFA alone are toxic to naturally existing fish cells but will be non-toxic when combined with nervonic acid. In those embodiments, the PUFA are comprised at a concentration of at least 10 μg/ml or higher, and in particular, at a concentration from 10 μg/ml to 1000 μg/ml.

Exemplary PUFA that can be used in connection with culture medium methods and systems herein described comprise hexadecatrienoic acid (HTA) (C16:3 ω-3), linoleic acid (C18:2 ω-6), alpha linolenic acid (C18:3 ω-3), gamma linolenic acid (C18:3 ω-6), stearidonic acid (C18;4 ω-4), eicosadienoic acid (C20:2 ω-6), eicosatrienoic acid (ETE) (C20:3 ω-3), dihonom-gamma-linolenic acid (C20:3 ω-6), mead acid (C20:3 ω-9), arachidonic acid (C20:4 ω-6), eicosapentaenoic acid (EPA) (C20:5 ω-3, C20:5 ω-6), heneicosapentaenoic acid (HPA) (C21:5 ω-3), docosatetraenoic acid (C22:4 docosatetraenoic acid-6), DPA (C22:5 docosatetraenoic acid-3), Docasahexaenoic acid (DHA) (C22:6 Docasahexaenoic acid (DHA)-3), Tetracosapentaenoic acid (C24:5 ω-3) and Tetracosahexaenoic acid (Nisinic acid) (C24:6 Tetracosahexaenoic acid (Nisinic acid) (C24:6 ω-3), and any desired combinations thereof.

In some embodiments, PUFA are provided in connection with culture medium methods and systems herein described in a concentration of 100 ng/ml to 1 mg/ml. In some embodiments, PUFA can be provided in connection with culture medium methods and systems herein described such as linoleic acid are provided in a concentration of 50-75 μg/ml, PUFA such as alpha linolenic acid are provided in a concentration of 50-100 μg/ml, PUFA such as Eicosapentaenoic acid (EPA) are provided in a concentration of 10-75 μg/ml, and PUFA such as Docasahexaenoic acid (DHA) are provided in a concentration of 10-25 μg/ml.

In some embodiments, the lipids comprised in the lipid loading medium are SFA provided in a concentration at which the SFA alone are toxic to naturally existing fish cells and in particular at a concentration of at least 10 μg/ml or higher, more particularly, at a concentration from 10 μg/ml to 1000 μg/ml but will be non-toxic when combined with nervonic acid.

Exemplary SFA that can be used in connection with culture medium methods and systems herein described comprise capric acid (C10:0), undecylic acid (C11:0), lauric acid (C12:0), tridecylic acid (C13:0), myristic acid (C14:0), pentadecylic acid (C15:0), palmitic acid (C16:0), margaric acid (C17:0), stearic acid (C18:0), nonadecylic acid (C19:0), arachidic acid (C20:0), heneicosylic acid (C21:0), behenic acid (C22:0), tricosylic acid (C23:0), lignoceric acid (C24:0) and any desired combinations thereof.

In some embodiments of culture medium methods and systems herein described, SFA can be provided in a concentration of 100 ng/ml to 1 mg/ml. In some embodiments, SFA such as lauric acid, myristic acid, and stearic acid are preferably provided in a concentration of 25-75 μg/ml and SFA such as palmitic acid are provided in a concentration of 25-50 μg/ml.

In some embodiments, the culture medium methods and systems herein described can be used to achieve lipid loading, increased viability and/or cell differentiation of one or more cell types of an aquatic animal, performed in the presence of one or more lipids in amounts of at least 10 μg/ml such as any one of the lipids herein described alone or in any combination.

In some embodiments, the culture medium methods and systems herein described can be used to achieve lipid loading, increased viability and/or cell differentiation of one or more cell types of a terrestrial animal, performed in the presence of one or more lipids in amounts of at least 10 μg/ml such as any one of the lipids herein described alone or in any combination.

In particular, in some embodiments, the culture medium methods and systems herein described can comprise differentiation and lipid loading media that simultaneously serve to differentiate cells and to control the lipid contend of the cells.

In some embodiments, the culture medium methods and systems herein described can comprise a cell culture medium comprising one or more fatty acids at a concentration from 25 μg/ml to 1000 μg/ml, and preferably from 25 μg/ml to 100 μg/ml.

In some embodiments, the culture medium methods and systems herein described can comprise a cell culture medium comprising a nervonic acid at a concentration from 10 μg/ml to 1000 μg/ml, and preferably from 10 μg/ml to 100 μg/ml.

In some embodiments, the culture medium methods and systems herein described can comprise a cell culture medium comprising a monounsaturated fatty acid at a concentration from 10 μg/ml to 1000 μg/ml, and preferably from 10 μg/ml to 100 μg/ml.

In some embodiments, the culture medium methods and systems herein described can comprise a cell culture medium comprising linoleic acid, alpha linolenic acid, vaccenic acid, and palmitoleic acid each at a concentration from 10 μg/ml to 50 μg/ml.

In some embodiments, the culture medium methods and systems herein described can comprise a cell culture medium comprising linoleic acid, alpha linolenic acid, vaccenic acid, and palmitoleic acid each at a concentration of less than 10 μg/ml.

In some embodiments, the culture medium methods and systems herein described can comprise a cell culture medium comprising omega-s polyunsaturated fatty acids at a concentration from 10 μg/ml to 50 μg/ml.

In some embodiments, the culture medium methods and systems herein described can comprise a cell culture medium comprising a basal media and serum at a concentration between 4-10%.

In some embodiments, the method and system herein described can be performed to increase viability of cells in a cellular biomass of an aquatic animal by culturing the cellular biomass in presence of the toxic fatty acids in presence of an effective amount of nervonic acid. In these embodiments, the nervonic acid is typically provided in a concentration between 10-1000 μg/ml, preferably between 10-100 μg/ml, and preferably at 50-75 μg/ml.

In some embodiments, the culture media herein described are configured to provide a controllable set of lipids to be uploaded in a cell of an aquatic animal in the sense of the disclosure.

In some embodiments, the culture media herein described are configured to provide a controllable set of lipids to be uploaded in a cell of a terrestrial animal.

In some embodiments, the cells are cultured in a medium in the presence of serum. The term “serum” as used herein refers to the liquid fraction of whole blood that is collected after the blood is allowed to clot. The clot can be removed by, for example, centrifugation, and the resulting supernatant is designated as serum. The serum can be provided at a concentration from 0% to 4%, or higher if desired. Serums that are suitable for culturing cells from aquatic animals are well-known and include bovine serum, such as fetal bovine serum (FBS).

Similar serums are well-known for being suitable for culturing cells from terrestrial animals.

In some embodiments, the cells are cultured in a medium that is serum free. In some embodiments, the cells described herein are cultured in a differentiation medium and/or the lipid loading medium containing no serum.

In some embodiments, the culture medium methods and systems herein described can comprise a cell culture medium used for viability and proliferation of cells from aquatic animals in accordance with embodiments of the present disclosure and typically comprising basal media and optionally 4-10% serum-dependent on the species from which the cells originate.

In some embodiments, the culture medium methods and systems herein described can comprise a cell culture medium used for viability and proliferation of cells from terrestrial animals in accordance with embodiments of the present disclosure and typically comprising basal media and optionally 4-10% serum-dependent on the species from which the cells originate.

In some embodiments, a lipid loading media used for lipid uploading into cells herein described, typically comprises a basal media with 0-4% serum in combination with one or more lipids to be uploaded into the cells. In some embodiments, the media contains desired lipids at a concentration of at least 10 μg/ml and cells are cultured in the media for about 6 or 7 days. If desired, the media can contain lower concentrations of lipids (for example 1-2 μg/ml of each desired lipid) and cells are cultured in the media for a period of time to achieve the desired degree of lipid loading, which, in embodiments, is longer than 6 or 7 days. Typically, the lipids in the media comprise fatty acids such as a single fatty acid or fatty acid mixtures comprising saturated, monounsaturated and/or polyunsaturated fatty acids as will be understood by a skilled person.

This disclosure also relates to a differentiation media used for cell differentiation according to embodiments of the present disclosure that typically comprises basal media with 0-4% serum. In particular embodiments, the differentiation media is not supplemented with dexamethasone, biotin, T3, pantothenate, IBMX, and insulin, as these are typically used in media to load desired lipids into undifferentiated cells, such as myoblasts and preadipocytes. Compared to the previous protocols, which showed limited lipid loading over the 14-day period of the differentiation protocol, the differentiation and lipid loading media without dexamethasone, biotin, T3, pantothenate, IBMX, and insulin are able to produce fish cells that morphologically change from elongated to rounded with increased cell size to store large lipid droplets.

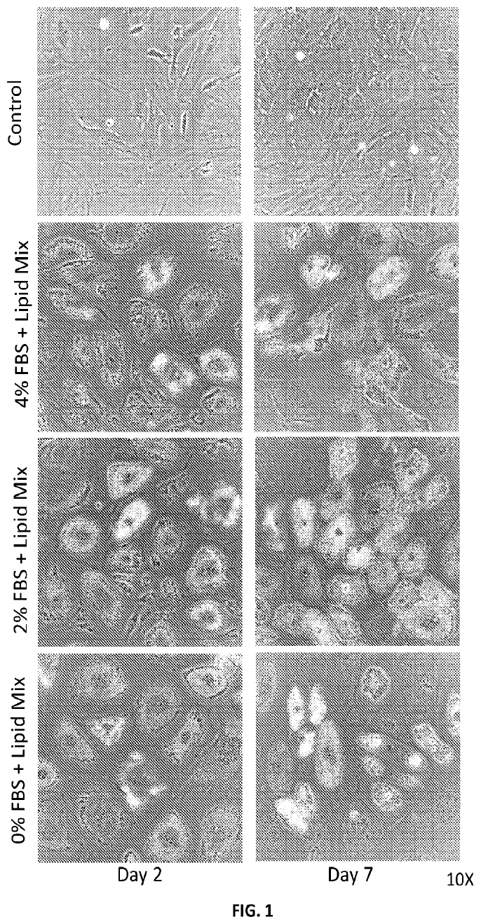

For example, as shown in Example 1, a differentiation of preadipocytes into adipocytes in media with reduced or no serum in the presence of a complex fatty acid mixture and in the absence of dexamethasone, biotin, T3, pantothenate, IBMX, and insulin resulted in uptake and storage of the fatty acids in cultured carp preadipocytes (Example 1 and FIG. 1). In particular, the carp preadipocytes in the presence of fatty acids achieved morphological changes by two days that were maintained throughout the seven-day culture period.

In embodiments, cells used in methods and systems of the disclosure, can be primary cells that are isolated from a desired aquatic species or cell lines that are derived from a desired aquatic species. In embodiments, cells used in methods and systems of the disclosure, can be primary cells that are isolated from a desired terrestrial animal species or cell lines that are derived from a desired terrestrial animal species. Preferably, the cells are not genetically modified. In some embodiments, the aquatic animal cells can be harvested from any desired aquatic animal, in particular, from any fish, mollusk and crustacean, using any suitable methods. Similarly, in some embodiments, the terrestrial animal cells can be harvested from any desired terrestrial animal, such as any mammal, bird, reptile, amphibian, or insect, using any suitable methods. A number of methods are well-known in the art, such as enzymatic and mechanical dissociation of tissue, as will be understood by a person skilled in the art. For example, detailed information on how to isolate preadipocytes from fish can be found in Vegusdal et al 2003, “An in vitro method for studying the proliferation and differentiation of Atlantic salmon preadipocytes,” as will be understood by a person skilled in the art. Information on the isolation of other cell types from fish, or any cell type from any other animal, will be readily available to a person of ordinary skill in the art.

The harvested cells used herein can be grown in an adherent 2D tissue culture or in 3D cell suspension culture as will be understood by a person skilled in the art.

In embodiments, herein described, the harvested cells from an aquatic animal used herein are then cultured in a media for proliferation, differentiation, and/or lipid loading as will be understood by a person of ordinary skill in the art.

In embodiments, herein described, the harvested cells from a terrestrial animal used herein are then cultured in a media for proliferation, differentiation, and/or lipid loading as will be understood by a person of ordinary skill in the art.

Embodiments of the present disclosure also include cells obtained with any one of the methods and systems herein described.

In particular, the cells obtained following culture in media for lipid loading, as described herein, comprise an increased amount of desired lipids compared to cells directly harvested from the corresponding living species. The amount of desired lipids in the cells cultured in the lipid loading media is a function of culture time, the concentration of the lipids that are added to the media, and cell viability and proliferation. As described herein, the inclusion of nervonic acid in the media enhances uptake of lipids for aquatic animals. Typically, the cells will contain at least about twice the amount of the desired lipids, measured as g/g total fat, in comparison to cells of the corresponding aquatic animal species. Typically, the cells will contain at least about twice the amount of the desired lipids, measured as g/g total fat, in comparison to cells of the corresponding animal species. For example, myoblasts, myocytes, preadipocytes, adipocytes or fibroblasts from a desired fish species that are cultured in a lipid loading media that contains omega-3 fatty acids (e.g., EPA, DPA and/or DHA) in accordance with this disclosure, can contain about two times, about three times, about four times, about five times, about 10 times or about 100 times the amount of omega-3 fatty acids in comparison to the corresponding cells or meat from a wild caught fish of the same species.

In embodiments, the cells can contain a percentage of the desired lipids on a g/g total fat basis that is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 percentage points greater than the percentage naturally found in a wild caught or farm-raised animal of the corresponding species. (For clarity, a number of percentage points greater means that the percentage of total fat made up by the desired lipids is greater by the number of percentage points.) As an example, if the percentage of total fat made up of the desired lipids in a wild-caught fish is 25% and the percentage of total fat made up of the desired lipids in cultured fish cells is 30%, the cultured cells contain 5 percentage points more of the desired lipids. As a further example, if the percentage of total fat made up of the desired lipids in a farm-raised steer, bull, cow, or heifer is 0% and the percentage of total fat made up of the desired lipids in cultured cattle cells is 5%, the cultured cells also contain 5 percentage points more of the desired lipids.

Further, in the case of food products intended to resemble fillets, cuts, organs, or other food products not consisting of a whole wild caught or farm-raised animal, the comparison is made between the cultured cells and the fillet, cut, organ, etc. of the animal, not the animal carcass as a whole.

Suitable methods for determining fatty acid content of cells, meat and other foods are well known in the art. For example, fatty acids can be extracted by a hydrolytic method. Fat is then extracted into ether and methylated to form fatty acid methyl esters (FAMEs). FAMEs can then be quantitatively analyzed by gas chromatography (GC), with the peaks indicating each quantified fatty acid. Conveniently, lipid extraction kits are commercially available to extract lipids from cells, biological fluids, tissues, and the like. Lipids that are extracted from fish or cells using such kits, or other suitable methods, can then be analyzed and quantified using any suitable methods, such as GC, HPL, mass spectrometry, and lipidomics. Lipids that are extracted from terrestrial animals or cells using such kits, or other suitable methods, can then be analyzed and quantified using any suitable methods, such as GC, HPL, mass spectrometry, and lipidomics.

Tables 2A and 2B below shows a list of representative fatty acid profiles of naturally occurring aquatic animals including fishes, crustaceans, and mollusks. Tables 2C and 2D below show a list of representative fatty acid profiles of naturally occurring terrestrial animals including poultry, game, and farm-raised livestock. Data is shown as fatty acid content in grams per 100 g fillet (g/g fillet). The highest represented fatty acids include 16:0 (palmitic acid), 18:1 (vaccenic or oleic acid), and omega-3 fatty acids (EPA, DHA, and DPA) for aquatic animals while omega-3 are near zero for terrestrial animals.

TABLE 2A
Representative fatty acid profiles of naturally occurring aquatic animals including fishes, crustaceans, and mollusks

| | | | | | | | Sea bass, | | | | Lobster, | Scallops, | Shrimp, |
|---|---|---|---|---|---|---|---|---|---|---|---|---|---|
| | | | | | | | baked or | | | | baked or | baked or | baked or |
| | | | | | | | broiled, | Tuna, | | | broiled, | broiled, | broiled, |
| Fatty | Flounder, | Halibut, | Herring, | Mackerel, | Pompano, | Salmon, | no added | fresh, | Squid, | Clams, | no added | no added | no added |
| acid | raw | raw | raw | raw | raw | raw | fat | raw | raw | raw | fat | fat | fat |
| 4:0 | 0.000 | 0.000 | 0.000 | 0.000 | 0.000 | 0.000 | 0.000 | 0.000 | 0.000 | 0.000 | 0.000 | 0.000 | 0.000 |
| 6:0 | 0.000 | 0.000 | 0.000 | 0.000 | 0.000 | 0.000 | 0.000 | 0.000 | 0.000 | 0.000 | 0.000 | 0.000 | 0.000 |
| 8:0 | 0.000 | 0.000 | 0.000 | 0.000 | 0.000 | 0.000 | 0.000 | 0.000 | 0.000 | 0.000 | 0.000 | 0.000 | 0.000 |
| 10:0 | 0.002 | 0.000 | 0.001 | 0.000 | 0.000 | 0.000 | 0.000 | 0.000 | 0.000 | 0.000 | 0.000 | 0.000 | 0.000 |
| 12:0 | 0.005 | 0.005 | 0.002 | 0.001 | 0.000 | 0.001 | 0.000 | 0.002 | 0.000 | 0.009 | 0.002 | 0.003 | 0.008 |
| 14:0 | 0.067 | 0.049 | 0.070 | 0.055 | 0.057 | 0.071 | 0.029 | 0.022 | 0.037 | 0.018 | 0.015 | 0.028 | 0.018 |
| 16:0 | 0.217 | 0.174 | 0.172 | 0.183 | 0.309 | 0.165 | 0.192 | 0.262 | 0.270 | 0.249 | 0.166 | 0.279 | 0.219 |
| 18:0 | 0.041 | 0.054 | 0.014 | 0.042 | 0.115 | 0.036 | 0.082 | 0.111 | 0.059 | 0.113 | 0.068 | 0.100 | 0.120 |
| 16:1 | 0.069 | 0.068 | 0.079 | 0.062 | 0.078 | 0.063 | 0.078 | 0.027 | 0.008 | 0.045 | 0.046 | 0.031 | 0.031 |
| 18:1 | 0.275 | 0.229 | 0.193 | 0.191 | 0.282 | 0.220 | 0.175 | 0.205 | 0.047 | 0.136 | 0.238 | 0.106 | 0.173 |
| 20:1 | 0.046 | 0.133 | 0.092 | 0.078 | 0.000 | 0.157 | 0.000 | 0.020 | 0.045 | 0.016 | 0.041 | 0.017 | 0.005 |
| 22:1 | 0.001 | 0.014 | 0.107 | 0.107 | 0.000 | 0.016 | 0.000 | 0.002 | 0.007 | 0.000 | 0.002 | 0.003 | 0.000 |
| 18:2 | 0.035 | 0.034 | 0.017 | 0.018 | 0.017 | 0.028 | 0.014 | 0.047 | 0.002 | 0.095 | 0.050 | 0.028 | 0.146 |
| 18:3 | 0.013 | 0.011 | 0.013 | 0.012 | 0.000 | 0.016 | 0.000 | 0.005 | 0.004 | 0.034 | 0.065 | 0.011 | 0.009 |
| 18:4 | 0.009 | 0.007 | 0.027 | 0.022 | 0.000 | 0.025 | 0.027 | 0.000 | 0.006 | 0.002 | 0.002 | 0.014 | 0.000 |
| 20:4 | 0.012 | 0.014 | 0.008 | 0.014 | 0.034 | 0.005 | 0.000 | 0.037 | 0.009 | 0.025 | 0.039 | 0.017 | 0.053 |
| 20:5 | 0.105 | 0.066 | 0.090 | 0.075 | 0.024 | 0.064 | 0.096 | 0.030 | 0.150 | 0.097 | 0.154 | 0.145 | 0.104 |
| n-3 | | | | | | | | | | | | | |
| 22:5 | 0.022 | 0.016 | 0.007 | 0.018 | 0.028 | 0.016 | 0.045 | 0.010 | 0.004 | 0.016 | 0.009 | 0.011 | 0.009 |
| n-3 | | | | | | | | | | | | | |
| 22:6 | 0.083 | 0.128 | 0.110 | 0.121 | 0.055 | 0.116 | 0.259 | 0.218 | 0.351 | 0.145 | 0.102 | 0.209 | 0.108 |
| n-3 | | | | | | | | | | | | | |
| Total | 0.210 | 0.210 | 0.207 | 0.214 | 0.107 | 0.196 | 0.401 | 0.257 | 0.505 | 0.258 | 0.265 | 0.365 | 0.220 |
| n-3 | | | | | | | | | | | | | |

TABLE 2B
Representative fatty acid profiles of naturally occurring
aquatic animals including fishes, crustaceans, and mollusks

| | | | | | Eel, |
|---|---|---|---|---|---|
| | Seriola | Coryphaena | Lutjanus | Thunnus | mixed |
| Fatty | lalandi, | hippurus, | campechanus, | orientalis, | species, |
| acid | raw | raw | raw | raw | raw |
| 4:0 | 0.000 | 0.000 | 0.000 | 0.000 | 0.000 |
| 6:0 | 0.000 | 0.000 | 0.000 | 0.000 | 0.000 |
| 8:0 | 0.000 | 0.000 | 0.000 | 0.000 | 0.000 |
| 10:0 | 0.000 | 0.000 | 0.000 | 0.000 | 0.000 |
| 12:0 | 1.29 | 0.001 | 0 | 0 | 0 |
| 14:0 | 1.052 | 0.014 | 0.043 | 0.139 | 0.58 |
| 15:0 | 0.0185 | 0 | 0 | 0 | 0 |
| 16:0 | 6.974 | 0.12 | 0.176 | 0.81 | 1.585 |
| 18:0 | 2.485 | 0.046 | 0.0755 | 0.307 | 0.191 |
| 19:0 | 0.79 | 0 | 0 | 0 | 0 |
| 20:0 | 0.0085 | 0 | 0 | 0 | 0 |
| 21:0 | 0.009 | 0 | 0 | 0 | 0 |
| 22:0 | 0.027 | 0 | 0 | 0 | 0 |
| 24:0 | 0.011 | 0 | 0 | 0 | 0 |
| 14:1 | 0.0055 | 10 | 0 | 0 | 0 |
| 15:1 | 0.004 | 0 | 0 | 0 | 0 |
| 16:1 | 5.09 | 0.023 | 0.055 | 0.162 | 1.255 |
| 18:1 | 2.18 | 0 | 0.12 | 0 | 0 |
| vaccenic | | | | | |
| acid | | | | | |
| 18:1 | 6.276 | 0.086 | 0.165 | 0.924 | 2.772 |
| oleic | | | | | |
| acid | | | | | |
| 20:1 | 0.1435 | 0.003 | 0.021 | 0.277 | 3 |
| 22:1 | 0.032 | 0.009 | 0 | 0.237 | 0 |
| 18:2 | 3.42 | 0 | 0.0245 | 0.053 | 0.196 |
| 18:3 | 0.926 | 0.005 | 0.004 | 0 | 0.432 |
| 18:4 | 0.53 | 0.005 | 0.009 | 0.039 | 0 |
| 20:4 | 0.626 | 0.002 | 0.0445 | 0.043 | 0.095 |
| 20:5 n-3 | 1.462 | 0.02 | 0.05 | 0.28 | 0.08 |
| 22:5 n-3 | 0.525 | 0.009 | 0.045 | 0.12 | 0.07 |
| 22:6 n-3 | 2.675 | 0.088 | 0.265 | 0.89 | 0.06 |
| Total n-3 | 4.662 | 0.117 | 0.36 | 1.29 | 0.21 |

TABLE 2C
Representative fatty acid profiles of naturally occurring terrestrial animals

| | Duck, wild, | | Quail, | | | Beef, | | | |
|---|---|---|---|---|---|---|---|---|---|
| | meat and | Lamb, | meat | Rabbit, | | grass-fed, | | | Chicken, |
| Fatty | skin, | ground, | only, | wild, | Goat, | ground, | Horse, | Deer, | ground, |
| acid | raw | raw | raw | raw | raw | raw | raw | raw | raw |
| 4:0 | 0.0 | 0.0 | n/a | n/a | n/a | 0.0 | n/a | 0.0 | 0.0 |
| 6:0 | 0.0 | 0.0 | n/a | n/a | n/a | 0.0 | n/a | 0.0 | 0.0 |
| 8:0 | 0.0 | 0.0 | n/a | n/a | n/a | 0.001 | n/a | 0.0 | 0.0 |
| 10:0 | 0.0 | 0.06 | 0.0 | n/a | 0.0 | 0.005 | n/a | 0.0 | 0.0 |
| 12:0 | 0.04 | 0.1 | 0.03 | n/a | 0.0 | 0.009 | 0.01 | 0.0 | 0.0 |
| 14:0 | 0.09 | 0.93 | 0.03 | 0.06 | 0.03 | 0.345 | 0.17 | 0.02 | 0.041 |
| 16:0 | 3.48 | 5.13 | 0.78 | 0.52 | 0.33 | 2.75 | 1.08 | 0.41 | 1.79 |
| 18:0 | 1.27 | 3.22 | 0.46 | 0.11 | 0.33 | 1.98 | 0.16 | 0.51 | 0.456 |
| 16:1 | 0.61 | 0.68 | 0.24 | 0.08 | 0.04 | 0.347 | 0.31 | 0.03 | 0.536 |
| 18:1 | 6.06 | 8.62 | 1.02 | 0.54 | 0.94 | 4.36 | 1.3 | 0.63 | 3.04 |
| 20:1 | 0.13 | 0.0 | 0.01 | n/a | n/a | 0.015 | n/a | 0.0 | 0.025 |
| 22:1 | 0 | 0.0 | 0.0 | n/a | n/a | 0.0 | n/a | 0.0 | 0.0 |
| 18:2 | 1.85 | 1.36 | 0.93 | 0.36 | 0.1 | 0.426 | 0.29 | 0.31 | 1.32 |
| 18:3 | 0.17 | 0.42 | 0.02 | 0.09 | 0.02 | 0.071 | 0.36 | 0.07 | 0.071 |
| 18:4 | 0.0 | 0.0 | n/a | n/a | n/a | 0.002 | n/a | 0.0 | 0.0 |
| 20:4 | 0.0 | 0.08 | 0.12 | n/a | 0.06 | 0.012 | n/a | 0.1 | 0.074 |
| 20:5 | 0.0 | 0.0 | 0.01 | n/a | n/a | 0.001 | n/a | 0.0 | 0.008 |
| n-3 | | | | | | | | | |
| 22:5 | 0.0 | 0.0 | 0.02 | n/a | n/a | 0.016 | n/a | 0.0 | 0.008 |
| n-3 | | | | | | | | | |
| 22:6 | 0.0 | 0.0 | 0.0 | n/a | n/a | 0.0 | n/a | 0.0 | 0.023 |
| n-3 | | | | | | | | | |
| Total | 0.0 | 0.0 | 0.03 | n/a | n/a | 0.016 | n/a | n/a | 0.039 |
| n-3 | | | | | | | | | |

TABLE 2D
Representative fatty acid profiles of naturally occurring terrestrial animals

| | | | Turkey, | | | | | | |
|---|---|---|---|---|---|---|---|---|---|
| | | | whole, | Pheasant, | | | | | |
| | Wild | Water | meat | meat | | | | | |
| Fatty | boar, | Buffalo, | only, | only, | Antelope, | Caribou, | Elk, | Moose, | Squirrel, |
| acid | raw | raw | raw | raw | raw | raw | raw | raw | raw |
| 4:0 | n/a | n/a | 0.0 | n/a | n/a | n/a | n/a | n/a | n/a |
| 6:0 | n/a | n/a | 0.0 | n/a | n/a | n/a | n/a | n/a | n/a |
| 8:0 | n/a | n/a | 0.0 | n/a | n/a | n/a | n/a | n/a | n/a |
| 10:0 | n/a | n/a | 0.003 | 0.0 | 0.0 | n/a | 0.0 | n/a | n/a |
| 12:0 | n/a | n/a | 0.006 | 0.05 | 0.0 | n/a | 0.0 | n/a | n/a |
| 14:0 | 0.04 | 0.01 | 0.013 | 0.03 | 0.02 | 0.09 | 0.03 | 0.0 | 0.02 |
| 16:0 | 0.58 | 0.25 | 0.29 | 0.75 | 0.34 | 0.68 | 0.35 | 0.09 | 0.3 |
| 18:0 | 0.33 | 0.19 | 0.135 | 0.36 | 0.37 | 0.45 | 0.14 | 0.13 | 0.05 |
| 16:1 | 0.17 | 0.03 | 0.043 | 0.2 | 0.01 | 0.08 | 0.13 | 0.01 | 0.1 |
| 18:1 | 1.13 | 0.37 | 0.422 | 0.96 | 0.48 | 0.93 | 0.23 | 0.14 | 1.08 |
| 20:1 | n/a | n/a | 0.006 | 0.0 | 0.0 | n/a | 0.0 | n/a | n/a |
| 22:1 | n/a | n/a | 0.0 | 0.0 | n/a | n/a | n/a | n/a | n/a |
| 18:2 | 0.38 | 0.16 | 0.339 | 0.54 | 0.25 | 0.23 | 0.17 | 0.14 | 0.92 |
| 18:3 | 0.02 | 0.04 | 0.017 | 0.08 | 0.07 | 0.03 | 0.04 | 0.03 | 0.02 |
| 18:4 | n/a | n/a | 0.0 | n/a | n/a | n/a | n/a | n/a | n/a |
| 20:4 | 0.08 | 0.07 | 0.031 | 0.0 | 0.12 | 0.14 | 0.09 | 0.07 | n/a |
| 20:5 | n/a | n/a | 0.0 | 0.0 | n/a | n/a | n/a | n/a | n/a |
| n-3 | | | | | | | | | |
| 22:5 | n/a | n/a | 0.003 | 0.0 | n/a | 0.02 | n/a | n/a | n/a |
| n-3 | | | | | | | | | |
| 22:6 | n/a | n/a | 0.002 | 0.0 | n/a | 0.05 | n/a | n/a | n/a |
| n-3 | | | | | | | | | |
| Total | n/a | n/a | 0.005 | 0.0 | n/a | 0.07 | n/a | n/a | n/a |
| n-3 | | | | | | | | | |

In some embodiments, the methods and system herein describe increased fatty acid content in a cell of an aquatic animal, by culturing the cell in a culture medium comprising the individual and/or mixture of fatty acids.

In some embodiments, the methods and system herein describe increased fatty acid content in a cell of a terrestrial animal, by culturing the cell in a culture medium comprising the individual and/or mixture of fatty acids.

In some embodiments, a cell of an aquatic animal obtained using the methods and system herein described have one or more fatty acid contents higher than the fatty acid contents of naturally occurring aquatic animals listed in Tables 2A and 2B.

In some embodiments, a cell of a terrestrial animal obtained using the methods and system herein described have one or more fatty acid contents higher than the fatty acid contents of naturally occurring terrestrial animals listed in Tables 2C and 2D.

For example, a cell of flounder obtained using the methods and system herein described can have a palmitic acid (C16:0) content higher than 0.217 g/100 g fillet, a cell of halibut herein obtained can have a palmitic acid (C16:0) content higher than 0.174 g/100 g fillet, a cell of herring herein obtained can have a palmitic acid (C16:0) content higher than 0.172 g/100 g fillet, and a cell of mackerel herein obtained can have a palmitic acid (C16:0) content higher than 0.183 g/100 g fillet (see Tables 2A and 2B).

Similarly, a cell of flounder herein obtained can have a vaccenic or oleic acid (C18:1) content higher than 0.275 g/100 g fillet, a cell of halibut herein obtained can have a vaccenic or oleic acid (C18:1) content higher than 0.229 g/100 g fillet, a cell of herring herein obtained can have a vaccenic or oleic acid (C18:1) content higher than 0.193 g/100 g fillet, and a cell of mackerel herein obtained can have a vaccenic or oleic acid (C18:1) content higher than 0.191 g/100 g fillet (see Tables 2A and 2B).

Similarly, a cell of flounder herein obtained can have an Eicosapentaenoic acid (EPA) (C20:5 n-3) content higher than 0.105 g/100 g fillet, a cell of halibut herein obtained can have an EPA content higher than 0.066 g/100 g fillet, a cell of herring herein obtained can have an EPA content higher than 0.090 g/100 g fillet, and a cell of mackerel herein obtained can have an EPA content higher than 0.075 g/100 g fillet (see Tables 2A and 2B).

Similarly, a cell of flounder herein obtained can have a DPA (C22:5 n-3) content higher than 0.022 g/100 g fillet, a cell of halibut herein obtained can have a DPA content higher than 0.016 g/100 g fillet, a cell of herring herein obtained can have a DPA content higher than 0.007 g/100 g fillet, and a cell of mackerel herein obtained can have a DPA content higher than 0.018 g/100 g fillet (see Tables 2A and 2B).

Similarly, a cell of flounder herein obtained can have a DHA (C22:6 n-3) content higher than 0.083 g/100 g fillet, a cell of halibut herein obtained can have a DHA content higher than 0.128 g/100 g fillet, a cell of herring herein obtained can have a DHA content higher than 0.110 g/100 g fillet, and a cell of mackerel herein obtained can have a DHA content higher than 0.121 g/100 g fillet (see Tables 2A and 2B).

In some embodiments, a cell of an aquatic animal herein obtained can have a total omega-3 polyunsaturated fatty acid (total n-3) content higher than that of naturally occurring aquatic animals. For example, a cell of flounder or halibut herein obtained can have a total omega-3 polyunsaturated fatty acid content higher than 0.210 g/100 g fillet, a cell of herring herein obtained can have a total omega-3 polyunsaturated fatty acid content higher than 0.207 g/100 g fillet, and a cell of mackerel herein obtained can have a total omega-3 polyunsaturated fatty acid content higher than 0.214 g/100 g fillet (see Tables 2A and 2B).

In some embodiments, a cell of a terrestrial animal herein obtained can have a total omega-3 polyunsaturated fatty acid (total n-3) content higher than that of naturally occurring terrestrial animal.

It is to be understood that the described embodiments of cells of flounder, halibut, herring, and mackerel with certain lipid contents are exemplary in nature and not limiting, and that this disclosure encompasses corresponding or similar embodiments of cells from other species.

In all naturally occurring species listed in Tables 2A and 2B, a significant fraction of fatty acids was saturated fat with at least 15% content, while lipid loaded cells herein obtained can be loaded with only unsaturated or monounsaturated fat. The maximum concentration of each fatty acid is limited to ˜30% in almost all naturally occurring aquatic animals of Tables 2A and 2B, whereas cells herein obtained can have more than 30% of a single fatty acid as the fat content.

In some of these embodiments, culture media, methods and system herein described to culture a preadipocyte cell.

In particular, in embodiments herein described in connection with culture of adipocytes, the culture medium can comprise basal medium supplemented with 25 to 1000 μg/ml of a lipid, and 0 to 4% serum, in an effective amount to control lipid content and increase viability, differentiation and/or lipid uptake of the preadipocyte cell.

In some embodiments, the lipids comprised in the culture medium for preadipocyte of the instant disclosure comprise one or more MUFA, one or more PUFA one or more SFA, and/or one or more sterols alone or in any combinations. In preferred embodiments, the adipocyte of the instant disclosure comprises a controlled amount of PUFA, more preferably comprising omega-3 fatty acid and/or a fat-soluble vitamin.

In exemplary embodiments herein described in connection with preadipocyte, a method to culture a preadipocyte cell of an aquatic animal, comprises culturing the preadipocyte cell in a preadipocyte culture medium according to the present disclosure comprises one or more fatty acids in an effective concentration to result in uptake of the lipids by the preadipocyte cell. Typically, in these embodiments, the lipid can be provided in a concentration between 25 μg/ml up to 1000 μg/ml, preferably between 25 μg/ml up to 100 μg/ml. In particular, in some of those embodiments, the selected fatty acid concentration is able to induce a rounded morphology with lipid droplets observed in fish cells without showing a reduced cell confluency indicating potential toxicity (see Example 2).

Similarly, a preadipocyte cell of a terrestrial animal can be cultured by similar methods and the use of a similar medium.

In some embodiments, preadipocyte cells of an aquatic animal obtainable with culture media, methods and systems of the present disclosure comprise a lipid in an amount from 0.1% to 90%. In preferred embodiments, the fatty acids of the preadipocyte cell herein described contains about 50% SFA, 25% PUFA, preferably including Omega 3, and 25% MUFA.

In some embodiments, the culture medium, methods and the system of the disclosure can be used to increase lipid content and/or cell viability in a cell of an aquatic animal, by culturing the cell of the aquatic animal cells in a culture medium comprising the lipid and an effective amount of nervonic acid.

In particular, in preferred embodiments, methods and systems and related compositions and in particular culture media can be used to control lipid content and/or increase viability of myoblasts and/or fibroblasts cells of an aquatic animal.

In particular, in preferred embodiments, methods and systems and related compositions and in particular culture media can be used to control lipid content and/or increase viability of myoblasts and/or fibroblasts cells of a terrestrial animal.

In some of those embodiments, nervonic acid can be combined with saturated fatty acids or polyunsaturated fatty acids, such as DHA and EPA, which when used individually show low lipid accumulation and some level of toxicity, but show high cell numbers and high lipid accumulation when used in combination with nervonic acid (see Examples 6-7).

Accordingly, in some embodiments, nervonic acid when in combination with other fatty acids, can cause increased lipid uptake and lipid accumulation. Accordingly, the use of nervonic acid can enhance or enable lipid loading of fatty acids, including those that have some toxicity to cultured aquatic animal cells, as well as contribute additional nutritional qualities to aquatic animal cell-cultured products, such as fish products, and seafood products as well as additional products identifiable by a skilled person.

Accordingly, in some embodiments, nervonic acid when in combination with other fatty acids, can cause increased lipid uptake and lipid accumulation. Accordingly, the use of nervonic acid can enhance or enable lipid loading of fatty acids, as well as contribute additional nutritional qualities to terrestrial animal cell-cultured products.

In some embodiments, nervonic acid is provided in a concentration between 10-1000 μg/ml, preferably between 10-100 μg/ml, and preferably at 50 μg/ml.

In some embodiments, the culture medium, methods, systems and compositions comprising nervonic acid including culture media, can provide a myoblast cell and/or a fibroblast cell of an aquatic animal comprising desired fatty acids (e.g., omega-3 fatty acids) in an amount of at least about 1% relative to total fat. In some embodiments, the culture medium, methods, systems and compositions comprising nervonic acid including culture media, can provide a myoblast cell and/or a fibroblast cell of a terrestrial animal comprising desired fatty acids (e.g., omega-3 fatty acids) in an amount of at least about 1% relative to total fat. In some embodiments, the culture medium, methods, systems and compositions comprising nervonic acid including culture media, can provide a myoblast cell and/or a fibroblast cell of an aquatic animal comprising desired fatty acids (e.g., omega-3 fatty acids) in an amount of at least about 1% relative to total fat. For example, myoblasts, myocytes, preadipocytes, adipocytes or fibroblasts from a desired fish species that are cultured in a lipid loading media that contains desired fatty acids and an effective amount of nervonic acid in accordance with this disclosure, can contain about two times, about three times, about four times, about five times, about 10 times or about 100 times the amount of the desired fatty acids in comparison to the corresponding cells or meat from a wild caught fish of the same species.

In some embodiments, the culture medium, methods, systems and compositions comprising nervonic acid including culture media, can provide a myoblast cell and/or a fibroblast cell of a terrestrial animal comprising desired fatty acids (e.g., omega-3 fatty acids) in an amount of at least about 1% relative to total fat.

In some embodiments, methods and systems of the disclosure provide a fibroblast cell of an aquatic animal and related biomass comprising said cell, the fibroblast cell comprising a lipid in an amount of at least of at least 1%-90%. In some embodiments, methods and systems of the disclosure provide a fibroblast cell of a terrestrial animal and related biomass comprising said cell, the fibroblast cell comprising a lipid in an amount of at least of at least 1%-90%.

In some embodiments, methods and systems of the disclosure provide a myoblast cell of an aquatic animal and related biomass comprising said cell, the myoblast cell comprising a lipid in an amount of at least of at least 1%. In some embodiments, methods and systems of the disclosure provide a myoblast cell of a terrestrial animal and related biomass comprising said cell, the myoblast cell comprising a lipid in an amount of at least of at least 1%.

In some embodiments, nervonic acid can further be used to increase viability of myoblasts, fibroblasts and additional cells of an aquatic animal as will be understood by a skilled person upon reading of the present disclosure.

Accordingly, in some embodiments, the method and system herein described can increase viability of cells in a cellular biomass of an aquatic animal by culturing the cellular biomass in presence of an effective amount of nervonic acid. In some embodiments, the culture media, method and systems herein described increase polyunsaturated fatty acid content in a cell of an aquatic animal, by culturing the cell in a culture medium comprising the polyunsaturated fatty acids and an effective amount of nervonic acid. In some embodiments, the culture media, method and systems herein described increase polyunsaturated fatty acid content in a cell of a terrestrial animal, by culturing the cell in a culture medium comprising the polyunsaturated fatty acids and an effective amount of nervonic acid.

In some embodiments, the concentration of monounsaturated fatty acids is increased by at least about 1% to about 300% or more in comparison to the amount present in wild caught animal (e.g., fish) of the same species. For example, the concentrations of monounsaturated fatty acids such as palmitoleic acid, oleic acid, and vaccenic acid can be increased to include amounts above any one of the naturally occurring amounts such as the ones indicated in Tables 2A and 2B. In some embodiments, the concentration of monounsaturated fatty acids is increased by at least about 1% to about 300% or more in comparison to the amount present in wild caught or farm-raised terrestrial animal of the same species, such as the ones indicated in Tables 2C and 2D.

In some embodiments, the concentration of polyunsaturated fatty acids is increased by at least about 1% to about 300% or more in comparison to the amount present in wild caught animal (e.g., fish) of the same species. For example, the concentration of polyunsaturated linolenic acid and omega-3 polyunsaturated fatty acids can be increased to include amounts of above any one of the naturally occurring amounts such as the ones indicated in Tables 2A and 2B. In some embodiments, the concentration of polyunsaturated fatty acids is increased by at least about 1% to about 300% or more in comparison to the amount present in wild caught or farm-raised terrestrial animal of the same species, such as the ones indicated in Tables 2C and 2D.

In some embodiments, the concentration of saturated fatty acids is increased by at least about 1% to about 300% or more in comparison to the amount present in wild caught animal (e.g., fish) of the same species. For example, the concentration of lauric acid, myristic acid, palmitic acid and/or stearic acid can be increased above any one of the naturally occurring amounts such as the ones indicated in Tables 2A and 2B. In some embodiments, the concentration of saturated fatty acids is increased by at least about 1% to about 300% or more in comparison to the amount present in wild caught or farm-raised terrestrial animal of the same species, such as the ones indicated in Tables 2C and 2D.

In some embodiments, a culture media, method and system are described for uploading vaccenic acid in an aquatic animal cell. The method comprises culturing the aquatic animal cell in the presence of vaccenic acid for a time and under conditions resulting in uptake of vaccenic acid by the aquatic animal cell.

In some embodiments, a culture media, method and system are described for uploading vaccenic acid in a terrestrial animal cell. The method comprises culturing the terrestrial animal cell in the presence of vaccenic acid for a time and under conditions resulting in uptake of vaccenic acid by the aquatic animal cell.

The term “vaccenic acid” also is a term of art and refers to the compound known as C18:1 and exists as a trans-stereoisomer ((11E)-11-octadecenoid acid) and a cis-stereoisomer ((11Z)-11-octadecenoic acid). Vaccenic acid exists as a solid and is considered to be insoluble in water and relatively neutral. Vaccenic acid can be produced through the biohydrogenation of linoleic acid and α-linolenic acid by microorganisms in the rumen and is found naturally in foods such as dairy and ruminant meat products.

In some embodiments, the cells obtained herein comprise fish preadipocytes having increased concentration of omega-3 polyunsaturated fatty acids, such as DHA and EPA, with respect to the naturally occurring amount such as the ones indicated in Tables 2A and 2B.

In some embodiments, the cells obtained herein comprise fish myoblasts and/or fibroblasts having increased concentration of omega-3 polyunsaturated fatty acids such as DHA and EPA with respect to the naturally occurring amount such as the ones indicated in Tables 2A and 2B.

In some embodiments, the concentration of omega-3 polyunsaturated fatty acids is increased by at least about 1% to about 300% or more in comparison to the amount present in wild caught animal (e.g., fish) of the same species such as the amounts indicated in Tables 2A and 2B.

In some embodiments, the cells obtained herein comprise terrestrial animal preadipocytes having increased concentration of omega-3 polyunsaturated fatty acids, such as DHA and EPA, with respect to the naturally occurring amount such as the ones indicated in Tables 2C and 2D.

In some embodiments, the cells obtained herein comprise terrestrial animal myoblasts and/or fibroblasts having increased concentration of omega-3 polyunsaturated fatty acids such as DHA and EPA with respect to the naturally occurring amount such as the ones indicated in Tables 2C and 2D.

In some embodiments, the concentration of omega-3 polyunsaturated fatty acids is increased by at least about 1% to about 300% or more in comparison to the amount present in wild caught or farm-raised terrestrial animal of the same species such as the amounts indicated in Tables 2C and 2D.

In some embodiments, the terrestrial animal cells obtained herein comprise myoblasts, fibroblasts, and/or preadipocytes having an increased concentration of omega-3 polyunsaturated fatty acids in comparison to the amount present in the corresponding cell type in wild caught or farm raised terrestrial animals of the same species. The increased concentration of omega-3 polyunsaturated fatty acids can be by at least about 1 percentage point or more, such as 2 percentage points, 3 percentage points, 4 percentage points, 5 percentage points, 6 percentage points, 7 percentage points, 8 percentage points, 9 percentage points, 10 percentage points, 11 percentage points, 12 percentage points, 13 percentage points, 14 percentage points, 15 percentage points, 16 percentage points, 17 percentage points, 18 percentage points, 19 percentage points, 20 percentage points, 21 percentage points, 22 percentage points, 23 percentage points, 24 percentage points, 25 percentage points, 26 percentage points, 27 percentage points, 28 percentage points, 29 percentage points, 30 percentage points, 31 percentage points, 32 percentage points, 33 percentage points, 34 percentage points, 35 percentage points, 36 percentage points, 37 percentage points, 38 percentage points, 39 percentage points, 40 percentage points, 41 percentage points, 42 percentage points, 43 percentage points, 44 percentage points, 45 percentage points, 46 percentage points, 47 percentage points, 48 percentage points, 49 percentage points, 50 percentage points, 51 percentage points, 52 percentage points, 53 percentage points, 54 percentage points, 55 percentage points, 56 percentage points, 57 percentage points, 58 percentage points, 59 percentage points, 60 percentage points, 61 percentage points, 62 percentage points, 63 percentage points, 64 percentage points, 65 percentage points, 66 percentage points, 67 percentage points, 68 percentage points, 69 percentage points, 70 percentage points, 71 percentage points, 72 percentage points, 73 percentage points, 74 percentage points, 75 percentage points, 76 percentage points, 77 percentage points, 78 percentage points, 79 percentage points, 80 percentage points, 81 percentage points, 82 percentage points, 83 percentage points, 84 percentage points, 85 percentage points, 86 percentage points, 87 percentage points, 88 percentage points, 89 percentage points, 90 percentage points, or more. The increase can be based on a comparison to the amounts set forth in Tables 2C and 2D.

Generally, terrestrial animal cells not obtained by the media, methods, and systems disclosed herein are essentially free of omega-3 polyunsaturated fatty acids. Accordingly, the 1 or more percentage point increase in omega-3 polyunsaturated fatty acids can be achieved by adding omega-3 polyunsaturated fatty acids while the concentrations of previously-present fatty acids remain unchanged.

In some embodiments, the terrestrial animal cells obtained herein comprise myoblasts, fibroblasts, and/or preadipocytes having an increased concentration of monounsaturated fatty acids in comparison to the amount present in the corresponding cell type in wild caught or farm raised terrestrial animals of the same species. The increased concentration of monounsaturated fatty acids can be by at least about 1 percentage point or more, such as 2 percentage points, 3 percentage points, 4 percentage points, 5 percentage points, 6 percentage points, 7 percentage points, 8 percentage points, 9 percentage points, 10 percentage points, 11 percentage points, 12 percentage points, 13 percentage points, 14 percentage points, 15 percentage points, 16 percentage points, 17 percentage points, 18 percentage points, 19 percentage points, 20 percentage points, 21 percentage points, 22 percentage points, 23 percentage points, 24 percentage points, 25 percentage points, 26 percentage points, 27 percentage points, 28 percentage points, 29 percentage points, 30 percentage points, 31 percentage points, 32 percentage points, 33 percentage points, 34 percentage points, 35 percentage points, 36 percentage points, 37 percentage points, 38 percentage points, 39 percentage points, 40 percentage points, 41 percentage points, 42 percentage points, 43 percentage points, 44 percentage points, 45 percentage points, 46 percentage points, 47 percentage points, 48 percentage points, 49 percentage points, 50 percentage points, 51 percentage points, 52 percentage points, 53 percentage points, 54 percentage points, 55 percentage points, 56 percentage points, 57 percentage points, 58 percentage points, 59 percentage points, 60 percentage points, 61 percentage points, 62 percentage points, 63 percentage points, 64 percentage points, 65 percentage points, 66 percentage points, 67 percentage points, 68 percentage points, 69 percentage points, 70 percentage points, 71 percentage points, 72 percentage points, 73 percentage points, 74 percentage points, 75 percentage points, 76 percentage points, 77 percentage points, 78 percentage points, 79 percentage points, 80 percentage points, 81 percentage points, 82 percentage points, 83 percentage points, 84 percentage points, 85 percentage points, 86 percentage points, 87 percentage points, 88 percentage points, 89 percentage points, 90 percentage points, or more. The increase can be based on a comparison to the amounts set forth in Tables 2C and 2D.

In some embodiments, the terrestrial animal cells obtained herein comprise myoblasts, fibroblasts, and/or preadipocytes having an increased concentration of unsaturated fatty acids in comparison to the amount present in the corresponding cell type in wild caught or farm raised terrestrial animals of the same species. The increased concentration of unsaturated fatty acids can be by at least about 1 percentage point or more, such as 2 percentage points, 3 percentage points, 4 percentage points, 5 percentage points, 6 percentage points, 7 percentage points, 8 percentage points, 9 percentage points, 10 percentage points, 11 percentage points, 12 percentage points, 13 percentage points, 14 percentage points, 15 percentage points, 16 percentage points, 17 percentage points, 18 percentage points, 19 percentage points, 20 percentage points, 21 percentage points, 22 percentage points, 23 percentage points, 24 percentage points, 25 percentage points, 26 percentage points, 27 percentage points, 28 percentage points, 29 percentage points, 30 percentage points, 31 percentage points, 32 percentage points, 33 percentage points, 34 percentage points, 35 percentage points, 36 percentage points, 37 percentage points, 38 percentage points, 39 percentage points, 40 percentage points, 41 percentage points, 42 percentage points, 43 percentage points, 44 percentage points, 45 percentage points, 46 percentage points, 47 percentage points, 48 percentage points, 49 percentage points, 50 percentage points, 51 percentage points, 52 percentage points, 53 percentage points, 54 percentage points, 55 percentage points, 56 percentage points, 57 percentage points, 58 percentage points, 59 percentage points, 60 percentage points, 61 percentage points, 62 percentage points, 63 percentage points, 64 percentage points, 65 percentage points, 66 percentage points, 67 percentage points, 68 percentage points, 69 percentage points, 70 percentage points, 71 percentage points, 72 percentage points, 73 percentage points, 74 percentage points, 75 percentage points, 76 percentage points, 77 percentage points, 78 percentage points, 79 percentage points, 80 percentage points, 81 percentage points, 82 percentage points, 83 percentage points, 84 percentage points, 85 percentage points, 86 percentage points, 87 percentage points, 88 percentage points, 89 percentage points, 90 percentage points, or more. The increase can be based on a comparison to the amounts set forth in Tables 2C and 2D.

In some embodiments, the terrestrial animal cells obtained herein comprise myoblasts, fibroblasts, and/or preadipocytes having a decreased concentration of saturated fatty acids in comparison to the amount present in the corresponding cell type in wild caught or farm raised terrestrial animals of the same species. The decreased concentration of saturated fatty acids can be by at least about 1 percentage point or more, such as 2 percentage points, 3 percentage points, 4 percentage points, 5 percentage points, 6 percentage points, 7 percentage points, 8 percentage points, 9 percentage points, 10 percentage points, 11 percentage points, 12 percentage points, 13 percentage points, 14 percentage points, 15 percentage points, 16 percentage points, 17 percentage points, 18 percentage points, 19 percentage points, 20 percentage points, 21 percentage points, 22 percentage points, 23 percentage points, 24 percentage points, 25 percentage points, 26 percentage points, 27 percentage points, 28 percentage points, 29 percentage points, 30 percentage points, 31 percentage points, 32 percentage points, 33 percentage points, 34 percentage points, 35 percentage points, 36 percentage points, 37 percentage points, 38 percentage points, 39 percentage points, 40 percentage points, 41 percentage points, 42 percentage points, 43 percentage points, 44 percentage points, 45 percentage points, 46 percentage points, 47 percentage points, 48 percentage points, 49 percentage points, 50 percentage points, 51 percentage points, 52 percentage points, 53 percentage points, 54 percentage points, 55 percentage points, or more. The decrease can be based on a comparison to the amounts set forth in Tables 2C and 2D.

In some embodiments, the terrestrial animal cells obtained herein comprise myoblasts, fibroblasts, and/or preadipocytes having both an increased concentration of omega-3 polyunsaturated fatty acids up to 90 percentage points or more, and a decreased concentration of saturated fatty acids up to 55 percentage points or more, in comparison to the amount present in the corresponding cell type in wild caught or farm raised terrestrial animals of the same species.

Food products comprising terrestrial animal cells as described above can have an omega-3 PUFA content at least 1 percentage point higher and/or a UFA content at least 1 percentage point lower than a food product of identical composition except for the cells of the terrestrial animal being from a wild-caught or farm-raised terrestrial animal of the same species.

In the methods and systems herein described, the lipid-loaded cells (e.g., aquatic animal cells, terrestrial animal cells) can be harvested by cell separation techniques, such as settling or tangential flow filtration. The harvested cells can then then be assembled in cell-cultured food products using various methods, including extrusion and bioprinting, as will be understood by a person skilled in the art, to form cubes, strips or fillets. The cubes, strips or fillets are composed of myoblasts, adipocytes, fibroblasts or combinations of these cell types and optionally a suitable matrix.

Cells obtained using the media, methods and systems described herein can be used to create a variety of cell-cultured food products, including products that look, feel and taste substantially like, for example, whole animals and/or cuts of wild caught or farm-raised fish, seafood, beef, pork, or poultry of the same species. Suitable methods for preparing such food products are known in the art and include combining cells of the desired type (e.g., myocytes, adipocytes, fibroblasts) and optionally plant cells, fungal cells, other non-animal cells, plant protein, fungal protein, other non-animal protein, and/or a suitable matrix to create a product that resembles, for example, a fish fillet, a steak or other cut of beef, a cut of pork, a cut of poultry, etc. Such products can be homogenous, for example with cells of different types and species, protein from different source, and/or matrix materials evenly distributed throughout the product, or heterogenous, for example with cells of different types preferentially located in a portion or portions of the product, such as a layered structure.

Generally, the cell-cultured food product is an aquatic or terrestrial animal food product that contains cells cultured in accordance with this disclosure.

In embodiments, the cell-cultured food product is a fish food product that contains cells cultured in accordance with this disclosure. For example, the cell-cultured food product can contain aquatic animal cells, preferably fish cells. For example, the cell-cultured food product can consist essentially of or consist of fish cells and optionally a suitable matrix, and the fish cells are selected from the group consisting of myoblasts, myocytes, fibroblasts, preadipocytes, adipocytes, keratinocytes, and combinations thereof that are loaded with one or more desired lipids, for example by culturing the cells in accordance with this disclosure. Accordingly, the food product can have a lower amount of saturated fat (g saturated fat/g total fat) in comparison to wild caught fish of the same species. The food product can have a higher amount of unsaturated fat (e.g., g PUFA and/or MUFA/g total fat) compared to wild caught fish of the same species. In embodiments, the food product can have a higher amount of omega-3 fatty acids (g omega-3/g total fat), such as DHA, EPA, ALA and combinations thereof, in comparison to wild caught fish of the same species, and further comprise a higher amount of nervonic acid compared to wild caught fish of the same species. Optionally, the food product of such embodiments, also has a higher amount of palmitoleic acid, vaccenic acid, oleic acid, linoleic acid lauric acid, myristic acid, palmitic acid, steric acid, and any combination thereof compared to wild caught fish of the same species. In embodiments, the food product consists essentially of or consists of aquatic animal cells that are derived or obtained from a single animal species, and optionally a suitable matrix. Preferred food products consist essentially of or consists of cells that are derived from aquatic species disclosed herein, including carp, yellowtail, mahi-mahi, bluefin tuna, yellowfin tuna, red snapper, cod, Patagonian tooth fish, and the like.

In particular, a cell-cultured fish food product contains higher levels of total lipids than conventionally sourced fish. The resulting fish product contains higher levels of polyunsaturated fatty acids, such as omega-3s fatty acids, than conventionally sourced fish.

In more particular examples, a cell-cultured fish food product contains less C16:00 and/or C18:00 fatty acids (as a percentage of total fat or by weight of the product) in comparison to wild caught fish of the same species. Optionally, or in addition, the cell-cultured fish food product contains more C18:01 fatty acids and/or omega-3 fatty acids, such as ALA, EPA and/or DHA (as a percentage of total fat or by weight of the product) in comparison to wild caught fish of the same species. The embodiments herein described also include systems to perform the methods herein described. In particular, in some embodiments, the system can comprise one or more MUFA in combination with basal culture medium, and/or one or more cell types of an aquatic animal in the sense of the disclosure possibly comprised within an aquatic cell biomass. In some embodiments, the system can comprise a culture medium of the disclosure comprising one or more MUFA in combination with one or more cell types of an aquatic animal in the sense of the disclosure possibly comprised within an aquatic cell biomass.

In some embodiments, the system can comprise one or more PUFA, SFA and/or sterols in combination with basal culture medium, and/or one or more cell types of an aquatic animal in the sense of the disclosure possibly comprised within an aquatic cell biomass. In some embodiments, the system can comprise a culture medium of the disclosure comprising one or more PUFA, SFA and/or sterols in combination with one or more cell types of an aquatic animal in the sense of the disclosure possibly comprised within an aquatic cell biomass.

In some embodiments, the system can comprise one or more PUFA, SFA and/or sterols in combination with nervonic acid and further in combination with basal culture medium, and/or one or more cell types of an aquatic animal.

In some embodiments, the system can comprise one or more PUFA in combination with nervonic acid and further in combination with basal culture medium, and/or one or more cell types of an aquatic animal in the sense of the disclosure possibly comprised within an aquatic cell biomass. In some embodiments, the system can comprise a culture medium of the disclosure comprising one or more PUFA in combination with nervonic acid and/or one or more cell types of an aquatic animal in the sense of the disclosure possibly comprised within an aquatic cell biomass. In some embodiments, the system can comprise a culture medium of the disclosure comprising one or more PUFA and nervonic acid, in combination with one or more cell types of an aquatic animal in the sense of the disclosure possibly comprised within an aquatic cell biomass.

In some embodiments, the system can comprise omega-3, in combination with nervonic acid and further in combination with basal culture medium, and/or one or more cell types of an aquatic animal in the sense of the disclosure possibly comprised within an aquatic cell biomass. In some embodiments, the system can comprise a culture medium of the disclosure comprising omega-3, in combination with nervonic acid and/or one or more cell types of an aquatic animal in the sense of the disclosure possibly comprised within an aquatic cell biomass. In some embodiments, the system can comprise a culture medium of the disclosure comprising omega-3 and nervonic acid, in combination with one or more cell types of an aquatic animal in the sense of the disclosure possibly comprised within an aquatic cell biomass.

In some embodiments, the system can comprise one or more lipid, in combination with basal culture medium, and/or one or more cell types of an aquatic animal in the sense of the disclosure possibly comprised within an aquatic cell biomass. In embodiments wherein the lipid comprises or consists of one or more PUFA, SFA and/or sterols, the system further comprises nervonic acid. In some embodiments, the system can comprise, a culture medium of the disclosure comprising one or more lipid herein described, in combination with one or more cell types of an aquatic animal in the sense of the disclosure possibly comprised within an aquatic cell biomass. In embodiments wherein the lipid comprises or consists of one or more PUFA, SFA and/or sterols, the culture medium and/or the system further comprises nervonic acid.

Although the foregoing discussion has focused on media, methods, and systems for culturing cells for cell-cultured food products, the media, methods, and systems can be used for culturing cells for any purpose. For example, the media, methods, and systems described herein are suitable for a wide variety of life science applications, including but not limited to in vitro fertilization and pharmaceutical applications that require cell culture of eucaryotic cells (e.g., production of antibodies and therapeutic peptides and proteins, CAR-T cell production and stem cell culture, IPSCs, and primary stem cells). The cells can be from species such as human, Chinese hamster, African green monkey, dog, cabbage looper (Trichoplusia ni), or fall armyworm (Spodoptera frugiperda), among others.

The suitability of the media, methods, and systems for particular life science applications are evaluated by comparing the performance of relevant cell types in conventional media, methods, and system with their performance in the media, methods, and systems described and claimed herein. Exemplary cell types and applications are shown in Table 3 below.

TABLE 3
Life science applications for various cell types

| Application | Cell type |
|---|---|
| Antibody and therapeutic | Chinese hamster ovary |
| protein production | (CHO) |
| | Vero |
| | HEK |
| | MRC-5 |
| | COS-7 |
| | COS-5 |
| | MDCK |
| | High five (Tn-5) |
| | Sf9 or Sf21 |
| Tissue engineering | Embryonic stem cells |
| | Pluripotent stem cells |
| | Tissue-specific stem cells |
| | Mesenchymal stem cells |
| R&D, pharma | CAR-T cell production |
| | (autologous and allogenic) |
| | Caco-2 intestinal epithelium |
| IVF | Embryo production and |
| | culture |
| R&D, pharma | Healthy and pathogenic |
| | human and/or animal |
| | primary cells and cell lines |
| R&D, pharma | Embryonic, pluripotent and |
| | iPSCs, Cells from directed |
| | differentiation of stem cells |

In some embodiments, the systems herein described can be provided in the form of kits of parts. In a kit of parts, the culture media, nervonic acid and/or vaccenic acid can be provided in various combinations one with another and with lipids and/or cells. In the kits of parts, the components can be comprised in the kit independently, possibly included in a composition together with suitable vehicle carriers or auxiliary agents.

Additional components can also be included and comprise reference standards and further components identifiable by a skilled person upon reading of the present disclosure.

In some embodiments, the kit can comprise fish myoblasts and/or fibroblasts and nervonic acid. The kit of parts further comprises one or more other fatty acids, herein described, such as omega-3 polyunsaturated fatty acids.

In some embodiments, the kit comprises fish preadipocytes and one or more fatty acids herein described. In some embodiments, the kit comprises preadipocytes from one or more terrestrial animals, and one or more fatty acids herein described. The kit of parts further comprises basal media, culture media, differentiation media, and/or lipid loading media necessary for loading the preadipocytes with one or more fatty acids, herein described as will be understood by a person skilled in the art.

In embodiments herein described, the components of the kit can be provided with suitable instructions and other necessary reagents in order to perform the methods here disclosed. The kit will normally contain the compositions in separate containers. Instructions, for example written or audio instructions, on paper or electronic support such as tapes, CD-ROMs, flash drives, or by indication of a Uniform Resource Locator (URL), which contains a PDF, HTML, or other electronic copy of the instructions for carrying out the assay, will usually be included in the kit. The kit can also contain, depending on the particular method used, other packaged reagents and materials (e.g. wash buffers and the like).

Further details concerning the compositions, methods and systems herein described will become more apparent hereinafter from the following detailed disclosure of examples by way of illustration only, with reference to an experimental section.

### EXAMPLES

The cell-cultured food products, and related cells, compositions, methods, and systems herein described are further illustrated in the following examples, which are provided by way of illustration and are not intended to be limiting.

### Materials and Methods

### Cell Lines and Culture Conditions

Cell lines were procured from commercial vendors or developed by Applicants as shown in Table 4. Culture conditions were as shown in Table 5.

TABLE 4
Cell Lines

| Organism | Species | Cell Line ID | Cell (tissue) type | Source |
|---|---|---|---|---|
| Largemouth | Micropterus | Bass myoblasts | Myoblasts | Proprietary |
| bass | salmoides | | (Muscle) | |
| Yellowtail | Seriola lalandi | Yellowtail | Myoblasts | Proprietary |
| | | myoblasts | (muscle) | |
| Carp | Hypophthalmichthys | Carp | Preadipocytes | Proprietary |
| | moltrix | preadipocytes | (VAT) | |
| Bluegill | Lepomis | BF-2 | Fibroblasts (caudal | Proprietary |
| | macrochirus | | trunk) | |
| Atlantic | Salmo salar | ASK | Epithelial (Kidney) | ATCC |
| Salmon | | | | |
| Rainbow trout | Oncorhynchus | Rtgill-W1 | Epithelial (gill) | ATCC |
| | mykiss | | | |
| Yellowfin tuna | Thunnus albacares | Yellowfin tuna | Fibroblasts (heart) | Proprietary |
| | | fibroblasts | | |
| Bluefin tuna | Thunnus orientalis | Bluefin tuna | Myoblasts | Proprietary |
| | | myoblasts | (muscle) | |
| Bluefin tuna | Thunnus orientalis | Bluefin tuna | Preadipocytes (SC | Proprietary |
| | | preadipocytes | adipose) | |
| Bluefin tuna | Thunnus orientalis | Bluefin tuna | Fibroblasts | Proprietary |
| | | fibroblasts | (muscle) | |
| Red Snapper | Lutjanus | Red snapper | Myoblasts | Proprietary |
| | campechanus | myoblasts | (Muscle) | |
| Mahi-Mahi | Coryphaena | Mahi-mahi | Myoblasts | Proprietary |
| | hippurus | myoblasts | (muscle) | |
| Mahi-Mahi | Coryphaena | Mahi-mahi | Fibroblasts | Proprietary |
| | hippurus | fibroblasts | (muscle) | |
| Mozambique | Oreochromis | OmB | Fibroblast-like | ATCC |
| Tilapia | massambicus | | (brain) | |
| Fall Army | Spodoptera | SF21 | Epithelial (pupal | Sigma (ECACC) |
| Worm | frugiperda | | ovarian tissue) | |
| Chicken (red | Gallus | UMNSAH/DF-1 | Fibroblast | ATCC |
| junglefowl) | | | (Embryo) | |
| Japanese Quail | Coturnix japonica | QNR/K2 | Mixed | ATCC |
| | | | Morphology | |
| | | | (Neuroretina) | |
| Pig | Sus scrofa | PK15 | Epithelial (Kidney) | ATCC |
| Dog (cocker | Canis familiaris | MDCK | Epithelial (Kidney) | Sigma (ECACC) |
| spaniel) | | | | |
| Rabbit | Oryctolagus | RAB-9 | Fibroblasts (Skin) | Sigma (ECACC) |
| | cuniculus | | | |
| Bovine | Bos taurus | Bovine Muscle | Satellite Cells | Accegen |
| | | Satellite Cells | (muscle; primary) | |

TABLE 5
Cell Culture Conditions including temperature, atmospheric
conditions (Atm), and Cell culture media

| | | | Temp | | |
|---|---|---|---|---|---|
| Organism | Species | Cell Line ID | (° C.) | Atm | Media |
| Largemouth | Micropterus salmoides | Bass myoblasts | 23 | Air | CO2 Independent |
| bass | | | | | Media + 10% FBS |
| Yellowtail | Seriola lalandi | Yellowtail | 23 | Air | DMEM/F12 + 10% |
| | | myoblasts | | | FBS |
| Carp | Hypophthalmichthys | Carp | 23 | Air | DMEM/F12 + 4% |
| | moltrix | preadipocytes | | | FBS |
| Bluegill | Lepomis macrochirus | BF-2 | 23 | Air | MEM + 10% FBS |
| | | | | | or L-15 + 10% FBS |
| Atlantic | Salmo salar | ASK | 23 | Air | L-15 + 20% FBS |
| Salmon | | | | | |
| Rainbow trout | Oncorhynchus mykiss | Rtgill-W1 | 23 | Air | L-15 + 20% FBS |
| Yellowfin tuna | Thunnus albacares | Yellowfin tuna | 27 | Air | DMEM/F12 + 3% |
| | | fibroblasts | | | FBS |
| Bluefin tuna | Thunnus orientalis | Bluefin tuna | 27 | Air | DMEM/F12 + 10% |
| | | myoblasts | | | FBS |
| Bluefin tuna | Thunnus orientalis | Bluefin tuna | 27 | Air | DMEM/F12 + 10% |
| | | preadipocytes | | | FBS |
| Bluefin tuna | Thunnus orientalis | Bluefin tuna | 27 | Air | DMEM/F12 + 10% |
| | | fibroblasts | | | FBS |
| Red Snapper | Lutjanus campechanus | Red snapper | 27 | Air | DMEM/F12+ 10% |
| | | myoblasts | | | FBS |
| Mahi-Mahi | Coryphaena hippurus | Mahi-mahi | 27 | Air | DMEM/F12 + 10% |
| | | myoblasts | | | FBS |
| Mahi-Mahi | Coryphaena hippurus | Mahi-mahi | 27 | Air | CO2 Independent |
| | | fibroblasts | | | Media + 10% FBS |
| Mozambique | Oreochromis | OmB | 27 | Air | L-15 + 10% FBS |
| Tilapia | massambicus | | | | |
| Fall Army | Spodoptera frugiperda | SF21 | 27 | Air | SF90011 + 10% |
| Worm | | | | | FBS |
| Chicken (red | Gallus | UMNSAH/DF-1 | 37 | 5% | DMEM HG + 10% |
| junglefowl) | | | | CO2 | FBS |
| Japanese Quail | Coturnix japonica | QNR/K2 | 37 | 5% | DMEM HG + 10% |
| | | | | CO2 | FBS |
| Pig | Sus scrofa | PK15 | 37 | 5% | EMEM (EBSS) + |
| | | | | CO2 | 10% FBS |
| Dog (cocker | Canis familiaris | MDCK | 37 | 5% | EMEM (EBSS) + |
| spaniel) | | | | CO2 | 10% FBS + 1% |
| | | | | | NEAA |
| Rabbit | Oryctolagus cuniculus | RAB-9 | 37 | 5% | EMEM (EBSS) + |
| | | | | CO2 | 10% FBS + 1% |
| | | | | | NEAA |
| Bovine | Bos taurus | Bovine Muscle | 37 | 5% | Accegen Cat#: |
| | | Satellite Cells | | CO2 | Media Kit, #ABM- |
| | | | | | M0048 |

### Reagents

### Fatty Acid Stock Solutions

50 mg/ml fatty acid stock solutions were prepared using either powdered fatty acids or concentrated fatty acid solutions. Powdered fatty acids were dissolved in 200 proof ethanol (not 100% denatured) at a concentration of 50 mg/mL. Concentrated fatty acid solutions were diluted using 200 proof ethanol to a final concentration of 50 mg/mL.

### BSA-Conjugated Fatty Acid Stock Solutions

1 mL aliquots of 10 mg/ml stock solutions of BSA-conjugated fatty acids were prepared as follows.
Add 800 μl 7.5% BSA to 1.5 ml tubes and place in 37° C. water bath until brought to temperature.
Place 50 mg/ml stocks of FAs to 37° C. water bath to warm.
Add 25 μl of FA stock at a time to the BSA tube until FA enters solution, then repeat until up to 200 μl are added.
Incubate in 37° C. water bath for 1 hour after all FAs have been added to the BSA tubes and the solution is fully dissolved.
Filter with 0.2 μm filter and syringe into 1.5 mL tube
Store @−20° ° C.(Recommended if not using within 48 hours)

### Cell Media

Media for cell lines procured from ATCC and Sigma (EACC, ECACC) were prepared as directed in the accompanying product sheets. CO2-independent media base was prepared as shown in Table 6. Proprietary media formulations were prepared according to proprietary methods.

TABLE 6
CO₂ independent media base

| | | | Volume |
|---|---|---|---|
| Reagent | Vendor | Catalog # | added (mL) |
| CO₂ Independent | Fisher | 18045088 | 500 |
| Media | | | |
| Fetal Bovine Serum | Fisher | Cat# MT35015CV | 50 |
| | | (Corning lot# 35015164) | |
| Antibiotic- | Fisher | 15240112 | 5 |
| antimycotic | | | |
| Gentamicin | Fisher | 15750078 | 0.5 |
| (50 mg/mL) | | | |
| HEPES (1M) | Fisher | 15630080 | 5 |
| Glutamax | Fisher | 35050079 | 5 |
| MEM Non-essential | Fisher | 11140050 | 2.5 |
| amino acids | | | |

### BCA Protein Assay

Pierce BCA Protein assays kits were obtained from ThermoScientific (San Diego, California; catalog numbers 23225 and 23227, Document Part No. 2161296, publication number MAN0011430 v. B.0) and assays were performed according to the protocol provided with the kit.

### Lipidomics Analysis

### Collection of Cells and Fatty Acids for Lipidomics Analysis

Media was manually removed from cells grown in 6 well plates. 1 ml PBS was added to each well to gently wash, and then removed manually. 0.5 ml of 10% methanol was then added to each well, while keeping plates at an angle. Samples were scraped starting from the top left and scraping to the bottom while moving across the well from left to right or right to left. The remaining cells were scraped towards the well bottom, then collected.

Samples were then pipetted into labeled amber glass 2 ml vials. An additional 0.5 ml of 10% methanol was then added to each well to collect any remaining sample and collected in the same vial.

Samples were pipetted to fully lyse the cells and a fraction removed for BCA analysis according to the Pierce BCA Protein Assay referenced above, then flash frozen and stored at −80° ° C. pending lipidomics analysis.

### Lipidomics Analysis Protocol

The fatty acid content of lipid-loaded and control cells was determined using methods described in Quehenberger et al. and Löfgren et al. (Quehenberger O, Armando A M, Dennis E A. High sensitivity quantitative lipidomics analysis of fatty acids in biological samples by gas chromatography-mass spectrometry. Biochim Biophys Acta. 2011 November; 1811(11):648-56. doi: 10.1016/j.bbalip.2011.07.006. Epub 2011 Jul. 20. PMID: 21787881; PMCID: PMC3205314) (Löfgren, L., Forsberg, GB. & Ståhlman, M. The BUME method: a new rapid and simple chloroform-free method for total lipid extraction of animal tissue. Sci Rep 6, 27688 (2016). https://doi.org/10.1038/srep27688).

Briefly, cells were homogenized into 1 mL 10% methanol and 200 uL of the homogenates were extracted by modified BUME. Extracts were brought to dryness and saponified using a 1:1 MeOH: KOH solution at 37 C for 1 hour. Fatty Acids were extracted by a bi-phasic solution of acidified methanol and isooctane, derivatized using PFBB, and analyzed by GC-MS on an Agilent 6890N gas chromatograph equipped with an Agilent 7683 autosampler. Fatty Acids were separated using a 15m ZB-1 column (Phenomenex) and monitored using SIM identification. Analysis was performed using MassHunter software.

### Example 1: Loading Complex Fatty Acid Mixtures into Fat-Derived Cells of Fishes

In this example, loading of a complex fatty acid mixture, comprised of saturated and unsaturated fatty acids including omega-3s, into fat-tissue-derived cells (preadipocytes) was demonstrated for Silver Carp (Hypophthalmichthys molitrix) and Bluefin tuna (Thunnus orientalis).

### Carp Cell Derivation

Silver carps (4-6 pounds total) were sourced for preadipocyte isolations. Cells were harvested from the visceral adipose tissue of silver carp by enzyme and mechanical dissociation. Typically, 24 g of tissue was processed, and cells were seeded from 1 g of tissue per well. Cells were cultured in growth media and tested for lipid loading up to passage 16.

### Bluefin Tuna Cell Derivation

Pacific bluefin tuna (12-100 pounds) were wild caught locally and identified visually, and by genome sequencing. Preadipocytes were harvested from subcutaneous fat. Typically, 6-24 g of tissue was processed via enzyme and mechanical dissociation and seeded as 0.5-1 g of tissue/well.

### Preadipocyte Differentiation Lipid Loading

A fish adipocyte differentiation protocol found in literature [Ref: Todorcevic et al 2010] was initially used to test silver carp preadipocyte differentiation into adipocytes with the capability of lipid storage. Cells were seeded at 5-7000 cells per cm² onto tissue culture polystyrene for two days and then treated for two days with “adipogenic medium” (differentiation medium), followed by 14 days of lipid loading with test media with 0.2% Sigma Lipid Mix supplementing every two days. The differentiation media found in the literature was composed as follows: basal media supplemented with dexamethasone, biotin, T3, pantothenate, IBMX, insulin and lipid mix.

The cells were then stained with Oil Red O and Hoechst using the following protocol. Cells were washed with 1×PBS and fixed in 4% PFA for 10 minutes at room temperature. The cells were washed twice with PBS and incubated with 60% isopropanol for 20 seconds. Cells were then incubated with Oil Red O for 10 minutes. Once the Oil Red O was removed, the cells were washed with 60% isopropanol to remove excess Oil Red O and then incubated with fresh 60% isopropanol for 30 seconds. The cells were then washed with distilled water for 20 seconds and stained with Hoechst for 15 minutes. With a final wash in distilled water, the cells were imaged.

Carp pre-adipocytes were seeded onto tissue culture polystyrene at a density of 5000 cells per cm². Cells were cultured in the presence of control media without fatty acid mixture or test media with fatty acid mixture (Sigma Lipid Mix, 1%) with varying serum levels (4, 2, 0%). Cell morphology was observed over a six-day period by brightfield microscopy for characteristic rounding and lipid loading.

The fatty acid mixture used in this example is a commercial product purchased from Millipore Sigma, catalog #L5146, composed of 4.5 g/L cholesterol, 10 g/L cod liver oil fatty acids (methyl esters), 25 g/L polyoxyethylenesorbitan monooleate, and 2 g/L D-alpha-tocopherol acetate in ethanol. The serum used in this example is produced from fetal bovine blood and is processed for use in cell culture. Serum is a non-defined mixture that can vary from lot to lot as will be understood by a person skilled in the art.

Bluefin tuna preadipocytes were seeded onto tissue culture polystyrene at a density of 10,000 cells per cm². Cells were cultured in the presence of control media without fatty acid mixture or test media with fatty acid mixture (Sigma Lipid Mix, 1%) with varying serum levels (4, 2 or 0%). Cell morphology was observed over a six-day period by brightfield microscopy for characteristic rounding and lipid loading.

Immunofluorescent staining of cells was performed for cell nuclei by Hoechst, cell cytoskeleton by Phalloidin, and lipid droplets by BODIPY.

Media were aspirated, and cells were washed with 1×PBS. PBS was aspirated, then cells were fixed with 4% PFA for 10 minutes at room temperature. PFA was removed and then gently washed with 1×PBS. Cells were then permeabilized with 0.1% Triton-X-100 for 5 min and then blocked for 1 hour in 0.1% TBS/T with 5% chicken serum. Cells were then incubated with Phalloidin, BODIPY, and Hoechst for 1 hour in basal media without FBS. Cells were imaged in PBS. In the case that Phalloidin was not used, after fixing, the cells were incubated with BODIPY and Hoechst for 1 hour and then stained.

### Results

Preadipocytes from freshwater carp or saltwater bluefin tuna were isolated and expanded in vitro. Proliferating cells showed elongated cell morphology with increasing cell number over time. Initial testing with carp demonstrated that the methods previously used for lipid loading of preadipocytes that incorporated insulin or cAMP stimulation were not effective. Previously existing methods showed limited lipid loading over the 14-day period of this differentiation protocol. While a small number of cells stored some lipids (identified via Oil Red O staining), all cells did not morphologically round up to store large lipid droplets, as shown with the process according to this example.

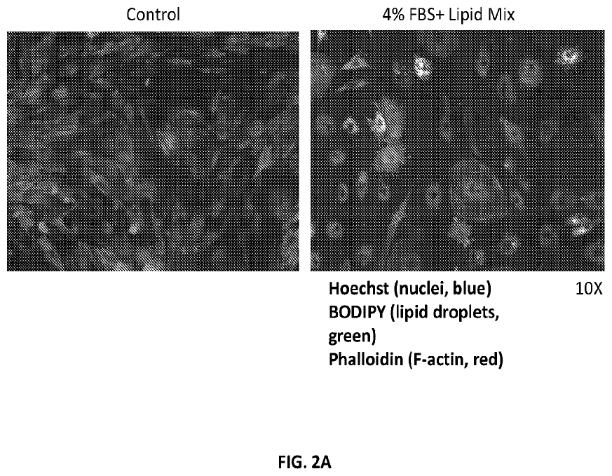

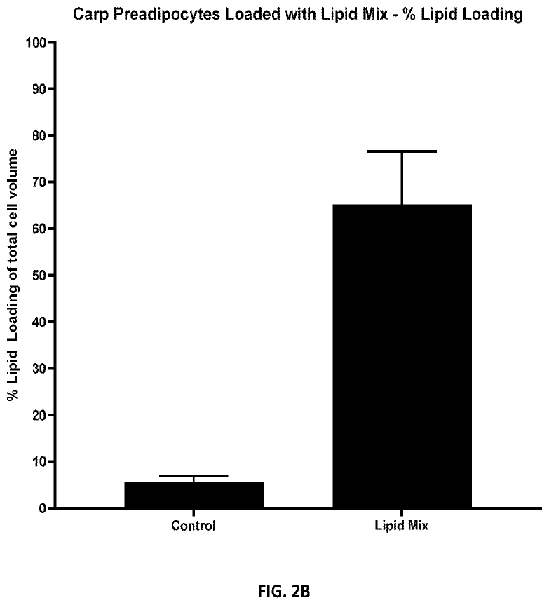

Here, media with reduced or no serum in the presence of a complex fatty acid mixture enabled uptake and storage of fatty acids by carp preadipocytes (FIG. 1). Maintained viability was exhibited by the continued presence of a cell monolayer. Characteristically of maturing adipocytes, cell morphology changed from elongated to rounded with increased cell size and the presence of lipid droplets. Compared to control cultures that showed no morphological changes over a seven-day period, carp preadipocytes in the presence of fatty acids achieved morphological changes by two days that were maintained throughout the seven day culture period. This suggests that the accumulation of fatty acids is more directly related to the addition of fatty acids rather than cell density or time in culture. Immunofluorescence was performed to confirm that the morphological changes of carp preadipocytes were caused by lipid droplet accumulation (FIGS. 2A-2B). Carp preadipocytes cultured in the presence of the fatty acid mixture were positive for both lipid droplets and cytoskeletal protein F-actin, while cells cultured in control conditions expressed cell cytoskeleton protein F-actin but were negative for lipid accumulation.

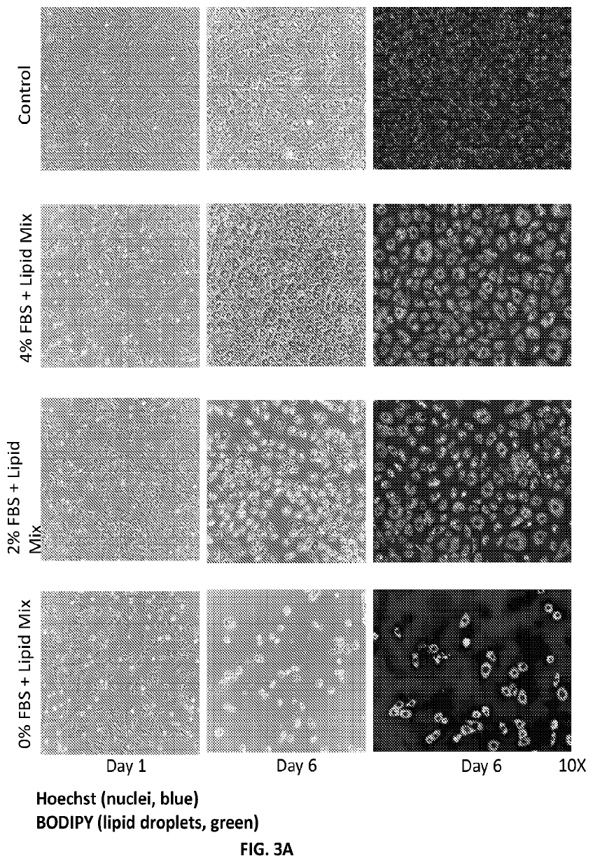

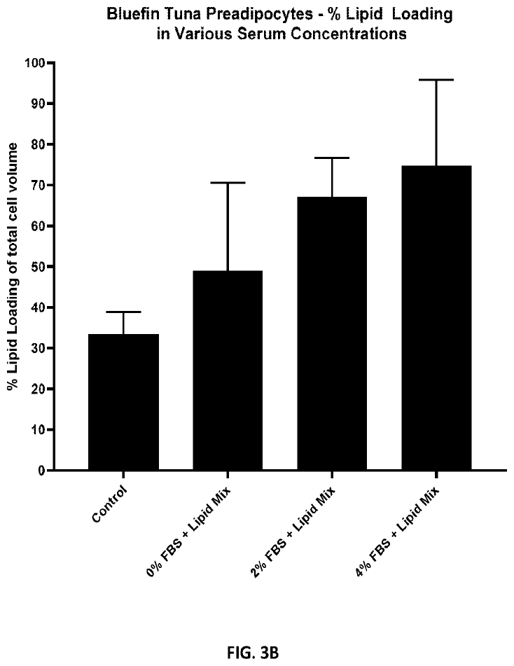

Preadipocytes from bluefin tuna accumulated lipids in the presence of fatty acid mixture similarly to those of carp (FIGS. 3A-3B). Relevant morphological changes included cell rounding and the accumulation of lipid droplets. Both carp and tuna are commercially used in the fishing industry as a food product though distinctly from freshwater or saltwater environments. Importantly, cells from either species accumulated lipids from the complex fatty acid mixture that contains a mixture of saturated and unsaturated fatty acids, including Omega-3s. While carp and tuna are not closely related taxonomically, they are both in the Osteichthyes superclass of bony fishes that may provide commonality in preadipocyte behaviors.

### Example 2: Loading Single Fatty Acids into Fat-Derived Cells of Fishes

In this example, loading of individual fatty acids of varying chemical structures was demonstrated for fat-tissue-derived cells of fishes. The accumulation of specific fatty acids such as Omega-3 versus saturated fatty acids allows for tuning of nutritional composition in the cells, as well as of the final 3D seafood product.

Preadipocytes derived from freshwater carp as described in Example 1 were evaluated for the ability to uptake individual fatty acids. Cells were seeded onto tissue culture polystyrene at a density of 5-7000 cells per cm². Cells were cultured in the presence of control media without fatty acids or test media with individual fatty acids at 25, 50, 75, or 100 μg/ml in the absence of serum. Individual fatty acids tested included saturated fatty acids (palmitoleic acid) and unsaturated fatty acids (linoleic acid). Cell morphology was observed over a six-day period by brightfield microscopy for characteristic rounding and lipid loading. As in Example 1, immunofluorescent staining of cells was performed for cell nuclei by DAPI, cell cytoskeleton by Phalloidin, and lipid droplets by BODIPY.

### Results

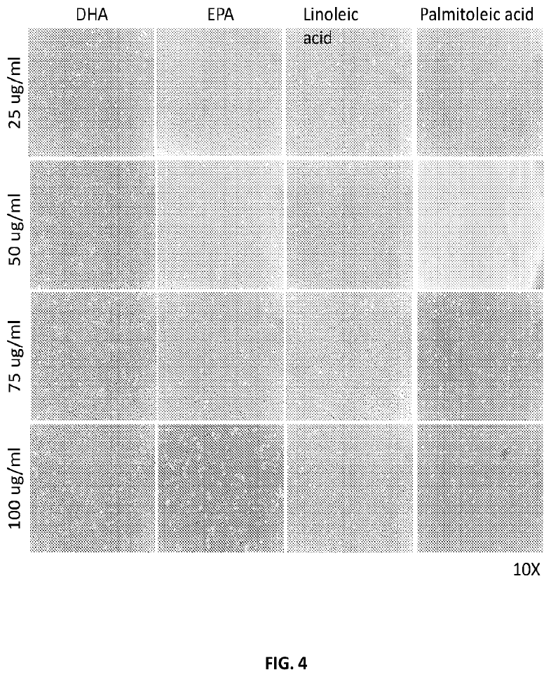

Preadipocytes of carp showed varying degrees of morphological changes and lipid accumulation dependent on the concentration and type of fatty acid used. Similarly to Example 1 with complex mixtures of fatty acids, single fatty acids at sufficient concentrations induced a rounded morphology that was sustained over a six-day period (FIG. 4). At the lowest concentration used of 25 μg/ml, no toxicity was observed, however, at higher concentrations of up to 100 μg/ml, palmitoleic acid showed a reduced cell confluency indicating potential toxicity.

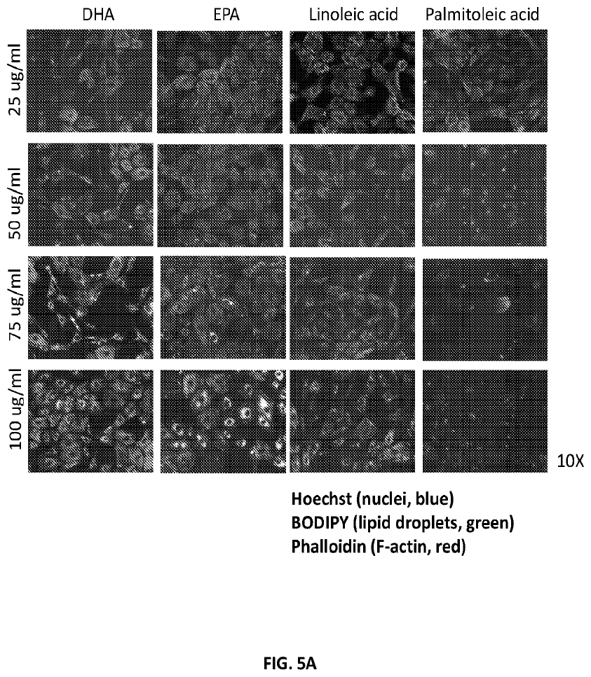

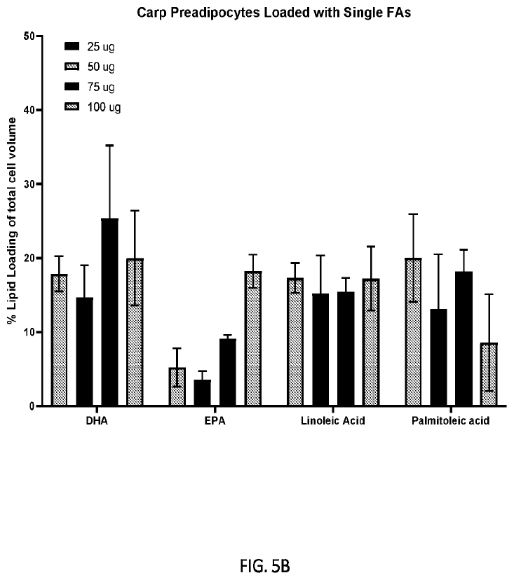

Confirmation of morphological changes correlating to lipid accumulation was observed by BODIPY staining of lipid droplets (FIGS. 5A-5B). Lipid droplets were observed in conditions at the lowest concentration tested of 25 μg/ml and tended to increase in frequency at higher concentrations when toxicity was not observed. Notably, both DHA and EPA, which are nutritionally important Omega-3 polyunsaturated fatty acids, were able to load readily into carp preadipocytes. Compared to what has been described in literature for fish primary preadipocyte cultures, typically the Sigma lipid mix is used for the differentiation process, not individual fatty acids, or combinations of fatty acids. Palmitoleic acid, a monounsaturated fatty acid, showed effective loading at 50 μg/ml despite reduced cell number. The loading efficiency of the different fatty acids is surprising given the naturally occurring ratio of fatty acids in carp that contain significant amounts of monounsaturated acids, including palmitoleic acid (https://www.ncbi.nlm.nih.gov/pmc/articles/PMC4325063/ and https://www.agriculturejournals.cz/publicFiles/84796.pdf). These data suggest process- and formulation-dependent effects of lipid accumulation for in vitro proliferated cells versus those in vivo.

### Example 3: Loading Complex Fatty Acid Mixtures into Connective Tissue-Derived Cells of Fish

In this example, loading of a complex fatty acid mixture, comprised of saturated and unsaturated fatty acids including omega-3s, into connective tissue cells (fibroblasts) was demonstrated for yellowtail (Seriola lalandi).

Fibroblasts of yellowtail were isolated from muscle tissue by enzymatic and mechanical dissociation. Cells did not fuse or differentiate into muscle as did myoblasts. Cells were expanded in 2D tissue culture in the presence of 10% FBS for several passages to generate cell cultures with stable growth rates. Stabilized cell cultures were used in lipid loading experiments.

Fibroblasts were seeded onto tissue culture polystyrene at a density of 5000 cells per cm2. Cells were cultured in the presence of control media without fatty acid mixture or test media with fatty acid mixture (Sigma Lipid Mix, 1%) in the presence of 4% serum. Cell morphology was observed over a seven-day period by brightfield microscopy for characteristic rounding and lipid loading.

Immunofluorescent staining of cells was performed for cell nuclei by Hoechst, cell cytoskeleton by Phalloidin, and lipid droplets by BODIPY as described in Example 1.

### Results

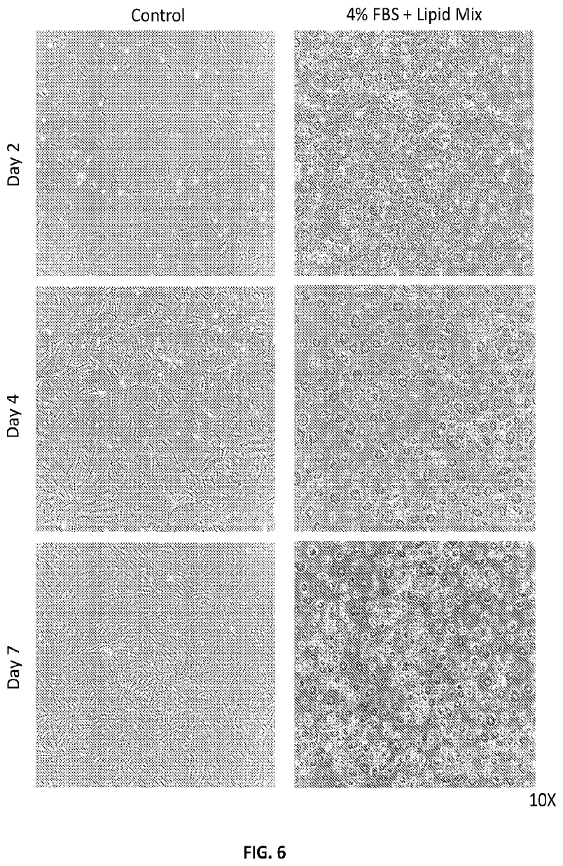

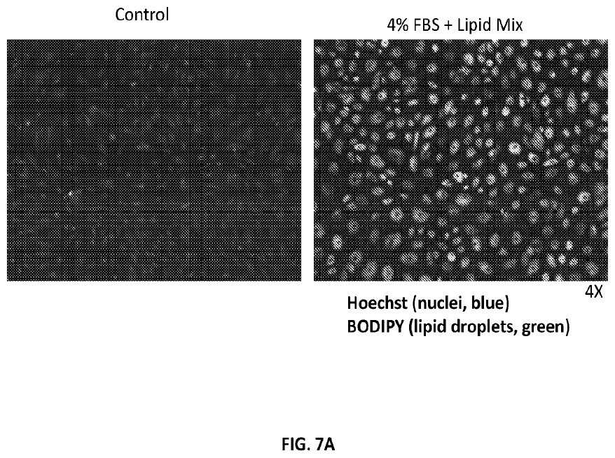

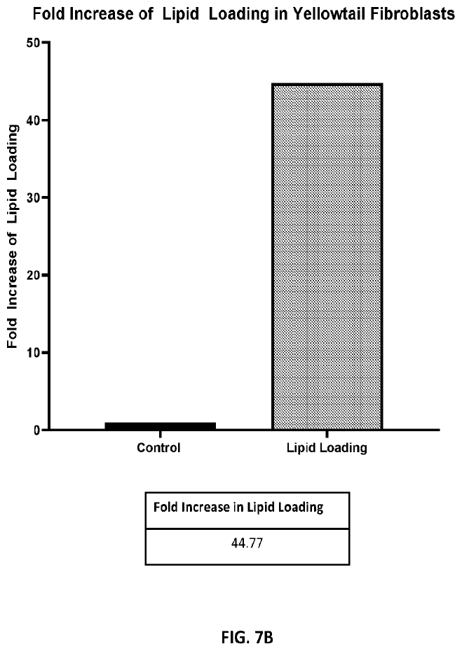

Control fibroblast cultures exhibited typical morphology with elongated cells throughout the seven-day culture period (FIG. 6). Confluent monolayers at day 7 show decreased cell size as cells pack more closely together. In contrast, fibroblasts treated with fatty acid mixture exhibit a rounded morphology with the appearance of lipid droplets as early as day 2 and throughout the entire culture period. Immunofluorescent staining of lipids confirmed the accumulation of lipids in the fibroblasts following treatment with fatty acid mixture (FIGS. 7A-7B). In contrast to the example, previous lipid loading studies did not evaluate fibroblasts as a storage mechanism for fatty acids. On the other hand, the approach according to embodiments of the disclosure as exemplified in the current example shows a unique method to load lipids into cells and a method of incorporating fatty acids into a cell-cultured seafood product without a fat-tissue-derived cell.

### Example 4: Loading Complex Fatty Acid Mixtures into Muscle-Derived Cells of Fish

In this example, loading of a complex fatty acid mixture, comprised of saturated and unsaturated fatty acids including omega-3s, into muscle precursor cells (myoblasts) was demonstrated for yellowtail (Seriola lalandi), mahi-mahi (Coryphaena hippurus), and Pacific bluefin tuna (Thunnus orientalis).

Myoblasts of yellowtail, mahi-mahi, or bluefin tuna were isolated from muscle tissue by enzymatic and mechanical/or manual dissociation. Cells were expanded in 2D tissue culture in the presence of 10% FBS for several passages to generate cell cultures with stable growth rates. Muscle forming capacity of the cells was confirmed by differentiation testing and formation of elongated and multinucleated cells upon switch to low serum conditions. Stabilized cell cultures with confirmed muscle function were used in lipid loading experiments.

Myoblasts were seeded onto tissue culture polystyrene at a density of 5000-10,000 cells per cm2. Cells were culture in the presence of control media without fatty acid mixture or test media with fatty acid mixture (Sigma Lipid Mix, 1%) in the presence of 4% serum. Cell morphology was observed for up to seven days by brightfield microscopy for characteristic rounding and lipid loading. Media was changed every other day with fresh lipids.

Immunofluorescent staining of cells was performed on day 6 or 7 for cell nuclei by Hoechst and lipid droplets by BODIPY as described in Example 1.

### Results

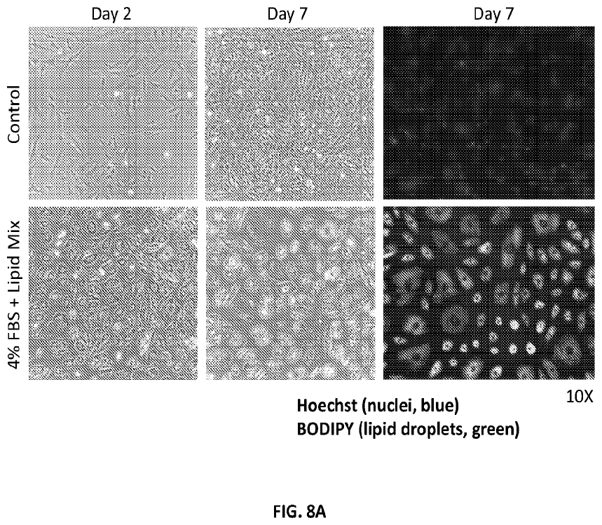

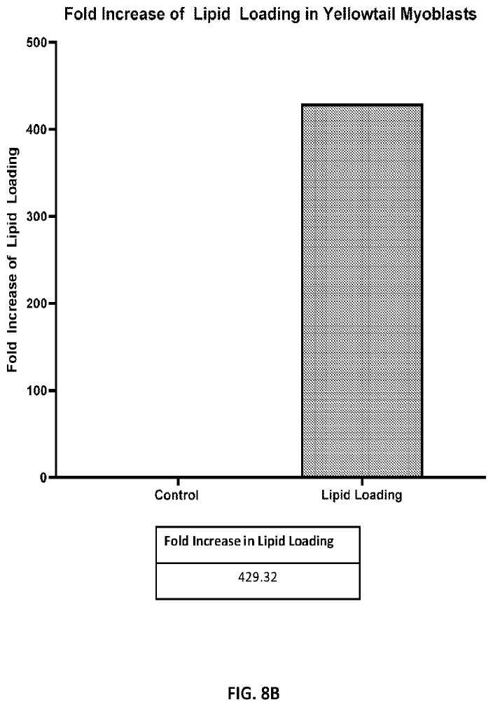

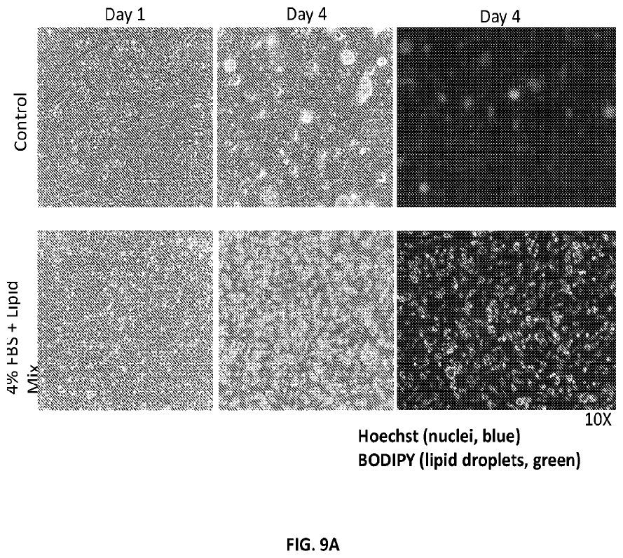

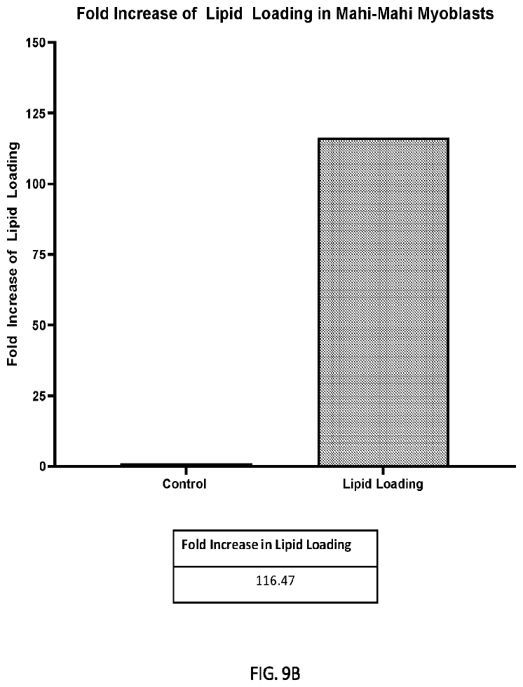

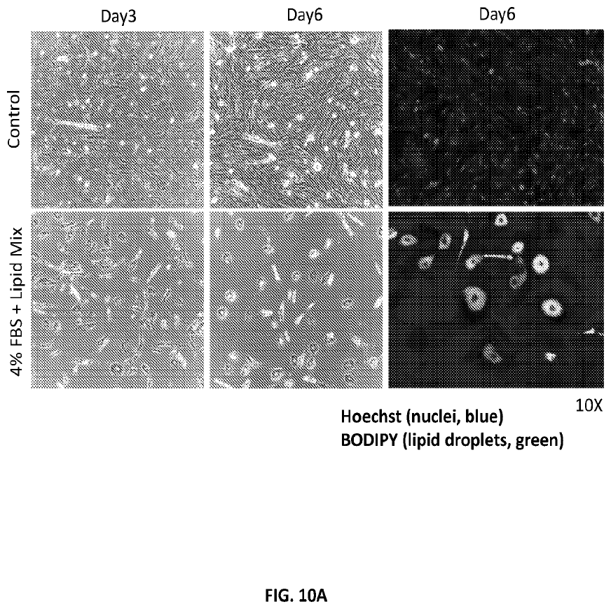

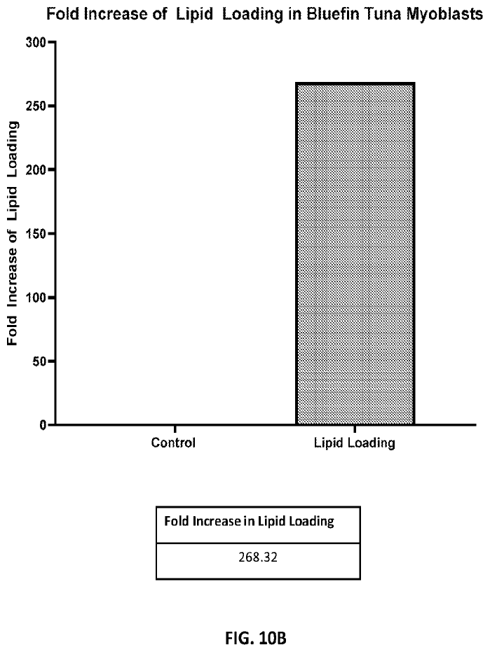

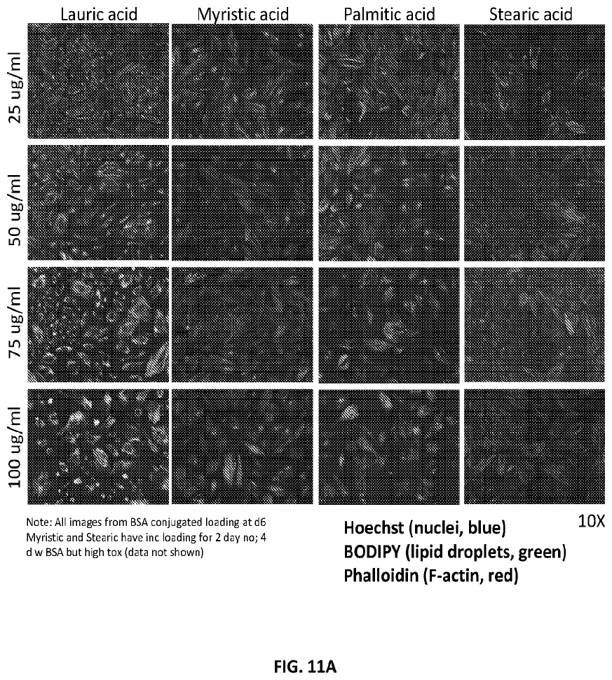

Loading of complex fatty acid mixtures was performed for myoblasts of three different fish species: yellowtail, mahi-mahi, and bluefin tuna. Differentiation experiments confirmed myogenic capacity prior to evaluating lipid loading. Similar to other cells types tested, morphological changes could be observed on day 1 after adding lipids to the media and maintained for several days (FIGS. 8A, 8B, 9A, 9B, 10A, and 10B). Over the culture period, the cells continued to round and enlarge, and filled with lipid droplets. Large lipid droplets could be observed by day 6 using brightfield microscopy and confirmed as lipid droplets by staining with BODIPY. Interestingly, some spontaneous differentiation into myotubes was observed for both mahi-mahi and bluefin tuna myoblasts with subsequent loading of cells with multinucleated or elongated myocyte-like morphologies.

These studies confirm the ability of muscle-derived cells from a wide range of species to lipid load, with or without differentiation to a muscle cell phenotype. The ability to load multinucleated cells suggests that fatty acid loading may occur at various stages of muscle differentiation and be incorporated into mature muscle fibers of a cell-cultured seafood product. All three species tested are in different classes and the closest relationship is within the Actinopterygii order which contains ray-finned fish, indicating wide applicability to the lipid loading of muscle-derived fish cells.

### Example 5: Loading Single Fatty Acids into Muscle-Derived Cells of Fish

In this example, loading of individual fatty acids of varying chemical structure was demonstrated for muscle-derived cells of fish. The accumulation of specific fatty acids, such as Omega-3 versus saturated fatty acids, allows for tuning of nutritional composition in the cells as well as the final 3D seafood product.

Myoblasts derived from yellowtail as described in Example 4 were evaluated for the ability to uptake individual fatty acids. Cells were seeded onto tissue culture polystyrene at a density of 6000 cells per cm². Cells were cultured in the presence of control media without fatty acids or test media with individual fatty acids conjugated to BSA at 25, 50, 75, or 100 μg/ml in the presence of 4% serum. Individual fatty acids tested included saturated fatty acids, monounsaturated fatty acids, and unsaturated fatty acids as shown in Table 7 with varying carbon chain length and location of double bonds along the carbon chain. Cell morphology was observed over a six-day period by brightfield microscopy for characteristic rounding and lipid loading. As in Example 1, immunofluorescent staining of cells was performed for cell nuclei by DAPI, cell cytoskeleton by Phalloidin, and lipid droplets by BODIPY.

TABLE 7
Individual fatty acids used in loading of fish cells including
saturated fatty acids (SFA), monounsaturated fatty acids
(MUFA), and polyunsaturated fatty acids (PUFA).

| SFA | | MUFA | | PUFA | |
|---|---|---|---|---|---|
| C12:0 | Lauric acid | C16:1 | Palmitoleic | C18:2 ω-6 | Linoleic |
| | | | acid | | acid |
| C14:0 | Myristic acid | C18:1 ω-7 | Vaccenic | C18:3 ω-3 | α- |
| | | | acid | | Linolenic |
| | | | | | acid |
| C16:0 | Palmitic acid | C18:1 ω-9 | Oleic acid | C20:5 ω-3 | EPA |
| C18:0 | Stearic acid | C24:1 ω-3 | Nervonic | C22:6 ω-3 | DHA |
| | | | acid | | |

### Results

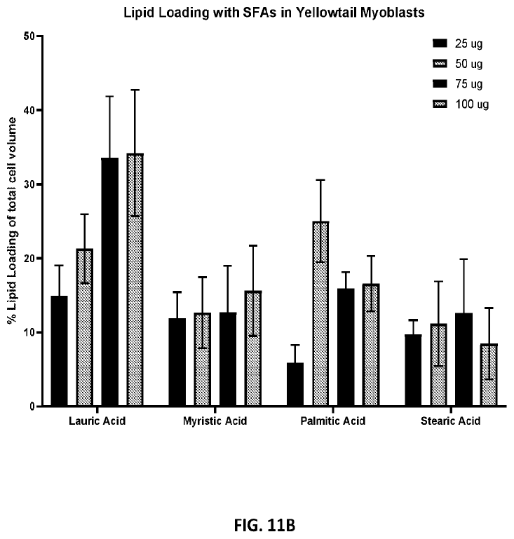

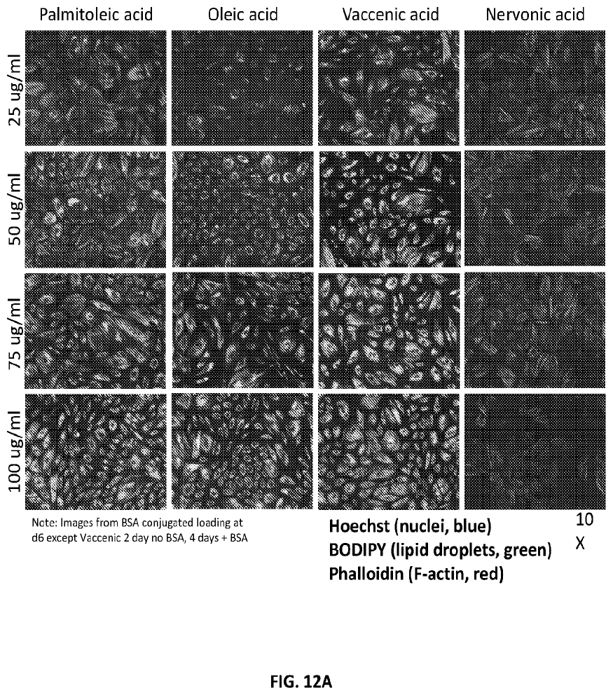

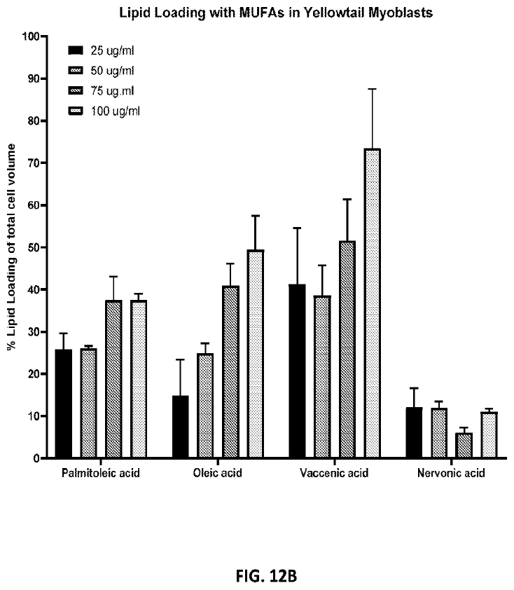

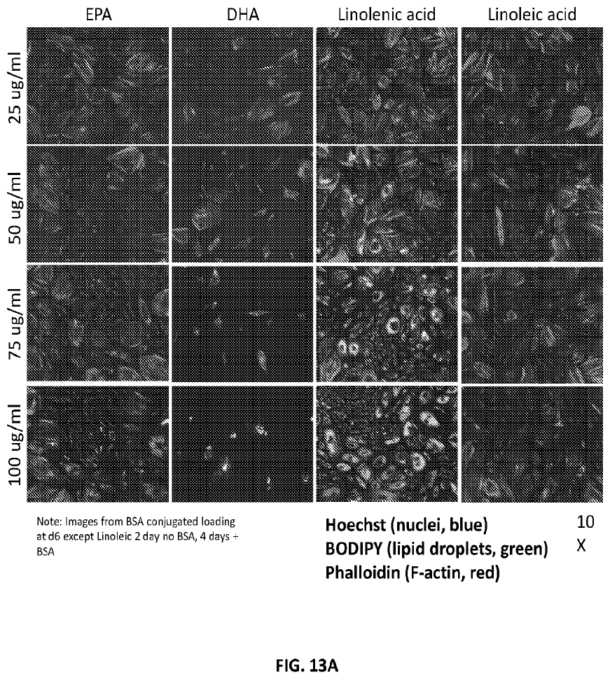

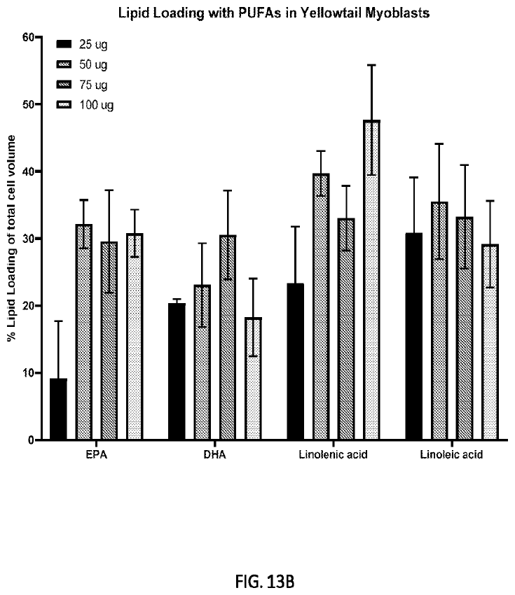

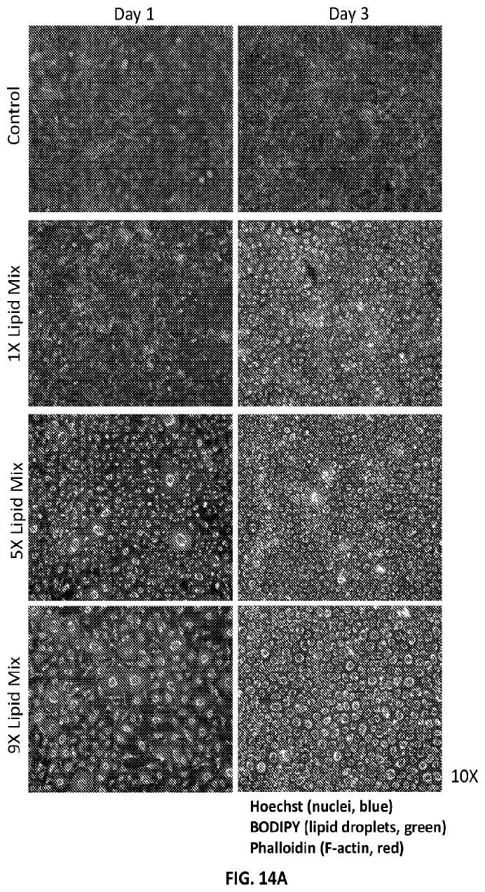

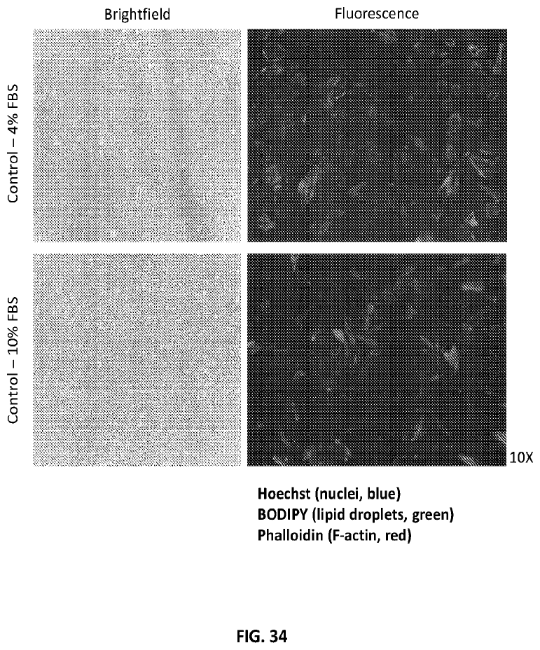

The amount of fatty acid accumulation and subsequent nutritional composition of the cells was controlled by fatty acid chemistry, concentration, and time. A literature search on yellowtail nutritional composition was performed and the top four each saturated, unsaturated and monounsaturated fatty acids were compiled and chosen for this example. Each major type of fatty acid was able to load into yellowtail myoblasts and maintained for up to 6 days and confirmed by BODIPY immunofluorescent staining (FIGS. 11A, 11B, 12A, 12B, 13A, and 13B). Negative control cultures cultured in the presence of serum and without additional lipids did not show lipid accumulation (FIG. 34).

Saturated fatty acid lauric acid showed significant accumulation at all concentrations tested while the remaining SFA, myristic acid, palmitic acid, and stearic acid, had limited accumulation with only small areas of uptake even at the highest concentration of 100 μg/ml. The majority of MUFA tested showed concentration-dependent lipid accumulation with significant lipid accumulation even at the lowest concentration tested. The exception to MUFA loading was nervonic acid which showed no lipid accumulation at any concentration tested. PUFA also showed lipid accumulation that was concentration-driven, particularly with respect to linolenic acid. Of note, DHA which is a relevant nutritional fatty acid, showed limited loading at all concentrations and toxicity as evidenced by altered cell morphology and decreased cell number above 25 μg/ml.

These data suggest that carbon length is not a key mediator of fatty acid loading as each of the 18 carbon fatty acids tested, stearic acid, vaccenic acid, oleic acid, linoleic, and linolenic acid each had different concentration dependent loading outcomes. Fatty acids of carbon length from 12 to 22 successfully loaded over a range of chemistries.

Cis-vaccenic acid was taken up very readily by fish cells despite it not being a major component in most fish. This fatty acid is a common fatty acid of bacterial lipids and is usually present in most plant and animal tissues. The ability of fish cells to take up a significant amount of fatty acid over what may typically be present supports the use of targeted fatty acid loading to alter the nutritional profile of cells and/or cell-cultured seafood products via lipid loading.

### Example 6: Loading Chemically Defined Fatty Acid Mixtures into Muscle-Derived Cells of Fish

In this example, loading of chemically defined fatty acid mixtures was demonstrated for muscle derived cells from fish with mixtures of fatty acids of varying chemical structures and concentrations.

The accumulation of specific fatty acids such as Omega-3 versus saturated fatty acids allows for tuning of nutritional composition in the cells as well as final 3D seafood product. Fatty acids not identified as a contributor to lipid accumulation when used individually demonstrated synergistic capabilities to enhance lipid accumulation or improve cell health to enable fatty acid accumulation beyond that of an individual fatty acid.

Myoblasts derived from yellowtail as described in Example 4 were evaluated for the ability to uptake fatty acids from a defined chemical composition. Cells were seeded onto tissue culture polystyrene at a density of 6000 cells per cm². Cells were cultured in the presence of control media without fatty acids or test media with fatty acids conjugated to BSA in the presence of 4% serum for two or six days. Concentrations were used for each individual fatty acid at 0, 10, 25, or 50 μg/ml. Combinations of fatty acids were determined using a design of experiments using a Taguchi L-27 design (Table 8). Fatty acids tested included monounsaturated fatty acids and polyunsaturated fatty acids as shown in Table 7, Example 5 with varying carbon chain length and location of double bonds along the carbon chain. The experimental design did not include SFA, in contrast to existing fish cells that do contain SFA, particularly palmitic acid and stearic acid.

Cells were fixed, stained and imaged at day two or day six. Cells were treated with NucBlue, BODIPY and Mitotracker for 1.5 hours and live imaging was performed. Cells were then fixed in 4% PFA for 15 minutes and then blocked overnight at 4 degrees Celsius in blocking buffer that contained 5% goat serum, 1% BSA, and 0.2% fish gelatin in PBS. Cells were incubated with Phalloidin for 1 hour at room temperature in blocking buffer (Phalloidin 1:800). Plates were washed twice with PBS prior to imaging. JMP statistical software was used to quantify the effect of each fatty acid on cell number, fatty acid accumulation, and cell health.

TABLE 8
Design for analysis of fatty acid impacts on cell
number, fatty acid accumulation, and cell health.

| | | Alpha- | | | | | | |
|---|---|---|---|---|---|---|---|---|
| Media | Linoleic | Linolenic | | | Nervonic | Vaccenic | Oleic | Palmitoleic |
| ID | acid | acid | DHA | EPA | acid | acid | acid | acid |
| 1 | 50 | 50 | 25 | 25 | 50 | 50 | 50 | 50 |
| 2 | 50 | 50 | 25 | 25 | 10 | 10 | 10 | 10 |
| 3 | 50 | 50 | 25 | 25 | 0 | 0 | 0 | 0 |
| 4 | 50 | 10 | 10 | 10 | 50 | 50 | 50 | 10 |
| 5 | 50 | 10 | 10 | 10 | 10 | 10 | 10 | 0 |
| 6 | 50 | 10 | 10 | 10 | 0 | 0 | 0 | 50 |
| 7 | 50 | 0 | 0 | 0 | 50 | 50 | 50 | 0 |
| 8 | 50 | 0 | 0 | 0 | 10 | 10 | 10 | 50 |
| 9 | 50 | 0 | 0 | 0 | 0 | 0 | 0 | 10 |
| 10 | 10 | 50 | 10 | 0 | 50 | 10 | 0 | 50 |
| 11 | 10 | 50 | 10 | 0 | 10 | 0 | 50 | 10 |
| 12 | 10 | 50 | 10 | 0 | 0 | 50 | 10 | 0 |
| 13 | 10 | 10 | 0 | 25 | 50 | 10 | 0 | 10 |
| 14 | 10 | 10 | 0 | 25 | 10 | 0 | 50 | 0 |
| 15 | 10 | 10 | 0 | 25 | 0 | 50 | 10 | 50 |
| 16 | 10 | 0 | 25 | 10 | 50 | 10 | 0 | 0 |
| 17 | 10 | 0 | 25 | 10 | 10 | 0 | 50 | 50 |
| 18 | 10 | 0 | 25 | 10 | 0 | 50 | 10 | 10 |
| 19 | 0 | 50 | 0 | 10 | 50 | 0 | 10 | 50 |
| 20 | 0 | 50 | 0 | 10 | 10 | 50 | 0 | 10 |
| 21 | 0 | 50 | 0 | 10 | 0 | 10 | 50 | 0 |
| 22 | 0 | 10 | 25 | 0 | 50 | 0 | 10 | 10 |
| 23 | 0 | 10 | 25 | 0 | 10 | 50 | 0 | 0 |
| 24 | 0 | 10 | 25 | 0 | 0 | 10 | 50 | 50 |
| 25 | 0 | 0 | 10 | 25 | 50 | 0 | 10 | 0 |
| 26 | 0 | 0 | 10 | 25 | 10 | 50 | 0 | 50 |
| 27 | 0 | 0 | 10 | 25 | 0 | 10 | 50 | 10 |
| 28 | C | C | C | C | C | C | C | C |
| 29 | 0 | 0 | 0 | 0 | 0 | 0 | 0 | 0 |

### Results

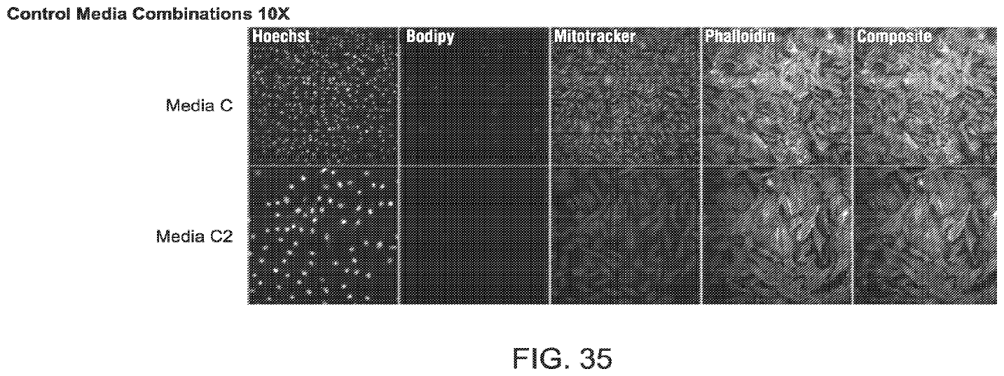

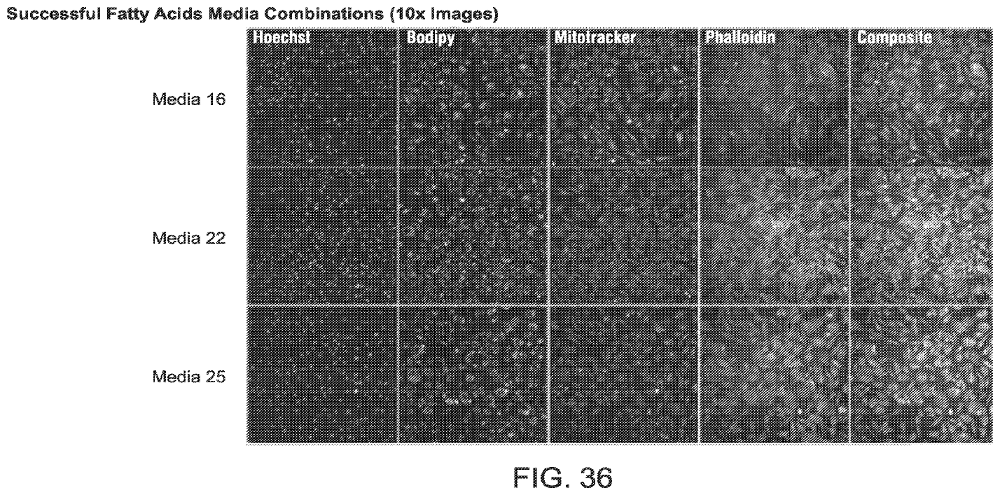

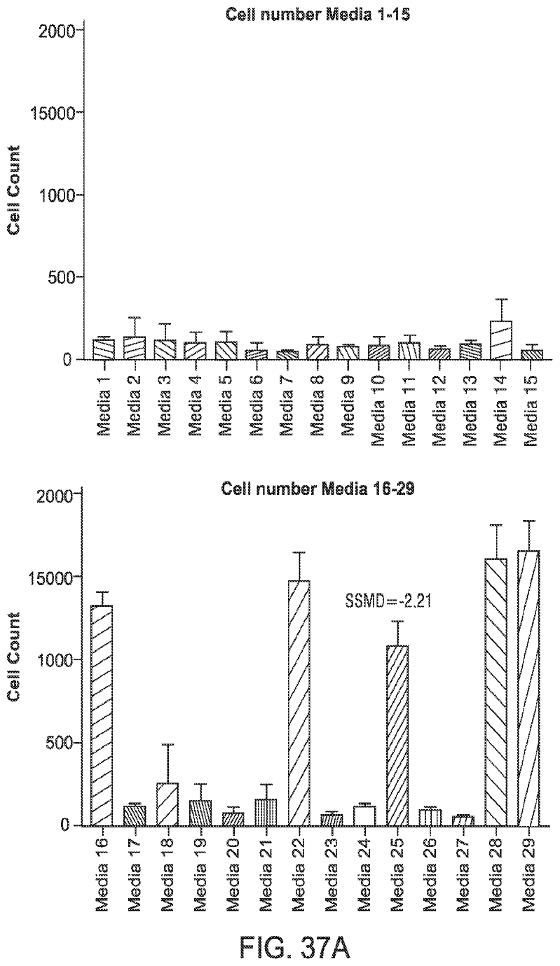

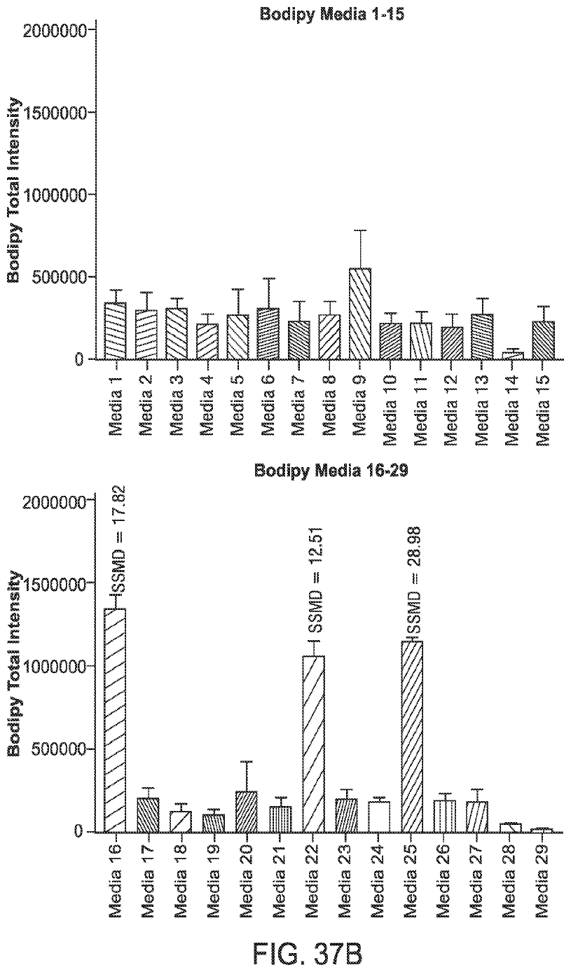

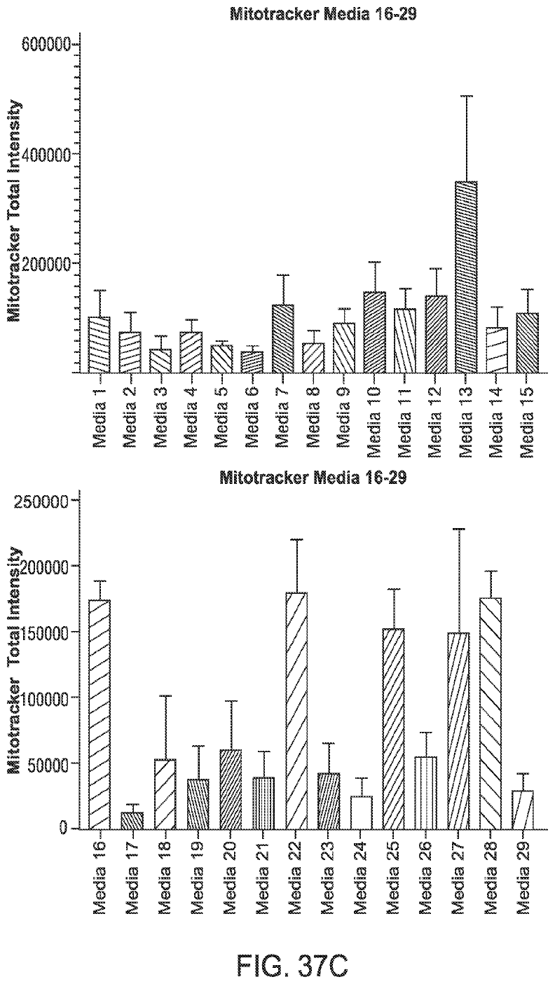

As in previous Examples 4 and 5, no fatty acid accumulation was detected in control conditions without addition of fatty acids (FIG. 35). All fatty acids tested resulted in some lipid accumulation as shown by BODIPY staining with highest levels achieved in conditions 16, 22, and 25 (FIG. 36, Table 9). The common factor in each of the combinations with highest fatty acid accumulation was the presence of nervonic acid at 50 μg/ml. The high accumulation of fatty acid in the presence of nervonic acid was surprising based on prior results showing nervonic acid itself does not load when used individually as shown in Example 5. Importantly, in each of the mixtures with high lipid accumulation, at least one unsaturated omega-3 fatty acid was present. While the lipid accumulation of each individual fatty acid was minimal at 25 μg/ml as shown in Example 4, significant loading was observed for total lipid droplet accumulation when the same fatty acids at low concentration were used in combination. Of note, combinations 22 and 25 use levels of fatty acids with very low loading observed for any individual concentration tested, whereas the combination resulted in comparatively high lipid accumulation. In addition, combinations with highest total lipid loading also showed higher cell number, indicating maintained cell viability compared to other conditions (FIG. 37). Statistical analysis of lipid accumulation identified nervonic acid as a key factor in increasing lipid accumulation and cell number. As shown in Table 9, all combinations with high fatty acid accumulation contained nervonic acid at 50 μg/ml. Compared to individual fatty acid testing in Example 5 that showed low lipid accumulation and some toxicity of DHA when used individually, high cell numbers and high lipid accumulation are shown when used in combination with nervonic acid.

Saturated fatty acid content in fish can be approximately 40% of the total lipid content. In the Western diet, SFA intake is very high as well, so limiting the amount of SFA content in our products will produce higher quality nutritional value for consumers.

One relevant component of combinations that promoted high lipid loading was the presence of nervonic acid. Nervonic acid is important in cerebrosides, which are fatty acids that are components of muscle and the central and peripheral nervous systems. Nervonic acid is also one of the major fatty acids found in brain sphingolipids. Nervonic acid has been found in breast milk and is recommended for pregnant and nursing women. It also can have neuroprotective effects and is commonly found in energy supplements. The use of nervonic acid in enhanced lipid loading of cells is, to the inventors' knowledge, not yet known. The use of nervonic acid may enable lipid loading of otherwise toxic fatty acids as well as contribute additional nutritional qualities to seafood products.

TABLE 9
Fatty acid mixtures with highest lipid loading

| | | Alpha- | | | | | | |
|---|---|---|---|---|---|---|---|---|
| Media | Linoleic | Linolenic | | | Nervonic | Vaccenic | Oleic | Palmitoleic |
| ID | acid | acid | DHA | EPA | acid | acid | acid | acid |
| 16 | 10 | 0 | 25 | 10 | 50 | 10 | 0 | 0 |
| 22 | 0 | 10 | 25 | 0 | 50 | 0 | 10 | 10 |
| 25 | 0 | 0 | 10 | 25 | 50 | 0 | 10 | 0 |

### Example 7: Loading of Fatty Acid Mixtures and Single Fatty Acids into Mammalian Cells

In this example, loading of complex fatty acid mixtures comprised of saturated and unsaturated fatty acids, including omega-3s, and single fatty acids, including polyunsaturated fatty acids such as omega-3s into muscle precursor cells (myoblasts) and fibroblasts, is demonstrated for mammalian cells.

Myoblasts or fibroblasts of cow, pork, goat, lamb, or deer are isolated from muscle tissue by enzymatic and mechanical/or manual dissociation. Cells are expanded in 2D tissue culture or 3D cell suspension culture.

Cells are cultured in the presence of control media without fatty acids or test media with individual fatty acids at 1, 5, 10, 15, 25, 50, 75, or 100 μg/ml in the absence or presence of reduced serum. Fatty acids are conjugated to BSA or unconjugated. Individual fatty acids include saturated fatty acids, monounsaturated fatty acids, and poly-unsaturated fatty acids with varying carbon chain lengths and location of double bonds along the carbon chains.

No lipid droplet accumulation is detected in control conditions without addition of fatty acids. All fatty acids tested result in some lipid accumulation, but this lipid accumulation of each individual fatty acid is minimal. However, significant lipid loading is observed for total lipid accumulation when the same fatty acids at low concentrations are used in various combinations.

Nervonic acid increases cell proliferation when added to the growth medium as the only fatty acid. In combination with other fatty acids, nervonic acid causes increased lipid uptake and lipid accumulation. The omega-3 polyunsaturated fatty acids, DHA and EPA, are not taken up well when tested alone. When nervonic acid is added to this mix, cells are able to proliferate faster and uptake and store these polyunsaturated fatty acids at higher levels.

### Example 8: Loading of Fatty Acid Mixtures and Single Fatty Acids into Cells from Avian Species

In this example, loading of complex fatty acid mixtures, comprised of saturated and unsaturated fatty acids including omega-3s, and single fatty acids including poly-unsaturated fatty acids such as omega-3s into muscle precursor cells (myoblasts) and fibroblasts is demonstrated for mammalian cells.

Myoblasts or fibroblasts of chicken, duck, goose, or turkey are isolated from muscle tissue by enzymatic and mechanical/or manual dissociation. Cells are expanded in 2D tissue culture or cell suspension culture.

Cells are cultured in the presence of control media without fatty acids or test media with individual fatty acids at 1, 5, 10, 15, 25, 50, 75, or 100 μg/ml in the absence or presence of reduced serum. Fatty acids are conjugated to BSA or unconjugated. Individual fatty acids include saturated fatty acids, monounsaturated fatty acids, and poly-unsaturated fatty acids with varying carbon chain lengths and location of double bonds along the carbon chains.

No lipid accumulation is detected in control conditions without addition of fatty acids. All fatty acids tested result in some lipid accumulation, but this lipid accumulation of each individual fatty acid is minimal. However, significant lipid loading is observed for total lipid accumulation when the same fatty acids at low concentrations are used in various combinations.

Nervonic acid increases cell proliferation when added to the growth medium as the only fatty acid. In combination with other fatty acids nervonic acid causes increased lipid uptake and lipid accumulation. The omega-3 polyunsaturated fatty acids, DHA and EPA, are not taken up well when tested alone. When nervonic acid is added to this mix, cells are able to proliferate faster and uptake and store these polyunsaturated fatty acids at higher levels.

### Example 9: Enrichment of Fish Products with Lipids

Fish myoblasts, preadipocytes or fibroblasts are grown in 2D tissue culture or cell suspension culture. The growth medium is supplemented with complex fatty acid mixtures, comprised of saturated, monounsaturated fatty acids and polyunsaturated fatty acids including omega-3s, or single fatty acids including polyunsaturated fatty acids such as omega-3 fatty acids. The lipid-loaded cells are harvested by cell separation techniques such as settling or tangential flow filtration. The harvested cells are assembled using various methods, including extrusion and bioprinting, to form cubes, strips or fillets. The cubes, strips or fillets are composed of myoblasts or adipocytes or fibroblasts or combinations of these cell types. The resulting fish product contains higher levels of total lipids than conventionally sourced fish. The resulting fish product contains higher levels of polyunsaturated fatty acids, such as omega-3 fatty acids, than conventionally sourced fish.

In another embodiment, fish myoblasts, preadipocytes or fibroblasts are grown in 2D tissue culture or cell suspension culture and then harvested by centrifugation. The cells are then mixed with differentiation medium supplemented with complex fatty acid mixtures, comprised of saturated, monounsaturated fatty acids and polyunsaturated fatty acids including omega-3s, or single fatty acids including polyunsaturated fatty acids such as omega-3 fatty acids.

### Example 10: Dose-Dependent Loading of Complex FA Mixtures into Cells Derived from Adipose, Muscle and Connective Tissues of Multiple Species

In this example, loading of a complex fatty acid mixture, comprised of saturated and unsaturated fatty acids including omega-3s, into muscle, adipose and connective tissue-derived cells from multiple terrestrial and aquatic species (Table 10 below) was demonstrated.

Pre-adipocytes from Silver Carp and Bluefin tuna were prepared as described in Example 1 above. Bluefin fibroblast cells were prepared as for Bluefin myoblasts. Yellowtail fibroblasts were prepared as described in Example 3 above. Mahi-mahi myoblasts were prepared as in Example 4 above. The remaining cells were obtained from ATCC or from commercial sources and cultured according to information provided by the supplier (Tables 4-5 above).

### Cell Culture and Lipid Loading

Cells were seeded onto polystyrene six well plates. Myoblast cells were seeded at a density of 5000-10,000 cells per cm² Preadipocytes were seeded at a density of 5-7000 cells per cm².

The cells were then cultured in the presence of control media without fatty acid mixture or test media with fatty acid mixture (Sigma Lipid Mix, 1%) in the presence of control (10% FBS) or reduced serum (4% FBS) media. For all cell types and species, media was changed every other day with fresh lipids.

### Cell Morphology and Immunofluorescent Staining

Cell morphology was observed over a period of up to seven days by brightfield microscopy for characteristic rounding and lipid loading. Immunofluorescent staining of cells was performed on day 6 or 7 for cell nuclei by Hoechst and lipid droplets by BODIPY as described in Example 1.

### Results

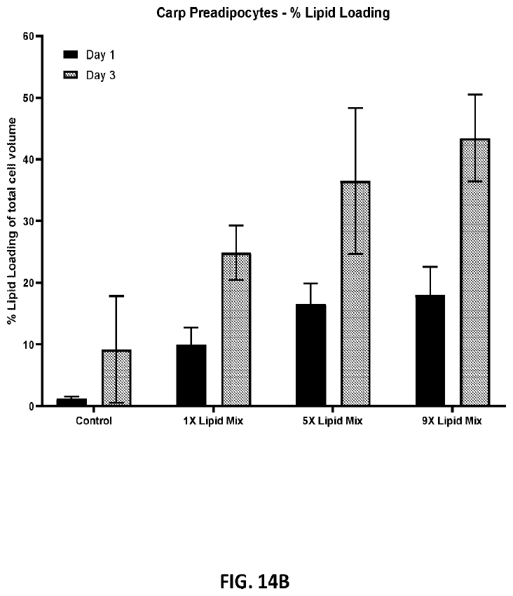

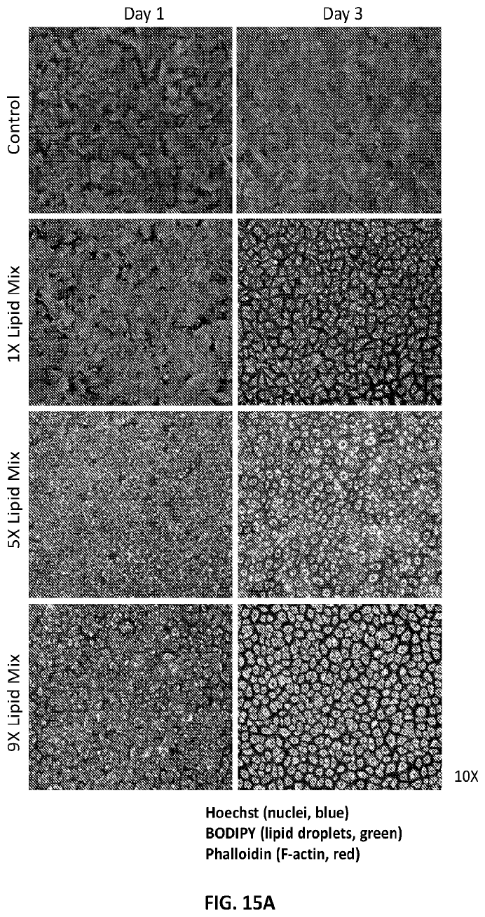

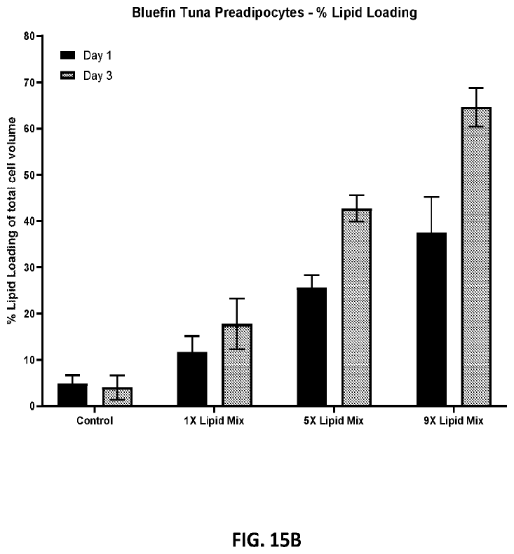

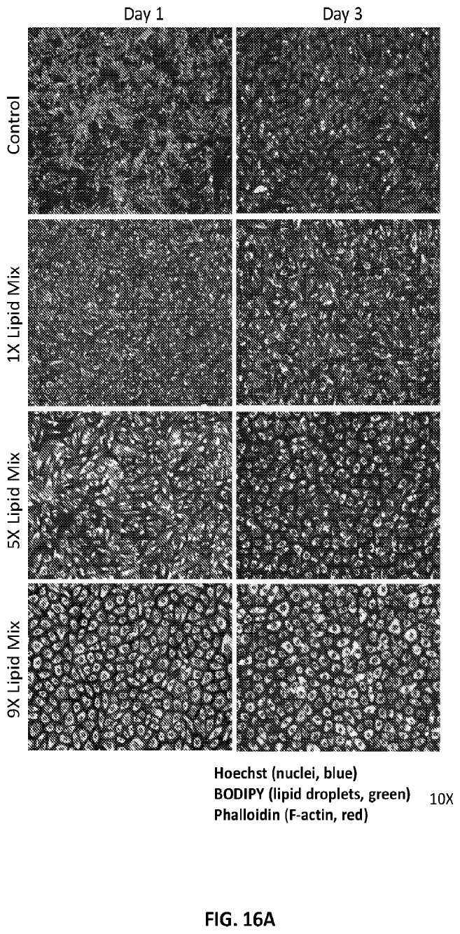

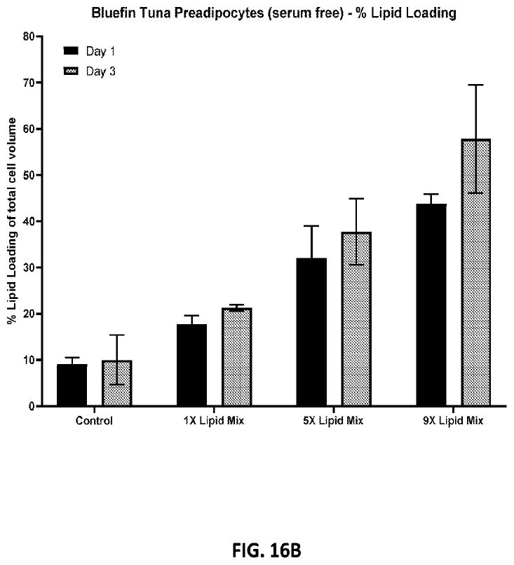

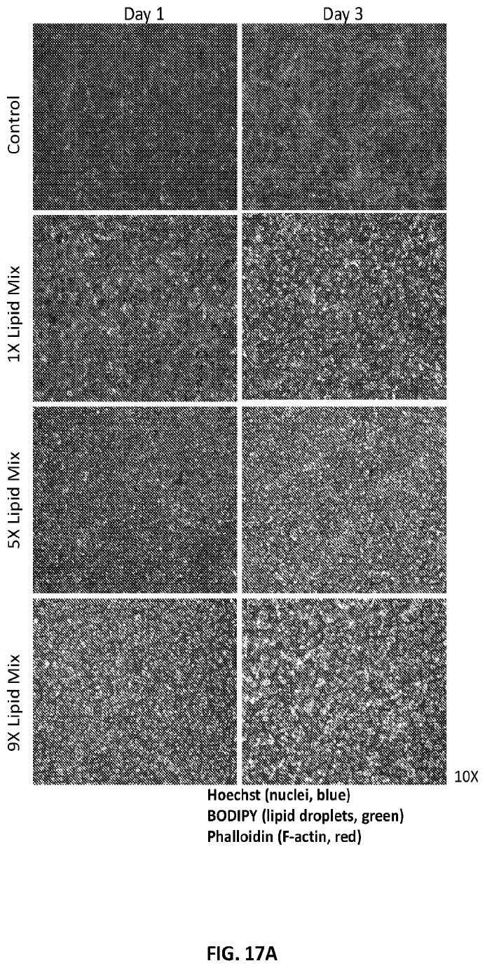

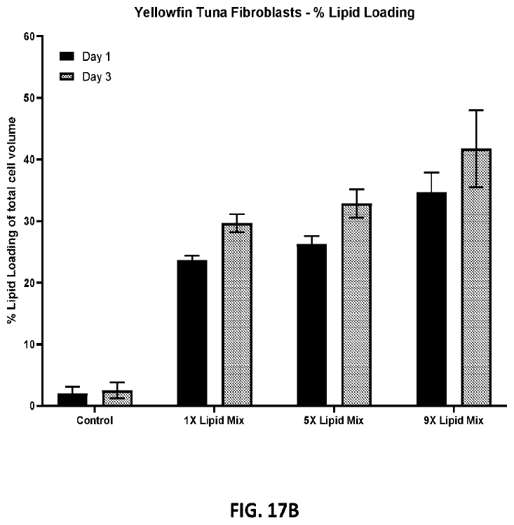

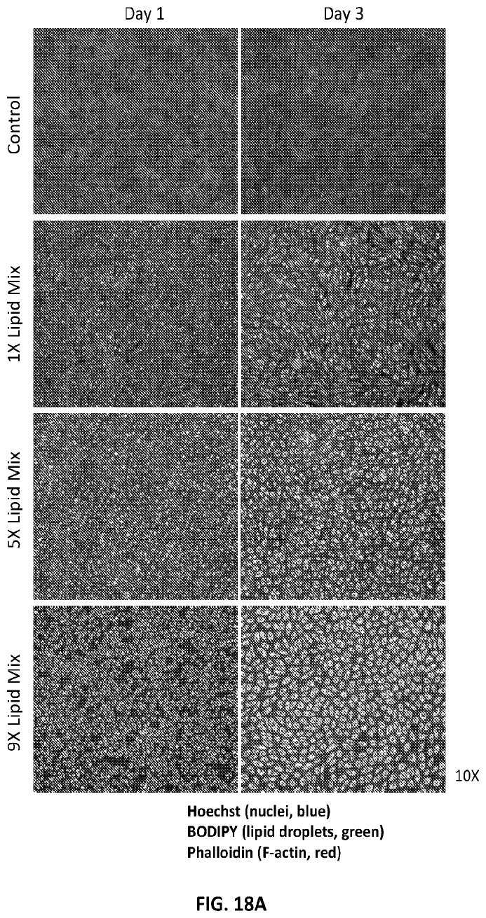

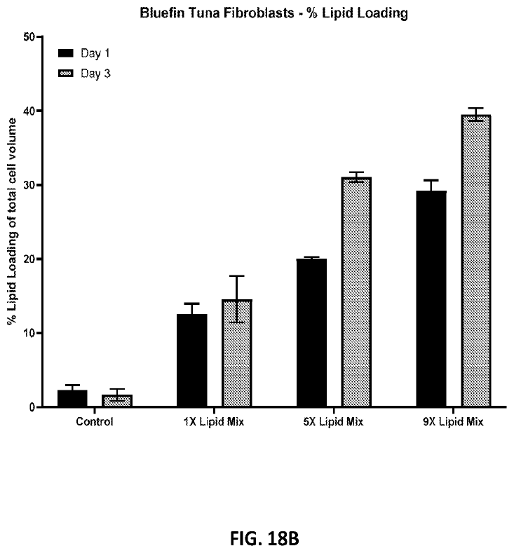

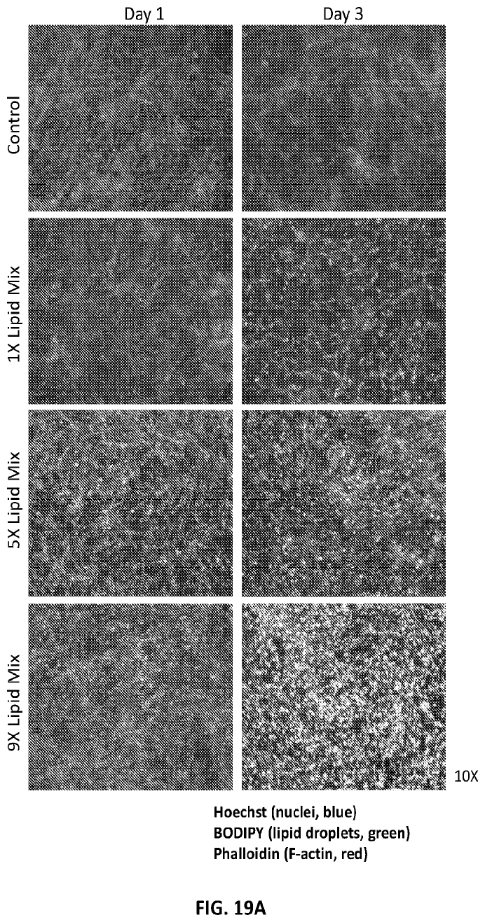

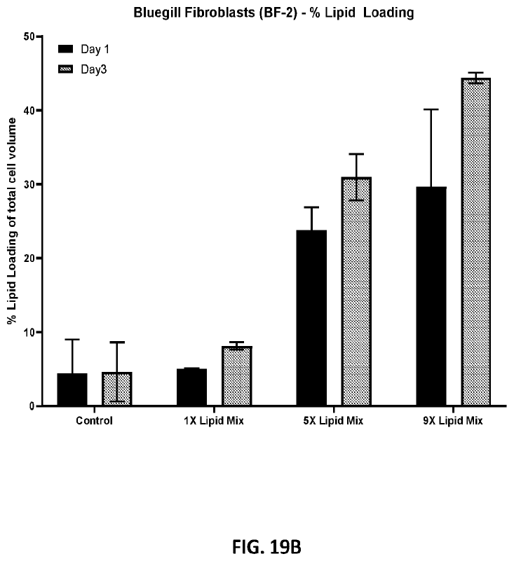

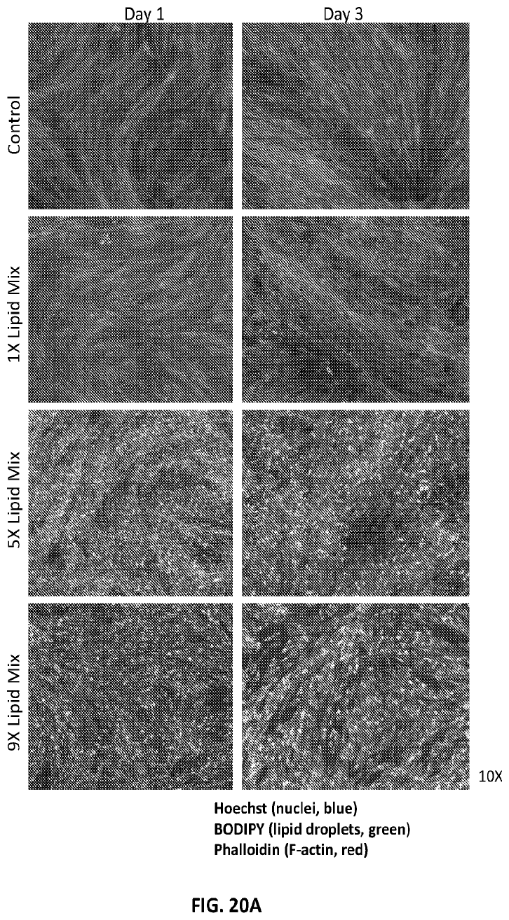

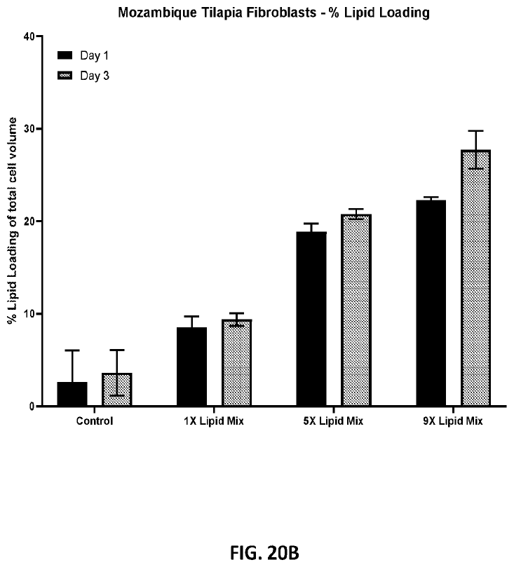

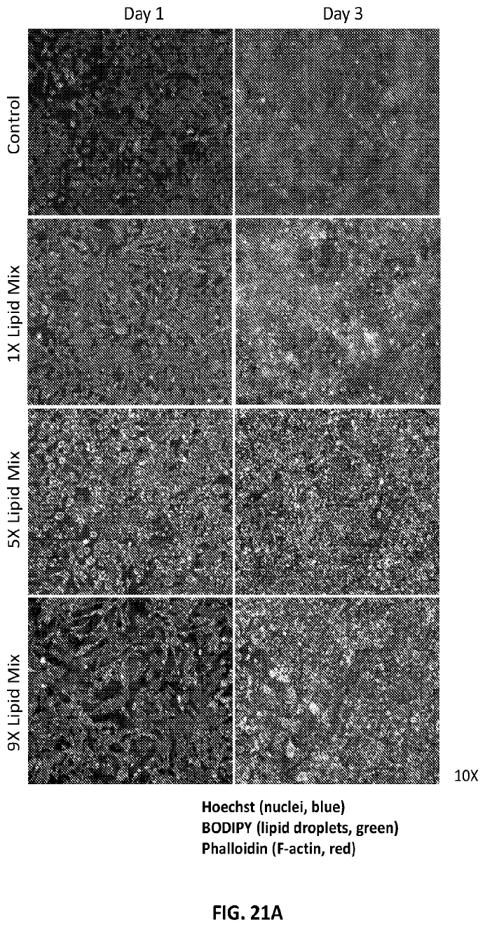

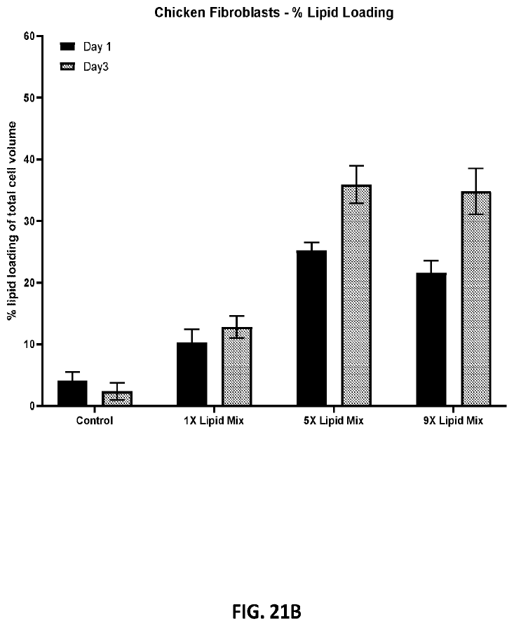

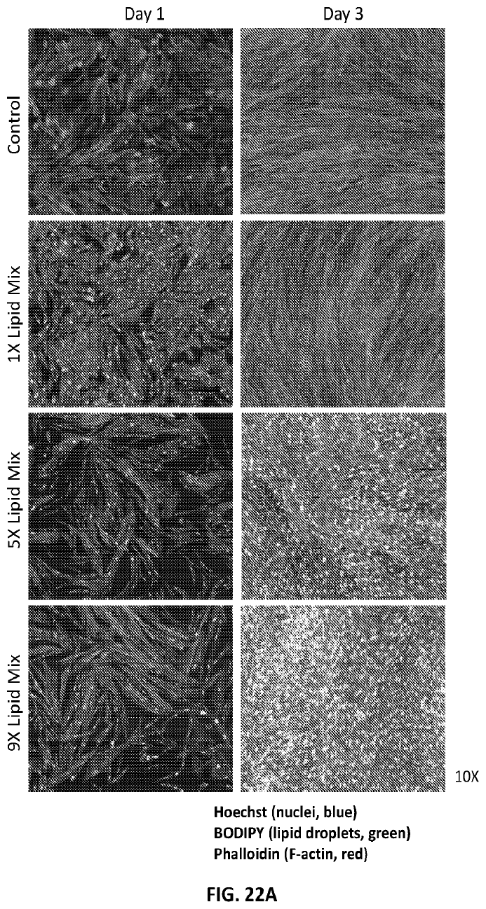

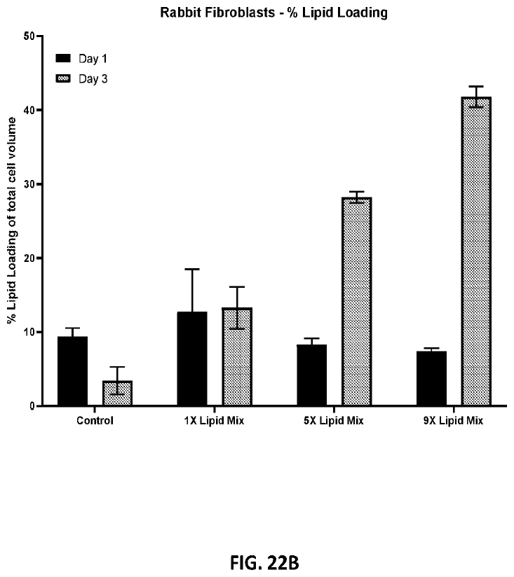

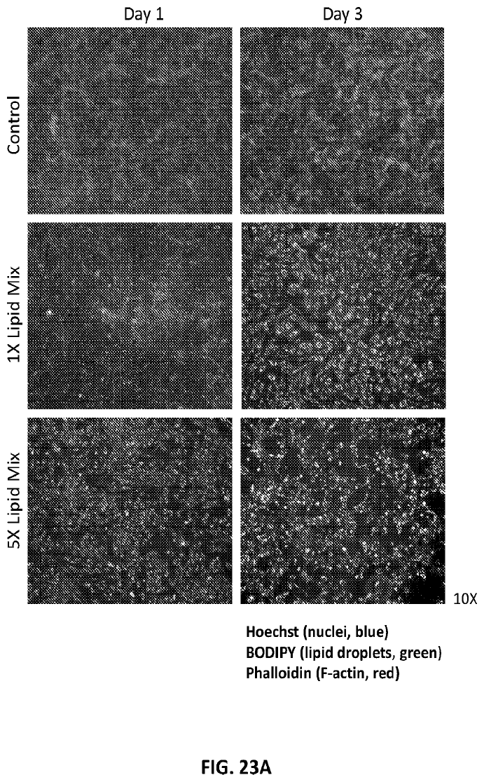

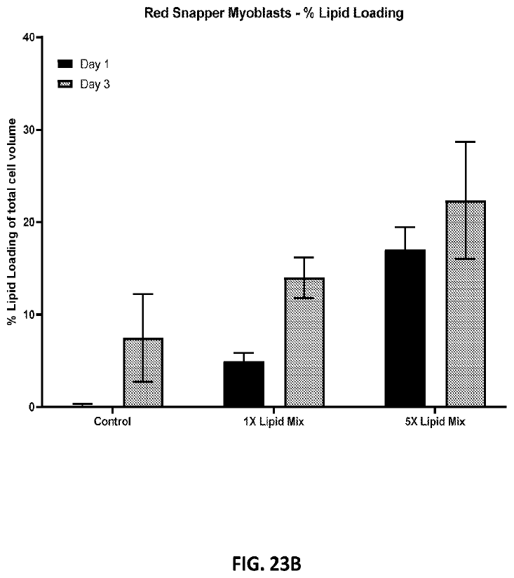

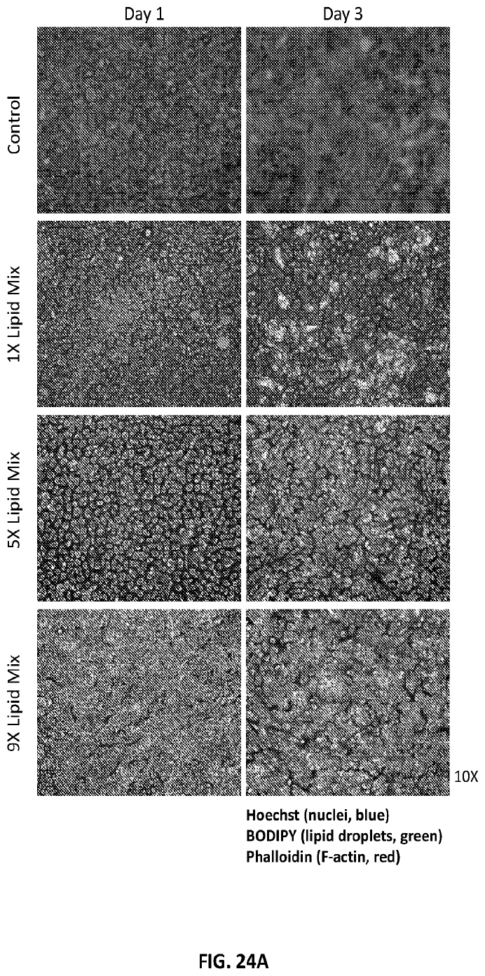

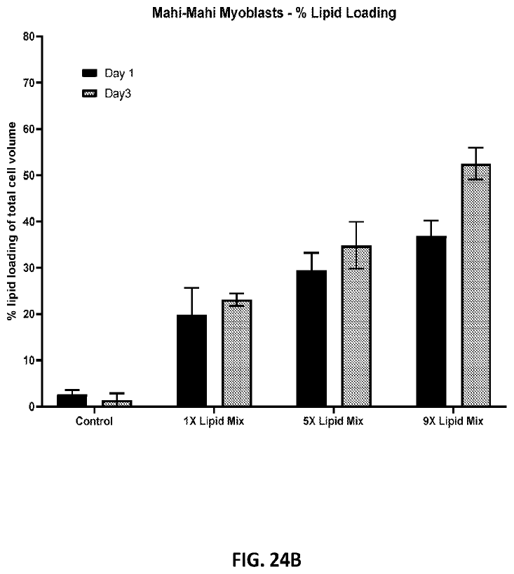

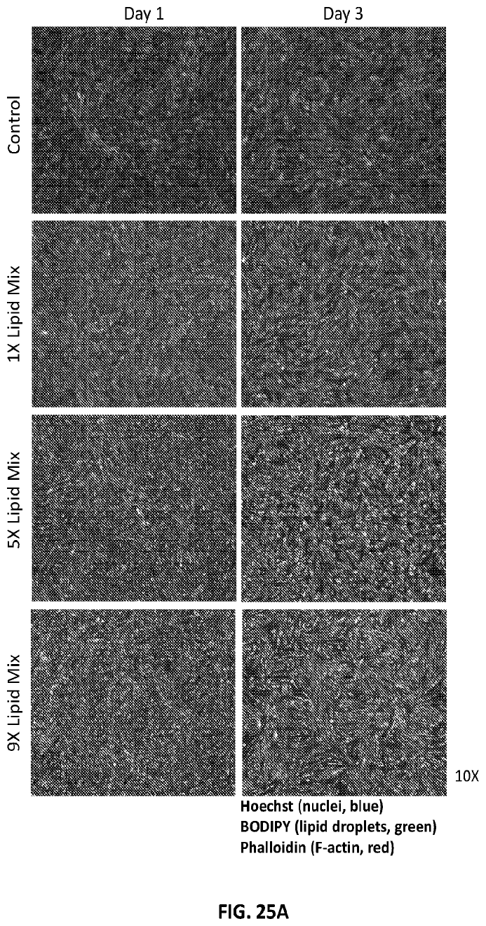

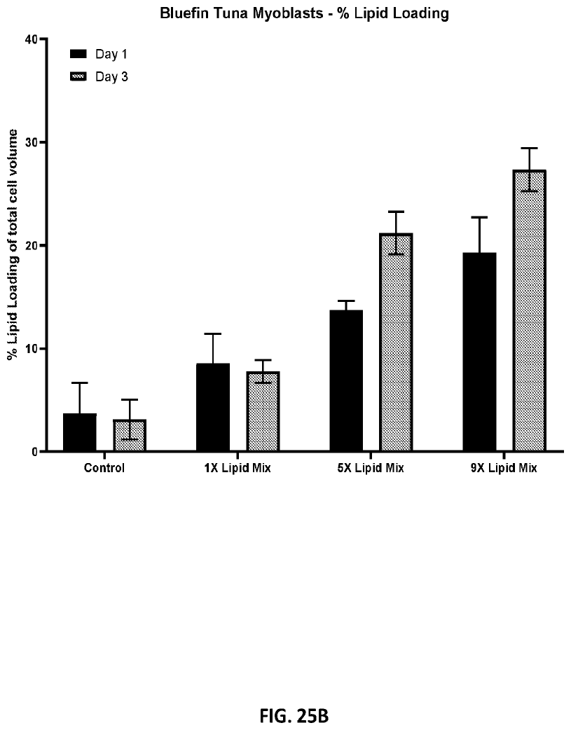

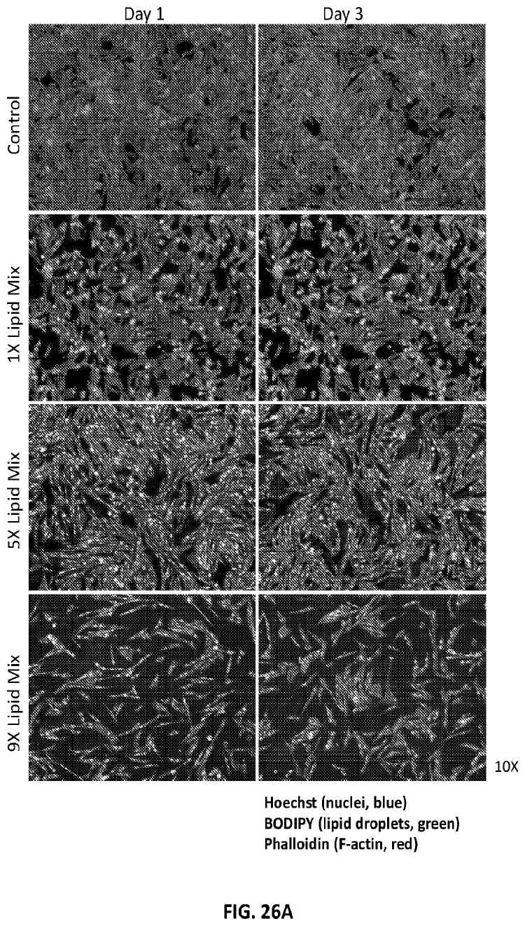

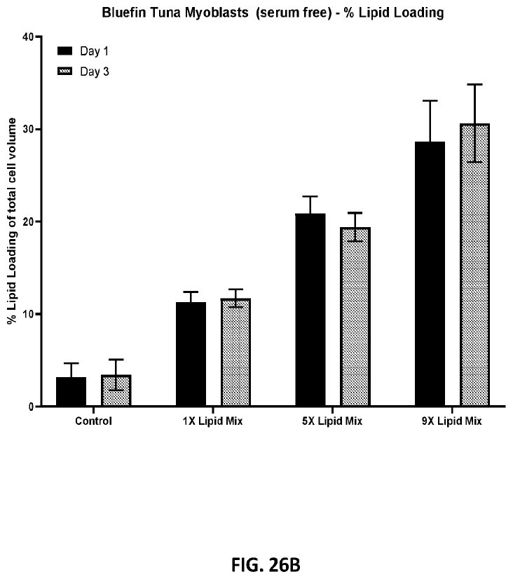

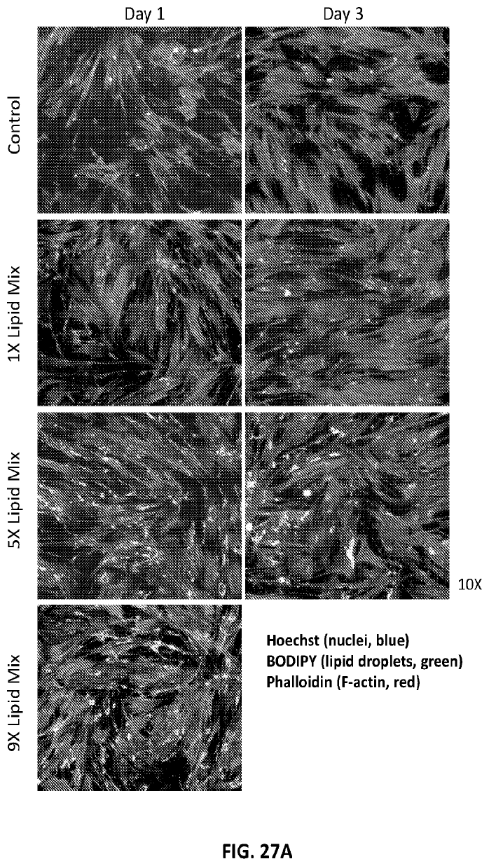

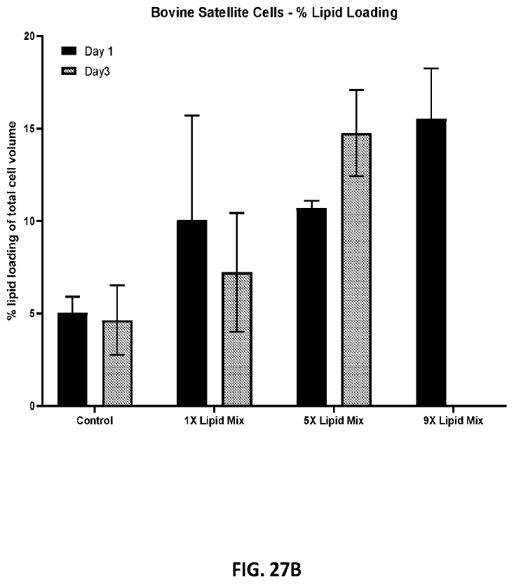

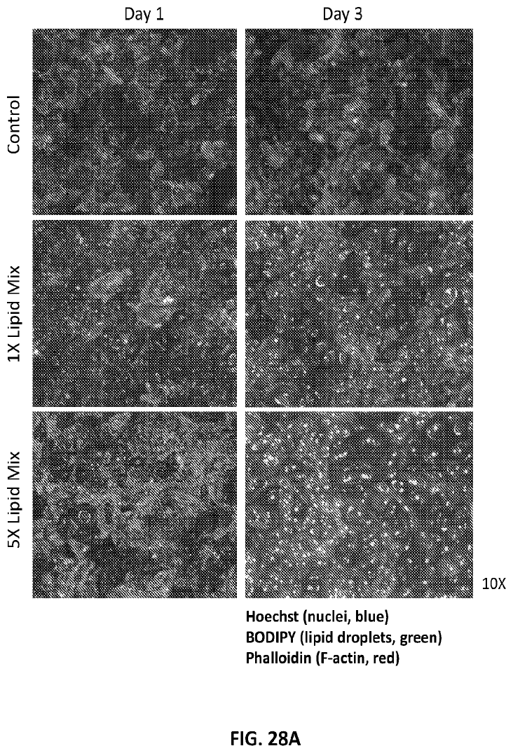

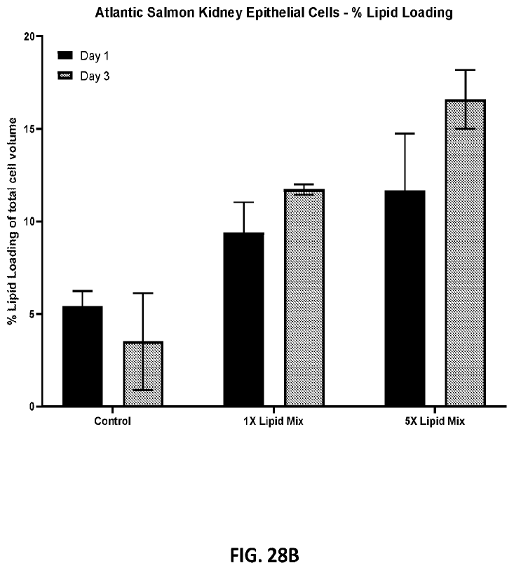

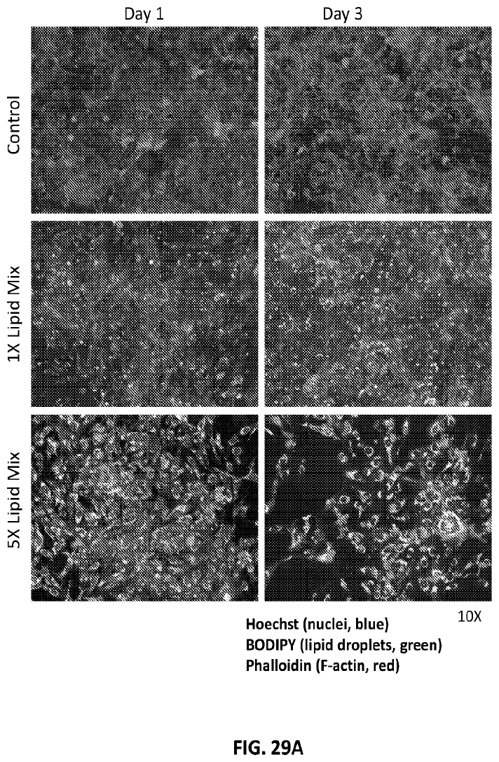

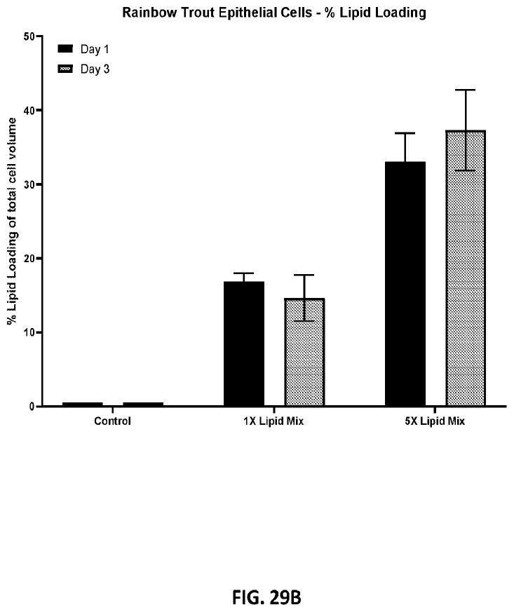

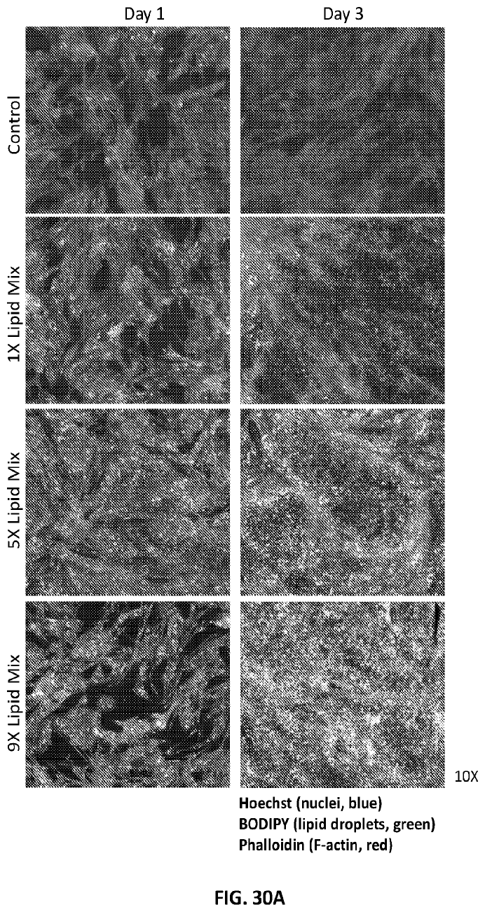

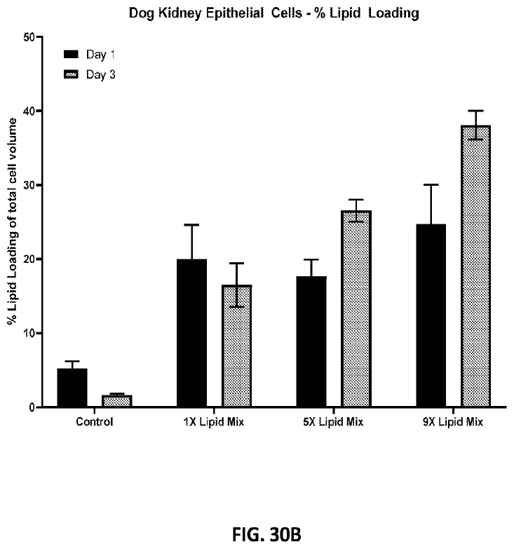

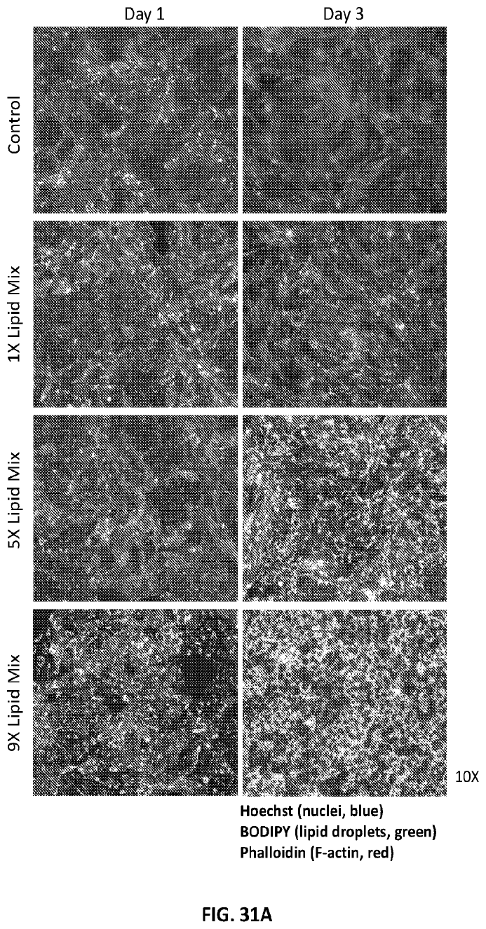

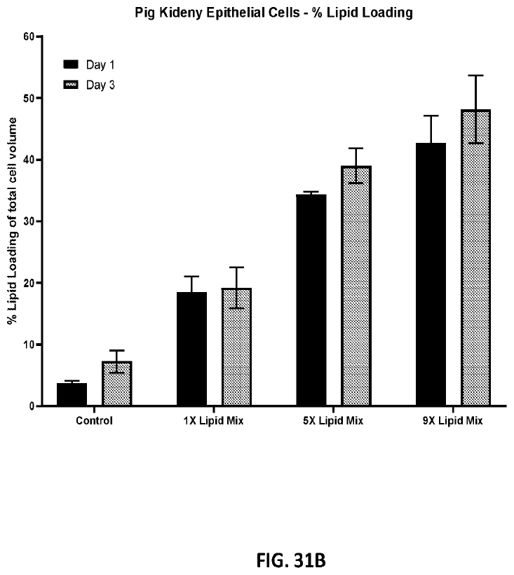

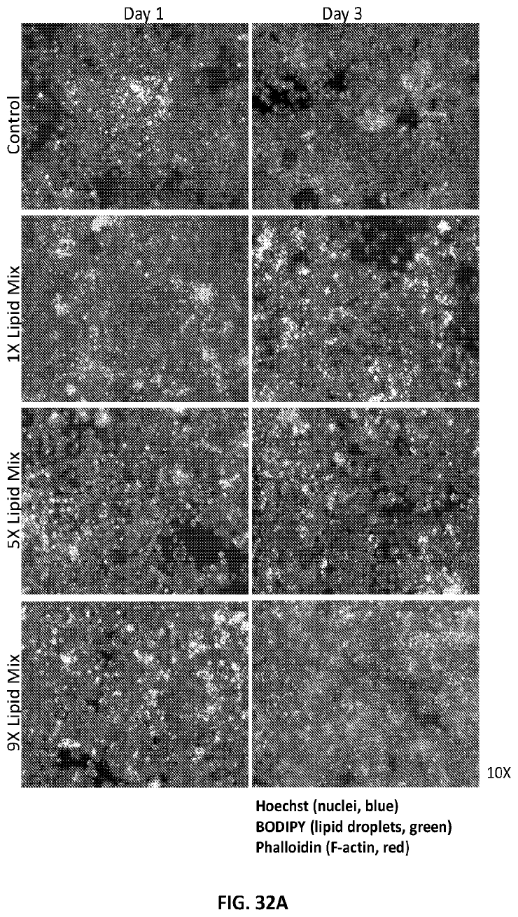

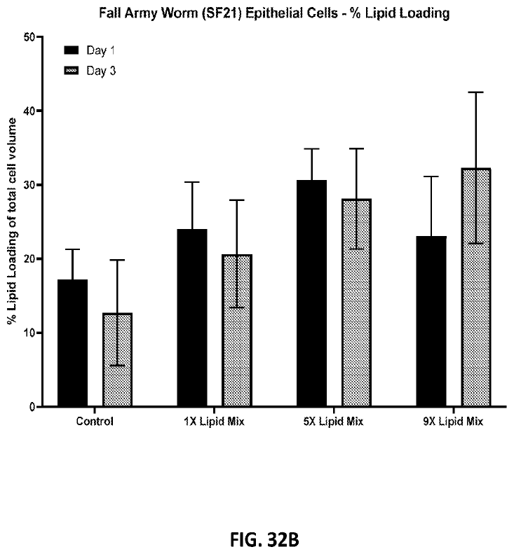

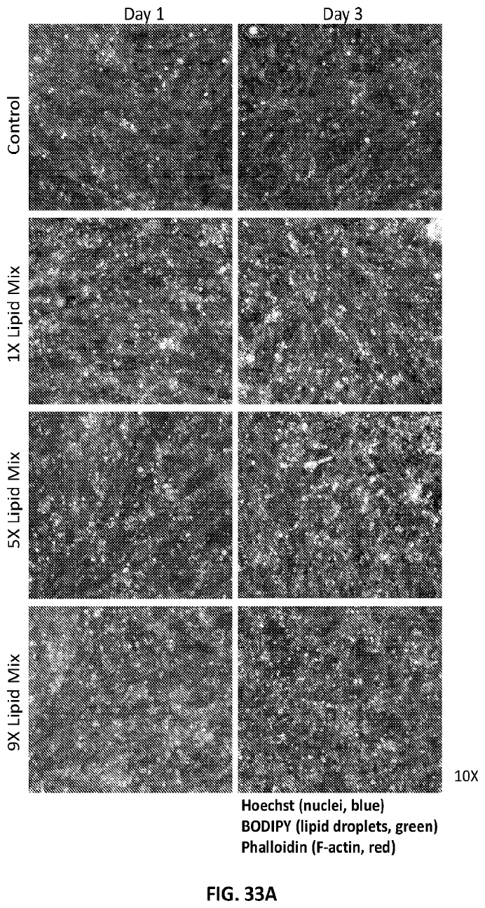

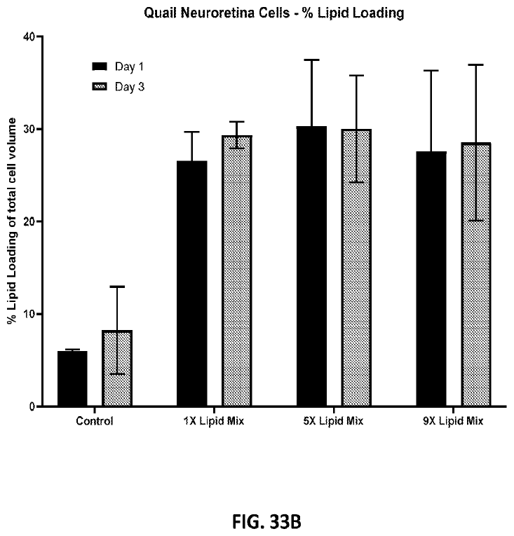

Immunofluorescent images and corresponding lipid quantification data for each sample are presented in FIG. 14A through FIG. 33B (Table 10 below). Quantification of lipid loading shows increased loading compared to control and naturally occurring levels for all species. For fibroblasts and myoblasts with typically <1% fat content, increases of over 70 percentage points are observed at 3 days. As shown in previous examples, longer durations indicate the potential increase to further rise up to 90% of cell volume. Surprisingly, many cell types that do not typically store fat in nature such as those from muscle, brain, skin, and embryonic tissue were able to load fat as effectively as preadipocytes using methods described herein.

TABLE 10
List of figures for each species used in
the dose dependent loading of lipids.

| | | FIG. | |
|---|---|---|---|
| | | Immuno- | |
| | | fluorescent | Quanti- |
| Species and cell type | Cell line ID | Staining | fication |
| Carp preadipocytes | | 14A | 14B |
| Bluefin preadipocytes | | 15A | 15B |
| Bluefin preadipocytes (serum | | 16A | 16B |
| free) | | | |
| Yellowfin fibroblasts | | 17A | 17B |
| Bluefin fibroblasts | | 18A | 18B |
| Bluegill fibroblasts | BF-2 | 19A | 19B |
| Tilapia brain derived cells | OmB | 20A | 20B |
| Chicken embryonic | UMNSAH/ | 21A | 21B |
| fibroblasts | DF-1 | | |
| Rabbit skin fibroblasts | RAB-9 | 22A | 22B |
| Red snapper muscle derived | | 23A | 23B |
| Mahi mahi myoblasts | | 24A | 24B |
| Bluefin myoblasts | | 25A | 25B |
| Bluefin myoblasts (serum | | 26A | 26B |
| free) | | | |
| Bovine muscle satellite cells | | 27A | 27B |
| Salmon kidney epithelial | ASK | 28A | 28B |
| Rainbow trout gill epithelial | Rtgill-W1 | 29A | 29B |
| cells | | | |
| Dog kidney epithelial cells | MDCK (NBL-2) | 30A | 30B |
| Pig kidney epithelial cells | PK15 | 31A | 31B |
| Fall army worm epithelial | SF21 | 32A | 32B |
| cells | | | |
| Quail neuroretina cells | QNR/K2 | 33A | 33B |

### Example 11: Lipidomics Analysis of Lipid-Loaded Cells

In this example, the fatty acid content of control and lipid-loaded cells were determined using the collection and lipid analysis protocols described above in order to demonstrate the range of achievable lipid profiles for a variety of cell types from both terrestrial and aquatic species. Twelve unique cell lines from eleven species from both aquatic and terrestrial across seven different cell types were evaluated for the ability to load lipids and create custom fatty acid profiles including bluefin tuna myoblasts, bluefin tuna preadipocytes, carp pre-adipocytes, red snapper muscle derived cells, yellowfin tuna heart fibroblasts, bluegill fibroblasts, tilapia brain derived cells, salmon kidney cells, mahi mahi myoblasts, chicken embryonic fibroblasts, pig kidney cells, and dog kidney cells. Cells were cultured and loaded with lipids using the protocols described above and lipid loading was performed using the concentrations for a range of conditions shown in Table 11 below. Conditions 3-6 were selected to show adjustment of profiles towards those of existing foods for cells from other species. Conditions 2 and 7-13 were selected to exhibit the range of total saturated fatty acid (SFA) or Omega-3 that might be achieved using lipid loading.

TABLE 11
Conditions used for lipid loading expressed in relative fraction of fatty acids

| | | | | | | 18:3 | | | Total | Total |
|---|---|---|---|---|---|---|---|---|---|---|
| Condition | 16:0 | 16:1 | 18:0 | 18:1 | 18:2 | N3 | 20:5 | 22:6 | SFA | Omega-3 |
| 1. Unloaded | 0 | 0 | 0 | 0 | 0 | 0 | 0 | 0 | 0 | 0 |
| control | | | | | | | | | | |
| 2. Even | 0.125 | 0.125 | 0.125 | 0.125 | 0.125 | 0.125 | 0.125 | 0.125 | 0.25 | 0.375 |
| distribution | | | | | | | | | | |
| 3. Fish | 0.25 | 0.1 | 0.1 | 0.3 | 0 | 0.05 | 0.1 | 0.1 | 0.35 | 0.25 |
| 4. Foie | 0.3 | 0.05 | 0.1 | 0.55 | 0 | 0 | 0 | 0 | 0.4 | 0 |
| Gras | | | | | | | | | | |
| 5. Beef | 0.3 | 0.05 | 0.2 | 0.35 | 0.1 | 0 | 0 | 0 | 0.5 | 0 |
| 6. Chicken | 0.25 | 0.05 | 0.05 | 0.45 | 0.2 | 0 | 0 | 0 | 0.3 | 0 |
| 7 | 0 | 0 | 0 | 0 | 0 | 0.33 | 0.33 | 0.33 | 0 | 0.99 |
| 8 | 0.1 | 0 | 0 | 0.2 | 0 | 0 | 0.35 | 0.35 | 0.1 | 0.7 |
| 9 | 0 | 0.25 | 0 | 0.25 | 0 | 0.25 | 0 | 0.25 | 0 | 0.5 |
| 10 | 0.17 | 0.17 | 0 | 0 | 0.17 | 0.17 | 0.17 | 0.17 | 0.17 | 0.51 |
| 11 | 0 | 0.17 | 0.17 | 0.17 | 0.17 | 0.17 | 0.17 | 0 | 0.17 | 0.34 |
| 12 | 0 | 0.25 | 0.25 | 0.3 | 0 | 0 | 0 | 0.2 | 0.25 | 0.2 |
| 13 | 0.2 | 0 | 0.2 | 0.2 | 0 | 0.2 | 0.2 | 0 | 0.4 | 0.4 |

### Results

Fatty acid compositions for the samples tested are presented in Tables 12-23 below. Data is expressed as relative percent concentration of total fatty acid for each condition tested as well as concentrations for the species of interest in nature. In addition to fatty acids shown, 17:1 heptadecenoic acid was measured but not detected in any sample and 22:3 was detected in a low number of samples as a minority fraction so data is not shown for either fatty acid. Data for each species is shown in tables below as follows: bluefin tuna myoblasts (Tables 12A, B, C), bluefin tuna preadipocytes (Tables 13A, B, C), carp pre-adipocytes (Tables 14A, B, C), red snapper muscle derived cells (Tables 15A, B, C), yellowfin tuna heart fibroblasts (Tables 16A, B, C), bluegill fibroblasts (Tables 17A, B, C), tilapia brain derived cells (Tables 18A, B, C), salmon kidney cells (Tables 19A, B, C), mahi mahi myoblasts (Tables 20A, B, C), chicken embryonic fibroblasts (Tables 21A, B, C), pig kidney cells (Tables 22A, B, C), and dog kidney cells (Tables 23A, B, C). Changes in fatty acid composition relative to control and those occurring in nature were observed for all species. The percentage point change from unloaded control cells for all species are shown in Tables 24A, B and 25A, B for saturated fatty acids (SFA) and Omega-3 fatty acids, respectively. Notably, substantial decreases in saturated fatty acids of over 55 percentage points and substantial increases in Omega-3 fatty acids over 70 percentage points were obtained. For terrestrial animals with typically zero—<10% Omega-3, the presence and significant increase in healthy fats represents a surprising ability to generate unique change in compositions not seen in nature.

TABLE 12A
Fatty acid relative percent compositions for fatty acids 12:0 to 18:4 of control
and lipid loaded bluefin tuna myoblasts and bluefin tuna in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.54 | 2.37 | 0.86 | 27.48 | 3.69 | 0.52 | 24.56 | 12.25 | 1.67 | 0.11 | 0.41 | 0.07 |
| | StDev | 0.06 | 0.17 | 0.06 | 3.08 | 1.00 | 0.04 | 4.37 | 2.49 | 0.20 | 0.02 | 0.10 | 0.01 |
| 2 | Average | 0.15 | 0.65 | 0.23 | 5.91 | 6.74 | 0.19 | 6.74 | 13.76 | 6.51 | 3.87 | 0.58 | 0.64 |
| | StDev | 0.03 | 0.07 | 0.02 | 0.79 | 0.12 | 0.01 | 1.29 | 0.43 | 0.16 | 0.07 | 0.01 | 0.05 |
| 3 | Average | 0.50 | 1.55 | 0.57 | 11.88 | 11.80 | 0.32 | 11.00 | 42.38 | 0.96 | 0.12 | 0.46 | 0.08 |
| | StDev | 0.38 | 0.71 | 0.36 | 3.33 | 2.58 | 0.10 | 1.29 | 1.21 | 0.10 | 0.06 | 0.11 | 0.01 |
| 4 | Average | 0.86 | 1.22 | 0.49 | 9.77 | 4.14 | 0.31 | 6.11 | 62.39 | 0.68 | 0.07 | 0.28 | 0.05 |
| | StDev | 1.19 | 0.30 | 0.28 | 2.37 | 1.21 | 0.03 | 1.99 | 2.77 | 0.07 | 0.03 | 0.05 | 0.03 |
| 5 | Average | 7.33 | 2.21 | 0.62 | 13.98 | 4.28 | 0.51 | 9.18 | 33.81 | 7.09 | 0.12 | 1.39 | 0.06 |
| | StDev | 11.38 | 0.72 | 0.19 | 1.41 | 2.54 | 0.17 | 0.33 | 9.71 | 1.64 | 0.02 | 0.81 | 0.02 |
| 6 | Average | 0.15 | 0.96 | 0.37 | 8.77 | 4.95 | 0.28 | 6.10 | 46.18 | 14.14 | 0.11 | 2.84 | 0.04 |
| | StDev | 0.04 | 0.08 | 0.06 | 0.84 | 0.06 | 0.04 | 0.91 | 1.15 | 0.46 | 0.01 | 0.20 | 0.01 |
| 7 | Average | 0.06 | 0.41 | 0.14 | 3.08 | 0.54 | 0.13 | 3.03 | 2.58 | 0.85 | 10.54 | 0.15 | 1.20 |
| | StDev | 0.03 | 0.06 | 0.05 | 0.66 | 0.06 | 0.02 | 0.78 | 0.25 | 0.05 | 0.36 | 0.03 | 0.23 |
| 8 | Average | 0.08 | 0.54 | 0.16 | 4.99 | 0.72 | 0.18 | 3.59 | 13.16 | 0.46 | 0.10 | 0.12 | 0.17 |
| | StDev | 0.06 | 0.06 | 0.02 | 0.66 | 0.05 | 0.01 | 0.41 | 0.08 | 0.05 | 0.09 | 0.00 | 0.01 |
| 9 | Average | 0.06 | 0.36 | 0.12 | 2.05 | 10.05 | 0.10 | 2.96 | 15.94 | 0.37 | 5.93 | 0.12 | 1.12 |
| | StDev | 0.02 | 0.06 | 0.02 | 0.13 | 0.17 | 0.01 | 0.35 | 1.02 | 0.01 | 0.13 | 0.00 | 0.05 |
| 10 | Average | 0.87 | 0.81 | 0.25 | 5.81 | 9.19 | 0.25 | 6.20 | 3.35 | 7.78 | 4.79 | 0.54 | 0.64 |
| | StDev | 1.32 | 0.13 | 0.04 | 0.37 | 1.61 | 0.06 | 0.38 | 0.16 | 0.24 | 0.96 | 0.12 | 0.28 |
| 11 | Average | 0.10 | 0.69 | 0.24 | 4.43 | 14.83 | 0.20 | 7.04 | 17.97 | 9.14 | 7.50 | 0.79 | 1.14 |
| | StDev | 0.02 | 0.10 | 0.04 | 0.69 | 0.38 | 0.03 | 1.78 | 1.16 | 0.33 | 0.37 | 0.10 | 0.08 |
| 12 | Average | 0.17 | 1.01 | 0.44 | 5.98 | 11.77 | 0.20 | 6.60 | 20.11 | 0.64 | 0.05 | 0.12 | 0.02 |
| | StDev | 0.10 | 0.53 | 0.32 | 1.83 | 0.51 | 0.08 | 1.51 | 0.80 | 0.05 | 0.05 | 0.01 | 0.01 |
| 13 | Average | 0.23 | 1.13 | 0.42 | 9.92 | 2.13 | 0.32 | 7.71 | 19.47 | 1.20 | 18.35 | 0.23 | 1.13 |
| | StDev | 0.21 | 0.22 | 0.12 | 0.25 | 0.05 | 0.06 | 0.38 | 3.17 | 0.53 | 3.06 | 0.08 | 0.05 |

| | | | | | | | | | | | | |
|---|---|---|---|---|---|---|---|---|---|---|---|---|
| Profile in Nature | 0.00 | 3.25 | 0.00 | 18.92 | 3.78 | 0.00 | 7.17 | 21.58 | 1.24 | 0.00 | 0.00 | .91 |

TABLE 12B
Fatty acid relative percent compositions for fatty acids 20:0 to 22:4 of control
and lipid loaded bluefin tuna myoblasts and bluefin tuna in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.25 | 0.27 | 0.07 | 0.01 | 2.06 | 0.14 | 5.37 | 0.72 | 0.17 | 0.06 | 0.04 | 3.57 |
| | StDev | 0.08 | 0.03 | 0.01 | 0.00 | 0.48 | 0.02 | 1.10 | 0.55 | 0.06 | 0.01 | 0.00 | 0.65 |
| 2 | Average | 0.05 | 0.13 | 0.10 | 0.11 | 0.62 | 0.05 | 2.10 | 9.28 | 0.02 | 0.03 | 0.05 | 1.93 |
| | StDev | 0.01 | 0.01 | 0.00 | 0.00 | 0.00 | 0.01 | 0.03 | 0.26 | 0.00 | 0.01 | 0.01 | 0.04 |
| 3 | Average | 0.11 | 0.35 | 0.04 | 0.01 | 1.02 | 0.10 | 4.07 | 0.35 | 0.11 | 0.09 | 0.03 | 2.28 |
| | StDev | 0.02 | 0.06 | 0.00 | 0.00 | 0.09 | 0.01 | 0.89 | 0.12 | 0.08 | 0.02 | 0.01 | 0.09 |
| 4 | Average | 0.09 | 0.47 | 0.03 | 0.01 | 0.84 | 0.08 | 2.72 | 0.20 | 0.07 | 0.17 | 0.03 | 2.16 |
| | StDev | 0.02 | 0.19 | 0.02 | 0.00 | 0.25 | 0.03 | 0.20 | 0.01 | 0.02 | 0.09 | 0.02 | 0.87 |
| 5 | Average | 0.22 | 0.46 | 0.21 | 0.01 | 1.63 | 0.09 | 3.43 | 0.22 | 0.15 | 0.14 | 0.22 | 3.48 |
| | StDev | 0.20 | 0.16 | 0.07 | 0.00 | 0.28 | 0.02 | 0.09 | 0.01 | 0.13 | 0.06 | 0.08 | 1.57 |
| 6 | Average | 0.06 | 0.31 | 0.21 | 0.00 | 1.32 | 0.07 | 2.98 | 0.15 | 0.04 | 0.11 | 0.20 | 2.22 |
| | StDev | 0.01 | 0.01 | 0.02 | 0.00 | 0.06 | 0.02 | 0.18 | 0.01 | 0.01 | 0.01 | 0.00 | 0.11 |
| 7 | Average | 0.02 | 0.04 | 0.02 | 0.14 | 0.34 | 0.03 | 1.19 | 21.76 | 0.01 | 0.01 | 0.01 | 1.16 |
| | StDev | 0.01 | 0.00 | 0.00 | 0.02 | 0.01 | 0.01 | 0.38 | 0.91 | 0.01 | 0.00 | 0.00 | 0.12 |
| 8 | Average | 0.03 | 0.11 | 0.02 | 0.00 | 0.39 | 0.04 | 1.82 | 14.93 | 0.01 | 0.02 | 0.01 | 1.42 |
| | StDev | 0.01 | 0.01 | 0.00 | 0.00 | 0.01 | 0.01 | 0.04 | 1.06 | 0.00 | 0.01 | 0.00 | 0.02 |
| 9 | Average | 0.02 | 0.13 | 0.02 | 0.16 | 0.37 | 0.04 | 1.25 | 0.65 | 0.01 | 0.04 | 0.01 | 1.15 |
| | StDev | 0.00 | 0.01 | 0.00 | 0.01 | 0.02 | 0.00 | 0.11 | 0.17 | 0.00 | 0.01 | 0.00 | 0.05 |
| 10 | Average | 0.05 | 0.06 | 0.16 | 0.19 | 0.65 | 0.05 | 1.94 | 9.42 | 0.02 | 0.02 | 0.07 | 2.19 |
| | StDev | 0.01 | 0.01 | 0.03 | 0.03 | 0.08 | 0.01 | 0.09 | 1.20 | 0.00 | 0.01 | 0.01 | 0.44 |
| 11 | Average | 0.03 | 0.14 | 0.13 | 0.18 | 0.72 | 0.06 | 2.71 | 15.25 | 0.01 | 0.03 | 0.09 | 2.60 |
| | StDev | 0.01 | 0.02 | 0.02 | 0.02 | 0.04 | 0.01 | 0.19 | 1.39 | 0.00 | 0.01 | 0.02 | 0.25 |
| 12 | Average | 0.06 | 0.21 | 0.03 | 0.00 | 0.94 | 0.07 | 2.95 | 1.07 | 0.03 | 0.04 | 0.01 | 1.92 |
| | StDev | 0.03 | 0.02 | 0.01 | 0.00 | 0.07 | 0.00 | 0.31 | 0.11 | 0.03 | 0.00 | 0.00 | 0.11 |
| 13 | Average | 0.05 | 0.21 | 0.04 | 0.16 | 1.22 | 0.10 | 4.33 | 16.55 | 0.02 | 0.03 | 0.01 | 3.02 |
| | StDev | 0.00 | 0.03 | 0.00 | 0.03 | 0.01 | 0.00 | 0.05 | 2.42 | 0.01 | 0.00 | 0.01 | 0.34 |

| | | | | | | | | | | | | |
|---|---|---|---|---|---|---|---|---|---|---|---|---|
| Profile in Nature | 0.00 | 6.47 | 0.00 | 0.00 | 0.00 | 0.00 | 1.00 | 6.54 | 0.00 | 5.54 | 0.00 | 0.00 |

TABLE 12C
Fatty acid relative percent compositions for fatty acids 22:5 N3 to 26:0 and total for SFA, MUFA,
PUFA, and Omega-3s of control and lipid loaded bluefin tuna myoblasts and bluefin tuna in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 4.23 | 0.88 | 6.79 | 0.07 | 0.27 | 0.14 | 0.34 | 57.44 | 16.42 | 26.14 | 11.85 |
| | StDev | 0.54 | 0.18 | 1.34 | 0.01 | 0.09 | 0.02 | 0.07 | 7.49 | 3.49 | 4.01 | 1.50 |
| 2 | Average | 5.44 | 0.37 | 33.60 | 0.01 | 0.04 | 0.08 | 0.04 | 14.02 | 20.73 | 65.24 | 52.19 |
| | StDev | 0.15 | 0.07 | 1.19 | 0.00 | 0.01 | 0.01 | 0.00 | 2.21 | 0.45 | 1.83 | 1.66 |
| 3 | Average | 2.87 | 0.62 | 5.58 | 0.04 | 0.20 | 0.18 | 0.15 | 26.42 | 54.80 | 18.58 | 8.91 |
| | StDev | 0.21 | 0.02 | 0.72 | 0.02 | 0.14 | 0.10 | 0.05 | 3.58 | 1.53 | 1.96 | 0.97 |
| 4 | Average | 2.37 | 0.54 | 3.58 | 0.03 | 0.10 | 0.07 | 0.10 | 19.14 | 67.23 | 13.63 | 6.22 |
| | StDev | 0.60 | 0.14 | 0.23 | 0.03 | 0.08 | 0.01 | 0.09 | 4.19 | 2.31 | 2.19 | 0.73 |
| 5 | Average | 3.42 | 0.76 | 4.69 | 0.03 | 0.09 | 0.13 | 0.09 | 34.42 | 38.82 | 26.76 | 8.42 |
| | StDev | 0.90 | 0.20 | 0.12 | 0.01 | 0.03 | 0.04 | 0.03 | 11.51 | 11.98 | 0.63 | 0.79 |
| 6 | Average | 2.50 | 0.55 | 4.17 | 0.02 | 0.05 | 0.10 | 0.05 | 16.85 | 51.65 | 31.51 | 6.93 |
| | StDev | 0.10 | 0.01 | 0.18 | 0.01 | 0.02 | 0.02 | 0.02 | 1.90 | 1.21 | 1.06 | 0.28 |
| 7 | Average | 5.40 | 0.28 | 46.85 | 0.01 | 0.02 | 0.00 | 0.03 | 6.91 | 3.18 | 89.91 | 84.55 |
| | StDev | 0.68 | 0.03 | 0.70 | 0.00 | 0.00 | 0.00 | 0.00 | 1.63 | 0.30 | 1.39 | 0.82 |
| 8 | Average | 6.00 | 0.32 | 50.54 | 0.00 | 0.01 | 0.06 | 0.01 | 9.60 | 14.07 | 76.33 | 71.57 |
| | StDev | 0.18 | 0.02 | 1.22 | 0.00 | 0.01 | 0.02 | 0.01 | 1.22 | 0.11 | 1.32 | 1.42 |
| 9 | Average | 1.19 | 0.27 | 55.38 | 0.00 | 0.01 | 0.08 | 0.01 | 5.70 | 26.25 | 68.05 | 63.15 |
| | StDev | 0.04 | 0.02 | 0.48 | 0.00 | 0.00 | 0.02 | 0.00 | 0.57 | 1.07 | 0.50 | 0.61 |
| 10 | Average | 6.19 | 0.40 | 37.97 | 0.01 | 0.02 | 0.08 | 0.01 | 14.29 | 12.70 | 73.02 | 58.37 |
| | StDev | 0.35 | 0.09 | 1.32 | 0.00 | 0.01 | 0.02 | 0.01 | 2.17 | 1.43 | 0.90 | 0.86 |
| 11 | Average | 9.84 | 0.47 | 3.58 | 0.01 | 0.02 | 0.01 | 0.01 | 12.78 | 32.98 | 54.24 | 36.18 |
| | StDev | 1.38 | 0.04 | 0.32 | 0.00 | 0.00 | 0.00 | 0.01 | 2.61 | 1.56 | 1.06 | 0.71 |
| 12 | Average | 2.20 | 0.48 | 42.46 | 0.02 | 0.06 | 0.26 | 0.07 | 14.63 | 32.40 | 52.97 | 45.78 |
| | StDev | 0.11 | 0.05 | 3.24 | 0.02 | 0.07 | 0.05 | 0.08 | 4.56 | 1.15 | 3.52 | 3.41 |
| 13 | Average | 5.58 | 0.72 | 5.38 | 0.02 | 0.04 | 0.23 | 0.04 | 19.89 | 22.08 | 58.03 | 45.86 |
| | StDev | 0.22 | 0.09 | 0.64 | 0.00 | 0.01 | 0.05 | 0.01 | 1.12 | 3.19 | 2.25 | 1.30 |

| | | | | | | | | | | | |
|---|---|---|---|---|---|---|---|---|---|---|---|
| Profile in Nature | 2.80 | 0.00 | 20.79 | 0.00 | 0.00 | 0.00 | 0.00 | 29.34 | 37.37 | 33.29 | 30.13 |

TABLE 13A
Fatty acid relative percent compositions for fatty acids 12:0 to 18:4 of control
and lipid loaded bluefin tuna preadipocytes and bluefin tuna in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.54 | 3.47 | 0.71 | 15.47 | 6.36 | 0.38 | 17.26 | 13.89 | 1.10 | 0.05 | 1.30 | 0.16 |
| | StDev | 0.12 | 0.37 | 0.09 | 0.65 | 0.37 | 0.01 | 0.19 | 0.27 | 0.01 | 0.00 | 0.11 | 0.02 |
| 2 | Average | 0.05 | 0.66 | 0.13 | 3.34 | 6.69 | 0.10 | 5.17 | 13.92 | 6.56 | 4.10 | 1.70 | 1.79 |
| | StDev | 0.00 | 0.11 | 0.01 | 0.61 | 0.01 | 0.01 | 0.78 | 0.42 | 0.49 | 0.50 | 0.22 | 0.07 |
| 3 | Average | 0.18 | 1.58 | 0.26 | 7.54 | 11.87 | 0.17 | 7.47 | 52.07 | 0.60 | 0.06 | 0.78 | 0.13 |
| | StDev | 0.01 | 0.04 | 0.04 | 0.22 | 0.43 | 0.00 | 0.25 | 1.15 | 0.07 | 0.01 | 0.02 | 0.01 |
| 4 | Average | 0.16 | 1.20 | 0.20 | 6.04 | 5.55 | 0.15 | 5.69 | 67.54 | 0.46 | 0.05 | 0.60 | 0.12 |
| | StDev | 0.03 | 0.07 | 0.01 | 0.14 | 0.06 | 0.01 | 0.07 | 0.49 | 0.07 | 0.01 | 0.01 | 0.00 |
| 5 | Average | 0.16 | 1.48 | 0.22 | 8.14 | 6.91 | 0.17 | 7.23 | 47.74 | 7.04 | 0.10 | 4.88 | 0.14 |
| | StDev | 0.01 | 0.02 | 0.01 | 0.42 | 0.03 | 0.00 | 0.27 | 0.29 | 0.33 | 0.00 | 0.02 | 0.01 |
| 6 | Average | 0.22 | 1.34 | 0.26 | 6.97 | 6.14 | 0.23 | 8.27 | 43.40 | 12.71 | 0.14 | 7.12 | 0.12 |
| | StDev | 0.11 | 0.17 | 0.02 | 1.39 | 1.04 | 0.12 | 4.36 | 7.30 | 0.11 | 0.01 | 0.39 | 0.01 |
| 7 | Average | 0.04 | 0.47 | 0.08 | 2.73 | 0.57 | 0.07 | 2.92 | 2.50 | 0.75 | 11.60 | 0.26 | 2.80 |
| | StDev | 0.01 | 0.01 | 0.01 | 0.07 | 0.07 | 0.01 | 0.30 | 0.20 | 0.02 | 0.15 | 0.02 | 0.18 |
| 8 | Average | 0.04 | 0.48 | 0.09 | 2.75 | 0.61 | 0.08 | 3.32 | 12.12 | 0.29 | 0.04 | 0.16 | 0.07 |
| | StDev | 0.01 | 0.03 | 0.01 | 0.32 | 0.01 | 0.00 | 0.96 | 0.67 | 0.01 | 0.00 | 0.00 | 0.00 |
| 9 | Average | 0.05 | 0.40 | 0.08 | 1.48 | 11.98 | 0.07 | 3.03 | 17.82 | 0.34 | 7.27 | 0.24 | 3.11 |
| | StDev | 0.01 | 0.04 | 0.01 | 0.30 | 2.09 | 0.04 | 0.48 | 3.02 | 0.04 | 0.09 | 0.00 | 0.04 |
| 10 | Average | 0.07 | 0.64 | 0.11 | 3.13 | 9.37 | 0.08 | 4.31 | 3.63 | 9.30 | 6.52 | 1.78 | 2.15 |
| | StDev | 0.02 | 0.17 | 0.03 | 0.79 | 0.17 | 0.01 | 0.52 | 0.81 | 0.99 | 1.25 | 0.35 | 0.18 |
| 11 | Average | 0.05 | 0.64 | 0.11 | 2.53 | 13.68 | 0.09 | 4.99 | 19.27 | 10.69 | 10.00 | 2.17 | 3.26 |
| | StDev | 0.01 | 0.08 | 0.00 | 0.27 | 0.45 | 0.00 | 0.65 | 0.01 | 0.34 | 1.06 | 0.36 | 0.24 |
| 12 | Average | 0.07 | 0.49 | 0.12 | 1.94 | 16.29 | 0.09 | 4.45 | 20.07 | 0.32 | 0.04 | 0.23 | 0.03 |
| | StDev | 0.04 | 0.07 | 0.04 | 0.48 | 4.37 | 0.05 | 1.39 | 4.91 | 0.04 | 0.01 | 0.02 | 0.00 |
| 13 | Average | 0.06 | 0.89 | 0.16 | 5.76 | 1.48 | 0.17 | 6.80 | 20.35 | 16.64 | 0.16 | 4.32 | 0.10 |
| | StDev | 0.00 | 0.15 | 0.00 | 0.23 | 0.20 | 0.01 | 0.95 | 0.13 | 0.77 | 0.06 | 0.52 | 0.01 |

| | | | | | | | | | | | | |
|---|---|---|---|---|---|---|---|---|---|---|---|---|
| Profile in Nature | 0.00 | 3.25 | 0.00 | 18.92 | 3.78 | 0.00 | 7.17 | 21.58 | 1.24 | 0.00 | 0.00 | .91 |

TABLE 13B
Fatty acid relative percent compositions for fatty acids 20:0 to 22:4 of control
and lipid loaded bluefin tuna preadipocytes and bluefin tuna in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.15 | 0.37 | 0.06 | 0.02 | 3.44 | 0.25 | 8.67 | 0.51 | 0.28 | 0.14 | 0.10 | 5.63 |
| | StDev | 0.04 | 0.03 | 0.01 | 0.02 | 0.01 | 0.02 | 0.02 | 0.01 | 0.08 | 0.02 | 0.02 | 0.00 |
| 2 | Average | 0.03 | 0.11 | 0.09 | 0.09 | 0.80 | 0.06 | 2.23 | 12.53 | 0.03 | 0.05 | 0.08 | 1.48 |
| | StDev | 0.00 | 0.00 | 0.00 | 0.00 | 0.07 | 0.01 | 0.03 | 0.39 | 0.01 | 0.00 | 0.01 | 0.04 |
| 3 | Average | 0.05 | 0.44 | 0.03 | 0.00 | 1.37 | 0.12 | 4.10 | 0.27 | 0.07 | 0.23 | 0.04 | 2.49 |
| | StDev | 0.00 | 0.04 | 0.00 | 0.00 | 0.16 | 0.01 | 0.45 | 0.02 | 0.01 | 0.01 | 0.01 | 0.25 |
| 4 | Average | 0.04 | 0.41 | 0.02 | 0.00 | 0.91 | 0.09 | 2.80 | 0.20 | 0.06 | 0.24 | 0.02 | 1.74 |
| | StDev | 0.00 | 0.04 | 0.00 | 0.00 | 0.07 | 0.00 | 0.18 | 0.00 | 0.00 | 0.02 | 0.00 | 0.11 |
| 5 | Average | 0.06 | 0.35 | 0.11 | 0.00 | 2.08 | 0.08 | 3.43 | 0.23 | 0.06 | 0.20 | 0.14 | 2.13 |
| | StDev | 0.00 | 0.03 | 0.01 | 0.00 | 0.12 | 0.00 | 0.15 | 0.01 | 0.00 | 0.03 | 0.01 | 0.12 |
| 6 | Average | 0.08 | 0.25 | 0.15 | 0.01 | 1.59 | 0.07 | 2.81 | 0.21 | 0.04 | 0.17 | 0.13 | 1.56 |
| | StDev | 0.05 | 0.02 | 0.00 | 0.01 | 0.28 | 0.00 | 0.08 | 0.03 | 0.00 | 0.06 | 0.03 | 0.22 |
| 7 | Average | 0.01 | 0.03 | 0.02 | 0.13 | 0.35 | 0.04 | 1.09 | 23.39 | 0.01 | 0.01 | 0.01 | 0.87 |
| | StDev | 0.01 | 0.00 | 0.00 | 0.01 | 0.03 | 0.00 | 0.20 | 0.53 | 0.00 | 0.00 | 0.00 | 0.08 |
| 8 | Average | 0.02 | 0.08 | 0.01 | 0.00 | 0.39 | 0.04 | 1.88 | 19.82 | 0.01 | 0.03 | 0.01 | 0.99 |
| | StDev | 0.01 | 0.01 | 0.00 | 0.00 | 0.01 | 0.00 | 0.11 | 0.32 | 0.01 | 0.00 | 0.00 | 0.01 |
| 9 | Average | 0.02 | 0.10 | 0.02 | 0.11 | 0.41 | 0.04 | 1.35 | 0.26 | 0.01 | 0.05 | 0.01 | 0.89 |
| | StDev | 0.01 | 0.01 | 0.01 | 0.01 | 0.06 | 0.01 | 0.22 | 0.06 | 0.00 | 0.00 | 0.00 | 0.10 |
| 10 | Average | 0.02 | 0.05 | 0.12 | 0.15 | 0.84 | 0.05 | 2.06 | 13.16 | 0.02 | 0.03 | 0.12 | 1.46 |
| | StDev | 0.00 | 0.00 | 0.01 | 0.01 | 0.08 | 0.02 | 0.04 | 1.08 | 0.01 | 0.01 | 0.00 | 0.11 |
| 11 | Average | 0.03 | 0.13 | 0.12 | 0.16 | 0.92 | 0.07 | 2.72 | 19.37 | 0.02 | 0.06 | 0.13 | 1.62 |
| | StDev | 0.00 | 0.01 | 0.01 | 0.02 | 0.04 | 0.01 | 0.18 | 0.22 | 0.00 | 0.00 | 0.02 | 0.17 |
| 12 | Average | 0.02 | 0.16 | 0.02 | 0.00 | 0.53 | 0.05 | 1.63 | 0.31 | 0.01 | 0.07 | 0.01 | 1.16 |
| | StDev | 0.00 | 0.01 | 0.00 | 0.00 | 0.03 | 0.01 | 0.02 | 0.02 | 0.01 | 0.02 | 0.01 | 0.16 |
| 13 | Average | 0.03 | 0.15 | 0.22 | 0.00 | 1.49 | 0.09 | 3.47 | 24.99 | 0.03 | 0.09 | 0.21 | 2.08 |
| | StDev | 0.01 | 0.00 | 0.01 | 0.00 | 0.09 | 0.00 | 0.03 | 0.02 | 0.00 | 0.01 | 0.02 | 0.04 |

| | | | | | | | | | | | | |
|---|---|---|---|---|---|---|---|---|---|---|---|---|
| Profile in Nature | 0.00 | 6.47 | 0.00 | 0.00 | 0.00 | 0.00 | 1.00 | 6.54 | 0.00 | 5.54 | 0.00 | 0.00 |

TABLE 13C
Fatty acid relative percent compositions for fatty acids 22:5 N3 to 26:0 and total for SFA, MUFA, PUFA,
and Omega-3s of control and lipid loaded bluefin tuna preadipocytes and bluefin tuna in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 6.10 | 1.28 | 9.63 | 0.10 | 0.58 | 1.07 | 0.92 | 39.87 | 21.84 | 38.29 | 16.29 |
| | StDev | 0.03 | 0.01 | 0.06 | 0.11 | 0.46 | 0.85 | 0.74 | 0.24 | 0.16 | 0.08 | 0.01 |
| 2 | Average | 4.02 | 0.28 | 33.59 | 0.00 | 0.04 | 0.19 | 0.08 | 9.64 | 20.96 | 69.41 | 54.23 |
| | StDev | 0.22 | 0.02 | 0.73 | 0.00 | 0.05 | 0.15 | 0.11 | 1.71 | 0.26 | 1.45 | 1.40 |
| 3 | Average | 2.80 | 0.61 | 4.47 | 0.00 | 0.01 | 0.21 | 0.00 | 17.32 | 64.82 | 17.86 | 7.59 |
| | StDev | 0.21 | 0.04 | 0.34 | 0.00 | 0.00 | 0.00 | 0.00 | 0.05 | 1.53 | 1.58 | 0.56 |
| 4 | Average | 2.01 | 0.43 | 3.14 | 0.01 | 0.01 | 0.11 | 0.01 | 13.56 | 73.84 | 12.59 | 5.41 |
| | StDev | 0.07 | 0.02 | 0.17 | 0.00 | 0.01 | 0.01 | 0.00 | 0.19 | 0.37 | 0.56 | 0.25 |
| 5 | Average | 2.39 | 0.53 | 3.84 | 0.00 | 0.01 | 0.15 | 0.00 | 17.52 | 55.35 | 27.13 | 6.56 |
| | StDev | 0.06 | 0.00 | 0.12 | 0.00 | 0.00 | 0.01 | 0.00 | 0.76 | 0.21 | 0.96 | 0.19 |
| 6 | Average | 1.80 | 0.54 | 3.34 | 0.02 | 0.02 | 0.22 | 0.18 | 17.55 | 50.17 | 32.28 | 5.48 |
| | StDev | 0.17 | 0.16 | 0.23 | 0.02 | 0.01 | 0.13 | 0.00 | 6.07 | 6.36 | 0.29 | 0.08 |
| 7 | Average | 3.82 | 0.12 | 45.22 | 0.01 | 0.03 | 0.00 | 0.04 | 6.40 | 3.11 | 90.49 | 84.03 |
| | StDev | 0.19 | 0.01 | 0.16 | 0.00 | 0.02 | 0.00 | 0.03 | 0.45 | 0.27 | 0.18 | 0.33 |
| 8 | Average | 4.34 | 0.18 | 52.12 | 0.00 | 0.00 | 0.03 | 0.00 | 6.79 | 12.86 | 80.35 | 76.32 |
| | StDev | 0.39 | 0.02 | 0.67 | 0.00 | 0.00 | 0.00 | 0.00 | 1.36 | 0.67 | 0.69 | 0.59 |
| 9 | Average | 0.92 | 0.18 | 49.70 | 0.01 | 0.00 | 0.04 | 0.00 | 5.16 | 29.99 | 64.85 | 58.15 |
| | StDev | 0.13 | 0.04 | 0.79 | 0.00 | 0.00 | 0.01 | 0.00 | 0.88 | 0.91 | 0.03 | 0.50 |
| 10 | Average | 4.46 | 0.23 | 36.05 | 0.00 | 0.00 | 0.07 | 0.00 | 8.39 | 13.15 | 78.46 | 60.18 |
| | StDev | 0.24 | 0.02 | 0.17 | 0.00 | 0.00 | 0.01 | 0.00 | 1.50 | 0.65 | 2.15 | 1.91 |
| 11 | Average | 4.60 | 0.30 | 2.18 | 0.01 | 0.04 | 0.01 | 0.06 | 8.52 | 33.14 | 58.34 | 36.15 |
| | StDev | 0.26 | 0.02 | 0.21 | 0.01 | 0.00 | 0.01 | 0.00 | 1.07 | 0.46 | 1.53 | 1.31 |
| 12 | Average | 1.15 | 0.25 | 50.42 | 0.00 | 0.00 | 0.07 | 0.00 | 7.17 | 36.67 | 56.16 | 51.92 |
| | StDev | 0.16 | 0.01 | 1.97 | 0.00 | 0.00 | 0.01 | 0.00 | 2.05 | 0.50 | 1.55 | 1.82 |
| 13 | Average | 7.28 | 0.38 | 2.51 | 0.00 | 0.00 | 0.10 | 0.00 | 13.90 | 22.18 | 63.93 | 34.94 |
| | StDev | 0.33 | 0.01 | 0.14 | 0.00 | 0.00 | 0.01 | 0.00 | 0.59 | 0.32 | 0.27 | 0.43 |

| | | | | | | | | | | | |
|---|---|---|---|---|---|---|---|---|---|---|---|
| Profile in Nature | 2.80 | 0.00 | 20.79 | 0.00 | 0.00 | 0.00 | 0.00 | 29.34 | 37.37 | 33.29 | 30.13 |

TABLE 14A
Fatty acid relative percent compositions for fatty acids 12:0 to 18:4
of control and lipid loaded carp preadipocytes and carp in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.15 | 0.61 | 0.28 | 11.35 | 1.18 | 0.51 | 13.17 | 14.05 | 10.20 | 0.37 | 0.80 | 0.06 |
| | StDev | 0.08 | 0.86 | 0.30 | 7.29 | 1.30 | 0.08 | 3.63 | 0.76 | 0.53 | 0.23 | 0.38 | 0.07 |
| 2 | Average | 0.18 | 0.38 | 0.14 | 5.83 | 3.01 | 0.35 | 8.86 | 11.86 | 6.68 | 1.35 | 0.33 | 0.14 |
| | StDev | 0.17 | 0.34 | 0.11 | 3.00 | 3.31 | 0.04 | 2.81 | 1.78 | 0.54 | 0.92 | 0.19 | 0.13 |
| 3 | Average | 0.12 | 0.62 | 0.18 | 5.80 | 4.84 | 0.31 | 9.48 | 17.94 | 3.92 | 0.86 | 0.40 | 0.18 |
| | StDev | 0.08 | 0.27 | 0.09 | 1.93 | 4.37 | 0.02 | 2.90 | 3.53 | 0.48 | 0.47 | 0.24 | 0.06 |
| 4 | Average | 0.68 | 1.36 | 0.25 | 9.45 | 6.10 | 0.39 | 20.49 | 35.46 | 5.74 | 0.50 | 0.60 | 0.07 |
| | StDev | 0.32 | 0.20 | 0.02 | 1.80 | 0.99 | 0.05 | 8.63 | 6.47 | 0.73 | 0.13 | 0.07 | 0.02 |
| 5 | Average | 0.30 | 0.82 | 0.23 | 6.95 | 4.19 | 0.43 | 12.45 | 31.81 | 12.40 | 0.41 | 0.99 | 0.09 |
| | StDev | 0.18 | 0.61 | 0.16 | 2.80 | 3.65 | 0.05 | 3.04 | 4.82 | 1.45 | 0.22 | 0.55 | 0.02 |
| 6 | Average | 0.12 | 0.72 | 0.26 | 8.46 | 3.17 | 0.43 | 10.68 | 36.48 | 15.90 | 0.37 | 0.72 | 0.04 |
| | StDev | 0.03 | 0.06 | 0.01 | 1.04 | 0.51 | 0.02 | 1.70 | 3.62 | 1.80 | 0.04 | 0.03 | 0.00 |
| 7 | Average | 0.09 | 0.44 | 0.22 | 5.70 | 0.85 | 0.37 | 7.48 | 5.24 | 4.11 | 7.72 | 0.20 | 0.17 |
| | StDev | 0.01 | 0.18 | 0.03 | 0.63 | 0.18 | 0.05 | 0.57 | 0.05 | 0.33 | 1.60 | 0.02 | 0.07 |
| 8 | Average | 0.12 | 0.70 | 0.23 | 5.27 | 0.68 | 0.26 | 5.74 | 15.88 | 2.86 | 0.19 | 0.16 | 0.04 |
| | StDev | 0.06 | 0.06 | 0.02 | 0.58 | 0.02 | 0.03 | 1.05 | 0.27 | 0.15 | 0.01 | 0.01 | 0.00 |
| 9 | Average | 0.06 | 0.44 | 0.19 | 4.94 | 9.91 | 0.25 | 6.35 | 12.86 | 2.76 | 3.78 | 0.14 | 0.13 |
| | StDev | 0.02 | 0.08 | 0.04 | 1.37 | 1.02 | 0.03 | 1.75 | 0.51 | 0.22 | 0.58 | 0.02 | 0.05 |
| 10 | Average | 0.10 | 0.81 | 0.34 | 10.41 | 9.69 | 0.41 | 11.27 | 5.21 | 8.37 | 3.21 | 0.27 | 0.11 |
| | StDev | 0.02 | 0.20 | 0.18 | 4.88 | 1.31 | 0.10 | 4.18 | 0.20 | 1.29 | 0.59 | 0.02 | 0.02 |
| 11 | Average | 0.09 | 0.98 | 0.31 | 10.82 | 7.27 | 0.64 | 9.43 | 17.55 | 9.42 | 3.67 | 0.26 | 0.17 |
| | StDev | 0.06 | 0.64 | 0.12 | 4.65 | 5.79 | 0.43 | 2.01 | 3.02 | 0.58 | 1.55 | 0.09 | 0.01 |
| 12 | Average | 0.10 | 0.60 | 0.21 | 5.07 | 10.11 | 0.29 | 7.06 | 16.23 | 3.15 | 0.18 | 0.23 | 0.03 |
| | StDev | 0.08 | 0.12 | 0.05 | 0.88 | 0.40 | 0.04 | 1.60 | 0.99 | 0.10 | 0.01 | 0.02 | 0.00 |
| 13 | Average | 0.10 | 0.83 | 0.33 | 9.32 | 1.08 | 0.47 | 10.71 | 19.65 | 5.22 | 5.42 | 0.33 | 0.25 |
| | StDev | 0.03 | 0.10 | 0.05 | 1.53 | 0.05 | 0.07 | 3.00 | 1.45 | 0.23 | 0.51 | 0.02 | 0.03 |

| | | | | | | | | | | | | |
|---|---|---|---|---|---|---|---|---|---|---|---|---|
| Profile in Nature | 0.00 | 2.58 | 0.00 | 14.84 | 14.80 | 0.00 | 4.18 | 25.98 | 11.68 | 6.10 | 0.00 | 1.31 |

TABLE 14B
Fatty acid relative percent compositions for fatty acids 20:0 to 22:4
of control and lipid loaded carp preadipocytes and carp in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.47 | 0.19 | 0.39 | 0.08 | 4.55 | 0.19 | 6.94 | 0.38 | 1.01 | 0.56 | 2.54 | 12.94 |
| | StDev | 0.03 | 0.02 | 0.07 | 0.02 | 1.38 | 0.10 | 0.50 | 0.03 | 0.60 | 0.44 | 1.91 | 7.33 |
| 2 | Average | 0.44 | 0.12 | 0.21 | 0.10 | 1.10 | 0.11 | 3.70 | 7.77 | 1.14 | 0.53 | 1.45 | 8.30 |
| | StDev | 0.46 | 0.04 | 0.01 | 0.05 | 0.68 | 0.05 | 0.03 | 1.71 | 1.70 | 0.73 | 1.79 | 5.34 |
| 3 | Average | 0.48 | 0.12 | 0.10 | 0.05 | 0.94 | 0.07 | 3.62 | 8.41 | 2.26 | 1.34 | 1.59 | 5.30 |
| | StDev | 0.60 | 0.06 | 0.05 | 0.03 | 0.19 | 0.04 | 0.52 | 1.46 | 3.71 | 2.25 | 2.47 | 4.49 |
| 4 | Average | 0.32 | 0.09 | 0.10 | 0.03 | 1.30 | 0.06 | 4.84 | 0.44 | 0.46 | 0.09 | 0.13 | 2.21 |
| | StDev | 0.16 | 0.01 | 0.02 | 0.00 | 0.16 | 0.01 | 0.38 | 0.04 | 0.26 | 0.02 | 0.02 | 0.29 |
| 5 | Average | 0.40 | 0.18 | 0.27 | 0.04 | 1.19 | 0.11 | 5.15 | 0.39 | 0.76 | 0.72 | 1.59 | 6.69 |
| | StDev | 0.43 | 0.09 | 0.10 | 0.02 | 0.71 | 0.09 | 0.51 | 0.09 | 1.07 | 1.10 | 2.16 | 5.96 |
| 6 | Average | 0.22 | 0.19 | 0.35 | 0.04 | 1.93 | 0.09 | 6.38 | 0.27 | 0.09 | 0.14 | 0.45 | 3.41 |
| | StDev | 0.07 | 0.01 | 0.03 | 0.02 | 0.91 | 0.02 | 3.08 | 0.03 | 0.01 | 0.02 | 0.06 | 0.21 |
| 7 | Average | 0.14 | 0.10 | 0.22 | 0.09 | 0.54 | 0.05 | 2.98 | 19.76 | 0.06 | 0.03 | 0.15 | 2.74 |
| | StDev | 0.01 | 0.07 | 0.14 | 0.01 | 0.42 | 0.02 | 0.19 | 1.19 | 0.00 | 0.01 | 0.01 | 0.46 |
| 8 | Average | 0.10 | 0.10 | 0.10 | 0.01 | 0.57 | 0.03 | 2.41 | 20.92 | 0.04 | 0.04 | 0.09 | 1.87 |
| | StDev | 0.02 | 0.01 | 0.00 | 0.00 | 0.04 | 0.01 | 0.09 | 1.28 | 0.01 | 0.00 | 0.01 | 0.17 |
| 9 | Average | 0.11 | 0.09 | 0.12 | 0.12 | 0.63 | 0.03 | 2.11 | 0.44 | 0.04 | 0.04 | 0.11 | 1.92 |
| | StDev | 0.03 | 0.01 | 0.03 | 0.02 | 0.07 | 0.00 | 0.29 | 0.05 | 0.00 | 0.01 | 0.03 | 0.49 |
| 10 | Average | 0.21 | 0.08 | 0.21 | 0.05 | 0.81 | 0.05 | 3.02 | 11.96 | 0.08 | 0.05 | 0.17 | 2.74 |
| | StDev | 0.09 | 0.01 | 0.00 | 0.01 | 0.07 | 0.01 | 0.13 | 3.05 | 0.01 | 0.01 | 0.02 | 0.17 |
| 11 | Average | 0.33 | 0.09 | 0.13 | 0.08 | 2.85 | 0.12 | 3.81 | 19.42 | 0.59 | 0.37 | 0.38 | 2.77 |
| | StDev | 0.33 | 0.03 | 0.09 | 0.04 | 3.51 | 0.12 | 0.49 | 2.80 | 0.95 | 0.56 | 0.33 | 0.15 |
| 12 | Average | 0.13 | 0.10 | 0.13 | 0.01 | 0.82 | 0.04 | 2.90 | 0.44 | 0.06 | 0.06 | 0.15 | 2.34 |
| | StDev | 0.04 | 0.01 | 0.02 | 0.00 | 0.04 | 0.01 | 0.03 | 0.02 | 0.01 | 0.01 | 0.01 | 0.17 |
| 13 | Average | 0.18 | 0.13 | 0.18 | 0.08 | 1.09 | 0.06 | 4.39 | 21.06 | 0.07 | 0.08 | 0.22 | 3.86 |
| | StDev | 0.04 | 0.02 | 0.00 | 0.01 | 0.06 | 0.01 | 0.26 | 2.68 | 0.01 | 0.01 | 0.00 | 0.05 |

| | | | | | | | | | | | | |
|---|---|---|---|---|---|---|---|---|---|---|---|---|
| Profile in Nature | 0.00 | 1.60 | 0.00 | 0.00 | 0.00 | 0.00 | 3.43 | 0.00 | 0.00 | 9.08 | 0.00 | 0.00 |

TABLE 14C
Fatty acid relative percent compositions for fatty acids 22:5 N3 to 26:0 and total for SFA,
MUFA, PUFA, and Omega-3s of control and lipid loaded carp preadipocytes and carp in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 8.24 | 1.79 | 6.04 | 0.04 | 0.24 | 0.33 | 0.28 | 28.12 | 16.31 | 55.58 | 15.03 |
| | StDev | 2.77 | 0.76 | 0.18 | 0.01 | 0.15 | 0.07 | 0.27 | 11.51 | 1.68 | 13.08 | 2.53 |
| 2 | Average | 10.07 | 0.87 | 24.67 | 0.03 | 0.06 | 0.16 | 0.05 | 17.46 | 15.69 | 66.85 | 43.86 |
| | StDev | 2.88 | 0.42 | 4.49 | 0.02 | 0.02 | 0.02 | 0.03 | 4.03 | 4.13 | 5.23 | 5.80 |
| 3 | Average | 7.25 | 0.61 | 23.01 | 0.02 | 0.06 | 0.09 | 0.07 | 19.41 | 24.33 | 56.26 | 39.54 |
| | StDev | 1.56 | 0.24 | 5.15 | 0.02 | 0.02 | 0.06 | 0.04 | 4.81 | 4.67 | 1.21 | 5.61 |
| 4 | Average | 3.41 | 0.57 | 4.21 | 0.02 | 0.18 | 0.08 | 0.38 | 33.97 | 41.82 | 24.21 | 8.56 |
| | StDev | 0.30 | 0.05 | 0.64 | 0.02 | 0.16 | 0.01 | 0.23 | 10.02 | 7.38 | 2.67 | 1.01 |
| 5 | Average | 5.23 | 1.06 | 4.77 | 0.03 | 0.08 | 0.19 | 0.12 | 22.53 | 37.10 | 40.37 | 10.79 |
| | StDev | 1.89 | 0.59 | 0.50 | 0.02 | 0.03 | 0.10 | 0.07 | 3.57 | 6.49 | 7.31 | 1.49 |
| 6 | Average | 3.82 | 0.79 | 4.30 | 0.03 | 0.08 | 0.02 | 0.07 | 21.16 | 40.00 | 38.84 | 8.76 |
| | StDev | 0.30 | 0.05 | 0.24 | 0.00 | 0.02 | 0.01 | 0.02 | 2.77 | 4.16 | 1.51 | 0.59 |
| 7 | Average | 7.33 | 0.43 | 32.57 | 0.02 | 0.05 | 0.21 | 0.03 | 14.51 | 6.43 | 79.06 | 67.38 |
| | StDev | 0.70 | 0.13 | 1.58 | 0.01 | 0.01 | 0.08 | 0.02 | 1.26 | 0.32 | 1.20 | 1.16 |
| 8 | Average | 7.03 | 0.30 | 34.00 | 0.02 | 0.05 | 0.14 | 0.05 | 12.57 | 16.83 | 70.60 | 62.14 |
| | StDev | 0.17 | 0.03 | 0.76 | 0.01 | 0.02 | 0.02 | 0.04 | 1.68 | 0.26 | 1.44 | 1.33 |
| 9 | Average | 1.98 | 0.34 | 49.84 | 0.02 | 0.05 | 0.13 | 0.04 | 12.48 | 23.04 | 64.48 | 56.04 |
| | StDev | 0.43 | 0.06 | 2.03 | 0.01 | 0.02 | 0.01 | 0.03 | 3.30 | 1.42 | 2.94 | 2.27 |
| 10 | Average | 6.54 | 0.41 | 22.94 | 0.04 | 0.09 | 0.22 | 0.10 | 23.87 | 15.24 | 60.88 | 44.66 |
| | StDev | 0.72 | 0.06 | 2.43 | 0.02 | 0.04 | 0.01 | 0.05 | 9.73 | 1.43 | 8.31 | 6.75 |
| 11 | Average | 5.97 | 0.35 | 1.87 | 0.04 | 0.06 | 0.16 | 0.04 | 23.32 | 25.45 | 51.23 | 30.93 |
| | StDev | 3.10 | 0.10 | 1.15 | 0.02 | 0.01 | 0.02 | 0.02 | 7.07 | 2.71 | 4.36 | 8.22 |
| 12 | Average | 2.42 | 0.43 | 46.29 | 0.03 | 0.08 | 0.20 | 0.11 | 13.74 | 26.70 | 59.56 | 49.33 |
| | StDev | 0.13 | 0.04 | 1.60 | 0.01 | 0.04 | 0.01 | 0.11 | 2.70 | 1.25 | 1.47 | 1.50 |
| 13 | Average | 10.32 | 0.62 | 3.52 | 0.03 | 0.08 | 0.25 | 0.07 | 22.19 | 21.18 | 56.63 | 40.31 |
| | StDev | 0.14 | 0.02 | 0.33 | 0.01 | 0.01 | 0.02 | 0.02 | 4.82 | 1.47 | 3.35 | 2.85 |

| | | | | | | | | | | | |
|---|---|---|---|---|---|---|---|---|---|---|---|
| Profile in Nature | 1.85 | 0.00 | 2.58 | 0.00 | 0.00 | 0.00 | 0.00 | 21.59 | 51.46 | 26.95 | 10.53 |

TABLE 15A
Fatty acid relative percent compositions for fatty acids 12:0 to 18:4 of control
and lipid loaded red snapper muscle derived cells and red snapper in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.22 | 2.71 | 0.55 | 19.62 | 5.21 | 0.67 | 19.23 | 20.33 | 2.56 | 1.35 | 0.84 | 0.46 |
| | StDev | 0.04 | 0.27 | 0.33 | 4.99 | 0.46 | 0.02 | 3.63 | 1.84 | 0.54 | 2.18 | 0.24 | 0.54 |
| 2 | Average | 0.08 | 0.82 | 0.16 | 9.48 | 6.66 | 0.25 | 12.20 | 13.42 | 4.96 | 2.85 | 0.45 | 0.37 |
| | StDev | 0.02 | 0.10 | 0.03 | 2.34 | 0.08 | 0.04 | 2.16 | 1.17 | 0.47 | 0.22 | 0.03 | 0.02 |
| 3 | Average | 0.12 | 1.20 | 0.30 | 10.96 | 6.19 | 0.32 | 9.99 | 24.08 | 1.32 | 2.89 | 0.22 | 0.69 |
| | StDev | 0.02 | 0.24 | 0.11 | 2.65 | 0.54 | 0.07 | 2.37 | 1.74 | 0.48 | 1.62 | 0.02 | 0.42 |
| 4 | Average | 0.37 | 1.04 | 0.19 | 9.10 | 5.05 | 0.25 | 7.28 | 61.99 | 1.26 | 0.25 | 0.31 | 0.09 |
| | StDev | 0.44 | 0.03 | 0.01 | 0.88 | 0.20 | 0.03 | 1.58 | 3.70 | 0.37 | 0.14 | 0.01 | 0.03 |
| 5 | Average | 0.19 | 1.22 | 0.22 | 8.81 | 5.74 | 0.30 | 8.33 | 48.41 | 8.68 | 0.16 | 1.65 | 0.08 |
| | StDev | 0.12 | 0.11 | 0.02 | 0.94 | 0.71 | 0.02 | 0.35 | 1.93 | 0.12 | 0.02 | 0.27 | 0.02 |
| 6 | Average | 0.13 | 1.07 | 0.20 | 7.10 | 5.16 | 0.26 | 6.21 | 48.60 | 14.05 | 0.23 | 2.18 | 0.08 |
| | StDev | 0.04 | 0.17 | 0.05 | 1.36 | 0.51 | 0.02 | 0.90 | 1.94 | 0.79 | 0.03 | 0.21 | 0.01 |
| 7 | Average | 0.21 | 0.71 | 0.12 | 4.66 | 0.93 | 0.21 | 5.18 | 6.15 | 0.70 | 7.25 | 0.15 | 0.38 |
| | StDev | 0.24 | 0.11 | 0.02 | 0.52 | 0.07 | 0.05 | 0.79 | 0.12 | 0.04 | 0.96 | 0.02 | 0.12 |
| 8 | Average | 0.58 | 0.88 | 0.20 | 6.95 | 1.00 | 0.27 | 6.44 | 17.30 | 0.65 | 0.10 | 0.13 | 0.02 |
| | StDev | 0.47 | 0.03 | 0.09 | 0.40 | 0.15 | 0.03 | 0.61 | 0.64 | 0.04 | 0.07 | 0.01 | 0.00 |
| 9 | Average | 1.90 | 1.01 | 0.18 | 6.70 | 6.16 | 0.33 | 9.03 | 7.14 | 6.22 | 2.42 | 0.30 | 0.10 |
| | StDev | 1.88 | 0.10 | 0.01 | 0.85 | 0.85 | 0.06 | 1.15 | 0.35 | 0.47 | 0.41 | 0.07 | 0.01 |
| 10 | Average | 2.10 | 1.37 | 0.37 | 8.68 | 8.01 | 0.38 | 11.05 | 18.65 | 8.03 | 4.09 | 0.50 | 0.33 |
| | StDev | 1.69 | 0.11 | 0.16 | 1.51 | 1.07 | 0.06 | 0.33 | 0.80 | 0.43 | 1.00 | 0.06 | 0.13 |
| 11 | Average | 10.19 | 1.67 | 0.40 | 10.22 | 4.63 | 0.43 | 11.22 | 14.94 | 0.59 | 0.05 | 0.09 | 0.01 |
| | StDev | 10.06 | 0.49 | 0.04 | 2.04 | 2.71 | 0.15 | 3.01 | 2.14 | 0.05 | 0.02 | 0.04 | 0.01 |
| 12 | Average | 1.91 | 1.30 | 0.38 | 12.37 | 1.42 | 0.42 | 12.24 | 23.98 | 1.00 | 4.97 | 0.19 | 0.41 |
| | StDev | 2.24 | 0.22 | 0.39 | 4.05 | 0.07 | 0.08 | 4.04 | 3.36 | 0.29 | 0.97 | 0.03 | 0.15 |
| 13 | Average | 1.02 | 1.60 | 0.56 | 26.06 | 4.21 | 0.61 | 20.48 | 11.23 | 2.11 | 0.19 | 0.32 | 0.05 |
| | StDev | 0.72 | 0.38 | 0.07 | 8.22 | 1.41 | 0.13 | 10.89 | 3.39 | 0.81 | 0.07 | 0.11 | 0.02 |

| | | | | | | | | | | | | |
|---|---|---|---|---|---|---|---|---|---|---|---|---|
| Profile in Nature | 0.00 | 3.92 | 0.00 | 16.04 | 5.01 | 0.00 | 6.88 | 25.97 | 2.23 | 0.36 | 0.00 | .82 |

TABLE 15B
Fatty acid relative percent compositions for fatty acids 20:0 to 22:4 of control
and lipid loaded red snapper muscle derived cells and red snapper in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.21 | 0.20 | 0.10 | 0.09 | 1.57 | 0.23 | 6.97 | 0.71 | 0.20 | 0.11 | 0.07 | 2.99 |
| | StDev | 0.01 | 0.01 | 0.03 | 0.10 | 0.61 | 0.09 | 2.53 | 0.43 | 0.13 | 0.04 | 0.03 | 1.17 |
| 2 | Average | 0.12 | 0.07 | 0.09 | 0.06 | 0.59 | 0.08 | 3.06 | 10.30 | 0.06 | 0.03 | 0.05 | 1.57 |
| | StDev | 0.02 | 0.01 | 0.01 | 0.00 | 0.02 | 0.01 | 0.07 | 0.87 | 0.01 | 0.01 | 0.01 | 0.04 |
| 3 | Average | 0.12 | 0.13 | 0.07 | 0.08 | 0.54 | 0.07 | 2.99 | 7.88 | 0.06 | 0.04 | 0.02 | 1.39 |
| | StDev | 0.02 | 0.02 | 0.03 | 0.05 | 0.10 | 0.01 | 0.52 | 1.35 | 0.03 | 0.01 | 0.00 | 0.27 |
| 4 | Average | 0.09 | 0.18 | 0.04 | 0.01 | 0.72 | 0.12 | 3.47 | 0.30 | 0.04 | 0.10 | 0.02 | 1.42 |
| | StDev | 0.01 | 0.01 | 0.01 | 0.00 | 0.02 | 0.01 | 0.10 | 0.09 | 0.00 | 0.01 | 0.00 | 0.11 |
| 5 | Average | 0.09 | 0.17 | 0.11 | 0.01 | 0.97 | 0.14 | 4.39 | 0.27 | 0.06 | 0.12 | 0.09 | 1.97 |
| | StDev | 0.00 | 0.01 | 0.01 | 0.00 | 0.03 | 0.01 | 0.18 | 0.03 | 0.01 | 0.01 | 0.01 | 0.16 |
| 6 | Average | 0.07 | 0.15 | 0.13 | 0.01 | 0.86 | 0.13 | 3.88 | 0.26 | 0.05 | 0.14 | 0.11 | 1.75 |
| | StDev | 0.02 | 0.01 | 0.01 | 0.00 | 0.05 | 0.01 | 0.04 | 0.00 | 0.02 | 0.04 | 0.01 | 0.10 |
| 7 | Average | 0.06 | 0.05 | 0.04 | 0.10 | 0.49 | 0.09 | 2.61 | 20.69 | 0.03 | 0.03 | 0.02 | 1.37 |
| | StDev | 0.01 | 0.01 | 0.00 | 0.02 | 0.04 | 0.01 | 0.14 | 1.27 | 0.01 | 0.01 | 0.00 | 0.23 |
| 8 | Average | 0.08 | 0.09 | 0.04 | 0.00 | 0.56 | 0.07 | 2.65 | 15.73 | 0.03 | 0.04 | 0.02 | 1.64 |
| | StDev | 0.01 | 0.01 | 0.01 | 0.00 | 0.06 | 0.03 | 0.11 | 0.25 | 0.01 | 0.01 | 0.00 | 0.27 |
| 9 | Average | 0.11 | 0.07 | 0.16 | 0.09 | 0.71 | 0.10 | 3.18 | 11.81 | 0.06 | 0.05 | 0.07 | 2.37 |
| | StDev | 0.03 | 0.01 | 0.02 | 0.00 | 0.03 | 0.01 | 0.05 | 0.39 | 0.01 | 0.01 | 0.01 | 0.16 |
| 10 | Average | 0.15 | 0.11 | 0.16 | 0.10 | 0.78 | 0.12 | 3.97 | 18.40 | 0.10 | 0.07 | 0.14 | 2.57 |
| | StDev | 0.04 | 0.04 | 0.04 | 0.02 | 0.13 | 0.01 | 0.32 | 0.66 | 0.03 | 0.03 | 0.06 | 0.77 |
| 11 | Average | 0.39 | 0.19 | 0.06 | 0.01 | 0.72 | 0.12 | 2.55 | 0.59 | 0.25 | 0.16 | 0.05 | 2.57 |
| | StDev | 0.26 | 0.14 | 0.04 | 0.00 | 0.22 | 0.04 | 0.20 | 0.05 | 0.17 | 0.15 | 0.05 | 1.43 |
| 12 | Average | 0.13 | 0.13 | 0.06 | 0.14 | 0.75 | 0.13 | 3.98 | 21.36 | 0.06 | 0.07 | 0.03 | 2.57 |
| | StDev | 0.03 | 0.04 | 0.03 | 0.04 | 0.14 | 0.03 | 0.29 | 1.55 | 0.01 | 0.02 | 0.01 | 0.81 |
| 13 | Average | 0.25 | 0.13 | 0.11 | 0.02 | 2.18 | 0.21 | 8.09 | 0.40 | 0.23 | 0.08 | 0.12 | 5.25 |
| | StDev | 0.05 | 0.05 | 0.05 | 0.01 | 0.91 | 0.10 | 3.20 | 0.16 | 0.15 | 0.03 | 0.04 | 2.21 |

| | | | | | | | | | | | | |
|---|---|---|---|---|---|---|---|---|---|---|---|---|
| Profile in Nature | 0.00 | 1.91 | 0.00 | 0.00 | 0.00 | 0.00 | 4.05 | 4.56 | 0.00 | 0.00 | 0.00 | 0.00 |

TABLE 15C
Fatty acid relative percent compositions for fatty acids 22:5 N3 to 26:0 and total for SFA, MUFA, PUFA,
and Omega-3s of control and lipid loaded red snapper muscle derived cells and red snapper in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 4.14 | 0.93 | 7.05 | 0.01 | 0.13 | 0.29 | 0.29 | 43.84 | 26.15 | 30.01 | 13.25 |
| | StDev | 1.64 | 0.35 | 2.53 | 0.01 | 0.08 | 0.14 | 0.22 | 8.00 | 2.07 | 6.50 | 2.11 |
| 2 | Average | 2.99 | 0.34 | 28.60 | 0.01 | 0.05 | 0.15 | 0.07 | 23.30 | 20.34 | 56.36 | 44.74 |
| | StDev | 0.15 | 0.02 | 1.94 | 0.00 | 0.01 | 0.03 | 0.03 | 4.66 | 1.18 | 3.63 | 3.12 |
| 3 | Average | 2.38 | 0.34 | 25.34 | 0.02 | 0.06 | 0.11 | 0.07 | 23.21 | 30.56 | 46.22 | 38.50 |
| | StDev | 0.48 | 0.05 | 5.06 | 0.01 | 0.05 | 0.04 | 0.08 | 4.68 | 1.90 | 5.18 | 5.26 |
| 4 | Average | 2.01 | 0.47 | 3.58 | 0.01 | 0.03 | 0.15 | 0.04 | 18.45 | 67.46 | 14.09 | 6.14 |
| | StDev | 0.14 | 0.03 | 0.41 | 0.00 | 0.01 | 0.01 | 0.01 | 2.79 | 3.68 | 1.34 | 0.77 |
| 5 | Average | 2.65 | 0.61 | 4.33 | 0.01 | 0.03 | 0.14 | 0.04 | 19.30 | 54.58 | 26.12 | 7.41 |
| | StDev | 0.14 | 0.04 | 0.19 | 0.00 | 0.02 | 0.03 | 0.03 | 1.35 | 1.92 | 0.61 | 0.27 |
| 6 | Average | 2.38 | 0.53 | 3.82 | 0.02 | 0.04 | 0.34 | 0.04 | 15.20 | 54.40 | 30.40 | 6.69 |
| | StDev | 0.03 | 0.05 | 0.10 | 0.01 | 0.02 | 0.05 | 0.03 | 2.27 | 2.45 | 0.30 | 0.12 |
| 7 | Average | 3.43 | 0.29 | 43.85 | 0.01 | 0.02 | 0.16 | 0.02 | 11.23 | 7.32 | 81.45 | 75.22 |
| | StDev | 0.30 | 0.05 | 0.62 | 0.00 | 0.00 | 0.01 | 0.00 | 1.74 | 0.06 | 1.77 | 1.83 |
| 8 | Average | 3.43 | 0.38 | 40.55 | 0.01 | 0.01 | 0.15 | 0.01 | 15.46 | 18.58 | 65.95 | 59.81 |
| | StDev | 0.43 | 0.03 | 0.04 | 0.00 | 0.01 | 0.01 | 0.00 | 0.69 | 0.76 | 0.69 | 0.27 |
| 9 | Average | 4.19 | 0.47 | 34.89 | 0.01 | 0.02 | 0.14 | 0.02 | 19.36 | 13.55 | 67.09 | 53.31 |
| | StDev | 0.11 | 0.02 | 1.81 | 0.00 | 0.00 | 0.02 | 0.00 | 3.97 | 1.16 | 2.88 | 2.52 |
| 10 | Average | 4.93 | 0.55 | 3.95 | 0.03 | 0.07 | 0.15 | 0.11 | 24.38 | 26.99 | 48.64 | 31.37 |
| | StDev | 0.93 | 0.16 | 1.18 | 0.02 | 0.06 | 0.05 | 0.01 | 0.91 | 1.17 | 1.70 | 1.07 |
| 11 | Average | 1.92 | 0.53 | 35.11 | 0.03 | 0.08 | 0.14 | 0.09 | 34.97 | 20.07 | 44.96 | 37.66 |
| | StDev | 0.61 | 0.18 | 4.07 | 0.01 | 0.04 | 0.01 | 0.04 | 5.94 | 4.59 | 2.28 | 3.60 |
| 12 | Average | 5.77 | 0.55 | 3.42 | 0.02 | 0.04 | 0.14 | 0.04 | 28.92 | 25.73 | 45.35 | 35.51 |
| | StDev | 1.17 | 0.14 | 0.41 | 0.01 | 0.03 | 0.02 | 0.04 | 7.43 | 3.47 | 4.03 | 2.88 |
| 13 | Average | 5.19 | 1.14 | 7.49 | 0.01 | 0.21 | 0.14 | 0.29 | 51.32 | 15.79 | 32.88 | 13.27 |
| | StDev | 2.11 | 0.47 | 2.86 | 0.00 | 0.16 | 0.06 | 0.18 | 17.75 | 4.85 | 13.06 | 5.18 |

| | | | | | | | | | | | |
|---|---|---|---|---|---|---|---|---|---|---|---|
| Profile in Nature | 4.10 | 0.00 | 24.15 | 0.00 | 0.00 | 0.00 | 0.00 | 26.83 | 32.89 | 40.27 | 33.17 |

TABLE 16A
Fatty acid relative percent compositions for fatty acids 12:0 to 18:4 of control
and lipid loaded yellowfin tuna fibroblasts and yellowfin tuna in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 1.02 | 1.60 | 0.56 | 26.06 | 4.21 | 0.61 | 20.48 | 11.23 | 2.11 | 0.19 | 0.32 | 0.05 |
| | StDev | 0.72 | 0.38 | 0.07 | 8.22 | 1.41 | 0.13 | 10.89 | 3.39 | 0.81 | 0.07 | 0.11 | 0.02 |
| 2 | Average | 0.13 | 0.49 | 0.17 | 5.11 | 9.18 | 0.15 | 4.54 | 11.04 | 7.56 | 6.31 | 0.13 | 0.12 |
| | StDev | 0.02 | 0.06 | 0.03 | 1.07 | 0.32 | 0.01 | 0.99 | 0.59 | 0.27 | 0.19 | 0.01 | 0.01 |
| 3 | Average | 0.17 | 0.61 | 0.19 | 6.14 | 8.36 | 0.16 | 4.38 | 25.13 | 0.74 | 2.74 | 0.08 | 0.10 |
| | StDev | 0.03 | 0.05 | 0.03 | 0.82 | 0.32 | 0.01 | 0.62 | 0.84 | 0.04 | 0.13 | 0.01 | 0.02 |
| 4 | Average | 0.16 | 0.73 | 0.21 | 7.65 | 5.59 | 0.19 | 3.53 | 68.96 | 0.89 | 0.14 | 0.12 | 0.02 |
| | StDev | 0.03 | 0.07 | 0.01 | 0.26 | 0.38 | 0.01 | 0.31 | 1.11 | 0.04 | 0.02 | 0.01 | 0.00 |
| 5 | Average | 0.20 | 1.05 | 0.29 | 9.82 | 6.78 | 0.27 | 5.26 | 48.95 | 11.38 | 0.23 | 0.47 | 0.02 |
| | StDev | 0.08 | 0.17 | 0.06 | 0.79 | 0.32 | 0.04 | 0.56 | 3.07 | 0.68 | 0.03 | 0.04 | 0.00 |
| 6 | Average | 0.16 | 0.71 | 0.22 | 8.05 | 5.55 | 0.23 | 4.72 | 56.49 | 10.21 | 0.21 | 0.41 | 0.02 |
| | StDev | 0.04 | 0.09 | 0.05 | 1.70 | 0.13 | 0.05 | 2.07 | 4.44 | 0.63 | 0.01 | 0.01 | 0.00 |
| 7 | Average | 0.14 | 0.41 | 0.14 | 4.39 | 0.68 | 0.15 | 3.06 | 2.98 | 0.50 | 14.00 | 0.06 | 0.15 |
| | StDev | 0.11 | 0.04 | 0.01 | 0.30 | 0.02 | 0.02 | 0.39 | 0.27 | 0.09 | 1.55 | 0.00 | 0.03 |
| 8 | Average | 0.07 | 0.40 | 0.12 | 4.05 | 0.71 | 0.12 | 2.26 | 14.59 | 0.44 | 0.17 | 0.05 | 0.04 |
| | StDev | 0.02 | 0.05 | 0.02 | 0.49 | 0.02 | 0.02 | 0.40 | 0.35 | 0.02 | 0.11 | 0.01 | 0.00 |
| 9 | Average | 0.10 | 0.33 | 0.12 | 2.37 | 11.31 | 0.09 | 2.38 | 14.80 | 0.48 | 10.87 | 0.06 | 0.15 |
| | StDev | 0.02 | 0.01 | 0.02 | 0.41 | 0.35 | 0.01 | 0.53 | 0.43 | 0.01 | 0.32 | 0.00 | 0.01 |
| 10 | Average | 0.07 | 0.41 | 0.13 | 3.40 | 10.71 | 0.10 | 2.81 | 2.87 | 10.28 | 8.69 | 0.12 | 0.13 |
| | StDev | 0.04 | 0.03 | 0.01 | 0.21 | 0.51 | 0.00 | 0.25 | 0.17 | 0.04 | 0.16 | 0.01 | 0.01 |
| 11 | Average | 0.12 | 0.50 | 0.16 | 3.51 | 11.92 | 0.13 | 3.40 | 16.96 | 12.55 | 10.43 | 0.16 | 0.17 |
| | StDev | 0.00 | 0.05 | 0.02 | 0.41 | 0.32 | 0.01 | 0.31 | 0.33 | 0.32 | 0.21 | 0.01 | 0.02 |
| 12 | Average | 0.20 | 0.52 | 0.18 | 3.74 | 14.76 | 0.15 | 4.20 | 18.59 | 0.59 | 0.14 | 0.07 | 0.01 |
| | StDev | 0.08 | 0.07 | 0.02 | 0.09 | 0.77 | 0.02 | 0.13 | 0.70 | 0.11 | 0.09 | 0.01 | 0.00 |
| 13 | Average | 0.18 | 0.76 | 0.23 | 10.66 | 1.37 | 0.27 | 7.26 | 4.12 | 20.91 | 0.26 | 0.25 | 0.04 |
| | StDev | 0.04 | 0.02 | 0.02 | 1.13 | 0.08 | 0.02 | 1.49 | 0.20 | 1.09 | 0.01 | 0.01 | 0.00 |

| | | | | | | | | | | | | |
|---|---|---|---|---|---|---|---|---|---|---|---|---|
| Profile in Nature | 0.23 | 2.09 | 0.70 | 24.65 | 2.56 | 0.93 | 10.47 | 19.30 | 4.42 | 0.47 | 0.00 | 0.00 |

TABLE 16B
Fatty acid relative percent compositions for fatty acids 20:0 to 22:4 of control
and lipid loaded yellowfin tuna fibroblasts and yellowfin tuna in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.25 | 0.13 | 0.11 | 0.02 | 2.18 | 0.21 | 8.09 | 0.40 | 0.23 | 0.08 | 0.12 | 5.25 |
| | StDev | 0.05 | 0.05 | 0.05 | 0.01 | 0.91 | 0.10 | 3.20 | 0.16 | 0.15 | 0.03 | 0.04 | 2.21 |
| 2 | Average | 0.05 | 0.04 | 0.09 | 0.11 | 0.53 | 0.07 | 2.53 | 14.32 | 0.02 | 0.02 | 0.05 | 1.70 |
| | StDev | 0.01 | 0.01 | 0.01 | 0.00 | 0.04 | 0.01 | 0.14 | 1.44 | 0.00 | 0.00 | 0.00 | 0.08 |
| 3 | Average | 0.05 | 0.08 | 0.03 | 0.06 | 0.54 | 0.07 | 2.66 | 12.15 | 0.02 | 0.04 | 0.02 | 1.69 |
| | StDev | 0.01 | 0.01 | 0.00 | 0.01 | 0.05 | 0.01 | 0.11 | 0.88 | 0.00 | 0.00 | 0.00 | 0.17 |
| 4 | Average | 0.05 | 0.24 | 0.04 | 0.01 | 0.72 | 0.09 | 2.98 | 0.23 | 0.03 | 0.17 | 0.04 | 1.76 |
| | StDev | 0.00 | 0.02 | 0.00 | 0.00 | 0.01 | 0.01 | 0.08 | 0.06 | 0.00 | 0.01 | 0.00 | 0.03 |
| 5 | Average | 0.07 | 0.20 | 0.21 | 0.01 | 1.06 | 0.11 | 3.92 | 0.23 | 0.04 | 0.11 | 0.24 | 2.31 |
| | StDev | 0.01 | 0.02 | 0.02 | 0.00 | 0.06 | 0.01 | 0.34 | 0.03 | 0.01 | 0.04 | 0.02 | 0.19 |
| 6 | Average | 0.05 | 0.21 | 0.17 | 0.01 | 0.88 | 0.09 | 3.28 | 0.18 | 0.03 | 0.14 | 0.18 | 2.00 |
| | StDev | 0.02 | 0.01 | 0.02 | 0.00 | 0.06 | 0.01 | 0.37 | 0.02 | 0.00 | 0.01 | 0.01 | 0.20 |
| 7 | Average | 0.03 | 0.02 | 0.02 | 0.11 | 0.38 | 0.05 | 1.92 | 21.17 | 0.01 | 0.01 | 0.01 | 1.27 |
| | StDev | 0.01 | 0.01 | 0.00 | 0.01 | 0.03 | 0.01 | 0.39 | 0.88 | 0.01 | 0.01 | 0.00 | 0.12 |
| 8 | Average | 0.02 | 0.05 | 0.02 | 0.00 | 0.35 | 0.05 | 1.45 | 24.61 | 0.01 | 0.01 | 0.01 | 1.06 |
| | StDev | 0.01 | 0.00 | 0.00 | 0.00 | 0.02 | 0.01 | 0.13 | 0.28 | 0.00 | 0.00 | 0.00 | 0.07 |
| 9 | Average | 0.03 | 0.05 | 0.03 | 0.23 | 0.38 | 0.04 | 1.58 | 0.51 | 0.01 | 0.02 | 0.01 | 0.94 |
| | StDev | 0.01 | 0.00 | 0.00 | 0.00 | 0.01 | 0.00 | 0.02 | 0.35 | 0.00 | 0.01 | 0.00 | 0.03 |
| 10 | Average | 0.03 | 0.02 | 0.10 | 0.12 | 0.42 | 0.06 | 2.24 | 16.05 | 0.01 | 0.01 | 0.05 | 1.42 |
| | StDev | 0.00 | 0.00 | 0.00 | 0.01 | 0.02 | 0.01 | 0.05 | 0.94 | 0.00 | 0.00 | 0.00 | 0.06 |
| 11 | Average | 0.03 | 0.05 | 0.10 | 0.12 | 0.52 | 0.08 | 3.10 | 25.13 | 0.01 | 0.03 | 0.09 | 1.70 |
| | StDev | 0.01 | 0.01 | 0.01 | 0.01 | 0.02 | 0.00 | 0.11 | 0.60 | 0.00 | 0.01 | 0.00 | 0.12 |
| 12 | Average | 0.04 | 0.08 | 0.03 | 0.01 | 0.48 | 0.05 | 2.03 | 0.48 | 0.02 | 0.04 | 0.02 | 1.28 |
| | StDev | 0.00 | 0.01 | 0.00 | 0.00 | 0.02 | 0.00 | 0.14 | 0.26 | 0.00 | 0.00 | 0.00 | 0.11 |
| 13 | Average | 0.05 | 0.04 | 0.19 | 0.01 | 0.67 | 0.08 | 3.72 | 29.94 | 0.02 | 0.01 | 0.16 | 2.62 |
| | StDev | 0.01 | 0.00 | 0.01 | 0.00 | 0.04 | 0.02 | 0.16 | 2.20 | 0.00 | 0.00 | 0.00 | 0.13 |

| | | | | | | | | | | | | |
|---|---|---|---|---|---|---|---|---|---|---|---|---|
| Profile in Nature | 0.47 | 1.86 | 0.23 | 0.23 | 0.23 | 0.00 | 3.49 | 2.79 | 0.23 | 0.23 | 0.00 | .47 |

TABLE 16C
Fatty acid relative percent compositions for fatty acids 22:5 N3 to 26:0 and total for SFA, MUFA, PUFA,
and Omega-3s of control and lipid loaded yellowfin tuna fibroblasts and yellowfin tuna in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 5.19 | 1.14 | 7.49 | 0.01 | 0.21 | 0.14 | 0.29 | 51.32 | 15.79 | 32.88 | 13.27 |
| | StDev | 2.11 | 0.47 | 2.86 | 0.00 | 0.16 | 0.06 | 0.18 | 17.75 | 4.85 | 13.06 | 5.18 |
| 2 | Average | 6.07 | 0.24 | 29.15 | 0.00 | 0.02 | 0.03 | 0.02 | 10.69 | 20.33 | 68.98 | 55.83 |
| | StDev | 0.24 | 0.02 | 0.80 | 0.00 | 0.01 | 0.00 | 0.01 | 2.15 | 0.42 | 2.52 | 2.04 |
| 3 | Average | 5.47 | 0.26 | 27.98 | 0.01 | 0.02 | 0.03 | 0.02 | 11.76 | 33.64 | 54.60 | 48.34 |
| | StDev | 0.42 | 0.03 | 1.18 | 0.00 | 0.01 | 0.00 | 0.01 | 1.57 | 0.92 | 1.92 | 1.59 |
| 4 | Average | 1.93 | 0.41 | 3.00 | 0.01 | 0.02 | 0.07 | 0.03 | 12.61 | 75.03 | 12.36 | 5.30 |
| | StDev | 0.03 | 0.01 | 0.07 | 0.00 | 0.00 | 0.00 | 0.00 | 0.65 | 0.76 | 0.11 | 0.10 |
| 5 | Average | 2.42 | 0.51 | 3.67 | 0.01 | 0.03 | 0.08 | 0.03 | 17.07 | 56.11 | 26.82 | 6.56 |
| | StDev | 0.19 | 0.04 | 0.27 | 0.01 | 0.01 | 0.01 | 0.01 | 1.69 | 2.77 | 1.50 | 0.50 |
| 6 | Average | 2.06 | 0.45 | 3.16 | 0.01 | 0.02 | 0.07 | 0.02 | 14.21 | 62.46 | 23.32 | 5.62 |
| | StDev | 0.17 | 0.05 | 0.25 | 0.01 | 0.01 | 0.01 | 0.01 | 3.98 | 4.31 | 1.13 | 0.43 |
| 7 | Average | 5.50 | 0.22 | 42.53 | 0.01 | 0.01 | 0.03 | 0.02 | 8.37 | 3.73 | 87.91 | 83.20 |
| | StDev | 0.52 | 0.04 | 0.42 | 0.00 | 0.00 | 0.00 | 0.01 | 0.84 | 0.29 | 1.10 | 1.51 |
| 8 | Average | 5.31 | 0.23 | 43.78 | 0.00 | 0.01 | 0.02 | 0.01 | 7.07 | 15.38 | 77.55 | 73.86 |
| | StDev | 0.17 | 0.03 | 0.98 | 0.00 | 0.00 | 0.00 | 0.00 | 0.98 | 0.34 | 0.74 | 0.98 |
| 9 | Average | 1.03 | 0.20 | 51.79 | 0.01 | 0.01 | 0.06 | 0.01 | 5.45 | 26.23 | 68.32 | 64.20 |
| | StDev | 0.10 | 0.01 | 0.87 | 0.00 | 0.00 | 0.00 | 0.00 | 0.97 | 0.09 | 0.91 | 0.96 |
| 10 | Average | 5.56 | 0.26 | 33.86 | 0.00 | 0.01 | 0.04 | 0.01 | 6.98 | 13.66 | 79.36 | 64.16 |
| | StDev | 0.21 | 0.02 | 0.55 | 0.00 | 0.00 | 0.00 | 0.01 | 0.48 | 0.53 | 0.96 | 1.08 |
| 11 | Average | 6.39 | 0.29 | 2.29 | 0.01 | 0.01 | 0.04 | 0.01 | 7.88 | 29.00 | 63.12 | 44.23 |
| | StDev | 0.35 | 0.02 | 0.32 | 0.00 | 0.01 | 0.01 | 0.01 | 0.82 | 0.50 | 0.71 | 0.38 |
| 12 | Average | 1.37 | 0.30 | 50.55 | 0.01 | 0.02 | 0.04 | 0.02 | 9.10 | 33.50 | 57.40 | 52.53 |
| | StDev | 0.11 | 0.01 | 1.54 | 0.00 | 0.00 | 0.01 | 0.00 | 0.05 | 0.91 | 0.95 | 1.11 |
| 13 | Average | 12.85 | 0.37 | 2.87 | 0.01 | 0.01 | 0.05 | 0.01 | 19.47 | 5.58 | 74.95 | 45.91 |
| | StDev | 0.50 | 0.04 | 0.55 | 0.00 | 0.01 | 0.01 | 0.01 | 2.73 | 0.29 | 3.02 | 1.79 |

| | | | | | | | | | | | |
|---|---|---|---|---|---|---|---|---|---|---|---|
| Profile in Nature | 0.93 | 0.00 | 20.47 | 0.00 | 0.23 | 1.86 | 0.00 | 40.00 | 26.28 | 33.72 | 24.65 |

TABLE 17A
Fatty acid relative percent compositions for fatty acids 12:0 to 18:4 of
control and lipid loaded bluegill fibroblasts and bluegill in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.30 | 2.05 | 0.61 | 15.97 | 2.56 | 0.59 | 27.36 | 7.39 | 1.46 | 0.14 | 0.81 | 0.11 |
| | StDev | 0.07 | 0.37 | 0.12 | 3.99 | 1.24 | 0.03 | 5.56 | 1.82 | 0.31 | 0.02 | 0.31 | 0.05 |
| 2 | Average | 0.16 | 0.90 | 0.29 | 7.51 | 6.05 | 0.31 | 14.62 | 8.40 | 5.02 | 3.09 | 0.35 | 0.14 |
| | StDev | 0.04 | 0.13 | 0.05 | 2.25 | 0.41 | 0.03 | 2.98 | 1.21 | 0.55 | 0.33 | 0.01 | 0.01 |
| 3 | Average | 0.16 | 1.12 | 0.36 | 9.84 | 5.13 | 0.33 | 12.70 | 17.57 | 0.61 | 1.05 | 0.27 | 0.14 |
| | StDev | 0.02 | 0.14 | 0.05 | 2.86 | 0.12 | 0.04 | 3.19 | 1.73 | 0.00 | 0.18 | 0.03 | 0.02 |
| 4 | Average | 0.20 | 1.23 | 0.42 | 11.16 | 6.17 | 0.42 | 17.80 | 19.35 | 0.85 | 1.24 | 0.37 | 0.15 |
| | StDev | 0.11 | 0.03 | 0.04 | 1.15 | 0.52 | 0.04 | 4.08 | 2.35 | 0.11 | 0.13 | 0.09 | 0.02 |
| 5 | Average | 0.18 | 1.21 | 0.45 | 11.41 | 3.96 | 0.46 | 16.99 | 21.84 | 7.78 | 0.13 | 0.62 | 0.02 |
| | StDev | 0.05 | 0.04 | 0.05 | 0.02 | 0.23 | 0.03 | 0.87 | 1.73 | 0.35 | 0.01 | 0.08 | 0.00 |
| 6 | Average | 0.13 | 1.13 | 0.35 | 9.25 | 3.45 | 0.41 | 14.36 | 27.53 | 12.19 | 0.14 | 1.10 | 0.03 |
| | StDev | 0.01 | 0.06 | 0.01 | 0.77 | 0.06 | 0.02 | 2.76 | 2.79 | 1.72 | 0.01 | 0.06 | 0.00 |
| 7 | Average | 0.11 | 0.78 | 0.34 | 5.51 | 0.63 | 0.24 | 8.11 | 2.92 | 0.49 | 12.38 | 0.21 | 0.36 |
| | StDev | 0.10 | 0.15 | 0.17 | 0.74 | 0.14 | 0.05 | 1.84 | 0.45 | 0.03 | 1.06 | 0.05 | 0.04 |
| 8 | Average | 0.11 | 1.17 | 0.36 | 8.31 | 1.05 | 0.34 | 9.65 | 12.16 | 0.60 | 0.11 | 0.30 | 0.13 |
| | StDev | 0.02 | 0.06 | 0.01 | 0.39 | 0.13 | 0.01 | 0.06 | 0.24 | 0.04 | 0.02 | 0.04 | 0.01 |
| 9 | Average | 0.08 | 0.91 | 0.29 | 6.45 | 7.35 | 0.29 | 11.08 | 3.38 | 7.04 | 5.22 | 0.38 | 0.18 |
| | StDev | 0.03 | 0.10 | 0.01 | 0.82 | 0.24 | 0.04 | 1.26 | 0.32 | 1.22 | 0.52 | 0.04 | 0.02 |
| 10 | Average | 0.04 | 0.61 | 0.20 | 3.99 | 8.06 | 0.17 | 7.87 | 10.82 | 0.54 | 5.89 | 0.21 | 0.24 |
| | StDev | 0.01 | 0.09 | 0.02 | 0.61 | 0.67 | 0.03 | 1.71 | 0.34 | 0.16 | 0.47 | 0.06 | 0.06 |
| 11 | Average | 0.07 | 1.00 | 0.32 | 7.51 | 12.38 | 0.31 | 15.02 | 12.52 | 2.36 | 5.88 | 0.43 | 0.34 |
| | StDev | 0.01 | 0.06 | 0.02 | 1.90 | 0.79 | 0.02 | 2.38 | 0.43 | 0.15 | 0.25 | 0.04 | 0.03 |
| 12 | Average | 0.10 | 1.84 | 0.94 | 5.90 | 9.39 | 0.34 | 9.05 | 12.13 | 0.61 | 0.07 | 0.30 | 0.03 |
| | StDev | 0.09 | 1.91 | 1.24 | 4.14 | 0.81 | 0.23 | 0.95 | 0.81 | 0.08 | 0.01 | 0.08 | 0.01 |
| 13 | Average | 0.18 | 1.30 | 0.41 | 9.90 | 1.34 | 0.49 | 14.29 | 13.80 | 1.27 | 7.51 | 0.51 | 0.34 |
| | StDev | 0.04 | 0.03 | 0.04 | 0.13 | 0.07 | 0.01 | 0.26 | 0.28 | 0.05 | 0.28 | 0.03 | 0.05 |

| | | | | | | | | | | | | |
|---|---|---|---|---|---|---|---|---|---|---|---|---|
| Profile in Nature | 0.00 | 1.82 | 0.00 | 20.05 | 8.20 | 0.00 | 4.44 | 10.02 | 1.59 | 1.71 | 0.00 | 1.14 |

TABLE 17B
Fatty acid relative percent compositions for fatty acids 20:0 to 22:4 of
control and lipid loaded bluegill fibroblasts and bluegill in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.32 | 0.08 | 0.06 | 0.01 | 1.23 | 0.09 | 8.75 | 0.31 | 0.29 | 0.03 | 0.03 | 10.07 |
| | StDev | 0.05 | 0.03 | 0.01 | 0.00 | 0.28 | 0.03 | 2.10 | 0.11 | 0.23 | 0.02 | 0.01 | 0.58 |
| 2 | Average | 0.14 | 0.09 | 0.08 | 0.02 | 0.63 | 0.05 | 4.22 | 9.99 | 0.08 | 0.05 | 0.06 | 6.37 |
| | StDev | 0.04 | 0.00 | 0.04 | 0.00 | 0.02 | 0.01 | 0.18 | 1.28 | 0.01 | 0.01 | 0.01 | 0.85 |
| 3 | Average | 0.13 | 0.15 | 0.04 | 0.02 | 0.62 | 0.05 | 4.33 | 7.73 | 0.07 | 0.07 | 0.02 | 5.98 |
| | StDev | 0.03 | 0.00 | 0.00 | 0.00 | 0.02 | 0.01 | 0.28 | 1.75 | 0.00 | 0.01 | 0.00 | 0.56 |
| 4 | Average | 0.16 | 0.15 | 0.06 | 0.02 | 0.92 | 0.06 | 6.61 | 9.04 | 0.08 | 0.08 | 0.03 | 8.35 |
| | StDev | 0.04 | 0.01 | 0.01 | 0.00 | 0.08 | 0.01 | 0.75 | 0.40 | 0.03 | 0.01 | 0.00 | 0.40 |
| 5 | Average | 0.15 | 0.14 | 0.10 | 0.01 | 1.03 | 0.06 | 6.15 | 0.25 | 0.10 | 0.09 | 0.09 | 10.16 |
| | StDev | 0.02 | 0.01 | 0.01 | 0.00 | 0.04 | 0.01 | 0.23 | 0.02 | 0.04 | 0.02 | 0.02 | 0.36 |
| 6 | Average | 0.12 | 0.16 | 0.12 | 0.01 | 0.94 | 0.07 | 6.23 | 0.23 | 0.07 | 0.09 | 0.12 | 7.57 |
| | StDev | 0.02 | 0.01 | 0.00 | 0.00 | 0.01 | 0.02 | 0.51 | 0.01 | 0.03 | 0.02 | 0.01 | 0.12 |
| 7 | Average | 0.08 | 0.04 | 0.03 | 0.09 | 0.43 | 0.06 | 3.08 | 19.19 | 0.06 | 0.02 | 0.02 | 3.66 |
| | StDev | 0.02 | 0.01 | 0.01 | 0.02 | 0.05 | 0.03 | 0.28 | 2.33 | 0.01 | 0.01 | 0.00 | 0.52 |
| 8 | Average | 0.09 | 0.13 | 0.04 | 0.01 | 0.59 | 0.04 | 4.43 | 14.01 | 0.05 | 0.04 | 0.02 | 5.16 |
| | StDev | 0.01 | 0.01 | 0.00 | 0.00 | 0.03 | 0.02 | 0.18 | 1.01 | 0.01 | 0.01 | 0.00 | 0.17 |
| 9 | Average | 0.08 | 0.05 | 0.12 | 0.04 | 0.60 | 0.04 | 4.01 | 12.98 | 0.04 | 0.02 | 0.06 | 5.53 |
| | StDev | 0.01 | 0.01 | 0.02 | 0.01 | 0.02 | 0.01 | 0.27 | 1.29 | 0.01 | 0.00 | 0.01 | 0.45 |
| 10 | Average | 0.07 | 0.10 | 0.03 | 0.05 | 0.44 | 0.03 | 3.00 | 0.43 | 0.03 | 0.04 | 0.02 | 3.70 |
| | StDev | 0.01 | 0.01 | 0.00 | 0.00 | 0.06 | 0.01 | 0.48 | 0.03 | 0.01 | 0.00 | 0.00 | 0.57 |
| 11 | Average | 0.10 | 0.11 | 0.07 | 0.05 | 0.77 | 0.07 | 5.98 | 10.90 | 0.04 | 0.04 | 0.05 | 7.77 |
| | StDev | 0.03 | 0.02 | 0.01 | 0.00 | 0.04 | 0.02 | 0.32 | 0.67 | 0.01 | 0.01 | 0.01 | 0.72 |
| 12 | Average | 0.10 | 0.11 | 0.04 | 0.01 | 0.60 | 0.05 | 4.08 | 0.43 | 0.07 | 0.06 | 0.02 | 5.10 |
| | StDev | 0.07 | 0.04 | 0.00 | 0.00 | 0.09 | 0.01 | 0.73 | 0.05 | 0.08 | 0.02 | 0.00 | 0.80 |
| 13 | Average | 0.10 | 0.13 | 0.06 | 0.06 | 0.81 | 0.08 | 6.96 | 13.88 | 0.05 | 0.05 | 0.03 | 8.94 |
| | StDev | 0.00 | 0.00 | 0.00 | 0.00 | 0.03 | 0.00 | 0.14 | 1.11 | 0.01 | 0.01 | 0.01 | 0.83 |

| | | | | | | | | | | | | |
|---|---|---|---|---|---|---|---|---|---|---|---|---|
| Profile in Nature | 0.00 | 3.42 | 0.00 | 0.00 | 0.00 | 0.00 | 7.40 | 11.28 | 0.00 | 0.00 | 0.00 | 0.00 |

TABLE 17C
Fatty acid relative percent compositions for fatty acids 22:5 N3 to 26:0 and total for SFA, MUFA,
PUFA, and Omega-3s of control and lipid loaded bluegill fibroblasts and bluegill in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 7.30 | 1.83 | 9.73 | 0.01 | 0.15 | 0.05 | 0.30 | 47.95 | 10.11 | 41.95 | 17.47 |
| | StDev | 0.67 | 0.09 | 1.95 | 0.01 | 0.09 | 0.00 | 0.15 | 8.39 | 2.99 | 5.43 | 2.68 |
| 2 | Average | 5.28 | 1.06 | 24.77 | 0.01 | 0.03 | 0.19 | 0.03 | 24.08 | 14.78 | 61.14 | 43.14 |
| | StDev | 0.38 | 0.22 | 1.41 | 0.00 | 0.01 | 0.04 | 0.02 | 5.38 | 1.62 | 3.79 | 2.82 |
| 3 | Average | 4.94 | 1.04 | 25.34 | 0.01 | 0.03 | 0.11 | 0.05 | 24.80 | 23.03 | 52.17 | 39.06 |
| | StDev | 0.26 | 0.14 | 2.74 | 0.00 | 0.01 | 0.04 | 0.00 | 6.28 | 1.90 | 4.40 | 4.77 |
| 4 | Average | 7.21 | 1.40 | 6.30 | 0.01 | 0.03 | 0.09 | 0.05 | 31.57 | 25.84 | 42.60 | 23.78 |
| | StDev | 0.35 | 0.04 | 0.60 | 0.00 | 0.01 | 0.02 | 0.01 | 5.13 | 2.78 | 2.43 | 1.43 |
| 5 | Average | 6.37 | 1.83 | 8.19 | 0.02 | 0.05 | 0.10 | 0.08 | 31.09 | 26.12 | 42.78 | 14.94 |
| | StDev | 0.34 | 0.15 | 1.09 | 0.01 | 0.03 | 0.03 | 0.05 | 1.04 | 1.58 | 1.95 | 1.42 |
| 6 | Average | 5.33 | 1.50 | 7.24 | 0.01 | 0.02 | 0.06 | 0.03 | 25.88 | 31.30 | 42.82 | 12.95 |
| | StDev | 0.21 | 0.10 | 0.77 | 0.00 | 0.01 | 0.03 | 0.01 | 2.90 | 2.80 | 0.43 | 0.92 |
| 7 | Average | 4.52 | 0.59 | 35.77 | 0.01 | 0.03 | 0.16 | 0.04 | 15.32 | 3.77 | 80.91 | 71.86 |
| | StDev | 0.34 | 0.10 | 1.21 | 0.01 | 0.02 | 0.04 | 0.02 | 2.95 | 0.57 | 3.07 | 3.64 |
| 8 | Average | 5.11 | 0.79 | 35.04 | 0.01 | 0.03 | 0.10 | 0.03 | 20.14 | 13.49 | 66.37 | 54.27 |
| | StDev | 0.19 | 0.09 | 0.40 | 0.00 | 0.00 | 0.03 | 0.00 | 0.26 | 0.14 | 0.36 | 0.53 |
| 9 | Average | 4.95 | 0.91 | 27.70 | 0.01 | 0.01 | 0.19 | 0.01 | 19.24 | 10.98 | 69.77 | 50.86 |
| | StDev | 0.20 | 0.17 | 0.62 | 0.00 | 0.01 | 0.08 | 0.01 | 2.12 | 0.48 | 2.54 | 2.21 |
| 10 | Average | 2.31 | 0.63 | 50.26 | 0.00 | 0.01 | 0.20 | 0.01 | 13.00 | 19.22 | 67.78 | 58.89 |
| | StDev | 0.28 | 0.09 | 1.97 | 0.00 | 0.01 | 0.06 | 0.01 | 2.27 | 0.81 | 1.57 | 2.19 |
| 11 | Average | 7.69 | 1.31 | 6.48 | 0.01 | 0.03 | 0.34 | 0.02 | 24.43 | 25.40 | 50.16 | 30.96 |
| | StDev | 0.63 | 0.16 | 0.47 | 0.01 | 0.02 | 0.15 | 0.02 | 4.30 | 1.24 | 3.06 | 1.80 |
| 12 | Average | 3.19 | 0.80 | 44.26 | 0.03 | 0.16 | 0.12 | 0.13 | 18.61 | 21.81 | 59.58 | 47.96 |
| | StDev | 0.51 | 0.11 | 7.57 | 0.05 | 0.21 | 0.04 | 0.22 | 8.87 | 0.20 | 9.00 | 7.95 |
| 13 | Average | 8.43 | 1.52 | 7.31 | 0.00 | 0.02 | 0.23 | 0.02 | 26.72 | 15.56 | 57.71 | 37.13 |
| | StDev | 0.53 | 0.07 | 0.52 | 0.00 | 0.01 | 0.03 | 0.01 | 0.33 | 0.31 | 0.64 | 0.35 |

| | | | | | | | | | | | |
|---|---|---|---|---|---|---|---|---|---|---|---|
| Profile in Nature | 3.99 | 0.00 | 24.94 | 0.00 | 0.00 | 0.00 | 0.00 | 26.31 | 21.64 | 52.05 | 41.91 |

TABLE 18A
Fatty acid relative percent compositions for fatty acids 12:0 to 18:4 of control
and lipid loaded tilapia brain derived cells and tilapia in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.18 | 0.91 | 0.38 | 18.67 | 3.90 | 0.45 | 19.58 | 13.29 | 2.51 | 0.92 | 1.02 | 0.24 |
| | StDev | 0.15 | 0.40 | 0.07 | 9.72 | 2.44 | 0.10 | 6.98 | 2.49 | 2.36 | 1.43 | 0.71 | 0.26 |
| 2 | Average | 0.06 | 0.26 | 0.17 | 8.72 | 4.74 | 0.30 | 8.17 | 22.83 | 4.24 | 1.92 | 0.44 | 0.37 |
| | StDev | 0.04 | 0.13 | 0.04 | 1.86 | 1.75 | 0.03 | 1.49 | 9.15 | 3.13 | 1.19 | 0.15 | 0.18 |
| 3 | Average | 0.08 | 0.44 | 0.25 | 10.64 | 4.23 | 0.33 | 8.97 | 35.37 | 0.65 | 0.58 | 0.39 | 0.25 |
| | StDev | 0.03 | 0.20 | 0.08 | 4.19 | 1.14 | 0.09 | 2.35 | 7.09 | 0.09 | 0.46 | 0.19 | 0.17 |
| 4 | Average | 0.08 | 0.53 | 0.23 | 10.40 | 2.95 | 0.28 | 7.52 | 48.69 | 2.61 | 0.05 | 1.23 | 0.06 |
| | StDev | 0.02 | 0.11 | 0.06 | 0.56 | 0.18 | 0.07 | 0.29 | 5.97 | 3.23 | 0.02 | 1.01 | 0.01 |
| 5 | Average | 0.07 | 0.27 | 0.16 | 9.08 | 1.67 | 0.36 | 7.94 | 32.93 | 6.09 | 0.04 | 1.66 | 0.02 |
| | StDev | 0.04 | 0.17 | 0.10 | 4.73 | 1.07 | 0.03 | 1.15 | 6.06 | 0.61 | 0.00 | 0.34 | 0.01 |
| 6 | Average | 0.09 | 0.25 | 0.16 | 5.42 | 1.44 | 0.33 | 7.41 | 26.99 | 6.70 | 3.75 | 1.59 | 0.47 |
| | StDev | 0.06 | 0.13 | 0.10 | 1.29 | 1.55 | 0.04 | 3.21 | 20.02 | 5.31 | 6.40 | 1.34 | 0.76 |
| 7 | Average | 0.31 | 0.30 | 0.15 | 5.46 | 0.31 | 0.42 | 9.03 | 9.11 | 0.86 | 5.20 | 0.20 | 0.51 |
| | StDev | 0.30 | 0.14 | 0.10 | 2.40 | 0.32 | 0.01 | 0.94 | 4.56 | 0.07 | 5.36 | 0.02 | 0.47 |
| 8 | Average | 0.45 | 0.26 | 0.08 | 4.82 | 0.33 | 0.40 | 9.48 | 15.47 | 0.67 | 0.68 | 0.10 | 0.06 |
| | StDev | 0.45 | 0.27 | 0.02 | 0.95 | 0.39 | 0.04 | 1.56 | 1.96 | 0.08 | 0.75 | 0.03 | 0.05 |
| 9 | Average | 0.07 | 0.16 | 0.14 | 5.72 | 5.40 | 0.35 | 6.73 | 17.78 | 2.50 | 3.51 | 0.26 | 0.32 |
| | StDev | 0.02 | 0.11 | 0.09 | 3.26 | 4.25 | 0.11 | 1.62 | 8.83 | 3.19 | 2.07 | 0.23 | 0.16 |
| 10 | Average | 0.08 | 0.42 | 0.29 | 10.97 | 9.28 | 0.43 | 9.67 | 11.35 | 7.16 | 4.09 | 0.65 | 0.67 |
| | StDev | 0.02 | 0.02 | 0.04 | 1.99 | 1.82 | 0.07 | 0.93 | 9.03 | 0.84 | 0.95 | 0.39 | 0.53 |
| 11 | Average | 0.16 | 0.41 | 0.25 | 9.51 | 7.45 | 0.40 | 11.33 | 17.39 | 5.34 | 3.29 | 0.55 | 0.63 |
| | StDev | 0.05 | 0.31 | 0.17 | 2.79 | 5.11 | 0.04 | 3.06 | 3.72 | 3.64 | 2.56 | 0.36 | 0.51 |
| 12 | Average | 0.30 | 0.23 | 0.14 | 6.18 | 2.49 | 0.41 | 10.70 | 17.25 | 0.76 | 0.83 | 0.16 | 0.13 |
| | StDev | 0.25 | 0.07 | 0.06 | 2.51 | 3.34 | 0.02 | 1.25 | 2.31 | 0.19 | 0.73 | 0.03 | 0.11 |
| 13 | Average | 0.14 | 0.91 | 0.43 | 24.45 | 0.74 | 0.44 | 30.70 | 12.28 | 0.71 | 1.78 | 0.17 | 0.41 |
| | StDev | 0.01 | 0.27 | 0.04 | 11.18 | 0.21 | 0.10 | 17.25 | 10.95 | 0.14 | 2.97 | 0.16 | 0.69 |

| | | | | | | | | | | | | |
|---|---|---|---|---|---|---|---|---|---|---|---|---|
| Profile in Nature | 0.00 | 3.68 | 0.00 | 29.42 | 6.87 | 0.00 | 7.43 | 26.30 | 10.96 | 2.29 | 0.00 | 0.21 |

TABLE 18B
Fatty acid relative percent compositions for fatty acids 20:0 to 22:4 of control
and lipid loaded tilapia brain derived cells and tilapia in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.23 | 0.16 | 0.06 | 0.01 | 1.34 | 0.27 | 8.25 | 2.78 | 0.19 | 0.04 | 0.03 | 4.41 |
| | StDev | 0.09 | 0.06 | 0.01 | 0.02 | 0.74 | 0.15 | 4.66 | 3.99 | 0.17 | 0.01 | 0.01 | 1.58 |
| 2 | Average | 0.10 | 0.17 | 0.08 | 0.03 | 0.87 | 0.13 | 4.86 | 7.67 | 0.05 | 0.05 | 0.03 | 4.22 |
| | StDev | 0.01 | 0.04 | 0.05 | 0.02 | 0.25 | 0.04 | 0.62 | 1.27 | 0.01 | 0.01 | 0.02 | 2.08 |
| 3 | Average | 0.09 | 0.20 | 0.05 | 0.01 | 0.93 | 0.17 | 5.73 | 4.42 | 0.03 | 0.05 | 0.02 | 3.25 |
| | StDev | 0.02 | 0.02 | 0.01 | 0.01 | 0.24 | 0.09 | 1.47 | 3.47 | 0.01 | 0.01 | 0.00 | 0.45 |
| 4 | Average | 0.09 | 0.26 | 0.06 | 0.00 | 1.23 | 0.28 | 6.97 | 0.26 | 0.04 | 0.07 | 0.03 | 3.76 |
| | StDev | 0.02 | 0.01 | 0.03 | 0.00 | 0.22 | 0.05 | 1.41 | 0.07 | 0.01 | 0.01 | 0.02 | 0.62 |
| 5 | Average | 0.24 | 0.45 | 0.22 | 0.00 | 2.07 | 0.32 | 8.25 | 0.30 | 0.13 | 0.13 | 0.17 | 9.90 |
| | StDev | 0.27 | 0.40 | 0.22 | 0.00 | 0.63 | 0.05 | 0.41 | 0.03 | 0.16 | 0.14 | 0.21 | 7.59 |
| 6 | Average | 0.36 | 0.47 | 0.28 | 0.05 | 1.50 | 0.19 | 5.76 | 4.31 | 0.17 | 0.19 | 0.27 | 8.47 |
| | StDev | 0.51 | 0.54 | 0.32 | 0.07 | 0.83 | 0.09 | 0.97 | 7.15 | 0.22 | 0.21 | 0.37 | 8.28 |
| 7 | Average | 0.76 | 0.51 | 0.14 | 0.16 | 1.36 | 0.19 | 4.90 | 11.40 | 0.39 | 0.40 | 0.08 | 8.93 |
| | StDev | 0.58 | 0.34 | 0.07 | 0.13 | 0.41 | 0.08 | 0.24 | 1.65 | 0.33 | 0.38 | 0.06 | 4.77 |
| 8 | Average | 1.15 | 0.78 | 0.17 | 0.09 | 1.51 | 0.26 | 5.10 | 6.44 | 0.50 | 0.39 | 0.08 | 10.27 |
| | StDev | 0.89 | 0.38 | 0.06 | 0.12 | 0.34 | 0.08 | 0.47 | 4.03 | 0.36 | 0.33 | 0.05 | 1.80 |
| 9 | Average | 0.20 | 0.38 | 0.11 | 0.13 | 1.20 | 0.19 | 5.57 | 2.49 | 0.09 | 0.16 | 0.06 | 6.69 |
| | StDev | 0.22 | 0.42 | 0.06 | 0.06 | 0.41 | 0.09 | 0.63 | 3.11 | 0.12 | 0.22 | 0.05 | 3.65 |
| 10 | Average | 0.08 | 0.11 | 0.10 | 0.07 | 0.89 | 0.13 | 5.20 | 7.11 | 0.02 | 0.03 | 0.04 | 4.12 |
| | StDev | 0.01 | 0.04 | 0.01 | 0.01 | 0.14 | 0.03 | 0.97 | 1.02 | 0.02 | 0.02 | 0.02 | 0.51 |
| 11 | Average | 0.24 | 0.22 | 0.10 | 0.04 | 1.07 | 0.17 | 5.50 | 6.25 | 0.10 | 0.08 | 0.05 | 6.42 |
| | StDev | 0.28 | 0.17 | 0.01 | 0.03 | 0.30 | 0.07 | 0.82 | 3.18 | 0.12 | 0.08 | 0.02 | 3.35 |
| 12 | Average | 0.77 | 0.65 | 0.12 | 0.10 | 1.44 | 0.22 | 5.39 | 3.77 | 0.37 | 0.34 | 0.10 | 11.82 |
| | StDev | 0.62 | 0.40 | 0.07 | 0.09 | 0.51 | 0.10 | 0.28 | 2.90 | 0.32 | 0.30 | 0.08 | 6.85 |
| 13 | Average | 0.27 | 0.09 | 0.04 | 0.04 | 0.83 | 0.09 | 6.70 | 2.99 | 0.13 | 0.02 | 0.01 | 3.81 |
| | StDev | 0.15 | 0.08 | 0.02 | 0.06 | 0.33 | 0.04 | 2.74 | 4.70 | 0.10 | 0.01 | 0.01 | 1.94 |

| | | | | | | | | | | | | |
|---|---|---|---|---|---|---|---|---|---|---|---|---|
| Profile in Nature | 0.00 | 1.39 | 0.21 | 0.69 | 0.00 | 0.00 | 1.60 | 0.00 | 0.00 | 0.00 | 0.00 | 0.00 |

TABLE 18C
Fatty acid relative percent compositions for fatty acids 22:5 N3 to 26:0 and total for SFA, MUFA,
PUFA, and Omega-3s of control and lipid loaded tilapia brain derived cells and tilapia in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 5.49 | 1.07 | 12.65 | 0.03 | 0.16 | 0.16 | 0.30 | 41.09 | 17.55 | 41.05 | 21.84 |
| | StDev | 1.64 | 0.51 | 6.25 | 0.02 | 0.10 | 0.08 | 0.19 | 16.51 | 4.15 | 12.58 | 11.30 |
| 2 | Average | 6.24 | 0.77 | 21.90 | 0.01 | 0.04 | 0.22 | 0.04 | 17.91 | 28.00 | 53.78 | 37.73 |
| | StDev | 2.49 | 0.32 | 3.02 | 0.00 | 0.02 | 0.08 | 0.03 | 3.52 | 9.68 | 11.37 | 6.16 |
| 3 | Average | 4.67 | 0.84 | 16.64 | 0.02 | 0.06 | 0.24 | 0.07 | 20.98 | 40.09 | 38.57 | 26.30 |
| | StDev | 0.41 | 0.25 | 8.48 | 0.01 | 0.01 | 0.07 | 0.02 | 6.75 | 6.42 | 10.55 | 12.72 |
| 4 | Average | 3.99 | 1.02 | 6.59 | 0.01 | 0.05 | 0.26 | 0.07 | 19.30 | 52.23 | 28.14 | 10.89 |
| | StDev | 0.79 | 0.23 | 1.57 | 0.00 | 0.01 | 0.11 | 0.01 | 0.67 | 5.76 | 5.71 | 2.44 |
| 5 | Average | 7.05 | 1.89 | 8.03 | 0.01 | 0.02 | 0.16 | 0.03 | 18.32 | 35.34 | 46.03 | 15.43 |
| | StDev | 2.34 | 0.84 | 0.24 | 0.00 | 0.02 | 0.06 | 0.02 | 5.39 | 6.63 | 12.00 | 2.54 |
| 6 | Average | 6.12 | 1.27 | 15.33 | 0.01 | 0.05 | 0.31 | 0.06 | 14.32 | 29.40 | 56.02 | 29.49 |
| | StDev | 2.22 | 1.00 | 16.85 | 0.00 | 0.02 | 0.15 | 0.03 | 2.48 | 21.32 | 21.08 | 30.91 |
| 7 | Average | 8.71 | 1.07 | 28.62 | 0.03 | 0.05 | 0.22 | 0.05 | 16.94 | 10.55 | 72.38 | 53.94 |
| | StDev | 2.45 | 0.41 | 2.99 | 0.01 | 0.01 | 0.11 | 0.03 | 1.35 | 4.57 | 5.51 | 7.39 |
| 8 | Average | 8.28 | 1.54 | 30.30 | 0.03 | 0.04 | 0.15 | 0.03 | 17.23 | 17.13 | 65.52 | 45.70 |
| | StDev | 1.92 | 0.31 | 2.96 | 0.01 | 0.02 | 0.04 | 0.02 | 3.65 | 1.75 | 2.38 | 4.84 |
| 9 | Average | 5.87 | 1.06 | 32.35 | 0.01 | 0.02 | 0.19 | 0.01 | 13.50 | 23.92 | 62.30 | 44.21 |
| | StDev | 2.96 | 0.44 | 3.78 | 0.00 | 0.00 | 0.07 | 0.00 | 4.53 | 10.48 | 7.09 | 1.11 |
| 10 | Average | 6.88 | 0.74 | 18.68 | 0.02 | 0.05 | 0.20 | 0.05 | 22.08 | 20.97 | 56.54 | 36.77 |
| | StDev | 2.00 | 0.07 | 11.64 | 0.00 | 0.02 | 0.12 | 0.02 | 2.85 | 9.41 | 6.68 | 8.97 |
| 11 | Average | 8.40 | 1.05 | 12.96 | 0.02 | 0.04 | 0.16 | 0.05 | 22.52 | 25.30 | 51.80 | 30.90 |
| | StDev | 1.96 | 0.39 | 13.60 | 0.01 | 0.00 | 0.12 | 0.01 | 2.91 | 7.59 | 6.10 | 6.70 |
| 12 | Average | 12.93 | 1.53 | 20.42 | 0.03 | 0.04 | 0.12 | 0.04 | 19.21 | 20.85 | 59.74 | 37.96 |
| | StDev | 8.39 | 0.69 | 14.41 | 0.00 | 0.01 | 0.03 | 0.02 | 2.48 | 4.89 | 6.40 | 2.44 |
| 13 | Average | 6.13 | 0.78 | 4.53 | 0.02 | 0.12 | 0.06 | 0.07 | 57.68 | 13.19 | 29.04 | 15.43 |
| | StDev | 4.73 | 0.34 | 1.90 | 0.01 | 0.07 | 0.06 | 0.06 | 28.66 | 11.21 | 17.69 | 13.57 |

| | | | | | | | | | | | |
|---|---|---|---|---|---|---|---|---|---|---|---|
| Profile in Nature | 2.98 | 0.00 | 5.97 | 0.00 | 0.00 | 0.00 | 0.00 | 40.53 | 34.56 | 24.91 | 11.24 |

TABLE 19A
Fatty acid relative percent compositions for fatty acids 12:0 to 18:4
of control and lipid loaded salmon kidney cells and salmon in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.17 | 1.17 | 0.48 | 25.32 | 0.95 | 0.46 | 34.36 | 7.65 | 0.85 | 0.09 | 0.12 | 0.02 |
| | StDev | 0.06 | 0.18 | 0.06 | 9.89 | 0.49 | 0.13 | 11.20 | 3.12 | 0.33 | 0.03 | 0.07 | 0.01 |
| 2 | Average | 0.16 | 1.16 | 0.58 | 11.04 | 5.43 | 0.43 | 18.96 | 12.23 | 4.44 | 3.29 | 0.11 | 0.09 |
| | StDev | 0.03 | 0.41 | 0.29 | 1.15 | 0.30 | 0.05 | 3.96 | 1.23 | 0.28 | 0.18 | 0.01 | 0.00 |
| 3 | Average | 0.18 | 0.94 | 0.38 | 11.51 | 4.51 | 0.39 | 22.89 | 17.43 | 0.73 | 1.32 | 0.09 | 0.06 |
| | StDev | 0.04 | 0.02 | 0.02 | 1.78 | 0.83 | 0.03 | 1.85 | 0.35 | 0.04 | 0.03 | 0.00 | 0.00 |
| 4 | Average | 0.23 | 1.02 | 0.45 | 17.15 | 2.61 | 0.44 | 21.37 | 27.47 | 0.82 | 0.09 | 0.10 | 0.02 |
| | StDev | 0.03 | 0.04 | 0.04 | 3.81 | 0.50 | 0.03 | 4.01 | 3.60 | 0.10 | 0.02 | 0.02 | 0.00 |
| 5 | Average | 0.17 | 1.19 | 0.56 | 14.21 | 2.62 | 0.50 | 18.99 | 23.32 | 4.45 | 0.10 | 0.22 | 0.02 |
| | StDev | 0.05 | 0.37 | 0.20 | 2.08 | 0.20 | 0.03 | 1.95 | 1.66 | 0.20 | 0.00 | 0.02 | 0.00 |
| 6 | Average | 0.16 | 0.99 | 0.45 | 13.99 | 2.51 | 0.42 | 16.58 | 27.46 | 7.58 | 0.13 | 0.25 | 0.02 |
| | StDev | 0.02 | 0.10 | 0.06 | 5.25 | 0.39 | 0.02 | 5.94 | 3.63 | 1.30 | 0.02 | 0.06 | 0.00 |
| 7 | Average | 0.23 | 0.83 | 0.37 | 8.79 | 0.71 | 0.38 | 14.66 | 5.93 | 1.25 | 12.05 | 0.08 | 0.11 |
| | StDev | 0.16 | 0.12 | 0.08 | 3.99 | 0.01 | 0.03 | 2.16 | 0.75 | 0.21 | 0.32 | 0.01 | 0.01 |
| 8 | Average | 0.26 | 1.87 | 0.69 | 16.63 | 1.48 | 0.46 | 15.89 | 16.12 | 1.21 | 0.16 | 0.10 | 0.05 |
| | StDev | 0.09 | 0.93 | 0.43 | 8.01 | 1.19 | 0.09 | 1.52 | 1.99 | 0.90 | 0.06 | 0.03 | 0.03 |
| 9 | Average | 0.33 | 1.52 | 0.71 | 16.47 | 3.16 | 0.55 | 22.09 | 8.89 | 2.62 | 1.55 | 0.14 | 0.04 |
| | StDev | 0.09 | 0.26 | 0.09 | 4.27 | 3.21 | 0.12 | 4.63 | 1.64 | 2.65 | 2.50 | 0.03 | 0.03 |
| 10 | Average | 0.26 | 1.06 | 0.51 | 9.84 | 6.91 | 0.38 | 15.24 | 10.74 | 6.19 | 4.87 | 0.11 | 0.09 |
| | StDev | 0.03 | 0.16 | 0.12 | 1.81 | 0.74 | 0.01 | 1.66 | 6.50 | 0.58 | 0.46 | 0.01 | 0.00 |
| 11 | Average | 0.43 | 1.39 | 0.71 | 9.49 | 6.96 | 0.42 | 14.12 | 18.59 | 3.97 | 2.92 | 0.12 | 0.06 |
| | StDev | 0.36 | 0.91 | 0.55 | 1.92 | 1.68 | 0.09 | 1.00 | 0.38 | 2.74 | 2.45 | 0.01 | 0.04 |
| 12 | Average | 0.28 | 0.93 | 0.42 | 8.31 | 6.56 | 0.39 | 12.89 | 19.22 | 0.96 | 2.03 | 0.11 | 0.05 |
| | StDev | 0.13 | 0.13 | 0.08 | 2.58 | 4.64 | 0.11 | 2.11 | 1.69 | 0.24 | 3.35 | 0.02 | 0.06 |
| 13 | Average | 0.34 | 1.85 | 0.90 | 20.02 | 1.47 | 0.59 | 22.67 | 15.09 | 1.16 | 3.73 | 0.13 | 0.09 |
| | StDev | 0.10 | 1.40 | 0.76 | 12.69 | 0.69 | 0.11 | 11.89 | 7.55 | 0.14 | 3.18 | 0.03 | 0.06 |

| | | | | | | | | | | | | |
|---|---|---|---|---|---|---|---|---|---|---|---|---|
| Profile in Nature | 0.10 | 7.11 | 0.00 | 16.52 | 6.31 | 0.00 | 3.60 | 22.02 | 2.80 | 1.60 | 0.00 | 2.5 |

TABLE 19B
Fatty acid relative percent compositions for fatty acids 20:0 to 22:4
of control and lipid loaded salmon kidney cells and salmon in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.27 | 0.04 | 0.05 | 0.01 | 1.11 | 0.12 | 9.74 | 0.40 | 0.14 | 0.01 | 0.01 | 4.30 |
| | StDev | 0.14 | 0.00 | 0.03 | 0.01 | 0.68 | 0.07 | 5.74 | 0.24 | 0.09 | 0.00 | 0.01 | 2.64 |
| 2 | Average | 0.13 | 0.05 | 0.10 | 0.08 | 1.18 | 0.14 | 9.47 | 5.49 | 0.09 | 0.03 | 0.03 | 4.13 |
| | StDev | 0.04 | 0.01 | 0.01 | 0.01 | 0.07 | 0.01 | 0.60 | 0.28 | 0.03 | 0.01 | 0.01 | 0.33 |
| 3 | Average | 0.11 | 0.08 | 0.05 | 0.04 | 1.15 | 0.11 | 9.06 | 4.82 | 0.06 | 0.02 | 0.01 | 4.04 |
| | StDev | 0.02 | 0.01 | 0.00 | 0.01 | 0.02 | 0.01 | 0.72 | 0.21 | 0.01 | 0.01 | 0.00 | 0.25 |
| 4 | Average | 0.14 | 0.14 | 0.05 | 0.01 | 1.39 | 0.11 | 9.82 | 0.50 | 0.04 | 0.05 | 0.01 | 4.28 |
| | StDev | 0.06 | 0.01 | 0.01 | 0.00 | 0.18 | 0.00 | 1.09 | 0.09 | 0.01 | 0.01 | 0.00 | 0.56 |
| 5 | Average | 0.12 | 0.12 | 0.12 | 0.01 | 1.46 | 0.17 | 11.71 | 0.49 | 0.08 | 0.04 | 0.04 | 5.02 |
| | StDev | 0.04 | 0.00 | 0.01 | 0.00 | 0.13 | 0.01 | 0.77 | 0.03 | 0.03 | 0.01 | 0.00 | 0.54 |
| 6 | Average | 0.11 | 0.14 | 0.14 | 0.01 | 1.30 | 0.14 | 10.34 | 0.45 | 0.05 | 0.04 | 0.05 | 4.22 |
| | StDev | 0.08 | 0.02 | 0.03 | 0.00 | 0.25 | 0.04 | 2.07 | 0.09 | 0.03 | 0.01 | 0.01 | 0.88 |
| 7 | Average | 0.07 | 0.05 | 0.05 | 0.13 | 0.99 | 0.10 | 7.57 | 10.89 | 0.04 | 0.02 | 0.01 | 3.53 |
| | StDev | 0.03 | 0.01 | 0.00 | 0.01 | 0.14 | 0.02 | 0.70 | 1.68 | 0.01 | 0.01 | 0.00 | 0.59 |
| 8 | Average | 0.11 | 0.25 | 0.13 | 0.01 | 1.12 | 0.11 | 8.05 | 6.69 | 0.05 | 0.05 | 0.01 | 3.46 |
| | StDev | 0.05 | 0.28 | 0.16 | 0.01 | 0.20 | 0.03 | 2.51 | 4.84 | 0.03 | 0.03 | 0.01 | 0.71 |
| 9 | Average | 0.14 | 0.05 | 0.07 | 0.03 | 1.46 | 0.16 | 12.38 | 2.52 | 0.12 | 0.01 | 0.01 | 5.31 |
| | StDev | 0.04 | 0.01 | 0.01 | 0.03 | 0.34 | 0.03 | 3.14 | 3.34 | 0.08 | 0.00 | 0.01 | 1.51 |
| 10 | Average | 0.08 | 0.06 | 0.10 | 0.07 | 1.13 | 0.12 | 9.34 | 7.44 | 0.04 | 0.03 | 0.03 | 4.00 |
| | StDev | 0.02 | 0.02 | 0.01 | 0.01 | 0.13 | 0.00 | 0.96 | 1.01 | 0.01 | 0.02 | 0.01 | 0.53 |
| 11 | Average | 0.09 | 0.10 | 0.08 | 0.04 | 1.27 | 0.14 | 10.33 | 5.66 | 0.05 | 0.04 | 0.02 | 4.38 |
| | StDev | 0.04 | 0.02 | 0.02 | 0.03 | 0.05 | 0.02 | 0.50 | 4.26 | 0.04 | 0.01 | 0.01 | 0.18 |
| 12 | Average | 0.04 | 0.08 | 0.06 | 0.05 | 1.30 | 0.14 | 11.11 | 3.32 | 0.02 | 0.03 | 0.01 | 4.82 |
| | StDev | 0.00 | 0.00 | 0.01 | 0.08 | 0.13 | 0.02 | 1.09 | 4.39 | 0.01 | 0.01 | 0.00 | 0.76 |
| 13 | Average | 0.17 | 0.06 | 0.06 | 0.10 | 1.18 | 0.13 | 8.93 | 5.32 | 0.09 | 0.03 | 0.01 | 4.08 |
| | StDev | 0.17 | 0.02 | 0.01 | 0.08 | 0.47 | 0.05 | 5.22 | 4.48 | 0.12 | 0.01 | 0.01 | 2.15 |

| | | | | | | | | | | | | |
|---|---|---|---|---|---|---|---|---|---|---|---|---|
| Profile in Nature | 0.00 | 15.72 | 0.00 | 0.00 | 0.00 | 0.00 | 0.50 | 6.41 | 0.00 | 1.60 | 0.00 | 0.00 |

TABLE 19C
Fatty acid relative percent compositions for fatty acids 22:5 N3 to 26:0 and total for SFA, MUFA,
PUFA, and Omega-3s of control and lipid loaded salmon kidney cells and salmon in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 5.13 | 0.98 | 5.76 | 0.02 | 0.13 | 0.03 | 0.11 | 62.63 | 8.69 | 28.68 | 11.38 |
| | StDev | 3.13 | 0.60 | 3.54 | 0.01 | 0.06 | 0.02 | 0.08 | 20.75 | 3.63 | 17.13 | 6.95 |
| 2 | Average | 5.26 | 0.82 | 14.76 | 0.03 | 0.11 | 0.12 | 0.09 | 32.78 | 17.86 | 49.37 | 28.79 |
| | StDev | 0.48 | 0.13 | 1.09 | 0.02 | 0.05 | 0.01 | 0.06 | 4.90 | 1.54 | 3.45 | 2.03 |
| 3 | Average | 5.02 | 0.78 | 13.88 | 0.02 | 0.07 | 0.14 | 0.11 | 36.66 | 22.19 | 41.16 | 25.03 |
| | StDev | 0.38 | 0.09 | 0.41 | 0.02 | 0.05 | 0.03 | 0.07 | 0.61 | 1.05 | 0.80 | 0.26 |
| 4 | Average | 5.26 | 0.98 | 5.18 | 0.02 | 0.05 | 0.12 | 0.06 | 40.98 | 30.38 | 28.64 | 11.04 |
| | StDev | 0.80 | 0.15 | 0.68 | 0.00 | 0.00 | 0.02 | 0.01 | 7.70 | 4.10 | 3.60 | 1.58 |
| 5 | Average | 6.25 | 1.22 | 6.48 | 0.03 | 0.08 | 0.11 | 0.11 | 36.04 | 26.20 | 37.76 | 13.31 |
| | StDev | 0.61 | 0.12 | 0.52 | 0.01 | 0.03 | 0.01 | 0.03 | 4.59 | 1.69 | 2.92 | 1.15 |
| 6 | Average | 5.41 | 1.04 | 5.68 | 0.02 | 0.05 | 0.20 | 0.06 | 32.88 | 30.35 | 36.77 | 11.68 |
| | StDev | 1.13 | 0.22 | 1.27 | 0.01 | 0.02 | 0.04 | 0.02 | 11.41 | 4.05 | 7.37 | 2.52 |
| 7 | Average | 4.55 | 0.64 | 25.66 | 0.02 | 0.04 | 0.21 | 0.06 | 25.47 | 6.92 | 67.61 | 53.14 |
| | StDev | 0.49 | 0.11 | 2.40 | 0.01 | 0.01 | 0.07 | 0.02 | 5.88 | 0.68 | 6.44 | 4.78 |
| 8 | Average | 4.37 | 0.77 | 19.66 | 0.02 | 0.06 | 0.10 | 0.07 | 36.11 | 18.00 | 45.89 | 30.88 |
| | StDev | 1.20 | 0.43 | 8.51 | 0.02 | 0.04 | 0.03 | 0.05 | 9.14 | 1.26 | 8.36 | 12.20 |
| 9 | Average | 6.55 | 1.23 | 11.54 | 0.03 | 0.11 | 0.06 | 0.13 | 42.19 | 12.17 | 45.63 | 22.17 |
| | StDev | 1.70 | 0.41 | 6.13 | 0.01 | 0.03 | 0.02 | 0.04 | 9.37 | 1.62 | 7.75 | 10.32 |
| 10 | Average | 5.16 | 0.80 | 15.06 | 0.02 | 0.06 | 0.18 | 0.05 | 27.55 | 17.92 | 54.53 | 32.54 |
| | StDev | 0.65 | 0.24 | 8.12 | 0.01 | 0.03 | 0.08 | 0.03 | 2.54 | 5.92 | 5.59 | 6.80 |
| 11 | Average | 5.50 | 0.83 | 11.90 | 0.02 | 0.07 | 0.20 | 0.08 | 26.87 | 25.88 | 47.24 | 25.98 |
| | StDev | 0.34 | 0.04 | 11.79 | 0.02 | 0.06 | 0.07 | 0.05 | 3.96 | 1.37 | 2.97 | 5.01 |
| 12 | Average | 6.05 | 0.90 | 19.72 | 0.01 | 0.03 | 0.14 | 0.04 | 23.35 | 26.02 | 50.63 | 31.11 |
| | StDev | 1.24 | 0.12 | 11.55 | 0.00 | 0.00 | 0.01 | 0.01 | 5.10 | 5.14 | 1.13 | 2.62 |
| 13 | Average | 5.43 | 0.80 | 5.14 | 0.03 | 0.12 | 0.12 | 0.14 | 46.92 | 16.77 | 36.31 | 19.63 |
| | StDev | 2.91 | 0.31 | 1.59 | 0.03 | 0.13 | 0.07 | 0.20 | 27.55 | 6.96 | 20.61 | 12.16 |

| | | | | | | | | | | | |
|---|---|---|---|---|---|---|---|---|---|---|---|
| Profile in Nature | 1.60 | 0.00 | 11.61 | 0.00 | 0.00 | 0.00 | 0.00 | 27.33 | 45.65 | 27.03 | 21.22 |

TABLE 20A
Fatty acid relative percent compositions for fatty acids 12:0 to 18:4 of
control and lipid loaded mahi mahi myoblasts and mahi mahi in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.42 | 2.83 | 1.28 | 30.63 | 2.98 | 0.65 | 32.12 | 7.90 | 1.50 | 0.12 | 0.27 | 0.05 |
| | StDev | 0.04 | 0.58 | 0.47 | 5.45 | 1.14 | 0.07 | 6.55 | 2.35 | 0.66 | 0.06 | 0.13 | 0.03 |
| 2 | Average | 0.15 | 0.57 | 0.16 | 8.61 | 5.91 | 0.23 | 10.34 | 10.89 | 6.15 | 4.23 | 0.26 | 0.27 |
| | StDev | 0.02 | 0.07 | 0.07 | 3.61 | 0.59 | 0.04 | 5.04 | 1.20 | 0.70 | 0.52 | 0.05 | 0.06 |
| 3 | Average | 0.12 | 1.21 | 0.32 | 12.22 | 11.04 | 0.31 | 9.41 | 46.23 | 1.29 | 0.15 | 0.28 | 0.07 |
| | StDev | 0.09 | 0.17 | 0.10 | 1.37 | 0.52 | 0.05 | 1.61 | 2.63 | 0.06 | 0.04 | 0.04 | 0.01 |
| 4 | Average | 0.61 | 1.13 | 0.33 | 12.32 | 4.41 | 0.30 | 8.22 | 57.18 | 1.06 | 0.11 | 0.20 | 0.04 |
| | StDev | 0.69 | 0.14 | 0.08 | 1.17 | 0.86 | 0.02 | 1.55 | 1.53 | 0.07 | 0.03 | 0.04 | 0.02 |
| 5 | Average | 0.59 | 1.36 | 0.68 | 15.62 | 5.74 | 0.37 | 11.49 | 36.31 | 9.92 | 0.17 | 1.43 | 0.05 |
| | StDev | 0.62 | 0.14 | 0.53 | 1.18 | 0.92 | 0.03 | 1.41 | 1.97 | 0.22 | 0.03 | 0.43 | 0.02 |
| 6 | Average | 0.15 | 1.01 | 0.19 | 10.95 | 4.86 | 0.28 | 7.69 | 40.19 | 18.00 | 0.23 | 1.95 | 0.04 |
| | StDev | 0.04 | 0.13 | 0.11 | 1.29 | 0.09 | 0.05 | 2.01 | 4.47 | 0.85 | 0.01 | 0.34 | 0.01 |
| 7 | Average | 0.26 | 0.60 | 0.22 | 7.11 | 0.56 | 0.21 | 9.27 | 2.44 | 1.10 | 10.95 | 0.10 | 0.37 |
| | StDev | 0.11 | 0.26 | 0.15 | 3.02 | 0.03 | 0.05 | 4.55 | 0.53 | 0.27 | 1.43 | 0.03 | 0.16 |
| 8 | Average | 0.26 | 0.74 | 0.22 | 8.85 | 0.73 | 0.27 | 7.12 | 10.87 | 0.51 | 0.09 | 0.07 | 0.03 |
| | StDev | 0.19 | 0.11 | 0.06 | 1.58 | 0.15 | 0.05 | 2.08 | 0.63 | 0.01 | 0.04 | 0.01 | 0.01 |
| 9 | Average | 0.10 | 0.48 | 0.15 | 3.68 | 10.62 | 0.14 | 4.55 | 14.85 | 0.51 | 8.68 | 0.07 | 0.67 |
| | StDev | 0.05 | 0.10 | 0.05 | 0.65 | 0.78 | 0.03 | 0.87 | 1.45 | 0.05 | 0.33 | 0.02 | 0.23 |
| 10 | Average | 0.17 | 0.99 | 0.36 | 8.07 | 9.63 | 0.23 | 8.71 | 3.38 | 7.81 | 6.29 | 0.34 | 0.48 |
| | StDev | 0.03 | 0.47 | 0.27 | 2.78 | 0.41 | 0.06 | 2.96 | 0.58 | 0.97 | 0.77 | 0.06 | 0.07 |
| 11 | Average | 0.59 | 1.00 | 0.34 | 8.57 | 13.46 | 0.29 | 11.72 | 12.38 | 8.27 | 8.16 | 0.36 | 0.55 |
| | StDev | 0.65 | 0.27 | 0.18 | 2.29 | 1.63 | 0.05 | 2.53 | 2.13 | 0.97 | 1.01 | 0.10 | 0.25 |
| 12 | Average | 0.95 | 0.69 | 0.26 | 5.67 | 12.52 | 0.21 | 8.50 | 16.05 | 0.55 | 0.08 | 0.08 | 0.01 |
| | StDev | 1.39 | 0.12 | 0.06 | 0.78 | 3.14 | 0.04 | 0.61 | 0.49 | 0.05 | 0.04 | 0.02 | 0.00 |
| 13 | Average | 0.23 | 1.00 | 0.31 | 12.68 | 1.67 | 0.37 | 12.41 | 15.19 | 14.74 | 0.18 | 0.54 | 0.03 |
| | StDev | 0.17 | 0.11 | 0.07 | 0.95 | 0.20 | 0.03 | 0.61 | 0.95 | 1.55 | 0.01 | 0.10 | 0.00 |

| | | | | | | | | | | | | |
|---|---|---|---|---|---|---|---|---|---|---|---|---|
| Profile in Nature | 0.23 | 3.25 | 0.00 | 27.84 | 5.34 | 0.00 | 10.67 | 19.95 | 0.00 | 1.16 | 0.00 | 1.16 |

TABLE 20B
Fatty acid relative percent compositions for fatty acids 20:0 to 22:4 of
control and lipid loaded mahi mahi myoblasts and mahi mahi in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.31 | 0.05 | 0.07 | 0.01 | 1.01 | 0.13 | 4.57 | 0.24 | 0.20 | 0.04 | 0.03 | 2.61 |
| | StDev | 0.08 | 0.01 | 0.03 | 0.01 | 0.48 | 0.06 | 2.17 | 0.12 | 0.04 | 0.03 | 0.01 | 1.27 |
| 2 | Average | 0.10 | 0.07 | 0.09 | 0.06 | 0.41 | 0.04 | 2.07 | 10.54 | 0.04 | 0.03 | 0.05 | 1.80 |
| | StDev | 0.04 | 0.01 | 0.01 | 0.01 | 0.03 | 0.01 | 0.18 | 1.16 | 0.01 | 0.01 | 0.01 | 0.20 |
| 3 | Average | 0.10 | 0.18 | 0.05 | 0.01 | 0.96 | 0.12 | 4.26 | 0.30 | 0.07 | 0.09 | 0.03 | 2.33 |
| | StDev | 0.02 | 0.00 | 0.01 | 0.00 | 0.03 | 0.01 | 0.16 | 0.08 | 0.02 | 0.02 | 0.00 | 0.08 |
| 4 | Average | 0.10 | 0.20 | 0.04 | 0.01 | 0.78 | 0.09 | 3.39 | 0.21 | 0.05 | 0.09 | 0.02 | 2.05 |
| | StDev | 0.01 | 0.02 | 0.01 | 0.00 | 0.11 | 0.02 | 0.23 | 0.01 | 0.01 | 0.01 | 0.01 | 0.45 |
| 5 | Average | 0.14 | 0.14 | 0.12 | 0.01 | 0.96 | 0.10 | 3.75 | 0.22 | 0.08 | 0.05 | 0.09 | 2.42 |
| | StDev | 0.01 | 0.01 | 0.01 | 0.00 | 0.08 | 0.02 | 0.16 | 0.01 | 0.02 | 0.02 | 0.01 | 0.33 |
| 6 | Average | 0.08 | 0.14 | 0.15 | 0.01 | 0.83 | 0.08 | 3.43 | 0.19 | 0.05 | 0.06 | 0.12 | 2.05 |
| | StDev | 0.02 | 0.02 | 0.01 | 0.00 | 0.07 | 0.01 | 0.63 | 0.03 | 0.01 | 0.02 | 0.03 | 0.23 |
| 7 | Average | 0.10 | 0.02 | 0.02 | 0.07 | 0.27 | 0.02 | 1.58 | 20.76 | 0.05 | 0.02 | 0.01 | 1.04 |
| | StDev | 0.05 | 0.00 | 0.00 | 0.02 | 0.02 | 0.02 | 0.13 | 1.76 | 0.03 | 0.01 | 0.00 | 0.11 |
| 8 | Average | 0.08 | 0.07 | 0.03 | 0.00 | 0.32 | 0.02 | 1.73 | 15.85 | 0.03 | 0.02 | 0.01 | 1.29 |
| | StDev | 0.03 | 0.00 | 0.00 | 0.00 | 0.01 | 0.01 | 0.11 | 0.65 | 0.01 | 0.01 | 0.00 | 0.13 |
| 9 | Average | 0.05 | 0.07 | 0.02 | 0.09 | 0.33 | 0.03 | 1.60 | 0.29 | 0.02 | 0.02 | 0.01 | 1.04 |
| | StDev | 0.01 | 0.01 | 0.00 | 0.00 | 0.02 | 0.01 | 0.17 | 0.09 | 0.01 | 0.01 | 0.00 | 0.04 |
| 10 | Average | 0.09 | 0.06 | 0.10 | 0.09 | 0.40 | 0.05 | 2.03 | 10.99 | 0.04 | 0.02 | 0.05 | 1.55 |
| | StDev | 0.04 | 0.03 | 0.02 | 0.01 | 0.04 | 0.00 | 0.11 | 1.21 | 0.03 | 0.02 | 0.01 | 0.21 |
| 11 | Average | 0.11 | 0.05 | 0.10 | 0.11 | 0.50 | 0.04 | 3.05 | 18.28 | 0.04 | 0.03 | 0.04 | 2.29 |
| | StDev | 0.04 | 0.01 | 0.02 | 0.01 | 0.05 | 0.01 | 0.20 | 1.07 | 0.03 | 0.01 | 0.02 | 0.45 |
| 12 | Average | 0.08 | 0.09 | 0.03 | 0.01 | 0.45 | 0.04 | 2.29 | 0.39 | 0.04 | 0.03 | 0.01 | 1.43 |
| | StDev | 0.04 | 0.01 | 0.01 | 0.00 | 0.04 | 0.00 | 0.22 | 0.12 | 0.03 | 0.02 | 0.01 | 0.37 |
| 13 | Average | 0.11 | 0.06 | 0.15 | 0.01 | 0.60 | 0.07 | 3.51 | 21.39 | 0.16 | 0.03 | 0.09 | 2.57 |
| | StDev | 0.03 | 0.01 | 0.01 | 0.00 | 0.04 | 0.01 | 0.44 | 0.28 | 0.20 | 0.01 | 0.03 | 0.18 |

| | | | | | | | | | | | | |
|---|---|---|---|---|---|---|---|---|---|---|---|---|
| Profile in Nature | 0.00 | 0.70 | 0.00 | 0.00 | 0.00 | 0.00 | 0.46 | 4.64 | 0.00 | 2.09 | 0.00 | 0.00 |

TABLE 20C
Fatty acid relative percent compositions for fatty acids 22:5 N3 to 26:0 and total for SFA, MUFA,
PUFA, and Omega-3s of control and lipid loaded mahi mahi myoblasts and mahi mahi in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 3.38 | 0.76 | 5.34 | 0.04 | 0.18 | 0.07 | 0.22 | 68.87 | 11.04 | 20.09 | 9.08 |
| | StDev | 1.65 | 0.38 | 2.64 | 0.04 | 0.09 | 0.03 | 0.13 | 13.22 | 3.55 | 9.70 | 4.47 |
| 2 | Average | 4.19 | 0.29 | 32.36 | 0.00 | 0.00 | 0.07 | 0.00 | 20.20 | 16.96 | 62.84 | 51.33 |
| | StDev | 0.45 | 0.07 | 3.81 | 0.00 | 0.00 | 0.01 | 0.00 | 8.85 | 1.77 | 7.11 | 5.88 |
| 3 | Average | 3.11 | 0.67 | 4.96 | 0.01 | 0.05 | 0.09 | 0.06 | 23.79 | 57.63 | 18.58 | 8.52 |
| | StDev | 0.07 | 0.01 | 0.21 | 0.00 | 0.00 | 0.01 | 0.00 | 3.31 | 3.14 | 0.41 | 0.31 |
| 4 | Average | 2.55 | 0.57 | 3.83 | 0.00 | 0.01 | 0.09 | 0.00 | 23.06 | 61.97 | 14.97 | 6.71 |
| | StDev | 0.40 | 0.11 | 0.26 | 0.00 | 0.00 | 0.01 | 0.00 | 2.51 | 1.62 | 1.56 | 0.65 |
| 5 | Average | 2.94 | 0.69 | 4.43 | 0.00 | 0.02 | 0.09 | 0.03 | 30.37 | 42.34 | 27.29 | 7.76 |
| | StDev | 0.26 | 0.10 | 0.38 | 0.00 | 0.03 | 0.02 | 0.03 | 1.91 | 2.78 | 0.99 | 0.61 |
| 6 | Average | 2.54 | 0.60 | 4.06 | 0.00 | 0.00 | 0.10 | 0.00 | 20.39 | 45.34 | 34.27 | 7.01 |
| | StDev | 0.34 | 0.13 | 0.77 | 0.00 | 0.00 | 0.02 | 0.00 | 3.39 | 4.58 | 2.34 | 1.13 |
| 7 | Average | 3.65 | 0.21 | 38.94 | 0.00 | 0.01 | 0.01 | 0.04 | 17.83 | 3.05 | 79.11 | 74.30 |
| | StDev | 0.59 | 0.03 | 4.85 | 0.00 | 0.00 | 0.00 | 0.00 | 8.22 | 0.51 | 8.71 | 8.56 |
| 8 | Average | 3.95 | 0.24 | 46.54 | 0.00 | 0.00 | 0.05 | 0.00 | 17.57 | 11.74 | 70.69 | 66.43 |
| | StDev | 0.44 | 0.02 | 2.27 | 0.00 | 0.00 | 0.00 | 0.00 | 3.80 | 0.63 | 3.27 | 3.19 |
| 9 | Average | 1.16 | 0.25 | 50.44 | 0.00 | 0.00 | 0.07 | 0.00 | 9.17 | 25.63 | 65.20 | 60.58 |
| | StDev | 0.05 | 0.03 | 1.33 | 0.00 | 0.00 | 0.02 | 0.00 | 1.70 | 1.18 | 0.96 | 1.33 |
| 10 | Average | 3.85 | 0.32 | 33.70 | 0.01 | 0.06 | 0.08 | 0.08 | 18.77 | 13.17 | 68.06 | 54.84 |
| | StDev | 0.52 | 0.03 | 3.28 | 0.01 | 0.05 | 0.01 | 0.06 | 6.74 | 0.30 | 6.95 | 5.70 |
| 11 | Average | 5.00 | 0.59 | 4.03 | 0.00 | 0.00 | 0.01 | 0.00 | 22.67 | 25.93 | 51.39 | 35.47 |
| | StDev | 0.47 | 0.04 | 0.34 | 0.00 | 0.00 | 0.00 | 0.00 | 5.48 | 2.44 | 3.07 | 1.92 |
| 12 | Average | 1.43 | 0.37 | 47.70 | 0.00 | 0.01 | 0.05 | 0.01 | 16.41 | 28.74 | 54.85 | 49.60 |
| | StDev | 0.28 | 0.04 | 1.00 | 0.00 | 0.01 | 0.00 | 0.01 | 2.67 | 2.62 | 1.27 | 1.01 |
| 13 | Average | 7.10 | 0.57 | 4.10 | 0.00 | 0.01 | 0.10 | 0.00 | 27.28 | 17.06 | 55.66 | 32.78 |
| | StDev | 0.60 | 0.08 | 0.59 | 0.00 | 0.00 | 0.03 | 0.00 | 1.15 | 0.81 | 0.41 | 0.81 |

| | | | | | | | | | | | |
|---|---|---|---|---|---|---|---|---|---|---|---|
| Profile in Nature | 2.09 | 0.00 | 20.42 | 0.00 | 0.00 | 0.00 | 0.00 | 42.00 | 28.07 | 29.93 | 28.31 |

TABLE 21A
Fatty acid relative percent compositions for fatty acids 12:0 to 18:4 of control
and lipid loaded chicken embryonic fibroblasts and chicken in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.11 | 1.53 | 0.38 | 25.74 | 3.44 | 0.54 | 26.75 | 18.26 | 1.26 | 0.03 | 0.01 | 0.00 |
| | StDev | 0.07 | 0.38 | 0.16 | 5.23 | 0.53 | 0.10 | 4.89 | 3.78 | 0.26 | 0.01 | 0.00 | 0.00 |
| 2 | Average | 0.19 | 1.20 | 0.29 | 10.06 | 11.13 | 0.42 | 13.71 | 16.34 | 8.94 | 2.97 | 0.02 | 0.03 |
| | StDev | 0.07 | 0.07 | 0.04 | 0.70 | 1.02 | 0.01 | 1.01 | 0.71 | 0.30 | 0.30 | 0.00 | 0.01 |
| 3 | Average | 0.15 | 1.12 | 0.30 | 9.59 | 9.11 | 0.38 | 10.94 | 33.41 | 0.91 | 1.02 | 0.01 | 0.02 |
| | StDev | 0.11 | 0.01 | 0.12 | 0.60 | 1.56 | 0.02 | 0.61 | 1.15 | 0.08 | 0.16 | 0.00 | 0.01 |
| 4 | Average | 0.14 | 1.15 | 0.26 | 9.72 | 5.43 | 0.45 | 10.67 | 53.14 | 0.99 | 0.02 | 0.01 | 0.00 |
| | StDev | 0.10 | 0.11 | 0.03 | 0.70 | 0.55 | 0.06 | 0.83 | 3.49 | 0.07 | 0.01 | 0.00 | 0.00 |
| 5 | Average | 0.08 | 1.23 | 0.26 | 11.37 | 5.21 | 0.43 | 13.68 | 41.13 | 7.36 | 0.06 | 0.04 | 0.00 |
| | StDev | 0.01 | 0.10 | 0.01 | 0.35 | 0.15 | 0.03 | 0.80 | 1.11 | 0.13 | 0.00 | 0.00 | 0.00 |
| 6 | Average | 0.16 | 1.06 | 0.26 | 10.59 | 3.66 | 0.50 | 12.64 | 41.32 | 10.88 | 0.04 | 0.05 | 0.00 |
| | StDev | 0.16 | 0.12 | 0.03 | 0.59 | 0.93 | 0.07 | 1.07 | 1.89 | 0.56 | 0.01 | 0.01 | 0.00 |
| 7 | Average | 0.08 | 1.01 | 0.21 | 11.31 | 0.87 | 0.42 | 10.51 | 5.36 | 0.81 | 13.98 | 0.01 | 0.12 |
| | StDev | 0.03 | 0.04 | 0.01 | 0.28 | 0.05 | 0.02 | 0.13 | 0.31 | 0.02 | 0.73 | 0.00 | 0.00 |
| 8 | Average | 0.17 | 0.98 | 0.19 | 10.50 | 1.13 | 0.39 | 9.61 | 23.05 | 0.74 | 0.05 | 0.01 | 0.02 |
| | StDev | 0.19 | 0.06 | 0.02 | 0.84 | 0.17 | 0.03 | 0.75 | 1.99 | 0.04 | 0.01 | 0.00 | 0.01 |
| 9 | Average | 0.38 | 0.99 | 0.21 | 7.58 | 15.58 | 0.36 | 11.37 | 23.36 | 0.93 | 5.74 | 0.01 | 0.06 |
| | StDev | 0.28 | 0.14 | 0.02 | 0.58 | 1.17 | 0.04 | 0.81 | 0.92 | 0.02 | 0.25 | 0.00 | 0.01 |
| 10 | Average | 0.09 | 1.09 | 0.25 | 9.46 | 13.21 | 0.40 | 14.06 | 5.61 | 12.11 | 5.17 | 0.03 | 0.07 |
| | StDev | 0.02 | 0.05 | 0.02 | 0.86 | 0.63 | 0.03 | 0.83 | 0.67 | 0.39 | 0.36 | 0.01 | 0.01 |
| 11 | Average | 0.22 | 1.29 | 0.42 | 10.27 | 12.20 | 0.41 | 13.84 | 18.90 | 11.02 | 3.77 | 0.03 | 0.05 |
| | StDev | 0.10 | 0.32 | 0.26 | 3.73 | 1.38 | 0.07 | 3.17 | 1.80 | 1.29 | 0.60 | 0.00 | 0.01 |
| 12 | Average | 0.22 | 1.00 | 0.20 | 6.26 | 14.98 | 0.27 | 9.71 | 31.96 | 0.94 | 0.07 | 0.01 | 0.01 |
| | StDev | 0.15 | 0.08 | 0.04 | 0.54 | 0.32 | 0.01 | 0.81 | 0.85 | 0.03 | 0.01 | 0.00 | 0.00 |
| 13 | Average | 0.39 | 1.09 | 0.23 | 9.43 | 1.11 | 0.43 | 10.30 | 27.46 | 1.06 | 5.26 | 0.01 | 0.05 |
| | StDev | 0.36 | 0.08 | 0.02 | 0.23 | 0.31 | 0.07 | 0.60 | 0.53 | 0.07 | 1.52 | 0.00 | 0.03 |

| | | | | | | | | | | | | |
|---|---|---|---|---|---|---|---|---|---|---|---|---|
| Profile in Nature | 0.11 | 0.96 | 0.00 | 23.15 | 6.11 | 0.00 | 6.43 | 39.98 | 20.90 | 1.07 | 0.00 | 0.00 |

TABLE 21B
Fatty acid relative percent compositions for fatty acids 20:0 to 22:4 of control
and lipid loaded chicken embryonic fibroblasts and chicken in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.18 | 0.28 | 0.12 | 0.01 | 0.97 | 0.40 | 6.96 | 0.34 | 0.12 | 0.18 | 0.02 | 4.38 |
| | StDev | 0.01 | 0.09 | 0.03 | 0.00 | 0.22 | 0.09 | 1.47 | 0.09 | 0.05 | 0.07 | 0.01 | 0.99 |
| 2 | Average | 0.06 | 0.22 | 0.38 | 0.43 | 0.71 | 0.14 | 4.96 | 6.01 | 0.02 | 0.18 | 0.11 | 3.28 |
| | StDev | 0.02 | 0.02 | 0.02 | 0.01 | 0.04 | 0.00 | 0.25 | 0.09 | 0.01 | 0.04 | 0.02 | 0.17 |
| 3 | Average | 0.07 | 0.36 | 0.08 | 0.18 | 0.65 | 0.16 | 5.18 | 6.07 | 0.04 | 0.37 | 0.02 | 3.38 |
| | StDev | 0.01 | 0.03 | 0.01 | 0.01 | 0.04 | 0.03 | 0.38 | 0.46 | 0.01 | 0.09 | 0.00 | 0.48 |
| 4 | Average | 0.08 | 0.85 | 0.09 | 0.01 | 0.76 | 0.27 | 6.23 | 0.35 | 0.04 | 0.69 | 0.02 | 3.72 |
| | StDev | 0.04 | 0.05 | 0.01 | 0.00 | 0.05 | 0.01 | 0.30 | 0.04 | 0.02 | 0.06 | 0.00 | 0.37 |
| 5 | Average | 0.07 | 0.61 | 0.42 | 0.01 | 1.06 | 0.22 | 6.58 | 0.23 | 0.03 | 0.57 | 0.14 | 4.10 |
| | StDev | 0.00 | 0.05 | 0.04 | 0.00 | 0.08 | 0.03 | 0.44 | 0.03 | 0.01 | 0.07 | 0.01 | 0.28 |
| 6 | Average | 0.12 | 0.89 | 0.83 | 0.01 | 1.12 | 0.18 | 5.37 | 0.18 | 0.07 | 0.89 | 0.32 | 4.58 |
| | StDev | 0.05 | 0.22 | 0.18 | 0.00 | 0.21 | 0.04 | 0.13 | 0.01 | 0.03 | 0.24 | 0.09 | 1.11 |
| 7 | Average | 0.07 | 0.10 | 0.07 | 0.97 | 0.42 | 0.11 | 3.63 | 13.71 | 0.02 | 0.10 | 0.02 | 2.32 |
| | StDev | 0.00 | 0.02 | 0.00 | 0.10 | 0.02 | 0.01 | 0.18 | 1.04 | 0.00 | 0.01 | 0.01 | 0.04 |
| 8 | Average | 0.05 | 0.22 | 0.07 | 0.01 | 0.48 | 0.13 | 3.69 | 11.09 | 0.02 | 0.18 | 0.01 | 2.84 |
| | StDev | 0.01 | 0.02 | 0.01 | 0.00 | 0.05 | 0.01 | 0.08 | 0.36 | 0.00 | 0.01 | 0.00 | 0.49 |
| 9 | Average | 0.07 | 0.27 | 0.07 | 0.61 | 0.58 | 0.14 | 4.96 | 1.45 | 0.02 | 0.26 | 0.02 | 2.67 |
| | StDev | 0.01 | 0.05 | 0.00 | 0.08 | 0.04 | 0.02 | 0.75 | 0.11 | 0.02 | 0.07 | 0.00 | 0.17 |
| 10 | Average | 0.09 | 0.12 | 0.48 | 0.58 | 0.67 | 0.14 | 4.55 | 7.43 | 0.04 | 0.13 | 0.16 | 2.98 |
| | StDev | 0.01 | 0.00 | 0.01 | 0.02 | 0.06 | 0.02 | 0.57 | 0.39 | 0.01 | 0.01 | 0.02 | 0.12 |
| 11 | Average | 0.09 | 0.24 | 0.44 | 0.45 | 0.67 | 0.14 | 4.96 | 6.83 | 0.04 | 0.23 | 0.14 | 3.27 |
| | StDev | 0.04 | 0.02 | 0.06 | 0.07 | 0.07 | 0.01 | 0.34 | 0.59 | 0.02 | 0.04 | 0.03 | 0.36 |
| 12 | Average | 0.06 | 0.38 | 0.07 | 0.01 | 0.66 | 0.17 | 5.32 | 0.88 | 0.03 | 0.39 | 0.01 | 2.83 |
| | StDev | 0.01 | 0.04 | 0.00 | 0.00 | 0.01 | 0.01 | 0.19 | 0.07 | 0.01 | 0.02 | 0.01 | 0.05 |
| 13 | Average | 0.07 | 0.34 | 0.09 | 0.78 | 0.64 | 0.16 | 4.61 | 10.05 | 0.03 | 0.27 | 0.03 | 3.30 |
| | StDev | 0.03 | 0.09 | 0.02 | 0.02 | 0.08 | 0.02 | 0.09 | 0.20 | 0.02 | 0.09 | 0.01 | 0.54 |

| | | | | | | | | | | | | |
|---|---|---|---|---|---|---|---|---|---|---|---|---|
| Profile in Nature | 0.00 | 1.18 | 0.00 | 0.00 | 0.00 | 0.00 | 0.11 | 0.00 | 0.00 | 0.00 | 0.00 | 0.00 |

TABLE 21C
Fatty acid relative percent compositions for fatty acids 22:5 N3 to 26:0 and total for SFA, MUFA,
PUFA, and Omega-3s of control and lipid loaded chicken embryonic fibroblasts and chicken in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 3.48 | 0.71 | 3.45 | 0.02 | 0.12 | 0.09 | 0.09 | 55.59 | 22.26 | 22.15 | 7.31 |
| | StDev | 0.73 | 0.16 | 0.65 | 0.01 | 0.06 | 0.09 | 0.04 | 9.23 | 4.54 | 4.70 | 1.47 |
| 2 | Average | 6.83 | 0.38 | 10.76 | 0.02 | 0.03 | 0.06 | 0.00 | 26.00 | 27.93 | 46.08 | 26.56 |
| | StDev | 0.26 | 0.03 | 0.59 | 0.00 | 0.01 | 0.07 | 0.00 | 1.62 | 1.27 | 0.70 | 1.08 |
| 3 | Average | 6.82 | 0.43 | 9.06 | 0.01 | 0.02 | 0.08 | 0.00 | 22.62 | 43.33 | 34.05 | 22.97 |
| | StDev | 0.70 | 0.04 | 0.39 | 0.00 | 0.01 | 0.05 | 0.00 | 1.29 | 0.63 | 1.60 | 1.01 |
| 4 | Average | 2.62 | 0.53 | 1.57 | 0.01 | 0.02 | 0.15 | 0.02 | 22.57 | 60.25 | 17.18 | 4.55 |
| | StDev | 0.25 | 0.03 | 0.11 | 0.01 | 0.01 | 0.07 | 0.01 | 1.82 | 2.88 | 1.05 | 0.34 |
| 5 | Average | 2.74 | 0.52 | 1.62 | 0.01 | 0.02 | 0.17 | 0.03 | 27.21 | 47.69 | 25.10 | 4.64 |
| | StDev | 0.20 | 0.05 | 0.14 | 0.00 | 0.00 | 0.09 | 0.01 | 0.66 | 0.94 | 1.12 | 0.36 |
| 6 | Average | 2.46 | 0.47 | 1.08 | 0.02 | 0.03 | 0.19 | 0.04 | 25.49 | 46.95 | 27.56 | 3.76 |
| | StDev | 0.41 | 0.08 | 0.02 | 0.00 | 0.01 | 0.13 | 0.04 | 1.78 | 2.13 | 1.54 | 0.42 |
| 7 | Average | 8.50 | 0.25 | 24.52 | 0.01 | 0.02 | 0.24 | 0.02 | 23.67 | 6.68 | 69.65 | 60.72 |
| | StDev | 0.11 | 0.00 | 0.66 | 0.00 | 0.01 | 0.14 | 0.02 | 0.16 | 0.25 | 0.26 | 0.48 |
| 8 | Average | 11.04 | 0.30 | 22.90 | 0.01 | 0.01 | 0.11 | 0.01 | 21.94 | 24.68 | 53.37 | 45.07 |
| | StDev | 1.08 | 0.03 | 1.00 | 0.00 | 0.00 | 0.10 | 0.01 | 1.79 | 2.21 | 0.43 | 0.35 |
| 9 | Average | 1.87 | 0.34 | 19.70 | 0.01 | 0.01 | 0.16 | 0.02 | 21.02 | 39.63 | 39.35 | 28.76 |
| | StDev | 0.15 | 0.01 | 1.90 | 0.00 | 0.00 | 0.10 | 0.00 | 1.10 | 1.00 | 1.82 | 2.14 |
| 10 | Average | 7.08 | 0.32 | 13.25 | 0.01 | 0.02 | 0.20 | 0.00 | 25.50 | 19.27 | 55.23 | 32.94 |
| | StDev | 0.53 | 0.04 | 0.77 | 0.00 | 0.01 | 0.06 | 0.00 | 1.67 | 0.36 | 1.99 | 1.28 |
| 11 | Average | 8.59 | 0.39 | 0.81 | 0.02 | 0.04 | 0.09 | 0.01 | 26.64 | 31.66 | 41.70 | 20.00 |
| | StDev | 1.22 | 0.04 | 0.07 | 0.02 | 0.04 | 0.03 | 0.01 | 7.67 | 3.22 | 4.46 | 2.29 |
| 12 | Average | 2.00 | 0.35 | 21.13 | 0.00 | 0.01 | 0.05 | 0.01 | 17.77 | 47.76 | 34.47 | 24.08 |
| | StDev | 0.05 | 0.03 | 0.48 | 0.00 | 0.01 | 0.04 | 0.01 | 1.41 | 1.23 | 0.69 | 0.58 |
| 13 | Average | 8.06 | 0.37 | 13.98 | 0.01 | 0.01 | 0.17 | 0.02 | 22.01 | 29.34 | 48.65 | 37.35 |
| | StDev | 0.42 | 0.03 | 0.71 | 0.00 | 0.00 | 0.01 | 0.00 | 0.97 | 0.67 | 1.07 | 1.83 |

| | | | | | | | | | | | |
|---|---|---|---|---|---|---|---|---|---|---|---|
| Profile in Nature | 0.00 | 0.00 | 0.00 | 0.00 | 0.00 | 0.00 | 0.00 | 30.65 | 47.27 | 22.08 | 1.07 |

TABLE 22A
Fatty acid relative percent compositions for fatty acids 12:0 to 18:4
of control and lipid loaded pig kidney cells and pig in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.17 | 1.81 | 0.54 | 26.69 | 3.78 | 0.53 | 18.83 | 16.76 | 0.73 | 0.01 | 0.05 | 0.00 |
| | StDev | 0.05 | 0.24 | 0.16 | 0.92 | 0.18 | 0.01 | 0.61 | 0.71 | 0.03 | 0.00 | 0.00 | 0.00 |
| 2 | Average | 0.06 | 0.87 | 0.16 | 10.89 | 11.38 | 0.19 | 8.50 | 12.76 | 6.63 | 3.73 | 0.22 | 0.30 |
| | StDev | 0.01 | 0.06 | 0.01 | 2.70 | 0.68 | 0.02 | 1.61 | 1.35 | 0.53 | 0.43 | 0.01 | 0.03 |
| 3 | Average | 0.12 | 0.97 | 0.19 | 11.35 | 8.64 | 0.20 | 7.55 | 24.13 | 0.46 | 1.22 | 0.04 | 0.14 |
| | StDev | 0.09 | 0.11 | 0.05 | 1.29 | 0.29 | 0.05 | 1.48 | 2.99 | 0.04 | 0.10 | 0.00 | 0.00 |
| 4 | Average | 0.09 | 0.84 | 0.12 | 10.86 | 3.98 | 0.17 | 4.15 | 67.49 | 0.34 | 0.03 | 0.04 | 0.01 |
| | StDev | 0.04 | 0.07 | 0.02 | 0.73 | 0.38 | 0.01 | 0.33 | 1.17 | 0.01 | 0.00 | 0.01 | 0.00 |
| 5 | Average | 0.32 | 1.16 | 0.15 | 15.51 | 6.08 | 0.25 | 8.65 | 41.29 | 4.60 | 0.04 | 0.36 | 0.01 |
| | StDev | 0.41 | 0.06 | 0.01 | 1.35 | 0.89 | 0.02 | 0.94 | 1.04 | 0.02 | 0.01 | 0.06 | 0.00 |
| 6 | Average | 0.15 | 1.07 | 0.16 | 13.34 | 5.64 | 0.19 | 5.88 | 46.59 | 9.18 | 0.07 | 0.70 | 0.01 |
| | StDev | 0.11 | 0.11 | 0.03 | 1.01 | 0.34 | 0.01 | 0.74 | 1.65 | 0.22 | 0.00 | 0.05 | 0.00 |
| 7 | Average | 0.10 | 0.60 | 0.09 | 8.46 | 1.70 | 0.13 | 4.93 | 4.63 | 0.34 | 9.55 | 0.03 | 0.48 |
| | StDev | 0.12 | 0.11 | 0.03 | 1.66 | 0.05 | 0.01 | 0.56 | 0.18 | 0.02 | 1.46 | 0.00 | 0.14 |
| 8 | Average | 0.07 | 0.73 | 0.11 | 10.29 | 2.07 | 0.17 | 5.14 | 14.42 | 0.32 | 0.05 | 0.03 | 0.03 |
| | StDev | 0.02 | 0.13 | 0.04 | 0.99 | 0.37 | 0.06 | 0.89 | 1.50 | 0.04 | 0.01 | 0.01 | 0.00 |
| 9 | Average | 0.05 | 0.62 | 0.07 | 5.89 | 13.76 | 0.09 | 3.90 | 20.71 | 0.36 | 6.07 | 0.04 | 0.49 |
| | StDev | 0.01 | 0.08 | 0.04 | 0.81 | 0.63 | 0.01 | 0.41 | 1.21 | 0.04 | 0.36 | 0.00 | 0.02 |
| 10 | Average | 0.10 | 0.91 | 0.21 | 8.48 | 10.32 | 0.15 | 6.61 | 5.93 | 8.83 | 4.78 | 0.22 | 0.30 |
| | StDev | 0.04 | 0.17 | 0.10 | 0.09 | 0.25 | 0.02 | 0.78 | 0.42 | 0.30 | 0.25 | 0.01 | 0.03 |
| 11 | Average | 0.09 | 1.05 | 0.14 | 9.41 | 10.53 | 0.30 | 7.47 | 18.35 | 8.71 | 4.56 | 0.29 | 0.38 |
| | StDev | 0.04 | 0.35 | 0.07 | 1.89 | 3.19 | 0.26 | 1.44 | 4.77 | 1.77 | 1.77 | 0.06 | 0.13 |
| 12 | Average | 0.04 | 0.61 | 0.07 | 5.54 | 13.17 | 0.09 | 4.14 | 29.26 | 0.32 | 0.04 | 0.03 | 0.01 |
| | StDev | 0.00 | 0.02 | 0.00 | 0.42 | 0.06 | 0.01 | 0.39 | 0.47 | 0.01 | 0.00 | 0.00 | 0.00 |
| 13 | Average | 0.07 | 0.99 | 0.13 | 13.26 | 2.38 | 0.23 | 7.48 | 29.03 | 0.55 | 6.26 | 0.06 | 0.59 |
| | StDev | 0.03 | 0.09 | 0.05 | 1.34 | 0.22 | 0.08 | 2.09 | 3.96 | 0.02 | 0.70 | 0.00 | 0.03 |

| | | | | | | | | | | | | |
|---|---|---|---|---|---|---|---|---|---|---|---|---|
| Profile in Nature | 0.12 | 1.41 | 0.00 | 24.03 | 3.03 | 0.00 | 13.10 | 46.03 | 10.16 | 0.97 | 0.00 | 0.00 |

TABLE 22B
Fatty acid relative percent compositions for fatty acids 20:0 to 22:4
of control and lipid loaded pig kidney cells and pig in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.25 | 0.36 | 0.09 | 0.01 | 0.63 | 1.05 | 9.54 | 0.24 | 0.16 | 0.14 | 0.02 | 5.79 |
| | StDev | 0.01 | 0.04 | 0.01 | 0.00 | 0.05 | 0.01 | 0.38 | 0.01 | 0.02 | 0.05 | 0.00 | 0.56 |
| 2 | Average | 0.07 | 0.13 | 0.19 | 0.42 | 0.71 | 0.17 | 3.60 | 5.83 | 0.02 | 0.05 | 0.03 | 2.19 |
| | StDev | 0.01 | 0.03 | 0.02 | 0.02 | 0.03 | 0.00 | 0.48 | 0.13 | 0.01 | 0.02 | 0.01 | 0.18 |
| 3 | Average | 0.09 | 0.24 | 0.05 | 0.20 | 0.35 | 0.20 | 3.32 | 4.73 | 0.04 | 0.10 | 0.01 | 2.31 |
| | StDev | 0.01 | 0.02 | 0.00 | 0.01 | 0.01 | 0.02 | 0.32 | 0.34 | 0.01 | 0.00 | 0.00 | 0.14 |
| 4 | Average | 0.05 | 0.57 | 0.04 | 0.00 | 0.31 | 0.95 | 3.39 | 0.13 | 0.01 | 0.19 | 0.01 | 2.04 |
| | StDev | 0.01 | 0.04 | 0.00 | 0.00 | 0.01 | 0.05 | 0.28 | 0.01 | 0.01 | 0.05 | 0.00 | 0.12 |
| 5 | Average | 0.07 | 0.49 | 0.29 | 0.01 | 1.51 | 0.67 | 7.37 | 0.14 | 0.03 | 0.19 | 0.06 | 4.37 |
| | StDev | 0.01 | 0.06 | 0.01 | 0.00 | 0.04 | 0.03 | 0.66 | 0.01 | 0.01 | 0.06 | 0.01 | 0.30 |
| 6 | Average | 0.04 | 0.32 | 0.34 | 0.01 | 1.67 | 0.39 | 6.38 | 0.12 | 0.02 | 0.11 | 0.05 | 3.00 |
| | StDev | 0.01 | 0.05 | 0.01 | 0.00 | 0.05 | 0.01 | 0.44 | 0.01 | 0.00 | 0.03 | 0.01 | 0.07 |
| 7 | Average | 0.05 | 0.06 | 0.03 | 0.47 | 0.24 | 0.11 | 2.43 | 12.17 | 0.01 | 0.02 | 0.01 | 1.58 |
| | StDev | 0.01 | 0.01 | 0.00 | 0.00 | 0.00 | 0.02 | 0.10 | 0.07 | 0.01 | 0.01 | 0.00 | 0.06 |
| 8 | Average | 0.04 | 0.09 | 0.03 | 0.01 | 0.22 | 0.15 | 3.18 | 10.52 | 0.01 | 0.03 | 0.00 | 1.58 |
| | StDev | 0.01 | 0.02 | 0.00 | 0.00 | 0.02 | 0.03 | 0.42 | 0.64 | 0.01 | 0.01 | 0.00 | 0.14 |
| 9 | Average | 0.03 | 0.13 | 0.03 | 0.45 | 0.25 | 0.14 | 2.78 | 1.92 | 0.01 | 0.04 | 0.00 | 1.24 |
| | StDev | 0.01 | 0.03 | 0.00 | 0.02 | 0.02 | 0.01 | 0.33 | 0.31 | 0.00 | 0.02 | 0.00 | 0.09 |
| 10 | Average | 0.06 | 0.08 | 0.20 | 0.44 | 0.71 | 0.14 | 3.44 | 6.58 | 0.02 | 0.03 | 0.03 | 2.02 |
| | StDev | 0.00 | 0.00 | 0.00 | 0.02 | 0.03 | 0.03 | 0.26 | 0.11 | 0.01 | 0.01 | 0.01 | 0.05 |
| 11 | Average | 0.27 | 0.40 | 0.40 | 0.63 | 1.10 | 0.23 | 4.19 | 8.09 | 0.15 | 0.29 | 0.12 | 4.02 |
| | StDev | 0.39 | 0.39 | 0.24 | 0.14 | 0.24 | 0.05 | 0.31 | 0.56 | 0.24 | 0.40 | 0.13 | 2.31 |
| 12 | Average | 0.06 | 0.26 | 0.03 | 0.01 | 0.24 | 0.21 | 2.61 | 0.30 | 0.03 | 0.13 | 0.01 | 1.38 |
| | StDev | 0.00 | 0.01 | 0.00 | 0.00 | 0.01 | 0.00 | 0.02 | 0.01 | 0.01 | 0.03 | 0.00 | 0.02 |
| 13 | Average | 0.07 | 0.21 | 0.05 | 0.72 | 0.39 | 0.22 | 4.38 | 10.34 | 0.03 | 0.09 | 0.01 | 2.86 |
| | StDev | 0.03 | 0.04 | 0.00 | 0.04 | 0.06 | 0.00 | 0.26 | 0.83 | 0.02 | 0.03 | 0.00 | 0.27 |

| | | | | | | | | | | | | |
|---|---|---|---|---|---|---|---|---|---|---|---|---|
| Profile in Nature | 0.00 | 0.87 | 0.00 | 0.00 | 0.00 | 0.00 | 0.28 | 0.00 | 0.00 | 0.00 | 0.00 | 0.00 |

TABLE 22C
Fatty acid relative percent compositions for fatty acids 22:5 N3 to 26:0 and total for SFA,
MUFA, PUFA, and Omega-3s of control and lipid loaded pig kidney cells and pig in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 4.67 | 1.27 | 4.94 | 0.04 | 0.21 | 0.26 | 0.16 | 49.40 | 21.31 | 29.05 | 9.87 |
| | StDev | 0.28 | 0.11 | 0.22 | 0.02 | 0.03 | 0.17 | 0.14 | 1.91 | 0.78 | 1.52 | 0.48 |
| 2 | Average | 8.74 | 0.32 | 21.45 | 0.01 | 0.03 | 0.13 | 0.02 | 20.83 | 24.46 | 54.57 | 39.74 |
| | StDev | 0.98 | 0.01 | 1.45 | 0.00 | 0.01 | 0.11 | 0.02 | 4.38 | 1.25 | 3.07 | 2.81 |
| 3 | Average | 9.25 | 0.36 | 23.15 | 0.01 | 0.08 | 0.23 | 0.04 | 20.61 | 33.34 | 45.83 | 38.36 |
| | StDev | 0.11 | 0.04 | 0.75 | 0.00 | 0.01 | 0.07 | 0.00 | 2.95 | 3.07 | 0.20 | 0.46 |
| 4 | Average | 1.59 | 0.40 | 1.70 | 0.00 | 0.02 | 0.28 | 0.01 | 16.32 | 72.51 | 10.98 | 3.44 |
| | StDev | 0.10 | 0.02 | 0.15 | 0.00 | 0.01 | 0.08 | 0.01 | 0.43 | 1.07 | 0.65 | 0.25 |
| 5 | Average | 2.70 | 0.66 | 2.62 | 0.00 | 0.03 | 0.11 | 0.03 | 26.19 | 48.16 | 25.42 | 5.50 |
| | StDev | 0.12 | 0.07 | 0.06 | 0.00 | 0.01 | 0.13 | 0.02 | 0.82 | 1.88 | 1.06 | 0.06 |
| 6 | Average | 1.83 | 0.47 | 1.94 | 0.01 | 0.03 | 0.15 | 0.01 | 20.89 | 52.80 | 26.15 | 3.96 |
| | StDev | 0.05 | 0.01 | 0.12 | 0.00 | 0.01 | 0.07 | 0.00 | 0.56 | 1.45 | 0.83 | 0.17 |
| 7 | Average | 10.83 | 0.23 | 40.45 | 0.00 | 0.02 | 0.08 | 0.02 | 14.40 | 6.49 | 78.99 | 73.01 |
| | StDev | 0.17 | 0.01 | 0.74 | 0.00 | 0.01 | 0.05 | 0.02 | 2.25 | 0.28 | 1.97 | 2.01 |
| 8 | Average | 11.02 | 0.24 | 39.18 | 0.00 | 0.02 | 0.03 | 0.02 | 16.60 | 16.63 | 66.56 | 60.77 |
| | StDev | 0.23 | 0.02 | 2.47 | 0.00 | 0.01 | 0.02 | 0.02 | 2.01 | 1.17 | 1.28 | 1.92 |
| 9 | Average | 2.08 | 0.23 | 38.41 | 0.00 | 0.02 | 0.07 | 0.02 | 10.68 | 34.70 | 54.52 | 48.48 |
| | StDev | 0.25 | 0.01 | 1.08 | 0.00 | 0.01 | 0.05 | 0.00 | 1.25 | 0.76 | 0.57 | 0.91 |
| 10 | Average | 10.01 | 0.27 | 28.75 | 0.01 | 0.06 | 0.09 | 0.02 | 16.63 | 16.46 | 66.76 | 50.12 |
| | StDev | 0.39 | 0.01 | 0.63 | 0.01 | 0.03 | 0.01 | 0.00 | 0.47 | 0.65 | 0.52 | 0.90 |
| 11 | Average | 15.40 | 0.64 | 2.34 | 0.00 | 0.02 | 0.13 | 0.01 | 18.91 | 29.70 | 51.21 | 30.38 |
| | StDev | 3.11 | 0.21 | 0.38 | 0.00 | 0.01 | 0.09 | 0.01 | 4.66 | 7.09 | 2.42 | 1.12 |
| 12 | Average | 1.43 | 0.28 | 39.33 | 0.00 | 0.04 | 0.24 | 0.02 | 10.63 | 43.05 | 46.22 | 41.09 |
| | StDev | 0.02 | 0.01 | 0.53 | 0.00 | 0.01 | 0.05 | 0.02 | 0.86 | 0.41 | 0.50 | 0.52 |
| 13 | Average | 16.03 | 0.54 | 2.35 | 0.01 | 0.06 | 0.22 | 0.07 | 22.41 | 31.93 | 45.44 | 34.98 |
| | StDev | 1.19 | 0.05 | 0.46 | 0.01 | 0.03 | 0.11 | 0.04 | 3.59 | 3.88 | 0.76 | 0.74 |

| | | | | | | | | | | | |
|---|---|---|---|---|---|---|---|---|---|---|---|
| Profile in Nature | 0.00 | 0.00 | 0.00 | 0.00 | 0.00 | 0.00 | 0.00 | 38.66 | 49.93 | 11.41 | 0.97 |

TABLE 23A
Fatty acid relative percent compositions for fatty acids 12:0 to 18:4
of control and lipid loaded dog kidney cells and dog in nature.

| | | | | | | | | | | | 18:3 | 18:3 | |
|---|---|---|---|---|---|---|---|---|---|---|---|---|---|
| Condition | | 12:0 | 14:0 | 15:0 | 16:0 | 16:1 | 17:0 | 18:0 | 18:1 | 18:2 | N3 | N6 | 18:4 |
| 1 | Average | 0.54 | 2.69 | 1.03 | 15.96 | 2.12 | 0.47 | 16.83 | 37.31 | 0.75 | 0.02 | 0.06 | 0.00 |
| | StDev | 0.16 | 1.64 | 1.49 | 10.64 | 0.66 | 0.28 | 10.78 | 15.61 | 0.26 | 0.01 | 0.02 | 0.00 |
| 2 | Average | 0.17 | 0.77 | 0.09 | 5.39 | 4.60 | 0.22 | 17.37 | 20.07 | 7.16 | 2.90 | 0.10 | 0.13 |
| | StDev | 0.04 | 0.11 | 0.01 | 0.16 | 0.65 | 0.01 | 1.15 | 1.47 | 0.46 | 0.43 | 0.01 | 0.02 |
| 3 | Average | 0.22 | 0.93 | 0.09 | 5.94 | 4.68 | 0.21 | 15.17 | 26.66 | 1.75 | 2.05 | 0.05 | 0.13 |
| | StDev | 0.06 | 0.10 | 0.00 | 1.14 | 0.69 | 0.02 | 2.76 | 1.28 | 0.11 | 0.33 | 0.00 | 0.03 |
| 4 | Average | 0.28 | 1.35 | 0.13 | 7.21 | 4.56 | 0.23 | 13.16 | 55.32 | 0.75 | 0.04 | 0.06 | 0.00 |
| | StDev | 0.12 | 0.38 | 0.04 | 0.66 | 0.86 | 0.00 | 0.73 | 2.92 | 0.05 | 0.00 | 0.01 | 0.00 |
| 5 | Average | 0.20 | 1.31 | 0.12 | 8.61 | 3.74 | 0.27 | 15.64 | 43.91 | 6.91 | 0.02 | 0.35 | 0.00 |
| | StDev | 0.06 | 0.17 | 0.03 | 1.32 | 0.36 | 0.04 | 2.06 | 3.18 | 0.21 | 0.01 | 0.02 | 0.00 |
| 6 | Average | 0.29 | 1.01 | 0.12 | 7.49 | 3.07 | 0.27 | 17.35 | 36.88 | 12.13 | 0.01 | 0.44 | 0.00 |
| | StDev | 0.08 | 0.10 | 0.01 | 0.73 | 0.86 | 0.02 | 3.12 | 1.49 | 0.98 | 0.00 | 0.10 | 0.00 |
| 7 | Average | 0.22 | 1.09 | 0.11 | 7.94 | 1.05 | 0.24 | 12.40 | 9.04 | 0.32 | 9.12 | 0.03 | 0.23 |
| | StDev | 0.01 | 0.08 | 0.01 | 1.61 | 0.05 | 0.02 | 1.81 | 0.21 | 0.02 | 1.03 | 0.00 | 0.03 |
| 8 | Average | 0.17 | 1.10 | 0.08 | 6.85 | 1.33 | 0.21 | 14.51 | 26.99 | 0.40 | 0.03 | 0.04 | 0.08 |
| | StDev | 0.05 | 0.09 | 0.01 | 0.57 | 0.07 | 0.01 | 0.16 | 1.93 | 0.02 | 0.00 | 0.00 | 0.02 |
| 9 | Average | 0.13 | 0.87 | 0.10 | 7.13 | 1.07 | 0.24 | 15.30 | 25.17 | 0.53 | 7.15 | 0.03 | 0.23 |
| | StDev | 0.03 | 0.16 | 0.01 | 0.79 | 0.15 | 0.02 | 2.34 | 1.65 | 0.05 | 0.75 | 0.01 | 0.04 |
| 10 | Average | 0.16 | 0.90 | 0.10 | 7.47 | 6.04 | 0.21 | 18.13 | 11.67 | 9.15 | 4.26 | 0.08 | 0.13 |
| | StDev | 0.00 | 0.07 | 0.01 | 1.41 | 0.18 | 0.01 | 2.38 | 0.65 | 0.40 | 0.18 | 0.01 | 0.02 |
| 11 | Average | 0.20 | 0.92 | 0.09 | 5.26 | 6.69 | 0.21 | 16.04 | 19.71 | 10.13 | 4.66 | 0.15 | 0.24 |
| | StDev | 0.04 | 0.07 | 0.00 | 0.20 | 0.30 | 0.01 | 0.94 | 0.41 | 0.47 | 0.16 | 0.02 | 0.04 |
| 12 | Average | 0.25 | 1.13 | 0.14 | 5.99 | 9.65 | 0.19 | 10.45 | 24.99 | 0.49 | 0.03 | 0.03 | 0.01 |
| | StDev | 0.09 | 0.27 | 0.07 | 0.78 | 2.74 | 0.01 | 1.68 | 4.24 | 0.01 | 0.00 | 0.01 | 0.01 |
| 13 | Average | 0.15 | 0.94 | 0.10 | 7.34 | 1.14 | 0.24 | 18.61 | 26.30 | 0.49 | 5.51 | 0.04 | 0.28 |
| | StDev | 0.03 | 0.15 | 0.01 | 0.32 | 0.26 | 0.01 | 1.43 | 2.63 | 0.07 | 0.46 | 0.01 | 0.05 |

| | | | | | | | | | | | | |
|---|---|---|---|---|---|---|---|---|---|---|---|---|
| Profile in Nature | 0.00 | 0.11 | 0.07 | 18.04 | 0.97 | 0.00 | 22.96 | 2.88 | 26.04 | 0.32 | 0.18 | 0.00 |

TABLE 23B
Fatty acid relative percent compositions for fatty acids 20:0 to 22:4
of control and lipid loaded dog kidney cells and dog in nature.

| | | | | | 20:3 | 20:3 | 20:3 | | | | | | |
|---|---|---|---|---|---|---|---|---|---|---|---|---|---|
| Condition | | 20:0 | 20:1 | 20:2 | N3 | N6 | N9 | 20:4 | 20:5 | 22:0 | 22:1 | 22:2 | 22:4 |
| 1 | Average | 0.48 | 1.30 | 0.03 | 0.00 | 1.21 | 3.99 | 4.48 | 1.63 | 0.40 | 0.60 | 0.26 | 1.78 |
| | StDev | 0.10 | 0.61 | 0.01 | 0.00 | 0.58 | 2.04 | 1.77 | 0.60 | 0.04 | 0.25 | 0.12 | 1.14 |
| 2 | Average | 0.17 | 0.55 | 0.13 | 0.15 | 1.10 | 0.99 | 3.58 | 17.49 | 0.11 | 0.35 | 0.02 | 0.92 |
| | StDev | 0.02 | 0.08 | 0.01 | 0.00 | 0.07 | 0.11 | 0.09 | 0.72 | 0.02 | 0.07 | 0.01 | 0.12 |
| 3 | Average | 0.16 | 0.56 | 0.03 | 0.09 | 0.72 | 1.00 | 3.56 | 19.49 | 0.12 | 0.34 | 0.01 | 0.84 |
| | StDev | 0.02 | 0.03 | 0.01 | 0.00 | 0.03 | 0.04 | 0.28 | 2.90 | 0.02 | 0.06 | 0.00 | 0.06 |
| 4 | Average | 0.22 | 1.31 | 0.02 | 0.00 | 0.99 | 2.99 | 4.12 | 1.69 | 0.14 | 0.69 | 0.17 | 1.02 |
| | StDev | 0.01 | 0.12 | 0.01 | 0.00 | 0.04 | 0.08 | 0.26 | 0.26 | 0.02 | 0.09 | 0.01 | 0.08 |
| 5 | Average | 0.23 | 1.12 | 0.14 | 0.00 | 3.52 | 2.09 | 5.22 | 1.19 | 0.13 | 0.71 | 0.03 | 1.13 |
| | StDev | 0.01 | 0.10 | 0.00 | 0.00 | 0.18 | 0.26 | 0.23 | 0.06 | 0.00 | 0.02 | 0.02 | 0.10 |
| 6 | Average | 0.24 | 1.02 | 0.26 | 0.00 | 5.29 | 1.52 | 6.08 | 0.99 | 0.18 | 0.70 | 0.12 | 1.54 |
| | StDev | 0.06 | 0.29 | 0.05 | 0.00 | 0.83 | 0.40 | 0.38 | 0.10 | 0.07 | 0.27 | 0.04 | 0.57 |
| 7 | Average | 0.12 | 0.28 | 0.02 | 0.11 | 0.34 | 0.42 | 2.35 | 22.76 | 0.06 | 0.17 | 0.00 | 0.68 |
| | StDev | 0.01 | 0.03 | 0.00 | 0.02 | 0.02 | 0.02 | 0.17 | 0.69 | 0.00 | 0.02 | 0.00 | 0.05 |
| 8 | Average | 0.15 | 0.47 | 0.01 | 0.00 | 0.57 | 0.91 | 3.86 | 30.12 | 0.10 | 0.31 | 0.00 | 1.02 |
| | StDev | 0.00 | 0.06 | 0.00 | 0.00 | 0.05 | 0.07 | 0.06 | 1.58 | 0.01 | 0.05 | 0.00 | 0.05 |
| 9 | Average | 0.12 | 0.52 | 0.01 | 0.25 | 0.66 | 0.96 | 3.15 | 10.76 | 0.07 | 0.29 | 0.01 | 0.75 |
| | StDev | 0.05 | 0.05 | 0.00 | 0.02 | 0.06 | 0.01 | 0.20 | 1.27 | 0.04 | 0.03 | 0.00 | 0.11 |
| 10 | Average | 0.17 | 0.36 | 0.14 | 0.15 | 0.82 | 0.72 | 3.05 | 17.70 | 0.10 | 0.24 | 0.02 | 0.79 |
| | StDev | 0.02 | 0.03 | 0.01 | 0.02 | 0.07 | 0.05 | 0.40 | 2.27 | 0.02 | 0.02 | 0.00 | 0.04 |
| 11 | Average | 0.15 | 0.48 | 0.17 | 0.20 | 1.47 | 0.87 | 3.80 | 20.43 | 0.10 | 0.25 | 0.05 | 0.96 |
| | StDev | 0.01 | 0.03 | 0.01 | 0.01 | 0.12 | 0.07 | 0.28 | 0.75 | 0.01 | 0.01 | 0.01 | 0.03 |
| 12 | Average | 0.13 | 0.48 | 0.02 | 0.00 | 0.66 | 1.17 | 3.23 | 9.26 | 0.08 | 0.23 | 0.00 | 0.79 |
| | StDev | 0.05 | 0.17 | 0.01 | 0.00 | 0.07 | 0.17 | 0.08 | 0.99 | 0.04 | 0.11 | 0.00 | 0.23 |
| 13 | Average | 0.18 | 0.55 | 0.01 | 0.25 | 0.63 | 0.90 | 3.51 | 24.27 | 0.11 | 0.35 | 0.00 | 0.98 |
| | StDev | 0.00 | 0.04 | 0.00 | 0.00 | 0.02 | 0.03 | 0.23 | 2.91 | 0.00 | 0.03 | 0.00 | 0.06 |

| | | | | | | | | | | | | |
|---|---|---|---|---|---|---|---|---|---|---|---|---|
| Profile in Nature | 0.13 | 0.23 | 0.19 | 0.11 | 0.94 | 0.00 | 17.42 | 2.80 | 0.15 | 0.60 | 0.00 | 0.27 |

TABLE 23C
Fatty acid relative percent compositions for fatty acids 22:5 N3 to 26:0 and total for SFA,
MUFA, PUFA, and Omega-3s of control and lipid loaded dog kidney cells and dog in nature.

| | | 22:5 | 22:5 | | | | | | Total | Total | Total | Total |
|---|---|---|---|---|---|---|---|---|---|---|---|---|
| Condition | | N3 | N6 | 22:6 | 23:0 | 24:0 | 24:1 | 26:0 | SFA | MUFA | PUFA | Omega-3 |
| 1 | Average | 0.89 | 0.46 | 3.00 | 0.10 | 0.40 | 0.24 | 0.38 | 39.28 | 41.56 | 18.57 | 5.53 |
| | StDev | 0.48 | 0.23 | 1.21 | 0.13 | 0.21 | 0.09 | 0.19 | 25.16 | 16.85 | 8.07 | 2.18 |
| 2 | Average | 2.89 | 0.17 | 11.69 | 0.01 | 0.13 | 0.20 | 0.11 | 24.54 | 25.77 | 49.44 | 34.97 |
| | StDev | 0.14 | 0.04 | 1.02 | 0.00 | 0.02 | 0.02 | 0.01 | 1.19 | 0.99 | 2.08 | 1.96 |
| 3 | Average | 2.80 | 0.20 | 11.36 | 0.02 | 0.15 | 0.27 | 0.13 | 23.13 | 32.52 | 44.06 | 35.69 |
| | StDev | 0.06 | 0.03 | 0.85 | 0.01 | 0.03 | 0.01 | 0.02 | 3.76 | 0.98 | 4.24 | 3.95 |
| 4 | Average | 0.47 | 0.31 | 1.99 | 0.02 | 0.10 | 0.24 | 0.11 | 22.94 | 62.11 | 14.57 | 4.18 |
| | StDev | 0.03 | 0.02 | 0.23 | 0.00 | 0.05 | 0.02 | 0.02 | 1.56 | 2.22 | 0.82 | 0.53 |
| 5 | Average | 0.50 | 0.29 | 1.90 | 0.02 | 0.12 | 0.12 | 0.09 | 26.74 | 49.60 | 23.29 | 3.61 |
| | StDev | 0.01 | 0.02 | 0.06 | 0.00 | 0.01 | 0.03 | 0.01 | 3.67 | 2.93 | 0.87 | 0.11 |
| 6 | Average | 0.51 | 0.30 | 1.53 | 0.02 | 0.11 | 0.15 | 0.13 | 27.21 | 41.82 | 30.72 | 3.05 |
| | StDev | 0.13 | 0.05 | 0.14 | 0.00 | 0.03 | 0.04 | 0.05 | 3.17 | 1.23 | 2.05 | 0.31 |
| 7 | Average | 4.27 | 0.16 | 25.91 | 0.01 | 0.10 | 0.07 | 0.13 | 22.42 | 10.61 | 66.73 | 62.06 |
| | StDev | 0.36 | 0.01 | 2.91 | 0.00 | 0.08 | 0.03 | 0.10 | 3.57 | 0.24 | 3.80 | 3.87 |
| 8 | Average | 7.76 | 0.23 | 1.74 | 0.03 | 0.19 | 0.20 | 0.21 | 23.60 | 29.31 | 46.77 | 39.66 |
| | StDev | 0.25 | 0.01 | 0.03 | 0.02 | 0.08 | 0.08 | 0.12 | 0.38 | 1.88 | 1.68 | 1.83 |
| 9 | Average | 0.83 | 0.19 | 22.74 | 0.02 | 0.11 | 0.19 | 0.08 | 24.17 | 27.24 | 48.28 | 41.48 |
| | StDev | 0.05 | 0.02 | 2.65 | 0.00 | 0.01 | 0.12 | 0.03 | 3.07 | 1.46 | 4.48 | 4.15 |
| 10 | Average | 2.74 | 0.18 | 13.92 | 0.01 | 0.12 | 0.10 | 0.11 | 27.47 | 18.41 | 53.88 | 38.62 |
| | StDev | 0.32 | 0.02 | 1.46 | 0.00 | 0.02 | 0.01 | 0.02 | 3.72 | 0.79 | 2.93 | 2.80 |
| 11 | Average | 4.17 | 0.22 | 1.46 | 0.03 | 0.17 | 0.21 | 0.22 | 23.38 | 27.34 | 48.99 | 30.72 |
| | StDev | 0.30 | 0.02 | 0.10 | 0.02 | 0.07 | 0.11 | 0.07 | 0.83 | 0.43 | 1.21 | 0.60 |
| 12 | Average | 0.96 | 0.53 | 28.28 | 0.03 | 0.17 | 0.18 | 0.18 | 18.73 | 35.52 | 45.45 | 38.53 |
| | StDev | 0.19 | 0.60 | 2.30 | 0.02 | 0.14 | 0.18 | 0.12 | 0.98 | 2.46 | 3.22 | 2.64 |
| 13 | Average | 4.80 | 0.17 | 1.38 | 0.02 | 0.14 | 0.15 | 0.12 | 27.94 | 28.51 | 43.24 | 35.96 |
| | StDev | 0.26 | 0.03 | 0.13 | 0.00 | 0.01 | 0.04 | 0.02 | 1.19 | 2.37 | 3.52 | 3.23 |

| | | | | | | | | | | | |
|---|---|---|---|---|---|---|---|---|---|---|---|
| Profile in Nature | 0.70 | 0.11 | 3.34 | 0.10 | 0.13 | 0.89 | 0.00 | 41.70 | 5.87 | 52.44 | 7.17 |

TABLE 24A
Percentage point change in total saturated fatty acid content from test conditions compared to unloaded
control condition 1 for aquatic species. Negative number indicates a decrease compared to control.

| | | | | | Red | | | | | |
|---|---|---|---|---|---|---|---|---|---|---|
| | | | Bluefin | | snapper | | | Tilapia | | |
| | | Bluefin | tuna | Carp | muscle | Yellowfin | | brain | Salmon | Mahi |
| | | tuna | pre- | pre- | derived | tuna | Bluegill | derived | kidney | mahi |
| Condition | | myoblasts | adipocytes | adipocytes | cells | fibroblast | fibroblasts | cells | cells | myoblasts |
| 2 | Average | −43.42 | −30.23 | −10.65 | −20.54 | −40.64 | −23.87 | −23.18 | −29.86 | −48.67 |
| | StDev | 2.21 | 1.71 | 4.03 | 4.66 | 2.15 | 5.38 | 3.52 | 4.90 | 8.85 |
| 3 | Average | −31.02 | −22.55 | −8.71 | −20.63 | −39.56 | −23.15 | −20.11 | −25.98 | −45.08 |
| | StDev | 3.58 | 0.05 | 4.81 | 4.68 | 1.57 | 6.28 | 6.75 | 0.61 | 3.31 |
| 4 | Average | −38.30 | −26.30 | 5.86 | −25.39 | −38.71 | −16.38 | −21.79 | −21.66 | −45.81 |
| | StDev | 4.19 | 0.19 | 10.02 | 2.79 | 0.65 | 5.13 | 0.67 | 7.70 | 2.51 |
| 5 | Average | −23.02 | −22.34 | −5.58 | −24.54 | −34.25 | −16.86 | −22.78 | −26.60 | −38.50 |
| | StDev | 11.51 | 0.76 | 3.57 | 1.35 | 1.69 | 1.04 | 5.39 | 4.59 | 1.91 |
| 6 | Average | −40.59 | −22.32 | −6.95 | −28.64 | −37.11 | −22.07 | −26.78 | −29.76 | −48.48 |
| | StDev | 1.90 | 6.07 | 2.77 | 2.27 | 3.98 | 2.90 | 2.48 | 11.41 | 3.39 |
| 7 | Average | −50.53 | −33.46 | −13.61 | −32.61 | −42.96 | −32.63 | −24.16 | −37.16 | −51.04 |
| | StDev | 1.63 | 0.45 | 1.26 | 1.74 | 0.84 | 2.95 | 1.35 | 5.88 | 8.22 |
| 8 | Average | −47.84 | −33.07 | −15.55 | −28.38 | −44.25 | −27.81 | −23.86 | −26.53 | −51.30 |
| | StDev | 1.22 | 1.36 | 1.68 | 0.69 | 0.98 | 0.26 | 3.65 | 9.14 | 3.80 |
| 9 | Average | −51.74 | −34.71 | −15.63 | −24.48 | −45.87 | −28.71 | −27.59 | −20.44 | −59.70 |
| | StDev | 0.57 | 0.88 | 3.30 | 3.97 | 0.97 | 2.12 | 4.53 | 9.37 | 1.70 |
| 10 | Average | −43.15 | −31.47 | −4.24 | −19.47 | −44.34 | −34.95 | −19.01 | −35.08 | −50.10 |
| | StDev | 2.17 | 1.50 | 9.73 | 0.91 | 0.48 | 2.27 | 2.85 | 2.54 | 6.74 |
| 11 | Average | −44.66 | −31.35 | −4.79 | −8.87 | −43.44 | −23.51 | −18.58 | −35.76 | −46.20 |
| | StDev | 2.61 | 1.07 | 7.07 | 5.94 | 0.82 | 4.30 | 2.91 | 3.96 | 5.48 |
| 12 | Average | −42.81 | −32.70 | −14.38 | −14.92 | −42.23 | −29.33 | −21.88 | −39.28 | −52.46 |
| | StDev | 4.56 | 2.05 | 2.70 | 7.43 | 0.05 | 8.87 | 2.48 | 5.10 | 2.67 |
| 13 | Average | −37.55 | −25.97 | −5.92 | 0.00 | −31.85 | −21.23 | 2.23 | −17.79 | −41.59 |
| | StDev | 1.12 | 0.59 | 4.82 | 17.75 | 2.73 | 0.33 | 17.40 | 23.95 | 1.15 |

TABLE 24B
Percentage point change in total saturated fatty acid
content from test conditions compared to unloaded control
condition 1 for terrestrial species. Negative number
indicates a decrease compared to control.

| | | Chicken embryonic | Pig kidney | Dog kidney |
|---|---|---|---|---|
| Condition | | fibroblasts | cells | cells |
| 2 | Average | −29.60 | −28.57 | −14.74 |
| | StDev | 1.62 | 4.38 | 1.19 |
| 3 | Average | −32.97 | −28.79 | −16.15 |
| | StDev | 1.29 | 2.95 | 3.76 |
| 4 | Average | −33.02 | −33.08 | −16.33 |
| | StDev | 1.82 | 0.43 | 1.56 |
| 5 | Average | −28.38 | −23.21 | −12.54 |
| | StDev | 0.66 | 0.82 | 3.67 |
| 6 | Average | −30.10 | −28.51 | −12.07 |
| | StDev | 1.78 | 0.56 | 3.17 |
| 7 | Average | −31.92 | −35.00 | −16.85 |
| | StDev | 0.16 | 2.25 | 3.57 |
| 8 | Average | −33.65 | −32.80 | −15.67 |
| | StDev | 1.79 | 2.01 | 0.38 |
| 9 | Average | −34.57 | −38.73 | −15.11 |
| | StDev | 1.10 | 1.25 | 3.07 |
| 10 | Average | −30.09 | −32.78 | −11.81 |
| | StDev | 1.67 | 0.47 | 3.72 |
| 11 | Average | −28.95 | −30.49 | −15.89 |
| | StDev | 7.67 | 4.66 | 0.83 |
| 12 | Average | −37.82 | −38.78 | −20.55 |
| | StDev | 1.41 | 0.86 | 0.98 |
| 13 | Average | −33.58 | −27.00 | −11.34 |
| | StDev | 0.97 | 3.59 | 1.19 |

TABLE 25A
Percentage point change in total Omega-3 fatty acid content from test conditions compared
to unloaded control condition 1. Negative number indicates a decrease compared to control.

| | | | | | Red | | | | | |
|---|---|---|---|---|---|---|---|---|---|---|
| | | | Bluefin | | snapper | | | Tilapia | | |
| | | Bluefin | tuna | Carp | muscle | Yellowfin | | brain | Salmon | Mahi |
| | | tuna | pre- | pre- | derived | tuna | Bluegill | derived | kidney | mahi |
| Condition | | myoblasts | adipocytes | adipocytes | cells | fibroblast | fibroblasts | cells | cells | myoblasts |
| 2 | Average | 40.34 | 37.94 | 28.83 | 31.49 | 42.57 | 25.67 | 15.89 | 17.41 | 42.24 |
| | StDev | 1.66 | 1.40 | 5.80 | 3.12 | 2.04 | 2.82 | 6.16 | 2.03 | 5.88 |
| 3 | Average | −2.94 | −8.70 | 24.51 | 25.25 | 35.07 | 21.59 | 4.46 | 13.66 | −0.57 |
| | StDev | 0.97 | 0.56 | 5.61 | 5.26 | 1.59 | 4.77 | 12.72 | 0.26 | 0.31 |
| 4 | Average | −5.63 | −10.88 | −6.47 | −7.10 | −7.97 | 6.31 | −10.95 | −0.33 | −2.37 |
| | StDev | 0.73 | 0.25 | 1.01 | 0.77 | 0.10 | 1.43 | 2.44 | 1.58 | 0.65 |
| 5 | Average | −3.44 | −9.73 | −4.24 | −5.83 | −6.71 | −2.53 | −6.41 | 1.94 | −1.32 |
| | StDev | 0.79 | 0.19 | 1.49 | 0.27 | 0.50 | 1.42 | 2.54 | 1.15 | 0.61 |
| 6 | Average | −4.92 | −10.81 | −6.27 | −6.55 | −7.65 | −4.53 | 7.65 | 0.30 | −2.07 |
| | StDev | 0.28 | 0.08 | 0.59 | 0.12 | 0.43 | 0.92 | 30.91 | 2.52 | 1.13 |
| 7 | Average | 72.70 | 67.74 | 52.35 | 61.97 | 69.93 | 54.39 | 32.10 | 41.77 | 65.22 |
| | StDev | 0.82 | 0.33 | 1.16 | 1.83 | 1.51 | 3.64 | 7.39 | 4.78 | 8.56 |
| 8 | Average | 59.72 | 60.03 | 47.12 | 46.56 | 60.59 | 36.79 | 23.86 | 19.50 | 57.35 |
| | StDev | 1.42 | 0.59 | 1.33 | 0.27 | 0.98 | 0.53 | 4.84 | 12.20 | 3.19 |
| 9 | Average | 51.30 | 41.86 | 41.01 | 40.06 | 50.93 | 33.39 | 22.37 | 10.79 | 51.49 |
| | StDev | 0.61 | 0.50 | 2.27 | 2.52 | 0.96 | 2.21 | 1.11 | 10.32 | 1.33 |
| 10 | Average | 46.52 | 43.89 | 29.64 | 18.13 | 50.89 | 41.42 | 14.93 | 21.16 | 45.75 |
| | StDev | 0.86 | 1.91 | 6.75 | 1.07 | 1.08 | 2.19 | 8.97 | 6.80 | 5.70 |
| 11 | Average | 24.33 | 19.86 | 15.91 | 24.41 | 30.96 | 13.49 | 9.05 | 14.61 | 26.39 |
| | StDev | 0.71 | 1.31 | 8.22 | 3.60 | 0.38 | 1.80 | 6.70 | 5.01 | 1.92 |
| 12 | Average | 33.93 | 35.63 | 34.30 | 22.27 | 39.26 | 30.49 | 16.11 | 19.73 | 40.51 |
| | StDev | 3.41 | 1.82 | 1.50 | 2.88 | 1.11 | 7.95 | 2.44 | 2.62 | 1.01 |
| 13 | Average | 34.01 | 18.65 | 25.28 | 0.00 | 32.65 | 19.66 | 0.56 | 9.01 | 23.69 |
| | StDev | 1.30 | 0.43 | 2.85 | 5.18 | 1.79 | 0.35 | 7.83 | 10.84 | 0.81 |

TABLE 25B
Percentage point change in total Omega-3 fatty acid
content from test conditions compared to unloaded
control condition 1 for terrestrial species. Negative
number indicates a decrease compared to control.

| | | Chicken embryonic | Pig kidney | Dog kidney |
|---|---|---|---|---|
| Condition | | fibroblasts | cells | cells |
| 2 | Average | 19.25 | 29.87 | 29.44 |
| | StDev | 1.08 | 2.81 | 1.96 |
| 3 | Average | 15.66 | 28.49 | 30.16 |
| | StDev | 1.01 | 0.46 | 3.95 |
| 4 | Average | −2.76 | −6.43 | −1.35 |
| | StDev | 0.34 | 0.25 | 0.53 |
| 5 | Average | −2.66 | −4.37 | −1.93 |
| | StDev | 0.36 | 0.06 | 0.11 |
| 6 | Average | −3.55 | −5.91 | −2.49 |
| | StDev | 0.42 | 0.17 | 0.31 |
| 7 | Average | 53.41 | 63.14 | 56.53 |
| | StDev | 0.48 | 2.01 | 3.87 |
| 8 | Average | 37.76 | 50.90 | 34.13 |
| | StDev | 0.35 | 1.92 | 1.83 |
| 9 | Average | 21.45 | 38.61 | 35.95 |
| | StDev | 2.14 | 0.91 | 4.15 |
| 10 | Average | 25.63 | 40.25 | 33.09 |
| | StDev | 1.28 | 0.90 | 2.80 |
| 11 | Average | 12.69 | 20.51 | 25.18 |
| | StDev | 2.29 | 1.12 | 0.60 |
| 12 | Average | 16.77 | 31.22 | 33.00 |
| | StDev | 0.58 | 0.52 | 2.64 |
| 13 | Average | 30.04 | 25.11 | 30.43 |
| | StDev | 1.83 | 0.74 | 3.23 |

### Example 12: Effect of Nervonic Acid FA Toxicity

Concentration effects of fatty acids previously shown to be toxic to aquatic fat-derived and muscle-derived cells (Example 2, Example 5, and Example 6, respectively) were evaluated, alone and in combination with nervonic acid. EPA, DHA, stearic acid, palmitic acid and nervonic acid were tested at concentrations of 0, 10, 25, and 50 ug/mL; each concentration of EPA, DHA, stearic acid and palmitic acid was also tested in combination with 10, 25 and 50 μg/ml of nervonic acid.

Cell lines were seeded per standard passaging protocols in 4×96 well plates. The cells were allowed to proliferate to approximately 80% confluence, and then transitioned to lipid loading medium containing 0/4% FBS and appropriate lipid compositions, with 6 wells/condition. Cells were monitored for 24-48 hours to evaluate any protective effect with the addition of nervonic acid.

Cells from terrestrial cell lines were imaged in the IncuCyte for a 1-time scan to collect the images and quantify confluence using the IncuCyte software. Following imaging, cell viability was determined by a commercially available ATP assay (CellTiter-Glo 2.0, Promega) where luminescent signal is proportionate to cell number.

Cells from aquatic species were placed in the IncuCyte and scanned every 6 hours up to 48 hours. Once imaging was completed, cellviability was determined by a commercially available ATP assay (CellTiter-Glo 2.0, Promega) where luminescent signal is proportionate to cell number.

### Results

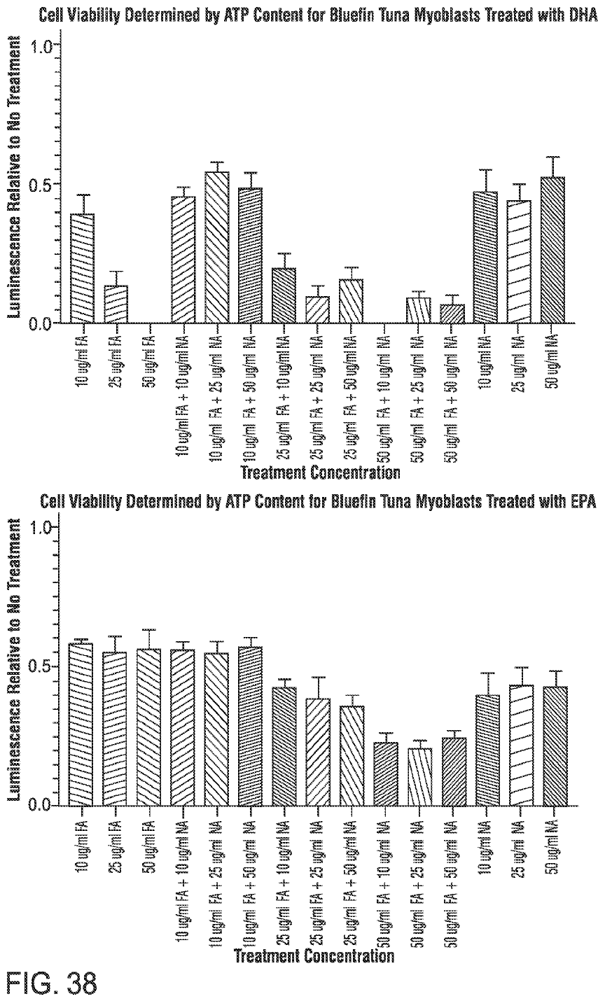

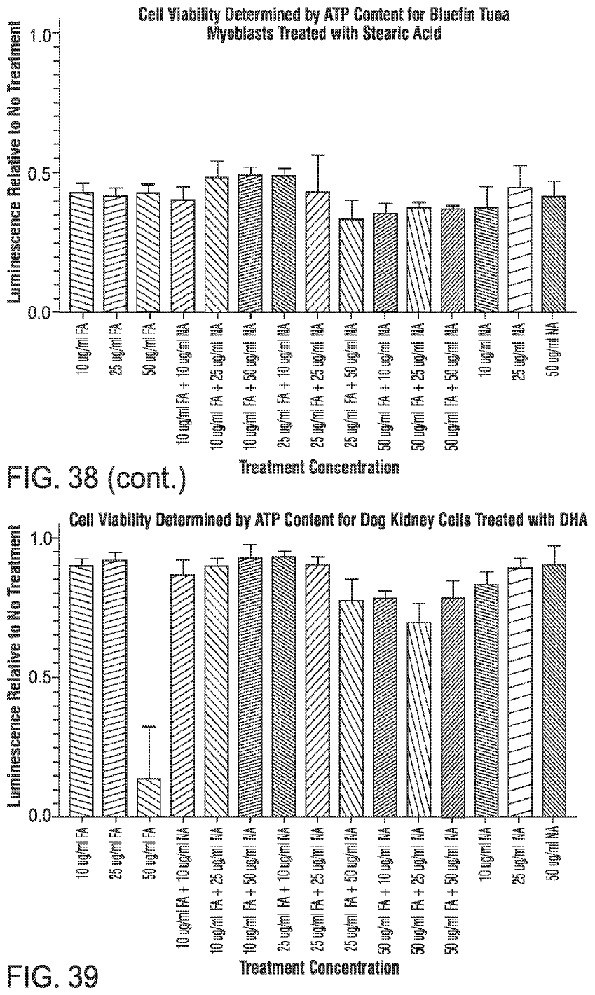

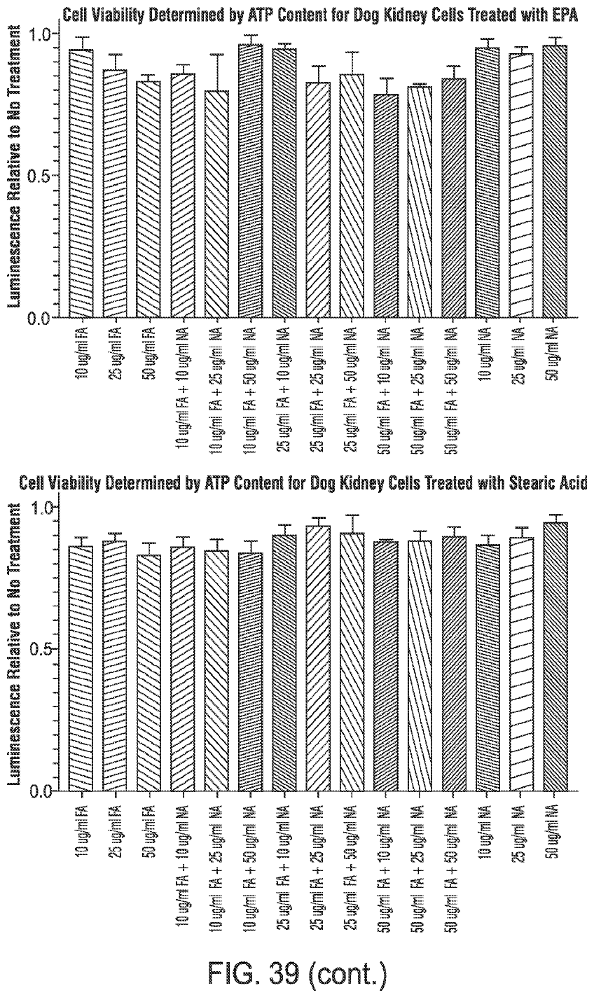

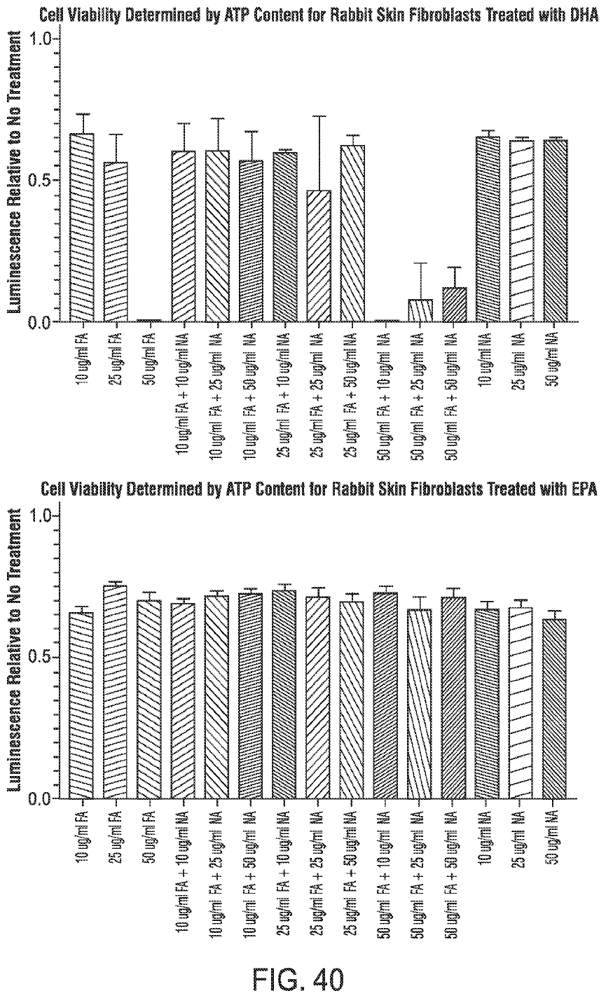

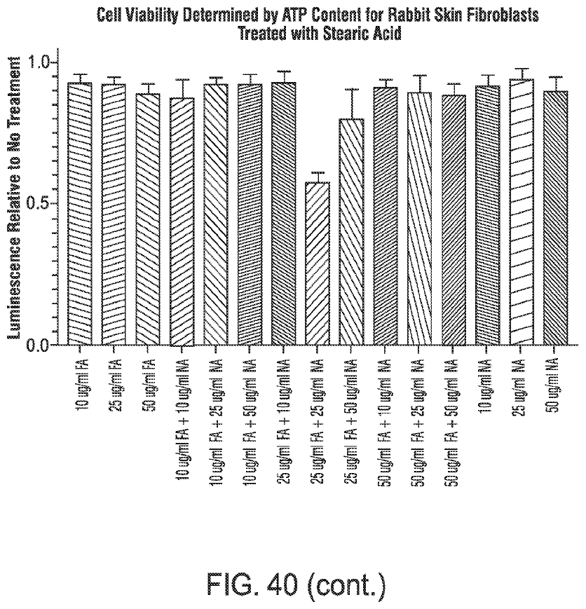

Two distinct phenomena were observed by the addition of nervonic acid to loading of another fatty acid. First, in the presence of toxic concentrations of DHA, nervonic acid provided beneficial effect on viability, resulting in an increase in cell ATP which is an indicator of cell number. FIGS. 38-40 show for a range of species that in combinations with DHA, versus other fatty acids, nervonic acid can rescue cells from toxicity. The degree of improvement was 70×, 5.5×, and 42× increase in cells for bluefin tuna myoblasts, dog kidney cells, and rabbit skin fibroblasts, respectively. Of note, this was also observed in Example 6 where all combinations that showed significant increase in cell number had both DHA and nervonic acid. The examples set forth above are provided to give those of ordinary skill in the art a complete disclosure and description of how to make and use the embodiments of loading various types of fatty acids alone or in combination with others to obtain fatty acid enriched meat cells with high cell numbers as well as increased lipid uptake and lipid accumulation, and related compositions, methods, and systems of the disclosure, and are not intended to limit the scope of what the inventors regard as their disclosure. Those skilled in the art will recognize how to adapt the features of the exemplified cells, compositions, methods and systems herein disclosed to additional cells, compositions, methods and systems according to various embodiments and scope of the claims.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, system elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the genetic circuits, genetic molecular components, and methods steps set forth in the present description. As will be obvious to one of skill in the art, methods and systems useful for the present methods and systems may include a large number of optional composition and processing elements and steps.

In particular, it will be understood that various modifications may be made without departing from the spirit and scope of the present disclosure. Accordingly, other embodiments are within the scope of the following claims.

### REFERENCES

Anne Vegusdal, Hilde Sundvold, Tor Gjøen, and Bente Ruyter. An in vitro Method for Studying the Proliferation and Differentiation of Atlantic Salmon Preadipocytes. 2003. Lipids, Vol. 38, no 3, 289-296.

1. A cultured cell of an animal, wherein the cultured cell has a) less saturated fat (SFA) content (g saturated fat/g total fat), and/or b) greater unsaturated fat (UFA) content (g polyunsaturated fatty acids (PUFA) and/or monounsaturated fat (MUFA)/g total fat), each of a) and b) in comparison to a wild caught or farm-raised animal of the same species as the cultured cells.

2. The cultured cell of an animal of claim 1, wherein the cell comprises one or more of myoblasts, myocytes, preadipocytes, adipocytes, fibroblasts, keratinocytes, epithelial cells, endothelial cells, embryonic derived cells, induced pluripotent stem cells, mesenchymal stem cells, and combinations thereof of the animal.

3. The cultured cell of an animal of claim 1, wherein the cell comprises an adipocyte comprising one or more polyunsaturated fatty acids, one or more saturated fatty acids, and/or one or more sterols in an amount from 0.01% to 1% by weight of the cell.

4. The cultured cell of an animal of claim 1, wherein the cell comprises a fibroblast comprising one or more polyunsaturated fatty acids, one or more saturated fatty acids, one or more monounsaturated fatty acids, and/or one or more sterols in an amount of at least 1% by weight of the cell.

5. The cultured cell of an animal of claim 1, wherein the cell comprises a myoblast comprising one or more polyunsaturated fatty acids, one or more saturated fatty acids, one or more monounsaturated fatty acids, and/or one or more sterols in an amount of at least 1% by weight of the cell.

6. The cultured cell of an animal of claim 1, wherein the animal is selected from the group consisting of an aquatic animal and a terrestrial animal, wherein the aquatic animal is a fish and/or a shellfish and wherein the terrestrial animal is a mammal, a bird, a reptile, an amphibian, or an insect.

7. (canceled)

8. The cultured cell of an aquatic animal of claim 6, wherein the aquatic animal comprises one or more of a cartilaginous fish, bony fish, ray-finned fish, lobe-finned fish, mollusks, mussels, cephalopods, crustaceans, and echinoderms.

9. (canceled)

10. The cultured cell of an aquatic animal of claim 6, wherein the aquatic animal comprises one or more of Thunnus orientalis, Lutjanus campechanus, Coryphaena hippurus, Seriola lalandi, Micropterus salmoides, Thunnus albacares, Lepomis macrochirus, Hypophthalmichthys molitrix, Salmo salar, Oncorbynchus mykiss, and Oreochromis massambicus.

11-19. (canceled)

20. The cultured cell of a terrestrial animal of claim 6, wherein the cell has an omega-3 PUFA content at least 1 percentage point higher than a cell of the same type from a wild-caught or farm-raised terrestrial animal of the same species and/or a SFA content at least 1 percentage point lower and/or a MUFA content at least 1 percentage point higher than a cell of the same type from wild-caught or farm-raised terrestrial animal of the same species.

21. (canceled)

22. A biomass comprising one or more cultured cells of claim 1.

23. A food product comprising one or more cultured cells of claim 1 and/or the biomass of claim 22, and optionally a suitable matrix.

24-25. (canceled)

26. The food product of claim 23, wherein the food product comprises cells of a terrestrial animal and has an omega-3 PUFA content at least 1 percentage point higher than that contained in a food product derived from cells of a wild-caught or farm-raised terrestrial animal of the same species.

27. The food product of claim 26, wherein the food product comprises cells of a terrestrial animal and has a SFA content at least 1 percentage point lower than that contained in a food product derived from the cells of a wild-caught or farm-raised terrestrial animal of the same species.

28-29. (canceled)

30. A culture medium comprising a cell basal medium for a cell of an animal supplemented with at least 0.1 μg/ml of one or more monounsaturated fatty acids, wherein the monounsaturated fatty acids comprise palmitoleic acid in a concentration from 1 to 100 μg/ml or 50 to 100 μg/ml, vaccenic acid in a concentration of 1 to 100 μg/ml or 50 to 75 μg/ml, oleic acid in a concentration from 1 to 100 μg/ml or 75 to 100 μg/ml, and/or MUFA, such as nervonic acid, in a concentration from 1 to 100 μg/ml or 50 to 75 μg/ml, and optionally up to 4% serum.

31-42. (canceled)

43. A method for culturing a cell, the method comprising culturing the cell of an animal in a culture medium comprising at least 0.1 μg/ml of one or more monounsaturated fatty acids for a time and under condition allowing uptake of the monounsaturated fatty acid by the cell of the animal.

44-54. (canceled)

55. A cell of an animal, the cell obtainable and/or obtained by the method of claim 43.

56-75. (canceled)

76. A food product comprising one or more of the cell of claim 55 and/or a biomass obtained by the method of claim 43.

77-83. (canceled)

84. A system for culturing a cell of an animal, the system comprising one or more monounsaturated fatty acids in combination with a culture medium for a cell of an animal and/or a cell of an animal, for simultaneous, combined or sequential use in the method to increase content of one or more monounsaturated fatty acids in a cell of claim 43.

85-111. (canceled)

112. A culture medium, for a cell of an animal, the culture medium comprising a cell basal medium for a cell of an animal supplemented with at least 0.1 μg/ml or at least 10 μg/ml of one or more polyunsaturated fatty acids, one or more saturated fatty acids, and/or one or more sterols and optionally nervonic acid in a concentration of at least 1 μg/mL or at least 10 μg/mL.

113-141. (canceled)

142. A method for culturing a cell of an animal, the method comprising culturing the cell of an animal in a culture medium comprising at least 0.1 μg/ml or 10 μg/ml of one or more polyunsaturated fatty acids, one or more saturated fatty acids, and/or one or more sterols, and further comprising nervonic acid in a concentration of at least 1 μg/ml or 10 μg/ml;
the culturing performed for a time and under conditions allowing uptake of the monounsaturated fatty acid by the cell of the animal.

143-170. (canceled)

171. A cell of an animal, the cell obtained by the method of claim 142.

172-192. (canceled)

193. A food product comprising one or more cells of an animal of claim 171 and/or a biomass obtained by the method of claim 142.

194-253. (canceled)

254. A culture medium, for a cell of an animal, the culture medium comprising a cell basal medium for a cell of an animal supplemented with at least 0.1 μg/ml or 10 μg/ml of a lipid, wherein when the lipid comprises one or more polyunsaturated fatty acids, one or more saturated fatty acids, and/or one or more sterols the culture medium further comprises a nervonic acid in a concentration of at least 0.1 μg/ml or 10 μg/ml.

255-280. (canceled)

281. A method for culturing a cell of an animal, the method comprising
culturing the cell of an animal in a culture medium comprising at least 0.1 μg/ml or 10 μg/ml of a lipid, the culturing performed for a time and under condition allowing uptake of a fatty acid of the lipid by the cell of the animal, wherein when the lipid comprises one or more polyunsaturated fatty acids, one or more saturated fatty acids, and/or one or more sterols, wherein the culture medium further comprises a nervonic acid in a concentration of at least 0.1 μg/ml or 10 μg/ml.

282-305. (canceled)

306. A cell of an animal, the cell obtainable and/or obtained by the method of claim 281.

307-327. (canceled)

328. A food product comprising one or more cells of an animal of claim 306.

329-385. (canceled)
